(19) United States
(12) Patent Application Publication
(10) Pub. No.: US 2023/0391779 A1
(43) Pub. Date: Dec. 7, 2023

(54) BICYCLIC COMPOUNDS, COMPOSITIONS AND USE THEREOF

(71) Applicant: Betta Pharmaceuticals Co., Ltd., Zhejiang (CN)

(72) Inventors: Xiaofeng XU, Beijing (CN); Jinheng GAO, Beijing (CN); Hongfei RONG, Beijing (CN); Xizhen SONG, Beijing (CN); Jie CHEN, Beijing (CN); Xiangyong LIU, Beijing (CN); Hongling SHEN, Beijing (CN); Jing GUO, Beijing (CN); Dan YAN, Beijing (CN); Hong LAN, Beijing (CN); Lieming DING, Zhejiang (CN); Jiabing WANG, Beijing (CN)

(73) Assignee: Betta Pharmaceuticals Co., Ltd., Zhejiang (CN)

(21) Appl. No.: 18/021,784

(22) PCT Filed: Aug. 17, 2021
(86) PCT No.: PCT/CN2021/112983

§ 371 Date: Feb. 16, 2023

(30) Foreign Application Priority Data
Aug. 17, 2020 (WO) PCTCN2020109491
Feb. 4, 2021 (WO) PCTCN2021075195

(51) Int. Cl. C07D 487/04; C07D 403/04; C07D 471/04; C07D 403/06; C07D 231/56; C07D 401/14; C07D 401/04; C07D 405/04; C07D 413/04; C07D 413/14; C07D 405/14; C07D 493/04; C07C 237/20; C07D 205/04; A61P 35/00

(52) U.S. Cl. CPC C07D 487/04; A61P 35/00; C07D 471/04; C07D 403/06; C07D 231/56; C07D 401/14; C07D 401/04; C07D 405/04; C07D 413/04; C07D 413/14; C07D 405/14; C07D 493/04; C07C 237/20; C07D 205/04; C07D 403/04

(57) ABSTRACT

The present invention relates to compounds of Formula (I), pharmaceutical compositions comprising such compounds and use thereof. The compounds are useful in treating, preventing or ameliorating diseases or disorders such as cancer.

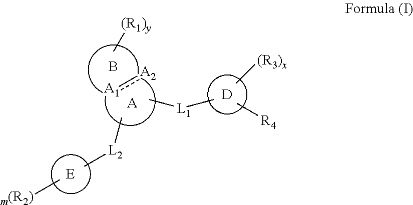

### FIELD OF THE INVENTION

The present invention relates to new compounds of general formula (I) as described and defined herein. The present invention also relates to pharmaceutical compositions comprising such compounds and the use of said compounds for the treatment or prophylaxis of diseases, in particular of cancer, pre-cancerous syndromes, congenital diseases and hyperproliferative disorders.

### BACKGROUND OF THE INVENTION

Under normal circumstances, the dynamic balance between cell proliferation and apoptosis maintains the normal size of tissues and organs and the stable of the environment in the body. When cell proliferation or apoptosis is out of control, cell malignant transformation can occur. The Hippo signaling pathway is a cell inhibitory growth pathway. It regulates the balance between cell proliferation and apoptosis through kinases cascade composed of a variety of tumor suppressor factors. It plays a key role in early embryonic development, organ size and regeneration, etc.

The Hippo pathway was originally discovered in Drosophila as a major developmental pathway that controls organ size and was later found to be conserved in mammals. In mammals, the Hippo signaling pathway can be divided into three parts: upstream regulatory elements (Merlin/NF2, GPCRS, etc.), core kinase cascade (MST1/2, LATS1/2 and regulatory protein SAV1 and MOB) and downstream effector molecule (YAP/TAZ). The tumor suppressor protein Neurofibromatosis 2 (NF2/merlin) or other upstream signals activate a complex of kinase(s) MST1/2 and scaffold protein SAV1. Activated MST1/2 promotes the phosphorylation of LATS1/2 and MOB. Subsequently, the phosphorylated LATS1/2 is able to regulate the pathway through phosphorylation of YAP/TAZ. The phosphorylated YAP/TAZ protein binds to 14-3-3 and β-TrCP, which mediate cytoplasmic retention and proteasomal degradation, respectively.

The unphosphorylated YAP/TAZ in the cytoplasm translocates to the nucleus and serves as transcriptional co-activators for TEAD1-4. Various cytokines including connective tissue growth factor (CTGF), cysteine-richangio-genic inducer 61 (CYR61), ankyrin Repeat Domain 1 (ANKRD1), baculoviral IAP repeat-containing protein 5 (BIRC5), brain-derived neurotrophic factor, and fibroblast growth factor1 worked as downstream substrates for YAP/TAZ stimulation. As a direct target gene for YAP/TAZ, CTGF can promote cell proliferation and anchorage for independent growth.

The human YAP gene is located on chromosome 11q13 and is widely expressed in various tissues except for peripheral blood cells. YAP contains multiple domains and specific amino acid sequences, including a TEAD-binding region, two WW domains, an N-terminal proline-rich domain, a C-terminal PDZ-binding motif, an SH3-binding motif, a coiled-coil domain and a transcription activation domain. YAP has two subtypes: YAP1 and YAP2. YAP1 contains one WW domain, and YAP2 contains two WW domains. The WW domain specifically recognizes the PPXY motif to mediate the formation of transcription complexes. YAP2 is the main form of YAP and has stronger transcriptional regulatory activity than YAP1. In addition, TAZ is homologous to YAP and has similar domains and functions as YAP, but lacks a porline-rich domain and a second WW domain.

TEAD family is the most important transcription factor of YAP and TAZ. The key site mutation of TEAD especially associated with the binding domains of YAP and TEAD significantly inhibit the expression and function of YAP-induced genes. Human TEAD family transcription factors have four members TEAD1/2/3/4, which are highly homology. TEADs have a TEA binding domain at the N-terminus, which serves as a site for binding to the DNA transcription promoter, and YAP/TAZ binding site at the C-terminus. The N-terminal domain of YAP/TAZ wraps the C-terminal domain of TEAD to form a spherical structure. The binding area of YAP/TAZ and TEAD is divided into three interfaces. The interface 1 is mediated by seven intermolecular hydrogen bonds between the peptide backbones of YAP β1 and TEAD β7 forming an antiparallel β sheet. The interface 2 is created by the YAP α1 helix which is close to a groove formed by TEAD α3 and α4. In the interface 3, the Ω-loop of YAP interacts with a deep pocket formed by β4, β11, β12, α1, and α4 of TEAD.

Normally, YAP/TAZ are only induced in certain tissues and under specific conditions (such as development, wound healing, etc.). The expression level in other tissues is low. It is described that some mutation of Hippo signaling components trigger the hyperactivition of YAP/TAZ, resulting in abnormal cell proliferation. Studies demonstrated that hyperactivition of YAP/TAZ subsequent to a deregulation of the Hippo pathway is widespread in cancers such as lung, liver, pancreas, breast cancer, and etc.

Among the cancer stem cells of a variety of solid tumors, YAP/TAZ is found to promote the survival of cancer stem cells, and is closely related to tumor metastasis and drug resistance mechanisms, and promotes the occurrence and development of a variety of tumors. During chemotherapeutic drug therapy, anti-microtubule drugs, antimetabolites and DNA damaging agents etc. can affect the Hippo signaling pathway, leading to YAP/TAZ activation and transcription, and thus drug resistance. The hyperactivities of YAP/TAZ can induce high expression of multiple drug transporters, which can transporting drugs outside the cell, and can also lead to upregulation of anti-apoptotic proteins such as Bcl and survivin, and inhibit cell apoptosis. Many studies have proved that PD-L1 is the direct transcription target of YAP/TAZ. Activated YAP/TAZ can increase the expression of PD-L1. Meanwhile, it can also induce the expression of cytokines IL-6, CSF1-3, TNFA, IL-3, CXCL1/2, CCL2, etc. to promote the recruitment and polarization of myeloid-derived suppressor cells (MDSC) and inactivate T cells, or induce T cell apoptosis. More studies have shown that down-regulation of the Hippo signaling pathway leads to activation of YAP/TAZ, which is also the main mechanism of multiple targeted drug resistance. Transcription activated by YAP/TAZ can overcome EGFR resistance through multiple mechanisms. For example, high AXL expression mediates NSCLC resistance to EGFR inhibitors; Inhibition of the pro-apoptotic protein BMF mediate resistance to EGFR/MEK inhibitors; Activation of the PI3K/AKT signaling pathway to evade targeted therapy. YAP-activated transcription can also mediate resistance to BRAF, KRAS, and MAPK inhibitors. YAP/TAZ activity is not only related to drug resistance, studies have shown that YAP gene amplification is related to driving cancer recurrence of colon cancer and pancreatic cancer.

Accordingly, the Hippo pathway plays an important role in organ or tissue size control. It has been linked to many aspects of tumorigenesis, including cell proliferation, cell differentiation, cell apoptosis, cell competition, tissue regeneration, cancer metastasis, and cancer therapy resistance. The deregulation of Hippo pathway can lead to the high expression and activation of YAP/TAZ in cytoplasm and cell nucleus, which can induce the development and metastasis of tumor and even drug resistance. The disruption of YAP/TAZ-TEAD interaction can abrogate the oncogenic property of YAP/TAZ. Therefor the inhibitor of protein-protein interaction of YAP/TAZ and TEAD provides a rationale for the treatment of these cancers.

### SUMMARY OF THE INVENTION

The present invention relates to compounds of Formula (I), or a stereoisomer, tautomer, pharmaceutically acceptable salt, prodrug, chelate, non-covalent complex, or solvate thereof,

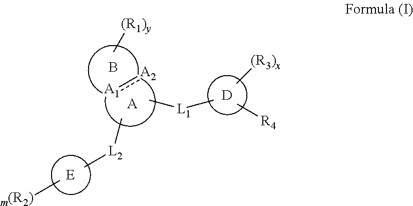

wherein,
[symbol] is a single bond or a double bond;
A₁ and A₂ are independently C or N;
ring B is selected from the group consisting of C₅₋₆ aryl, C₅₋₆ cycloalkyl, 5 to 6-membered heterocyclyl and 5 to 6-membered heteroaryl, wherein the 5 to 6-membered heterocyclyl and 5 to 6-membered heteroaryl comprising 1, 2, 3 or 4 heteroatoms independently selected from N, S and O;
ring A is selected from the group consisting of C₅₋₆ aryl, 5 to 6-membered heteroaryl, 5 to 6-membered heterocyclyl, the 5 to 6-membered heteroaryl and 5 to 6-membered heterocyclyl comprising 1-4 hetero atoms independently selected from N, S and O, wherein the C₅₋₆ aryl, 5 to 6-membered heteroaryl and 5 to 6-membered heterocyclyl are each optionally substituted with 0 to 3 substituents independently selected from the group consisting of oxo, ═NH, hydroxyl, halogen, CN, —NH(C₁₋₆ alkyl), —NH(C₁₋₆ alkyl), —N—(C₁₋₆ alkyl)₂, C₁₋₆ alkyl, C₂₋₆ alkenyl, C₂₋₆ alkynyl, C₁₋₆ haloalkyl, C₁₋₆ alkoxyl, C₁₋₆ haloalkoxyl, —OC(═O)Rₐ, —C(═O)NRₐR_{b},—C(═O)ORₐ, —C(═O)R_{c}, —S(═O)R_{b}, —S(═O)₂R_{b}, —S(═O)NRₐR_{b}, —S(═O)₂NRₐR_{b};
L₁ is bond, —O—, —S—, —NRₐ—, —(CH₂)ₜ—, —(CH₂)ₜ—NRₐ—,—NRₐ—(CH₂)ₜ—, —(CH₂)ₜ—O—, —O—(CH₂)ₜ—, —C(═O)—, —C(═O)NRₐ— or —NRₐ—C(═O)—;
ring E is C₅₋₆ aryl, 5 to 10-membered heteroaryl, C₃₋₈ cycloalkyl or 4 to 8-membered heterocyclyl, wherein the 5 to 10-membered heteroaryl and 4 to 8-membered heterocyclyl comprising 1-4 hetero atoms independently selected from N, S and O;
L₂ is bond, —O—, —S—, —NH—, —(CH₂)ₜ—O—, —O—(CH₂)ₜ—, —C(═O)—, —C₁₋₄ alkylene, —C₂₋₄ alkenylene, or —C₂₋₄ alkynylene;
ring D is C₅₋₁₀ aryl, 5 to 10-membered heteroaryl, C₃₋₁₀ cycloalkyl or 4 to 10-membered heterocyclyl, wherein the 5 to 10-membered heteroaryl or 4 to 10-membered heterocyclyl comprising 1, 2, 3 or 4 heteroatoms independently selected from N, S and O;
R₁ is H, oxo, hydroxyl, halogen, CN,—NO₂, —NR_{d}Rₑ, C₁₋₆ alkyl, C₂₋₆ alkenyl, C₂₋₆ alkynyl, C₁₋₆ haloalkyl, C₁₋₆ alkoxyl, —C(═O)NRₐR_{b},—C(═O)ORₐ, —C(═O)R_{c}, —S(═O)R_{b}, —S(═O)₂R_{b}, —S(═O)NRₐR_{b}, —S(═O)₂NRₐR_{b}, —O—(C═O)—Rₐ, —O—(C═O)—NRₐR_{b}, C₁₋₆ haloalkoxyl, C₃₋₆ cycloalkyl, 3 to 6-membered heterocyclyl, C₅₋₆ aryl or 5 to 6-membered heteroaryl, which C₁₋₆ alkyl, C₂₋₆ alkenyl, C₂₋₆ alkynyl, C₁₋₆ haloalkyl, C₁₋₆ alkoxyl, C₁₋₆ haloalkoxyl, C₃₋₆ cycloalkyl, 3 to 6-membered heterocyclyl, C₅₋₆ aryl and 5 to 6-membered heteroaryl are each optionally substituted with 0 to 3 substituents independently selected from the group consisting of OH, CN, halogen, C₁₋₆ alkyl, C₂₋₄ alkenyl, C₂₋₄ alkynyl, C₁₋₄ haloalkyl, C₁₋₄ alkoxyl, —NRₐR_{b}, —C(═O)NRₐR_{b}, —OC(═O)Rₐ, —C(═O)ORₐ, —C(═O)Rₐ, —S(═O)R_{b}, —S(═O)₂R_{b}, —S(═O)NRₐR_{b}, —S(═O)₂NRₐR_{b}, —NRₐC(═O)R_{b}, C₁₋₄ haloalkoxyl, C₃₋₆ cycloalkyl, 3 to 6-membered heterocycloalkyl, C₅₋₆ aryl and 5 to 6-membered heteroaryl; wherein the 5 to 6-membered heteroaryl, 3 to 6-membered heterocycloalkyl and 3 to 6-membered heterocyclyl optionally comprising 1, 2 or 3 hetero atoms independently selected from N, S and O;
R₂ is H, hydroxyl, halogen, CN, —NO₂, —NRₐR_{b}, oxo, —C(═O)NRₐR_{b}, —C(═O)ORₐ, —C(═O)Rₐ, —S(═O)R_{b}, —S(═O)₂R_{b}, —S(═O)NRₐR_{b}, —S(═O)₂NRₐR_{b}, —NRₐC(═O)R_{b}, SF₅, C₁₋₆ alkyl, C₂₋₆ alkenyl, C₂₋₆ alkynyl, C₁₋₆ alkoxyl, C₁₋₆ haloalkyl, C₃₋₆ cycloalkyl or 3 to 6-membered heterocyclyl comprising 1, 2 or 3 hetero atoms independently selected from N, S and O; wherein the C₁₋₆ alkyl, C₂₋₆ alkenyl, C₂₋₆ alkynyl, C₁₋₆ alkoxyl, C₃₋₆ cycloalkyl and 3 to 6-membered heterocyclyl are each optionally substituted with 0 to 3 substitutents independently selected from the group consisting of —ORₐ, halogen, CN, C₁₋₄ alkyl, C₁₋₆ haloalkyl, —NRₐR_{b}, oxo, —OC(═O)Rₐ, —C(═O)NRₐR_{b}, —C(═O)ORₐ, —C(═O)Rₐ, —S(═O)R_{b}, —S(═O)₂R_{b}, —S(═O)NRₐR_{b}, —S(═O)₂NRₐR_{b} and —NRₐC(═O)R_{b};
R₃ is H, oxo, halogen, —ORₐ, CN, —NO₂, —NRₐR_{b}, —NRₐC(═O)R_{b}, —C₁₋₄ alkylene-NRₐR_{b}, —C₁₋₄ alkylene-NRₐC(═O)R_{b}, —C(═O)R_{b}, —OC(═O)Rₐ, —C(═O)ORₐ, —C(═O)NRₐR_{b}, —S(═O)R_{b}, —S(═O)₂R_{b}, —S(═O)NRₐR_{b}, —S(═O)₂NRₐR_{b}, —C₁₋₄ alkylene-C(═O)NRₐR_{b}, C₁₋₆ alkyl, C₂₋₆ alkenyl, C₂₋₆ alkynyl, C₁₋₆ alkoxyl, C₁₋₆ haloalkyl, C₁₋₆ haloalkoxyl, 3 to 6-membered heterocyclyl, C₃₋₆ cycloalkyl, C₅₋₆ aryl or 5 to 6-membered heteroaryl, wherein the 5 to 6-membered heteroaryl and 3 to 6-membered heterocyclyl optionally comprising 1, 2 or 3 hetero atoms independently selected from N, S and O; which C₁₋₆ alkyl, C₂₋₆ alkenyl, C₂₋₆ alkynyl, C₁₋₆ haloalkyl, C₁₋₆ alkoxyl, C₁₋₆ haloalkoxyl, C₃₋₆ cycloalkyl, 3 to 6-membered heterocyclyl, C₅₋₆ aryl and 5 to 6-membered heteroaryl are each optionally substituted with 0 to 3 substitutents independently selected from the group consisting of oxo, hydroxyl, halogen, CN, —NO₂, C₁₋₆ alkyl, —C₁₋₄ alkylene-OH, C₁₋₆ haloalkyl, C₁₋₆ alkoxyl, —S(═O)R_{b}, —S(═O)₂R_{b}, —NRₐR_{b}, —C(═O)R_{b}, —OC(═O)Rₐ, —C(═O)ORₐ, —NRₐC(═O)R_{b}, —C(═O)NRₐR_{b}, —NRₐC(═O)R_{b}, —C₁₋₄ alkylene-NRₐR_{b}, —C₁₋₄ alkylene-NRₐC(═O)R_{b}, C₁₋₄ alkylene-C(═O)NRₐR_{b}, —C₁₋₄ alkylene-OH, C₃₋₆ cycloalkyl, 3 to 6-membered heterocycloalkyl, C₅₋₆ aryl and 5 to 6-membered heteroaryl;
R₄ is

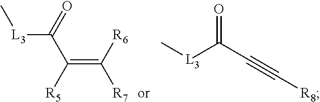

L₃ is bond, —NRₐ—, —(CH₂)ₜ—NRₐ—, —C₄₋₆ heterocyclyl or —C₄₋₆cycloalkyl-NRₐ—;
R₅, R₆, R₇ and R₈ are independently selected from the group consisting of H, halogen, —ORₐ, CN, —NRₐR_{b}, —C₁₋₆ alkylene-NRₐR_{b}, —C₁₋₆ alkylene-R_{c}, C₁₋₆ alkyl, C₂₋₆ alkenyl, C₂₋₆ alkynyl, C₁₋₆ haloalkyl, C₁₋₆ haloalkyl, C₁₋₆ alkoxyl, C₃₋₆ cycloalkyl, 3 to 6-membered heterocyclyl, C₅₋₆ aryl and 5 to 6-membered heteroaryl, which C₁₋₆ alkyl, C₂₋₆ alkenyl, C₂₋₆ alkynyl, C₁₋₆ haloalkyl, C₁₋₆ alkoxyl, C₁₋₆ haloalkoxyl, C₃₋₆ cycloalkyl, 3 to 6-membered heterocyclyl, C₅₋₆ aryl and 5 to 6-membered heteroaryl are each optionally substituted with 0 to 4 substitutents independently selected from the group consisting of OH, CN, halogen, C₁₋₆ alkyl, C₂₋₄ alkenyl, C₂₋₄ alkynyl, C₁₋₄ haloalkyl, C₁₋₄ alkoxyl, —NRₐR_{b}, C₃₋₆ cycloalkyl, 3 to 6-membered heterocyclyl, C₅₋₆ aryl and 5 to 6-membered heteroaryl, wherein the 5 to 6-membered heteroaryl and 3 to 6-membered heterocyclyl optionally comprising 1, 2 or 3 hetero atoms independently selected from N, S and O;
Rₐ and R_{b} are independently selected from the group consisting of H, CN, hydroxyl, halogen, C₁₋₆ alkyl, C₁₋₆ haloalkyl, C₁₋₄ alkoxyl, C₃₋₆ cycloalkyl, 3 to 6-membered heterocycloalkyl, C₅₋₆ aryl and 5 to 6-membered heteroaryl, wherein the C₁₋₆ alkyl, C₁₋₄ alkoxyl, C₃₋₆ cycloalkyl, 3 to 6-membered heterocycloalkyl, C₅₋₆ aryl and 5 to 6-membered heteroaryl are each optionally substituted with 0 to 4 substitutents independently selected from the group consisting of halogen, CN, —OH, oxo, C₁₋₆ alkyl, C₂₋₆ alkenyl, C₂₋₆ alkynyl, C₁₋₆ haloalkyl, C₁₋₃ alkoxyl, C₁₋₃ haloalkoxyl and C₅₋₆ aryl; wherein the 5 to 6-membered heteroaryl and 3 to 6-membered heterocycloalkyl optionally comprising 1, 2 or 3 hetero atoms independently selected from N, S and O;
R_{c} is 3 to 6-membered heterocyclyl optionally substituted with 0 to 4 substitutents independently selected from the group consisting of halogen, CN, —OH, oxo, C₁₋₆ alkyl, C₂₋₆ alkenyl, C₂₋₆ alkynyl, C₁₋₆ haloalkyl, C₁₋₃ alkoxyl and C₁₋₃ haloalkoxyl;
R_{d} and Rₑ are independently selected from the group consisting of C₁₋₆ alkyl, C₂₋₆ alkenyl, C₂₋₆ alkynyl, C₁₋₆ haloalkyl, C₁₋₆ alkoxyl, —C(═O)NR_{f}R_{f}, —C(═O)OR_{f}, —O—C(═O)R_{f}, —C(═O)R_{f}, —S(═O)R_{f}, —S(═O)₂R_{f}, —S(═O)NR_{f}R_{f}, —S(═O)₂NR_{f}R_{f}, —C₁₋₄ alkylene-NR_{f}R_{f}, —C₁₋₄ alkylene-NR_{f}C(═O)R_{f}, —C₁₋₄ alkylene-C(═O)NR_{f}R_{f};
R_{f} is H, C₁₋₆ alkyl, C₂₋₆ alkynyl, C₁₋₆ haloalkyl, C₁₋₆ haloalkoxyl or C₁₋₆ alkoxyl;
t is 1, 2, 3 or 4;
x, y and m are independently 0, 1, 2, 3, 4 or 5.

In some embodiments, compounds of Formula (I-1), or a stereoisomer, tautomer, pharmaceutically acceptable salt, prodrug, chelate, non-covalent complex, or solvate thereof,

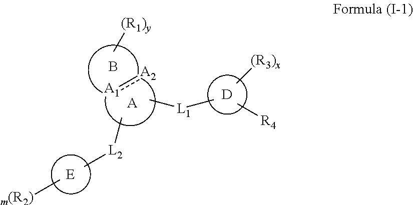

wherein,
[symbol] is a single bond or a double bond;
A₁ and A₂ are independently C or N;
ring B is selected from the group consisting of C₅₋₆ aryl, 5 to 6-membered heteroaryl comprising 1, 2, 3 or 4 heteroatoms independently selected from N, S and O;
ring A is selected from the group consisting of C₅₋₆ aryl, 5 to 6-membered heteroaryl, 5 to 6-membered heterocyclyl, the 5 to 6-membered heteroaryl and 5 to 6-membered heterocyclyl comprising 1-4 hetero atoms independently selected from N, S and O, wherein the 5 to 6-membered heterocyclyl and the 5 to 6-membered heteroaryl are optionally and independently substituted with one or more

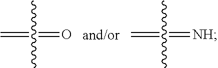

L₁ is bond, —O—, —S—, —NH—, —(CH₂)ₜ—, —(CH₂)ₜ—O—, —O—(CH₂)ₜ, —C(═O)—, —C(═O)NH— or —NH—C(═O)—;
ring E is C₅₋₆ aryl, 5 to 10-membered heteroaryl, C₃₋₈ cycloalkyl or 4 to 8-membered heterocyclyl, wherein the 5 to 10-membered heteroaryl and 4 to 8-membered heterocyclyl comprising 1-4 hetero atoms independently selected from N, S and O;
L₂ is bond, —O—, —S—, —NH—, —(CH₂)ₜ—O—, —O—(CH₂)ₜ—, —C(═O)—, —C₁₋₄ alkylene, —C₂₋₄ alkenylene, or —C₂₋₄ alkynylene;
ring D is C₅₋₁₀ aryl, 5 to 10-membered heteroaryl, C₃₋₁₀ cycloalkyl or 4 to 10-membered heterocyclyl, wherein the 5 to 10-membered heteroaryl or 4 to 10-membered heterocyclyl comprising 1, 2, 3 or 4 heteroatoms independently selected from N, S and O;
R₁ is H, oxo, hydroxyl, halogen, CN, —NH(C₁₋₆ alkyl), —N—(C₁₋₆ alkyl)₂, C₁₋₆ alkyl, C₂₋₄ alkenyl, C₂₋₄ alkynyl, C₁₋₄ haloalkyl, C₁₋₄ alkoxyl, —C(═O)NRₐR_{b},—C(═O)ORₐ, —OC(═O) Rₐ, —C(═O)R_{c}, —S(═O)R_{b}, —S(═O)₂R_{b}, —S(═O)NRₐR_{b}, —S(═O)₂NRₐR_{b}, C₁₋₄ haloalkoxyl, C₃₋₆ cycloalkyl, 3 to 6-membered heterocyclyl, C₅₋₆ aryl or 5 to 6-membered heteroaryl, which C₁₋₆ alkyl, C₂₋₄ alkenyl, C₂₋₄ alkynyl, C₁₋₄ haloalkyl, C₁₋₄ alkoxyl, C₁₋₄ haloalkoxyl, C₃₋₆ cycloalkyl, 3 to 6-membered heterocyclyl, C₅₋₆ aryl and 5 to 6-membered heteroaryl are each optionally substituted with 0 to 3 substituents independently selected from the group consisting of OH, CN, halogen, C₁₋₆ alkyl, C₂₋₄ alkenyl, C₂₋₄ alkynyl, C₁₋₄ haloalkyl, C₁₋₄ alkoxyl, —NRₐR_{b}, —C(═O)NRₐR_{b}, —S(═O)R_{b}, —S(═O)₂R_{b}, —S(═O)NRₐR_{b}, —S(═O)₂NRₐR_{b}, —NRₐC(═O)R_{b}, C₁₋₄ haloalkoxyl, C₃₋₆ cycloalkyl, 3 to 6-membered heterocycloalkyl, C₅₋₆ aryl and 5 to 6-membered heteroaryl; wherein the 5 to 6-membered heteroaryl and 3 to 6-membered heterocyclyl optionally comprising 1, 2 or 3 hetero atoms independently selected from N, S and O;
R₂ is H, hydroxyl, halogen, CN, C₁₋₆ alkyl, C₂₋₆ alkenyl, C₂₋₆ alkynyl, C₁₋₆ alkoxyl, C₁₋₆ haloalkyl, C₃₋₆ cycloalkyl or 3 to 6-membered heterocycloalkyl comprising 1, 2 or 3 hetero atoms independently selected from N, S and O; wherein the C₁₋₄ alkyl, C₂₋₄ alkenyl, C₂₋₄ alkynyl, C₁₋₄ alkoxyl, C₃₋₆ cycloalkyl and 3 to 6-membered heterocycloalkyl are each optionally substituted with 0 to 3 substitutents independently selected from the group consisting of hydroxyl, halogen, CN and C₁₋₄ alkyl;
R₃ is H, oxo, halogen, CN, —NO₂, —NRₐR_{b}, —NRₐC(═O)R_{b}, —C₁₋₄ alkylene-NRₐR_{b}, —C₁₋₄ alkylene-NRₐC(═O)R_{b}, —C(═O)R_{b}, —OC(═O) Rₐ, —C(═O)ORₐ, —C(═O)NRₐR_{b}, —S(═O)R_{b}, —S(═O)₂R_{b}, —S(═O)NRₐR_{b}, —S(═O)₂NRₐR_{b}, —C₁₋₄ alkylene-C(═O)NRₐR_{b}, C₁₋₆ alkyl, C₂₋₆ alkenyl, C₂₋₆ alkynyl, C₁₋₆ alkoxyl, C₁₋₆ haloalkyl, C₁₋₄ haloalkoxyl, 3 to 6-membered heterocycloalkyl, C₃₋₆cycloalkyl, C₅₋₆ aryl or 5 to 6-membered heteroaryl, wherein the 5 to 6-membered heteroaryl and 3 to 6-membered heterocycloalkyl optionally comprising 1, 2 or 3 hetero atoms independently selected from N, S and O; which C₁₋₆ alkyl, C₂₋₆ alkenyl, C₂₋₆ alkynyl, C₁₋₄ haloalkyl, C₁₋₄ alkoxyl, C₁₋₄ haloalkoxyl, C₃₋₆cycloalkyl, 3 to 6-membered heterocycloalkyl, C₅₋₆ aryl and 5 to 6-membered heteroaryl are each optionally substituted with 0 to 3 substitutents independently selected from the group consisting of oxo, hydroxyl, halogen, CN, —NO₂, C₁₋₆ alkyl, —C₁₋₄ alkylene-OH, C₁₋₆haloalkyl, C₁₋₆ alkoxyl, —S(═O)R_{b}, —S(═O)₂R_{b}, —NRₐR_{b}, —C(═O)R_{b}, —OC(═O) Rₐ, —C(═O)ORₐ, —NRₐC(═O)R_{b}, —C(═O)NRₐR_{b}, —NRₐC(═O)R_{b}, —C₁₋₄ alkylene-NRₐR_{b}, —C₁₋₄ alkylene-NRₐC(═O)R_{b}, C₁₋₄ alkylene-C(═O)NRₐR_{b}, —C₁₋₄ alkylene-OH, C₃₋₆ cycloalkyl, 3 to 6-membered heterocycloalkyl, C₅₋₆ aryl and 5 to 6-membered heteroaryl;
R₄ is

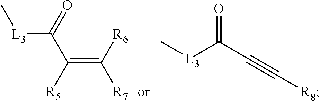

L₃ is bond, —NH—, —(CH₂)ₜ—NH—, —C₄₋₆ heterocyclyl or —C₄₋₆cycloalkyl-NH—;
R₅, R₆, R₇ and R₈ are independently selected from H, halogen, CN, —NRₐR_{b}, —C₁₋₆ alkylene-NRₐR_{b}, —C₁₋₆ alkylene-R_{c}, C₁₋₄ alkyl, C₂₋₄ alkenyl, C₂₋₄ alkynyl, C₁₋₄ alkoxyl, C₃₋₆ cycloalkyl, 3 to 6-membered heterocycloalkyl, C₅₋₆ aryl and 5 to 6-membered heteroaryl, which C₁₋₆ alkyl, C₂₋₄ alkenyl, C₂₋₄ alkynyl, C₁₋₄ haloalkyl, C₁₋₄ alkoxyl, C₁₋₄ haloalkoxyl, C₃₋₆ cycloalkyl, 3 to 6-membered heterocycloalkyl, C₅₋₆ aryl and 5 to 6-membered heteroaryl are each optionally substituted with 0 to 4 substitutents independently selected from the group consisting of OH, CN, halogen, C₁₋₆ alkyl, C₂₋₄ alkenyl, C₂₋₄ alkynyl, C₁₋₄ haloalkyl, C₁₋₄ alkoxyl, —NRₐR_{b}, C₃₋₆ cycloalkyl, 3 to 6-membered heterocycloalkyl, C₅₋₆ aryl and 5 to 6-membered heteroaryl, wherein the 5 to 6-membered heteroaryl and 3 to 6-membered heterocycloalkyl optionally comprising 1, 2 or 3 hetero atoms independently selected from N, S and O;
Rₐ and R_{b} are independently selected from the group consisting of H, CN, hydroxyl, halogen, C₁₋₆ alkyl, C₁₋₆ haloalkyl, C₁₋₄ alkoxyl, C₃₋₆ cycloalkyl, 3 to 6-membered heterocycloalkyl, C₅₋₆ aryl and 5 to 6-membered heteroaryl, wherein the C₁₋₆ alkyl, C₁₋₄ alkoxyl, C₃₋₆ cycloalkyl, 3 to 6-membered heterocycloalkyl, C₅₋₆ aryl and 5 to 6-membered heteroaryl are each optionally substituted with 0 to 4 substitutents independently selected from the group consisting of halogen, CN, —OH, oxo, C₁₋₆ alkyl, C₂₋₆ alkenyl, C₂₋₆ alkynyl, C₁-6 haloalkyl, C₁₋₃ alkoxyl, C₁₋₃ haloalkoxyl and C₅₋₆ aryl; wherein the 5 to 6-membered heteroaryl and 3 to 6-membered heterocycloalkyl optionally comprising 1, 2 or 3 hetero atoms independently selected from N, S and O;
R_{c} is 3 to 6-membered heterocycloalkyl optionally substituted with 0 to 4 substitutents independently selected from the group consisting of halogen, CN, —OH, oxo, C₁₋₆ alkyl, C₂₋₆ alkenyl, C₂₋₆ alkynyl, C₁₋₆ haloalkyl, C₁₋₃ alkoxyl or C₁₋₃ haloalkoxyl;
t is 1, 2, 3 or 4;
x, y and m are independently 0, 1, 2, 3, 4 or 5.

In some embodiments, ring B is selected from the group consisting of C₅₋₆ aryl, C₅₋₆ cycloalkyl, 5 to 6-membered heteroaryl comprising 1, 2, 3 or 4 N heteroatoms, and 5 to 6-membered heterocyclyl comprising 1, 2, 3 or 4 heteroatoms independently selected from N, S, O.

In some embodiments, ring B is selected from the group consisting of C₅₋₆ aryl, C₅₋₆ cycloalkyl, 5 to 6-membered heteroaryl comprising 1 or 2 N heteroatoms, and 5 to 6-membered heterocyclyl comprising 1 or 2 O heteroatoms, wherein the 5 to 6-membered heteroaryl and 5 to 6-membered heterocyclyl are optionally and independently substituted with one or more

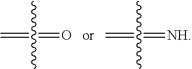

In some embodiments, ring B is selected from the group consisting of phenyl, cyclohexyl, 6-membered heteroaryl comprising 1 or 2 N heteroatoms, and 6-membered heterocyclyl comprising 1 or 2 O heteroatoms.

In some embodiments, ring B is C₅₋₆ aryl, oxo substituted or unsubstituted 5 to 6-membered heteroaryl comprising 1, 2, 3 or 4 N heteroatoms.

In some embodiments, ring B is C₅₋₆ aryl, oxo substituted or unsubstituted 5 to 6-membered heteroaryl comprising 1 or 2 N heteroatoms.

In some embodiments, ring B is phenyl or 6-membered heteroaryl comprising 1 or 2 N atoms.

In some embodiments, ring B is phenyl or 6-membered heteroaryl comprising 1 or 2 N atoms, wherein the 6-membered heteroaryl is optionally substituted with one or more

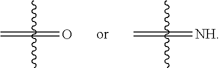

In some embodiments, ring A is selected from the group consisting of C₅₋₆ aryl, 5 to 6-membered heteroaryl, 5 to 6-membered heterocyclyl, 5 to 6-membered heteroaryl and 5 to 6-membered heterocyclyl comprising 1-4 hetero atoms independently selected from N, S and O, wherein the 5 to 6-membered heterocyclyl and the 5 to 6-membered heteroaryl are optionally and independently substituted with one or more

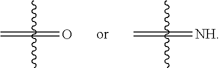

In some embodiments, ring A is C₅₋₆ aryl, 5 to 6-membered heteroaryl or 5 to 6-membered heterocyclyl, the 5 to 6-membered heteroaryl and 5 to 6-membered heterocyclyl comprising 1, 2 or 3 N heteroatoms, wherein the 5 to 6-membered heteroaryl and 5 to 6-membered heterocyclyl are optionally and independently substituted with one or more

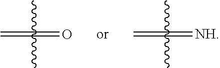

In some embodiments, ring A is C₅₋₆ aryl, 5 to 6-membered heteroaryl or 5 to 6-membered heterocyclyl, the 5 to 6-membered heteroaryl and 5 to 6-membered heterocyclyl comprising 1 or 2 N heteroatoms, wherein the 5 to 6-membered heteroaryl and 5 to 6-membered heterocyclyl are optionally and independently substituted with one or more 1=0 or

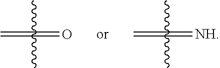

In some embodiments, ring A is phenyl, 5 to 6-membered heteroaryl or 5 to 6-membered heterocyclyl, the 5 to 6-membered heteroaryl and 5 to 6-membered heterocyclyl comprising 1, 2 or 3 N heteroatoms, wherein the 5 to 6-membered heteroaryl and 5 to 6-membered heterocyclyl are optionally and independently substituted with one or more

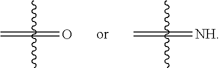

In some embodiments, ring A is phenyl or 5 to 6-membered heteroaryl comprising 1, 2 or 3 N heteroatoms, wherein the 5 to 6-membered heteroaryl may optionally be substituted with one or more

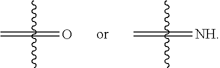

In some embodiments, ring A is phenyl or 5 to 6-membered heteroaryl comprising 1 or 2 N hetero atoms, wherein the 5 to 6-membered heteroaryl may optionally substituted with one or more

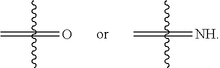

In some embodiments, ring A is C₆ phenyl or 5 to 6-membered heteroaryl comprising 1 or 2 N hetero atoms.

In some embodiments, ring E is C₅₋₆ aryl, 5 to 6-membered heteroaryl, C₃₋₈ cycloalkyl or 4 to 8-membered heterocyclyl, wherein the 5 to 6-membered heteroaryl and 4 to 8-membered heterocyclyl comprising 1, 2 or 3 heteroatoms independently selected from N, S and O.

In some embodiments, ring E is C₃₋₈ cycloalkyl, C₅₋₆ aryl or 5 to 6-membered heteroaryl comprising 1, 2 or 3 heteroatoms independently selected from N, S and O.

In some embodiments, ring E is C₃₋₆ cycloalkyl, phenyl or 5 to 6-membered heteroaryl comprising 1, 2 or 3 heteroatoms independently selected from N, S and O.

In some embodiments, ring E is C₃₋₆ cycloalkyl, phenyl or 5 to 6-membered heteroaryl comprising 1, 2 or 3 hetero atoms independently selected from N and S.

In some embodiments, L₁ and L₂ are all attached to ring A.

In some embodiments, L₁ is bond, —O—, —S—, —NRₐ—, —(CH₂)ₜ—, —(CH₂)ₜ—O—, —O—(CH₂)ₜ—, —C(═O)—, —C(═O)NRₐ— or —NRₐ—C(═O)—.

In some embodiments, L₁ is bond, —O—, —S—, —NH—, —(CH₂)ₜ—, —(CH₂)ₜ—O—, —C(═O)— or —C(═O)NH—.

In some embodiments, L₁ is bond, —NH—, —O—, —S—, —N—C₁₋₃ alkylene-, —(CH₂)ₜ— or —C(═O)—.

In some embodiments, L₁ is bond, —NH—, —N—C₁₋₃ alkylene-, —(CH₂)ₜ— or —C(═O)—.

In some embodiments, L₁ is bond, —NH—, —(CH₂)ₜ— or —C(═O)—.

In some embodiments, L₁ is bond, —NH—, —N—C₁₋₃ alkylene-, —CH₂— or —C(═O)—.

In some embodiments, L₁ is bond, —NH—, —CH₂— or —C(═O)—.

In some embodiments, L₁ is bond, —NH— or —C(═O)—.

Preferably, L₁ is bond.

In some embodiments, L₂ is bond, —O—, —S—, —NH—, —C(═O)—, —C₂₋₄ alkenylene, or —C₂₋₄ alkynylene.

In some embodiments, L₂ is bond, —O—, C₂₋₄ alkenylene, or C₂₋₄ alkynylene.

In some embodiments, L₂ is bond, C₂₋₄ alkenylene or C₂₋₄ alkynylene.

In some embodiments, L₂ is bond or —O—.

In some embodiments, L₂ is C₂₋₄ alkenylene or C₂₋₄ alkynylene.

In some embodiments, L₂ is bond, C₂₋₄ alkenylene, C₂₋₄ alkynylene, when ring E is phenyl or 5 to 6-membered heteroaryl comprising for 2 N heteroatoms; L₂ is C₂₋₄ alkenylene or C₂₋₄ alkynylene, when ring E is C₃₋₆ cycloalkyl.

In some embodiments, L₂ is bond, C₂₋₄ alkenylene, C₂₋₄ alkynylene, when ring E is phenyl or 6-membered heteroaryl comprising 1 or 2 N heteroatoms; L₂ is C₂₋₄ alkenylene or C₂₋₄ alkynylene, when ring E is C₃₋₆ cycloalkyl or 5-membered heteroaryl comprising for 2 N heteroatoms independently selected from N, S and O.

In some embodiments, L₂ is bond.

In some embodiments, ring D is C₅₋₆ aryl, C₅₋₁₀ heteroaryl, C₄₋₆ cycloalkyl or C₄₋₁₀ heterocyclyl, wherein the C₅₋₁₀ heteroaryl and C₄₋₁₀ heterocyclyl optionally comprising 1, 2 or 3 hetero atoms independently selected from N, S, or O.

In some embodiments, ring D is C₅₋₁₀ aryl, 5 to 10-membered heteroaryl, C₃₋₁₀ cycloalkyl or 4 to 10-membered heterocyclyl, wherein the 5 to 10-membered heteroaryl and 4 to 10-membered heterocyclyl comprising 1, 2 or 3 heteroatoms independently selected from N and O.

In some embodiments, ring D is C₅₋₆ aryl, 5 to 6-membered heteroaryl, 3 to 6-membered mono-cycloalkyl, 4 to 6-membered mono-heterocyclyl, 6 to 10-membered fused- or spiro-bicyclic heteroaryl, 6 to 10-membered fused- or spiro-bicyclic heterocyclyl, wherein the 5 to 6-membered heteroaryl, 4 to 6-membered heterocyclyl, 6 to 10-membered heteroaryl, 6 to 10-membered heterocyclyl comprising 1, 2 or 3 heteroatoms independently selected from N and O.

In some embodiments, ring D is C₅₋₆ aryl, 5 to 6-membered heteroaryl, C₃₋₆ cycloalkyl, 4 to 6-membered heterocyclyl, wherein the 5 to 6-membered heteroaryl and 4 to 6-membered heterocyclyl comprising 1, 2 or 3 heteroatoms independently selected from N, S and O.

In some embodiments, ring D is phenyl, C₃₋₆ cycloalkyl or 4 to 6-membered heterocyclyl comprising 1 or 2 heteroatoms independently selected from N and 0.

In some embodiments, ring D is phenyl or 4 to 5-membered heterocyclyl comprising 1 or 2 N heteroatoms.

In some embodiments, R₁ is H, oxo, hydroxyl, halogen, CN,—NO₂, —NR_{d}Rₑ, C₁₋₆ alkyl, C₂₋₆ alkenyl, C₂₋₆ alkynyl, C₁₋₆ haloalkyl, C₁₋₆ alkoxyl, —C(═O)NRₐR_{b},—C(═O)ORₐ, —C(═O)RR, —S(═O)R_{b}, —S(═O)₂R_{b}, —S(═O)NRₐR_{b}, —S(═O)₂NRₐR_{b}, —O—(C═O)—Rₐ, —O—(C═O)—NRₐR_{b}, C₁₋₆ haloalkoxyl, C₃₋₅ cycloalkyl, 3 to 5-membered heterocyclyl, which C₁₋₆ alkyl, C₂₋₆ alkenyl, C₂₋₆ alkynyl, C₁₋₆ haloalkyl, C₁₋₆ alkoxyl, C₁₋₆ haloalkoxyl, C₃₋₅ cycloalkyl, 3 to 5-membered heterocyclyl are each optionally substituted with 0 to 3 substituents independently selected from the group consisting of OH, CN, halogen, C₁₋₆ alkyl, C₂₋₄ alkenyl, C₂₋₄ alkynyl, C₁₋₄ haloalkyl, C₁₋₄ alkoxyl, —NRₐR_{b}, —C(═O)NRₐR_{b}, —C(═O)ORₐ, —OC(═O) Rₐ, —C(═O)Rₐ, —S(═O)R_{b}, —S(═O)₂R_{b}, —S(═O)NRₐR_{b}, —S(═O)₂NRₐR_{b}, —NRₐC(═O)R_{b}, C₁₋₄ haloalkoxyl.

In some embodiments, R₁ is H, oxo, hydroxyl, halogen, CN, —NRₐR_{b}, —NRₐC(═O)R_{b}, —NO₂, C₁₋₆ alkyl, C₂₋₄ alkenyl, C₂₋₄ alkynyl, C₁₋₄ haloalkyl, C₁₋₄ alkoxyl, —C(═O)NRₐR_{b}, —S(═O)R_{b}, —S(═O)₂R_{b}, —S(═O)NRₐR_{b}, —S(═O)₂NRₐR_{b}, C₁₋₄ haloalkoxyl, C₃₋₆ cycloalkyl, C₃₋₆ heterocycloalkyl, C₅₋₆ aryl or C₅₋₆ heteroaryl, wherein the C₅₋₆ heteroaryl and C₃₋₆ heterocycloalkyl optionally comprising 1, 2 or 3 hetero atoms independently selected from N, S, or O; the C₁₋₆ alkyl, C₂₋₄ alkenyl, C₂₋₄ alkynyl, C₁₋₄ haloalkyl, C₁₋₄ alkoxyl, C₁₋₄ haloalkoxyl, C₃₋₆ cycloalkyl, C₃₋₆ heterocycloalkyl, C₅₋₆ aryl and C₅₋₆ heteroaryl are each optionally substituted with one or more substitutents independently selected from OH, CN, halogen, C₁₋₆ alkyl, C₂₋₄ alkenyl, C₂₋₄ alkynyl, C₁₋₄ haloalkyl, C₁₋₄ alkoxyl, —NRₐR_{b}, —C(═O)NRₐR_{b}, —S(═O)R_{b}, —S(═O)₂R_{b}, —S(═O)NRₐR_{b}, —S(═O)₂NRₐR_{b}, —NRₐC(═O)R_{b}, C₁₋₄ haloalkoxyl, C₃₋₆ cycloalkyl, C₃₋₆ heterocycloalkyl, C₅₋₆ aryl or C₅₋₆ heteroaryl, wherein the C₅₋₆ heteroaryl and C₃₋₆ heterocycloalkyl optionally comprising 1, 2 or 3 hetero atoms independently selected from N, S, or O.

In some embodiments, R₁ is H, oxo, hydroxyl, halogen, CN,—NO₂, —NR_{d}Rₑ, C₁₋₆ alkyl, C₂₋₆ alkenyl, C₂₋₆ alkynyl, C₁₋₆ haloalkyl, C₁₋₆ alkoxyl, —C(═O)NRₐR_{b},—C(═O)ORₐ, —C(═O)RR, —S(═O)R_{b}, —S(═O)₂R_{b}, —S(═O)NRₐR_{b}, —S(═O)₂NRₐR_{b}, —O—(C═O)—Rₐ, —O—(C═O)—NRₐR_{b}, C₁₋₆ haloalkoxyl, C₃₋₅ cycloalkyl, 3 to 5-membered heterocycloalkyl comprising for 2 N heteroatoms independently selected from N, S and O.

In some embodiments, R₁ is H, oxo, hydroxyl, halogen, CN, —NR_{d}Rₑ, C₁₋₆ alkyl, C₁₋₆ haloalkyl, C₁₋₆ alkoxyl, —C(═O)NRₐR_{b},—C(═O)ORₐ, —C(═O)R_{c}, —S(═O)₂NRₐR_{b}.

In some embodiments, R₁ is H, oxo, hydroxyl, halogen, CN, —N—(C₁₋₆ alkyl)₂, C₁₋₆ alkyl, C₁₋₄ haloalkyl, C₁₋₄ alkoxyl, —C(═O)NRₐR_{b}, —C(═O)ORₐ or —C(═O)R_{c}.

In some embodiments, R₁ is H, oxo, halogen, CN, —N—(C₁₋₆ alkyl)₂, C₁₋₆ alkyl, C₁₋₄ haloalkyl, C₁₋₄alkoxyl, —C(═O)O—C₁₋₄alkyl, or —C(═O)R_{c}.

In some embodiments, R₁ is H, oxo, halogen, —N—(C₁₋₃ alkyl)₂, C₁₋₆ alkyl, C₁₋₄ haloalkyl, C₁₋₄ alkoxyl, —C(═O)O—C₁₋₄alkyl, or —C(═O)R_{c}.

In some embodiments, R₁ is H, oxo, halogen, —N—(CH₃)₂, C₁₋₆ alkyl, C₁₋₄ haloalkyl, or C₁-4 alkoxyl.

In some embodiments, R₁ is H, oxo, halogen, —NRₐR_{b} or C₁₋₆ alkyl, wherein Rₐ is H or C₁₋₆ alkyl, R_{b} is C₁₋₆ alkyl.

In some embodiments, R₁ is H, oxo, C₁₋₆ alkyl.

In some embodiments, R₁ is H, oxo, C₁₋₃ alkyl.

In some embodiments, R₂ is H, hydroxyl, halogen, CN, —NO₂, —NRₐR_{b}, oxo, —C(═O)NRₐR_{b}, —C(═O)ORₐ, —C(═O)Rₐ, —S(═O)R_{b}, —S(═O)₂R_{b}, —S(═O)NRₐR_{b}, —S(═O)₂NRₐR_{b}, —NRₐC(═O)R_{b},—SF₅, C₁₋₆ alkyl, C₂₋₆ alkenyl, C₂₋₆ alkynyl, C₁₋₆ alkoxyl, C₁₋₆ haloalkyl; wherein the C₁₋₆ alkyl, C₂₋₆ alkenyl, C₂₋₆ alkynyl, C₁₋₆ alkoxyl, C₃₋₆ cycloalkyl are each optionally substituted with 0 to 3 substitutents independently selected from the group consisting of —ORₐ, —NH₂, halogen, CN, C₁₋₄ alkyl, C₁₋₆ haloalkyl, —NRₐR_{b}, oxo, —C(═O)NRₐR_{b}, —C(═O)ORₐ, —OC(═O)Rₐ, —C(═O)Rₐ, —S(═O)R_{b}, —S(═O)₂R_{b}, —S(═O)NRₐR_{b}, —S(═O)₂NRₐR_{b} and —NRₐC(═O)R_{b}.

In some embodiments, R₂ is H, hydroxyl, halogen, CN, —NRₐR_{b}, —NO₂, C₁₋₄ alkyl, C₂₋₄ alkenyl, C₂₋₄ alkynyl, C₁₋₄ alkoxyl, C₃₋₆ cycloalkyl, or C₃₋₆ heterocycloalkyl, wherein the C₃₋₆ heterocycloalkyl optionally comprising 1, 2 or 3 hetero atoms independently selected from N, S, or O; the C₁₋₄ alkyl, C₂₋₄ alkenyl, C₂₋₄ alkynyl, C₁₋₄ alkoxyl, C₃₋₆ cycloalkyl and C₃₋₆ heterocycloalkyl are each optionally substituted with one or more substitutents independently selected from hydroxyl, halogen, CN, —NH₂ or C₁₋₄ alkyl.

In some embodiments, R₂ is H, hydroxyl, halogen, CN, C₁₋₆ alkyl, C₂₋₆ alkenyl, C₂₋₆ alkynyl, C₁₋₆ alkoxyl, —SF₅, wherein the C₁₋₆ alkyl, C₂₋₆ alkenyl, C₂₋₆ alkynyl and C₁₋₆ alkoxyl are each optionally substituted with 0 to 3 substitutents independently selected from the group consisting of-ORₐ, halogen, CN, C₁₋₄ alkyl, C₁₋₆ haloalkyl, —NRₐR_{b}, oxo, —C(═O)NRₐR_{b}, —C(═O)ORₐ, —OC(═O)Rₐ, —C(═O)Rₐ, —S(═O)R_{b}, —S(═O)₂R_{b}, —S(═O)NRₐR_{b}, —S(═O)₂NRₐR_{b} and —NRₐC(═O)R_{b}.

In some embodiments, R₂ is hydroxyl, halogen, CN, C₁₋₆ alkyl, C₂₋₆ alkenyl, C₂₋₆ alkynyl, C₁₋₆ alkoxyl, —SF₅, wherein the C₁₋₆ alkyl, C₂₋₆ alkenyl, C₂₋₆ alkynyl and C₁₋₆ alkoxyl are each optionally substituted with 0 to 3 substitutents independently selected from the group consisting of-ORₐ, halogen, CN, C₁₋₄ alkyl, C₁₋₆ haloalkyl, —NRₐR_{b}, oxo, —C(═O)NRₐR_{b}, —C(═O)ORₐ, —C(═O)Rₐ, —S(═O)R_{b}, —S(═O)₂R_{b}, —S(═O)NRₐR_{b}, —S(═O)₂NRₐR_{b} and —NRₐC(═O)R_{b}; wherein the Rₐ and R_{b} are independently selected from the group consisting of H, CN, hydroxyl, halogen, C₁₋₆ alkyl.

In some embodiments, R₂ is H, hydroxyl, halogen, CN, C₁₋₆ alkyl, C₂₋₆ alkenyl, C₂₋₆ alkynyl, C₁₋₆ alkoxyl, C₁₋₆ haloalkyl; wherein the C₁₋₄ alkyl, C₂₋₄ alkenyl, C₂₋₄ alkynyl, C₁₋₄ alkoxyl are each optionally substituted with 0 to 3 substitutents independently selected from the group consisting of hydroxyl, halogen, CN and C₁₋₄ alkyl.

In some embodiments, R₂ is H, CN, halogen, C₁₋₆ alkyl, C₁₋₆ haloalkyl, C₁₋₆ haloalkoxyl.

In some embodiments, R₂ is H, CN, halogen, C₁₋₆ alkyl, C₁₋₆ haloalkyl.

In some embodiments, R₂ is H, CN, halogen, C₁₋₃ alkyl, C₁₋₃ haloalkyl.

In some embodiments, R₂ is H, halogen, C₁₋₄ alkyl, C₁₋₄ haloalkyl.

In some embodiments, R₂ is H, C₁₋₃ haloalkyl.

Preferably, R₂ is H or —CF₃.

Preferably, R₂ is H.

In some embodiments, R₃ is H, halogen, —ORₐ, CN, —NRₐR_{b}, —C₁₋₄ alkylene-NRₐR_{b}, —C₁₋₄ alkylene-NRₐC(═O)R_{b}, —C(═O)R_{b}, —OC(═O) Rₐ, —C(═O)ORₐ, —C(═O)NRₐR_{b}, —S(═O)R_{b}, —S(═O)₂R_{b}, —S(═O)NRₐR_{b}, —S(═O)₂NRₐR_{b}, —C₁₋₄ alkylene-C(═O)NRₐR_{b}, C₁₋₆ alkyl, C₁₋₆ alkoxyl, C₁₋₆ haloalkyl, C₁₋₆ haloalkoxyl, 3 to 6-membered heterocyclyl, C₃₋₆ cycloalkyl, C₅₋₆ aryl or 5 to 6-membered heteroaryl, wherein the 5 to 6-membered heteroaryl and 3 to 6-membered heterocyclyl optionally comprising 1, 2 or 3 hetero atoms independently selected from N, S and O; which C₁₋₆ alkyl, C₁₋₆ haloalkyl, C₁₋₆ alkoxyl, C₁₋₆ haloalkoxyl, C₃₋₆ cycloalkyl, 3 to 6-membered heterocyclyl, C₅₋₆ aryl and 5 to 6-membered heteroaryl are each optionally substituted with 0 to 3 substitutents independently selected from the group consisting of oxo, hydroxyl, halogen, CN, C₁₋₆ alkyl, —C(═O)R_{b}, —NRₐR_{b}, —C(═O)R_{b}, —C(═O)ORₐ, —C(═O)NRₐR_{b}.

In some embodiments, R₃ is H, oxo, halogen, —ORₐ, CN, —NO₂, —NRₐR_{b}, —NRₐC(═O)R_{b}, C₁₋₄ alkylene-NRₐR_{b}, —C₁₋₄ alkylene-NRₐC(═O)R_{b}, —C(═O)R_{b}, —C(═O)ORₐ, —C(═O)NRₐR_{b}, —S(═O)R_{b}, —S(═O)₂R_{b}, —S(═O)NRₐR_{b}, —S(═O)₂NRₐR_{b}, —C₁₋₄ alkylene-C(═O)NRₐR_{b}, C₁₋₆ alkyl, C₂₋₆ alkenyl, C₂₋₆ alkynyl, C₁₋₆ alkoxyl, C₁₋₄ haloalkyl, C₁₋₄ haloalkoxyl, C₃₋₆ heterocycloalkyl, C₃₋₆cycloalkyl, C₅₋₆ aryl or C₅₋₆ heteroaryl, wherein the C₅₋₆ heteroaryl and C₃₋₆ heterocycloalkyl optionally comprising 1, 2 or 3 hetero atoms independently selected from N, S, or O; the C₁₋₆ alkyl, C₂₋₆ alkenyl, C₂₋₆ alkynyl, C₁₋₄ haloalkyl, C₁₋₄ alkoxyl, C₁₋₄ haloalkoxyl, C₃₋₆cycloalkyl, C₃₋₆ heterocycloalkyl, C₅₋₆ aryl and C₅₋₆ heteroaryl are each optionally substituted with one or more substitutents independently selected from oxo, hydroxyl, halogen, CN, —NO₂, C₁₋₆ alkyl, —C₁₋₄ alkylene-OH, C₁₋₄ haloalkyl, C₁₋₄ alkoxyl, —S(═O)₂R_{b}, —C(═O)R_{b}, —NRₐR_{b}, —C(═O)R_{b}, —C(═O)ORₐ, —NRₐC(═O)R_{b}, —C(═O)NRₐR_{b}, —NRₐC(═O)R_{b}, —C₁₋₄ alkylene-NRₐR_{b}, —C₁₋₄ alkylene-NRₐC(═O)R_{b}, C₁₋₄ alkylene-C(═O)NRₐR_{b}, —C₁₋₄ alkylene-OH, C₃₋₆ cycloalkyl, C₃₋₆ heterocycloalkyl, C₅₋₆ aryl or C₅₋₆ heteroaryl.

In some embodiments, R₃ is H, halogen, —ORₐ, CN, —C₁₋₄ alkylene-NRₐR_{b}, —C₁₋₄ alkylene-C(═O)NRₐR_{b}, C₁₋₆ alkyl, C₁₋₆ alkoxyl, C₁₋₆ haloalkyl, C₁₋₆ haloalkoxyl, 3 to 6-membered heterocyclyl, C₃₋₆ cycloalkyl, C₅₋₆ aryl or 5 to 6-membered heteroaryl, wherein the 5 to 6-membered heteroaryl and 3 to 6-membered heterocyclyl optionally comprising 1, 2 or 3 hetero atoms independently selected from N, S and O; which C₁₋₆ alkyl, C₃₋₆ cycloalkyl, 3 to 6-membered heterocyclyl, C₅₋₆ aryl and 5 to 6-membered heteroaryl are each optionally substituted with 0 to 3 substitutents independently selected from the group consisting of halogen, CN, C₁₋₆ alkyl, C₁₋₆ haloalkyl, —NRₐR_{b}, —C(═O)ORₐ, —C(═O)NRₐR_{b}.

In some embodiments, R₃ is H, halogen, —ORₐ, CN, —C₁₋₄ alkylene-NRₐR_{b}, —C₁₋₄ alkylene-C(═O)NRₐR_{b}, C₁₋₆ alkyl, C₁₋₆ alkoxyl, C₁₋₆ haloalkyl, 3 to 6-membered heterocyclyl, C₃₋₆ cycloalkyl or 5 to 6-membered heteroaryl, wherein the 5 to 6-membered heteroaryl and 3 to 6-membered heterocyclyl optionally comprising 1, 2 or 3 hetero atoms independently selected from N, S and O; which C₁₋₆ alkyl, C₃₋₆ cycloalkyl, 3 to 6-membered heterocyclyl and 5 to 6-membered heteroaryl are each optionally substituted with 0 to 3 substitutents independently selected from the group consisting of halogen, CN, C₁₋₆ alkyl, —NRₐR_{b}, —C(═O)ORₐ, —C(═O)NRₐR_{b}.

In some embodiments, R₃ is H, halogen, —ORₐ, CN, —C₁₋₄ alkylene-NRₐR_{b}, —C₁₋₄ alkylene-C(═O)NRₐR_{b}, C₁₋₆ alkyl, C₁₋₆ alkoxyl, C₁₋₆ haloalkyl, 3 to 6-membered heterocyclyl, C₃₋₆ cycloalkyl or 5 to 6-membered heteroaryl, wherein the 5 to 6-membered heteroaryl and 3 to 6-membered heterocyclyl optionally comprising 1, 2 or 3 hetero atoms independently selected from N, S and O; which C₁₋₆ alkyl, C₃₋₆ cycloalkyl, 3 to 6-membered heterocyclyl and to 6-membered heteroaryl are each optionally substituted with 0 to 3 substitutents independently selected from the group consisting of halogen, CN, C₁₋₆ alkyl, —NRₐR_{b}, —C(═O)ORₐ, —C(═O)NRₐR_{b}; wherein Rₐ and R_{b} are independently selected from the group consisting of H and C₁₋₆ alkyl.

In some embodiments, R₃ is H, halogen, CN, —ORₐ, C₁₋₆ alkyl, C₁₋₆ haloalkyl, C₃₋₆ cycloalkyl, 3 to 6-membered heterocycloalkyl or 5 to 6-membered heteroaryl, wherein the 5 to 6-membered heteroaryl and 3 to 6-membered heterocycloalkyl optionally comprising 1, 2 or 3 hetero atoms independently selected from N and O; the C₁₋₆ alkyl, C₃₋₆ cycloalkyl, 3 to 6-membered heterocycloalkyl and 5 to 6-membered heteroaryl are each optionally substituted with the group consisting of C₁₋₆ alkyl, C₁₋₄ haloalkyl or halogen.

In some embodiments, R₃ is H, halogen, CN, —ORₐ, C₁₋₆ alkyl, C₁₋₆ haloalkyl, C₃-6cycloalkyl, 3 to 6-membered heterocycloalkyl or 5 to 6-membered heteroaryl, wherein the 5 to 6-membered heteroaryl and 3 to 6-membered heterocycloalkyl optionally comprising 1, 2 or 3 hetero atoms independently selected from N and O; the C₁₋₆ alkyl, C₃₋₆ cycloalkyl, 3 to 6-membered heterocycloalkyl and 5 to 6-membered heteroaryl are each optionally substituted with the group consisting of C₁₋₆ alkyl, C₁₋₄ haloalkyl or halogen; wherein Rₐ and R_{b} are independently selected from the group consisting of H and C₁₋₆ alkyl.

In some embodiments, R₃ is H, halogen, CN, C₁₋₆ alkyl, C₁₋₄ haloalkyl, C₁₋₆ alkoxyl, C₁₋₄ haloalkyl, —NRₐR_{b}, C₃₋₆ heterocycloalkyl, C₃₋₆cycloalkyl or C₅₋₆ heteroaryl; the C₃₋₆ cycloalkyl, C₃₋₆ heterocycloalkyl and C₅₋₆ heteroaryl are each optionally substituted with one or more substitutents independently selected from H, halogen or C₁₋₆ alkyl.

In some embodiments, R₃ is H, halogen, CN, —O—C₁₋₃ alkyl, C₁₋₃ alkyl, C₁₋₃ haloalkyl, C₃₋₅ cycloalkyl, 5 to 6-membered heteroaryl or 4 to 6-membered heterocycloalkyl, wherein the 5 to 6-membered heteroaryl and 4 to 6-membered heterocycloalkyl are each optionally substituted with C₁₋₆ alkyl or halogen.

Preferably, R₃ is H, halogen, CN, C₁₋₃ alkyl, —ORₐ.

Preferably, R₃ is H.

In some embodiments, L₃ is bond, —NH—,—N—C₁₋₃ alkyl-, —(CH₂)ₜ—NH—, —C₄₋₆ heterocyclyl.

In some embodiments, L₃ is bond or —NH—.

In some embodiments, R₅, R₆ and R₇ are independently selected from the group consisting of H, halogen, —ORₐ, CN, —NRₐR_{b}, —C₁₋₆ alkylene-NRₐR_{b}, —C₁₋₆ alkylene-R_{c}, C₁₋₆ alkyl, C₁₋₆ alkoxyl, C₃₋₆ cycloalkyl, 3 to 6-membered heterocyclyl, which C₁₋₆ alkyl, C₁₋₆ alkoxyl, C₃₋₆ cycloalkyl, 3 to 6-membered heterocyclyl are each optionally substituted with 0 to 4 substitutents independently selected from the group consisting of CN, halogen, C₁₋₆ alkyl, C₁₋₄ haloalkyl, —NRₐR_{b}, C₃₋₆ cycloalkyl, 3 to 6-membered heterocyclyl, wherein the 5 to 6-membered heteroaryl and 3 to 6-membered heterocyclyl optionally comprising 1, 2 or 3 hetero atoms independently selected from N, S and O.

In some embodiments, R₅, R₆ and R₇ are independently selected from the group consisting of H, halogen, CN, —C₁₋₆ alkylene-NRₐR_{b}, —C₁₋₆ alkylene-R_{c}, C₁₋₆ alkyl, C₁₋₆ alkoxyl, C₃₋₆ cycloalkyl, 3 to 6-membered heterocyclyl comprising 1, 2 or 3 hetero atoms independently selected from N, S and O.

In some embodiments, R₅, R₆ and R₇ are independently selected from the group consisting of H, halogen, CN, —C₁₋₆ alkylene-NRₐR_{b}, —C₁₋₆ alkylene-R_{c}, C₁₋₆ alkyl, C₁₋₆ alkoxyl, C₃₋₆ cycloalkyl, 3 to 6-membered heterocyclyl comprising 1, 2 or 3 hetero atoms independently selected from N, S and O; wherein Rₐ and R_{b} are independently selected from the group consisting of H and C₁₋₆ alkyl.

In some embodiments, R₅, R₆ and R₇ are independently selected from the group consisting of H, halogen, CN, C₁₋₆ alkyl, —C₁₋₆ alkylene-NRₐR_{b}, and —C₁₋₆ alkylene-R_{c}.

In some embodiments, R₅, R₆ and R₇ are independently selected from the group consisting of H, halogen, CN, C₁₋₆ alkyl, —C₁₋₆ alkylene-NRₐR_{b}, and —C₁₋₆ alkylene-R_{c}; wherein Rₐ and R_{b} are independently selected from the group consisting of H and C₁₋₆ alkyl.

In some embodiments, R₅, R₆ and R₇ are independently selected from the group consisting of H, halogen, CN, C₁₋₄ alkyl, and —C₁₋₄ alkylene-NRₐR_{b}.

In some embodiments, wherein,
R₅ is H, CN, C₁₋₄ alkyl or halogen;
one of R₆ and R₇ is H, and the other is H, halogen, C₁₋₄ alkyl or —C₁₋₄ alkylene-N(C₁₋₃ alkyl)₂.

R₅ is H, C₁₋₄ alkyl or halogen;
one of R₆ and R₇ is H, and the other is H, halogen, C₁₋₄ alkyl or —C₁₋₄ alkylene-N(C₁₋₃ alkyl)₂.

In some embodiments, wherein,
R₅ is H, C₁₋₄ alkyl or halogen;
one of R₆ and R₇ is H, and the other is H, C₁₋₄ alkyl or halogen.

In some embodiments, R₈ is H, halogen, CN, C₁₋₄ alkyl or —C₁₋₄ alkylene-NRₐR_{b}.

In some embodiments, R₈ is H, halogen or C₁₋₄ alkyl.

In some embodiments, R₈ is H or C₁₋₄ alkyl.

In some embodiments, R₈ is halogen.

In some embodiments, R₈ is C₁₋₄ alkyl.

In some embodiments, Rₐ and R_{b} are independently selected from the group consisting of H, C₁₋₆ alkyl, C₃₋₆ cycloalkyl, 3 to 6-membered heterocycloalkyl, C₅₋₆ aryl and 5 to 6-membered heteroaryl, wherein the C₁₋₆ alkyl, C₃₋₆ cycloalkyl, 3 to 6-membered heterocycloalkyl, C₅₋₆ aryl and 5 to 6-membered heteroaryl may optionally be substituted with halogen, C₁₋₆haloalkyl or C₅₋₆ aryl, wherein the 5 to 6-membered heteroaryl and 3 to 6-membered heterocycloalkyl optionally comprising 1, 2 or 3 hetero atoms independently selected from N, S and O.

In some embodiments, Rₐ is selected from H, CN, C₁₋₆ alkyl, C₂₋₆ alkenyl, C₂₋₆ alkynyl, C₃₋₆ cycloalkyl or C₃₋₆ heterocyclyl.

In some embodiments, Rₐ is H or C₁₋₆ alkyl.

In some embodiments, R_{b} is H, hydroxyl, halogen, CN, C₁₋₆ alkyl, —O—C₁₋₆ alkyl, C₂₋₆ alkenyl, C₂₋₆ alkynyl, C₁₋₃ haloalkyl, C₁₋₆ alkoxyl, C₁₋₆ haloalkoxyl, C₃₋₄ cycloalkyl, C₃₋₄ heterocycloalkyl, C₅₋₆ aryl or C₅₋₆ heteroaryl.

In some embodiments, R_{b} is selected from the group consisting of H, C₁₋₆ alkyl, C₃₋₆ cycloalkyl, halogen substituted C₃₋₆ cycloalkyl, 3 to 6-membered heterocyclyl and halogen or C₁₋₄ haloalkyl substituted 3 to 6-membered heterocyclyl.

In some embodiments, R_{b} is H, C₁₋₆ alkyl or C₃₋₆ heterocyclyl.

In some embodiments, Rₐ and R_{b} are independently selected from the group consisting of H and C₁₋₆ alkyl.

In some embodiments, R_{d} is H, C₁₋₆ alkyl.

In some embodiments, Rₑ is C₁₋₆ alkyl, —C(═O)R_{c}, S(═O)₂R_{b}.

In some embodiments, R_{c} is 3 to 6-membered heterocycloalkyl which may be substituted with halogen or C₁₋₆ haloalkyl.

In some embodiments, t is 1.

In some embodiments, y is 1 or 2.

In some embodiments, m is 1 or 2.

In some embodiments, x is 1.

In some embodiments, ring A is selected from the group consisting of

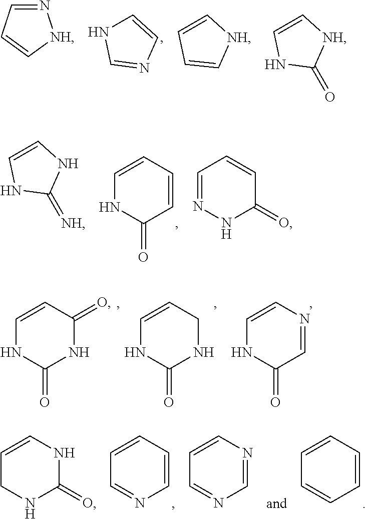

In some embodiments, ring B is selected from the group consisting of

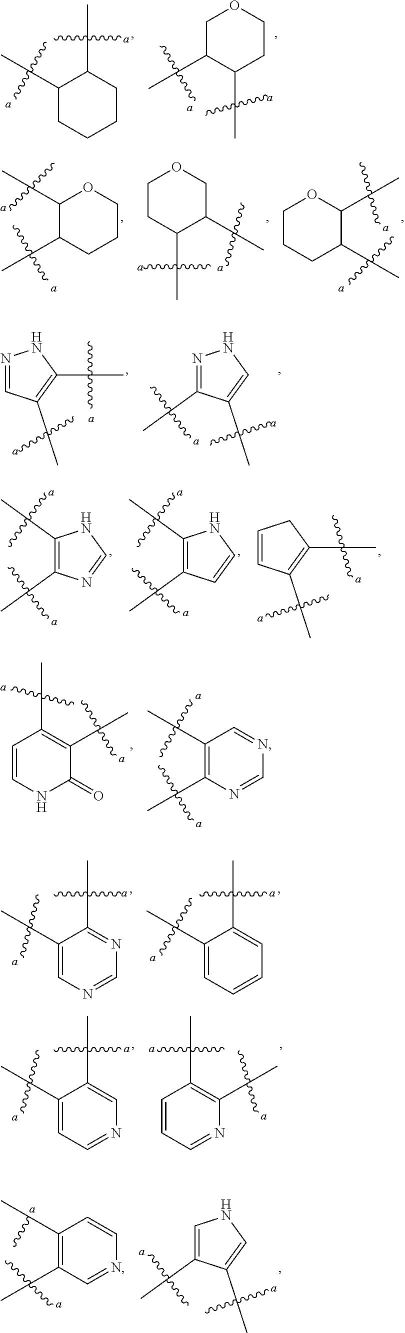

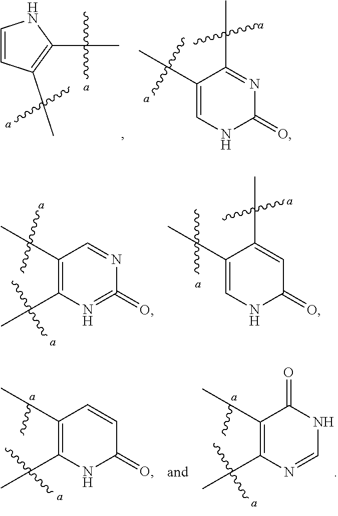

In some embodiments, ring B is selected from the group consisting of

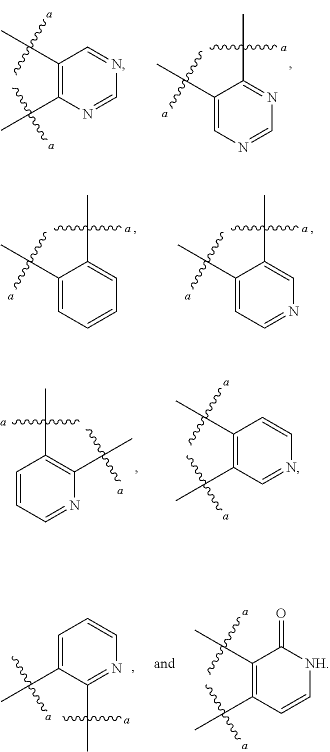

In some embodiments, ring

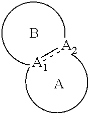

is selected from the group consisting or

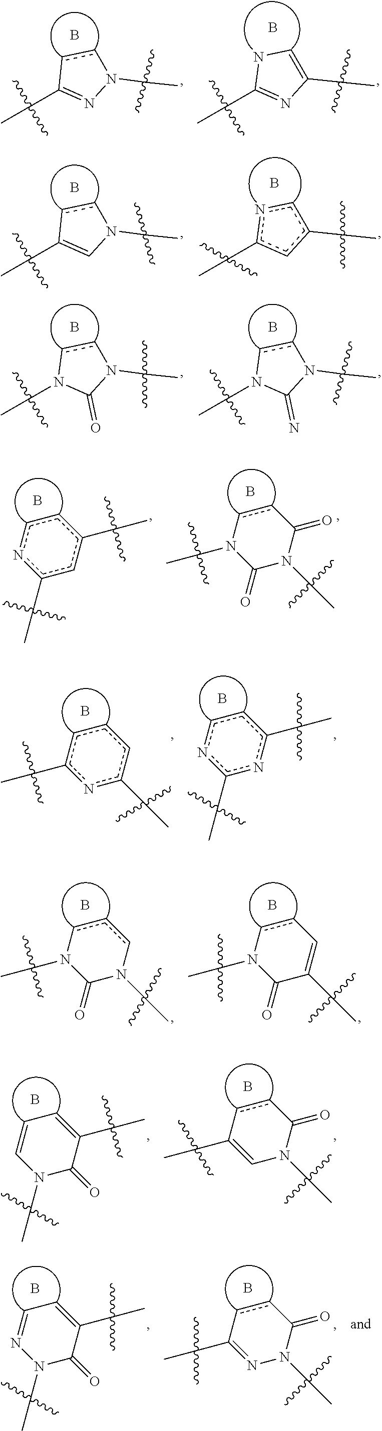

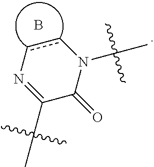

In some embodiments, ring

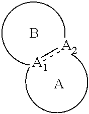

is selected from the group consisting of

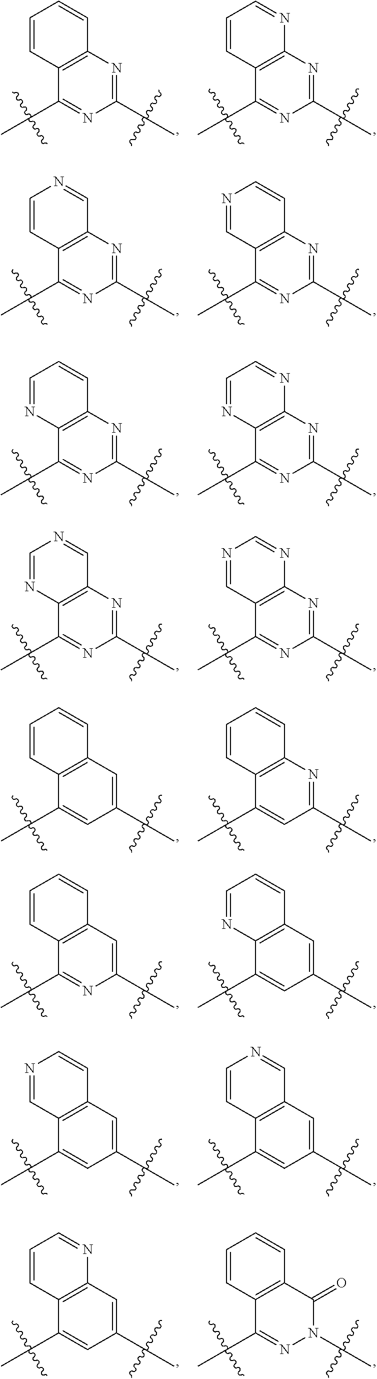

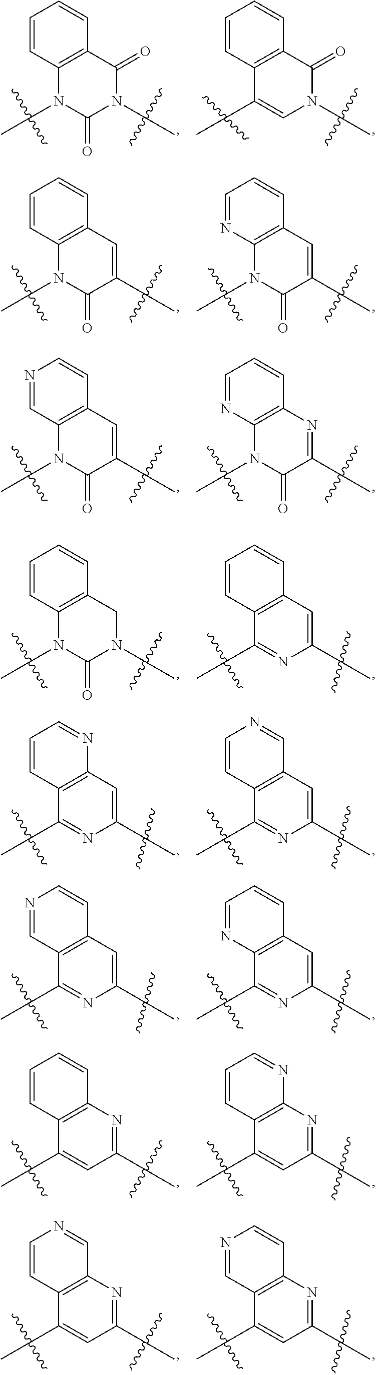

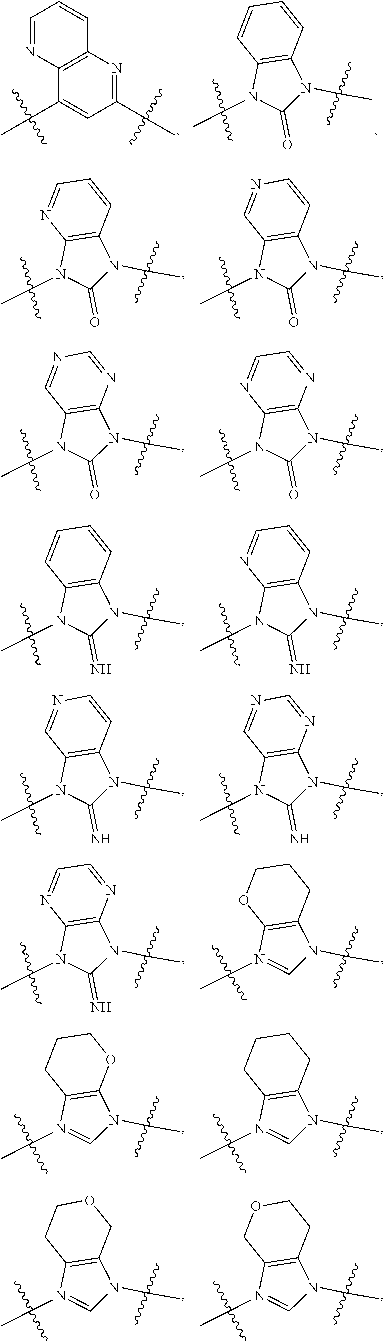

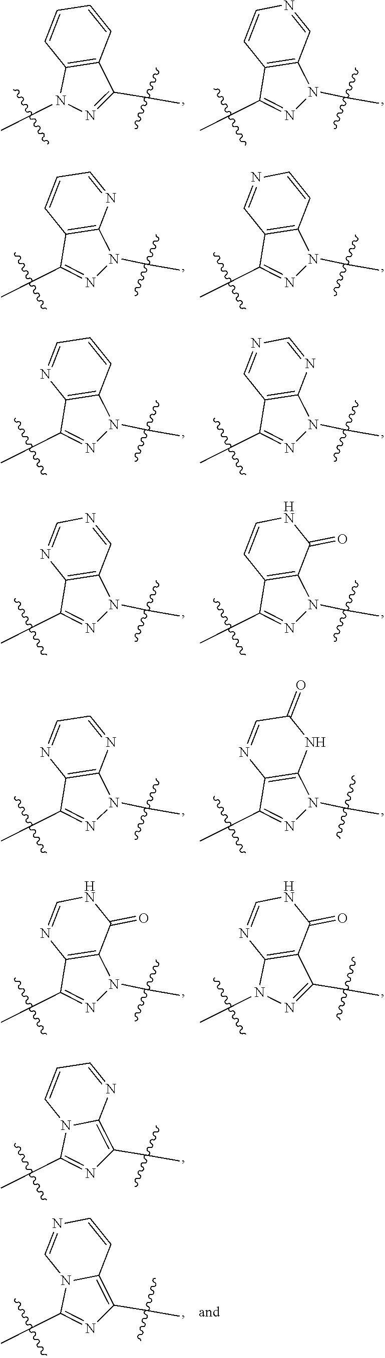

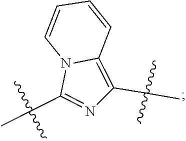 [SMILES: *c1nc(*)n2ccccc12]

wherein the

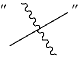

represents a site which is attached to L₁ or L₂.

In some embodiments, ring

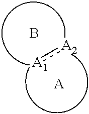

is selected from the group consisting of

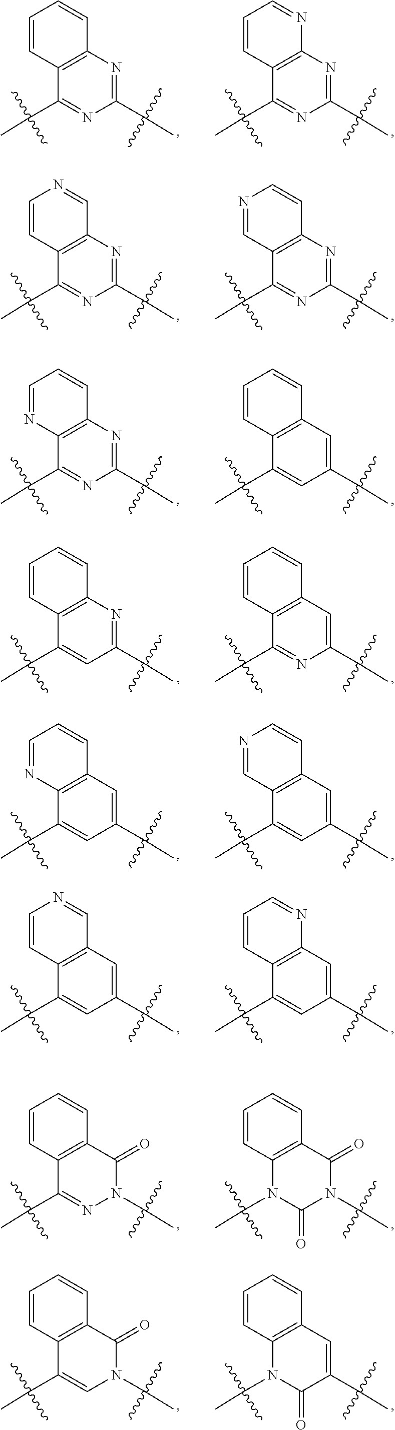

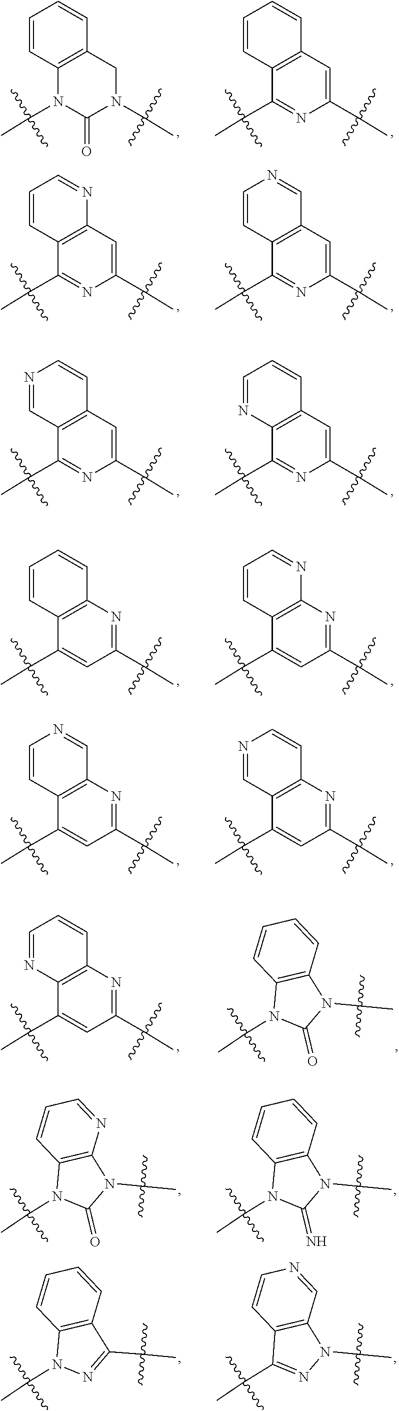

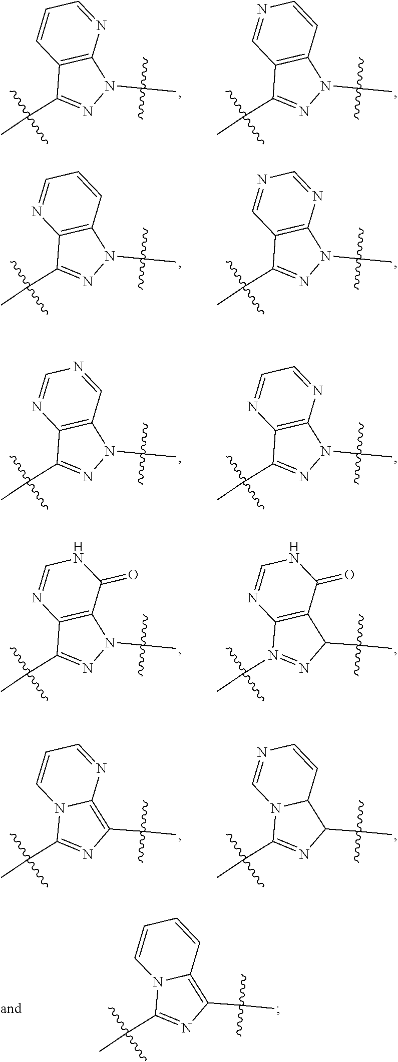

wherein the

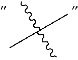

represents a site which is attached to L₁ or L₂.

In some embodiments, ring

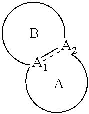

is selected from the group consisting of

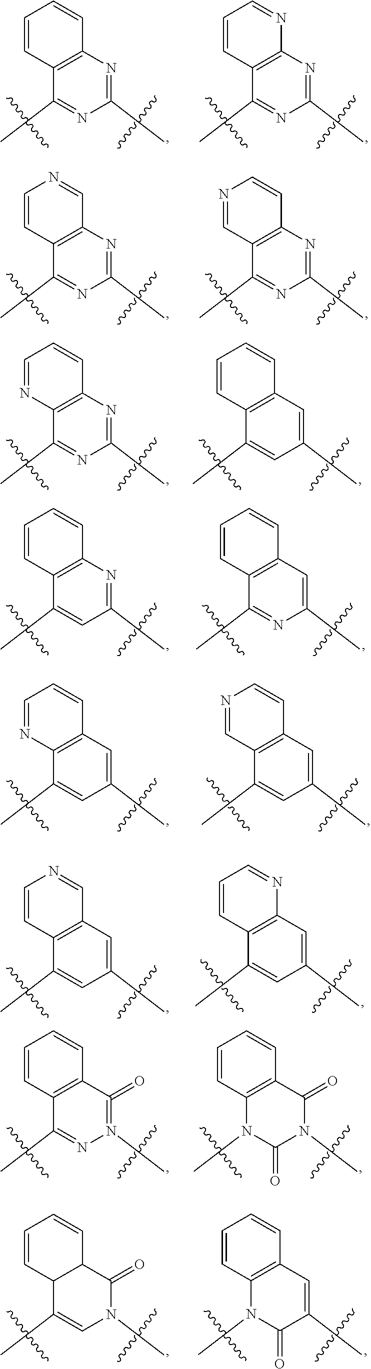

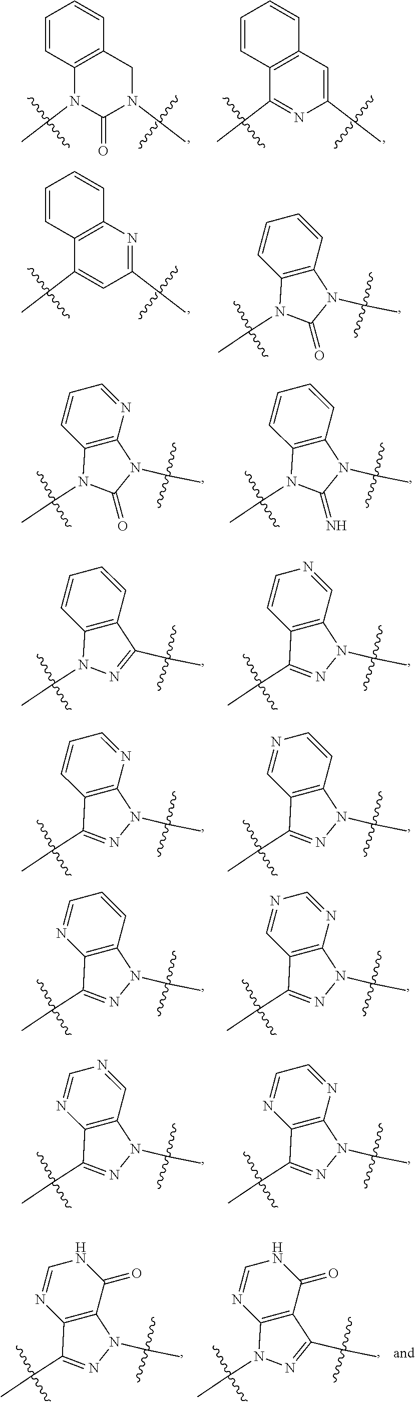

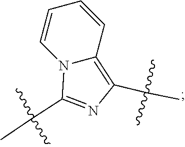 [SMILES: *c1nc(*)n2ccccc12]

wherein the

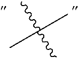

represents a site which is attached to L₁ or L₂.

In some embodiments, ring D is selected from the group consisting of

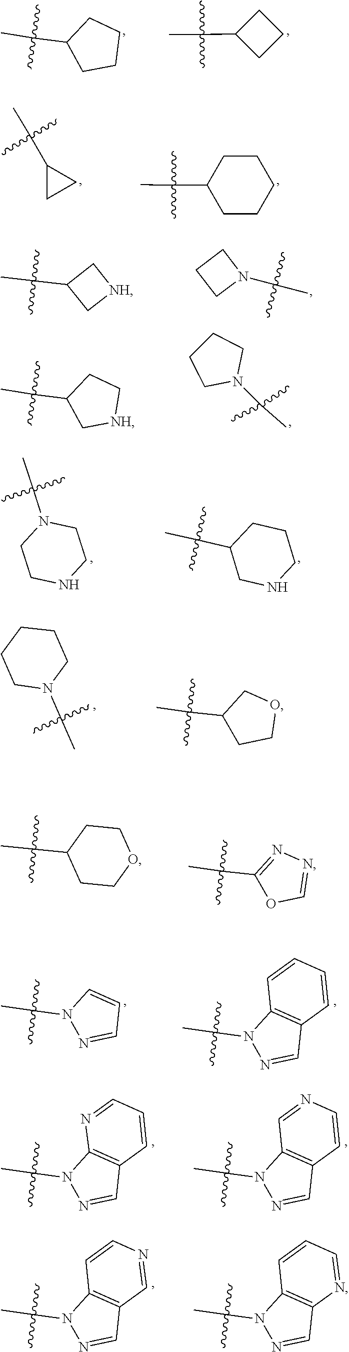

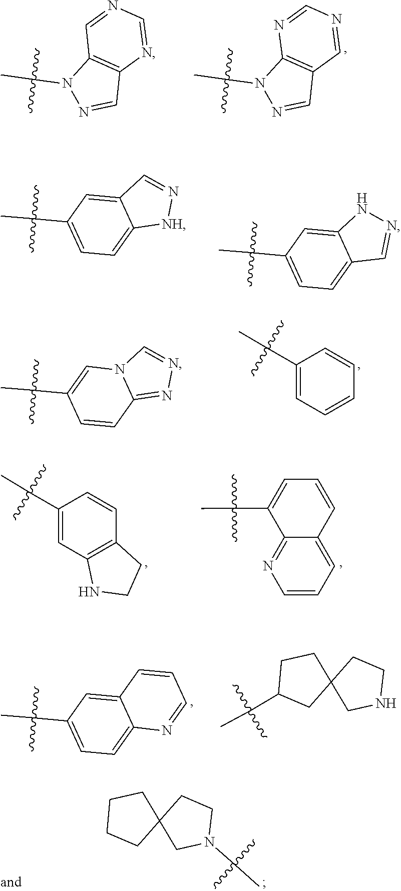

wherein the

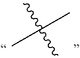

represents a site which is attached to L₁.

In some embodiments, ring D is selected from the group consisting of

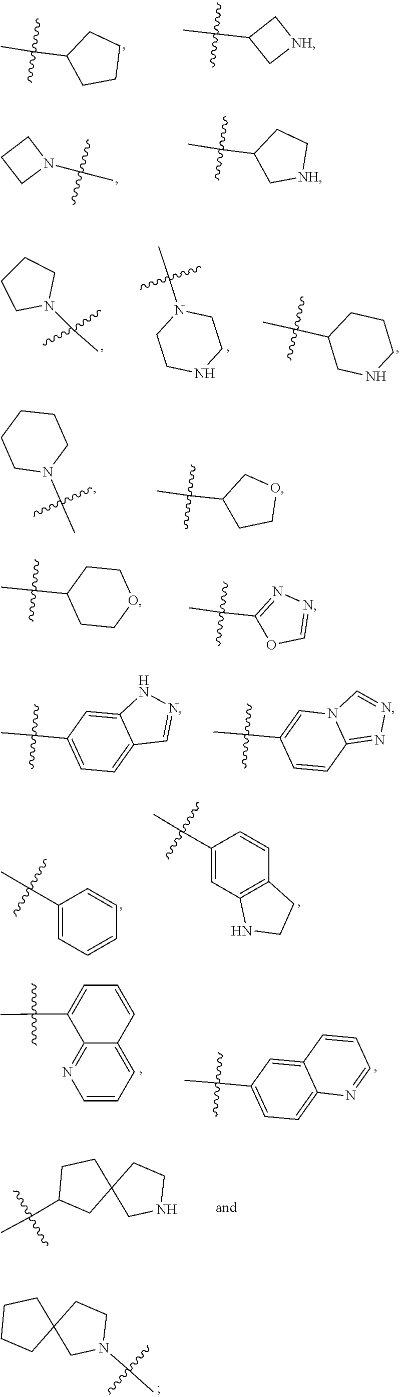

wherein the

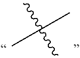

represents a site which is attached to L₁.

In some embodiments, ring E is selected from the group consisting of

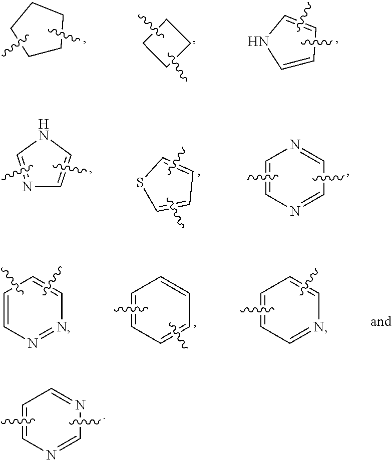

In some embodiments, the compound is of Formula (II-1) or Formula (II-2),

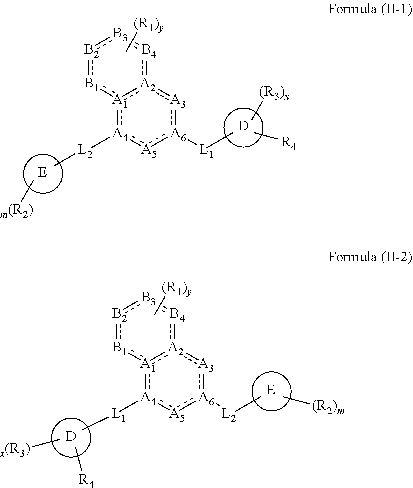

or a stereoisomer, tautomer, pharmaceutically acceptable salt, prodrug, chelate, non-covalent complex, or solvate thereof, wherein,
A₁, A₂, A₄ and A₆ are independently C or N;
A₃ is absent, CH, CH₂, C═O or N;
A₅ is CH, CH₂, C═O, C═NH or N;
B₁, B₂, B₃ and B₄ are independently selected from the group consisting of C, CH, CH₂, C═O, NH or N;
each ring E, ring D, L₁, L₂, R₁, R₂, R₃, R₄, m, y and x are as defined in embodiments and classes and subclasses herein.

In some embodiments, the compound is of Formula (III),

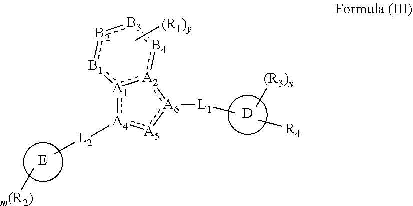

or a stereoisomer, tautomer, pharmaceutically acceptable salt, prodrug, chelate, non-covalent complex, or solvate thereof, wherein,
A₁, A₂, A₄ and A₆ are independently C or N;
As is CH, CH₂, N, C═O or C═NH;
B₁, B₂, B₃ and B₄ are independently selected from the group consisting of C, CH, CH₂, C═O, NH or N;
each ring E, ring D, L₁, L₂, R₁, R₂, R₃, R₄, m, y and x are as defined in embodiments and classes and subclasses herein.

In some embodiments of Formula (III), at least one of A₁, A₂, A₄, A₅ and A₆ is N.

In some embodiments, the compound is of Formula (IV-1) or Formula (IV-2),

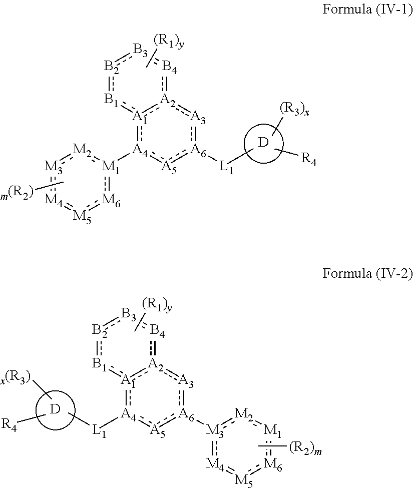

or a stereoisomer, tautomer, pharmaceutically acceptable salt, prodrug, chelate, non-covalent complex, or solvate thereof, wherein,
A₁, A₂, A₄ and A₆ are independently C or N;
A₃ is absent, CH₂, CH, C═O or N;
A₅ is CH, CH₂, C═O, C═NH or N;
B₁, B₂, B₃ and B₄ are independently selected from the group consisting of C, CH, CH₂, C═O, NH or N;
M₁, M₂, M₃, M₄, M₅ and M₆ are independently selected from the group consisting of C, CH or N;
each ring D, L₁, R₁, R₂, R₃, R₄, m, y and x are as defined in embodiments and classes and subclasses herein.

In some embodiments, the compound is of Formula (VI),

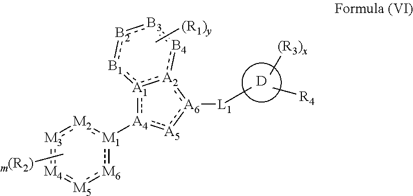

or a stereoisomer, tautomer, pharmaceutically acceptable salt, prodrug, chelate, non-covalent complex, or solvate thereof, wherein,
each A₁, A₂, A₃, A₄, A₅, A₆, B₁, B₂, B₃, B₄, M₁, M₂, M₃, M₄, M₅, M₆, ring D, L₁, R₁, R₂, R₃, R₄, m, y and x are as defined in embodiments and classes and subclasses herein.

In some embodiments, a compound of formula V, or a stereoisomer, tautomer, pharmaceutically acceptable salt, prodrug, chelate, non-covalent complex, or solvate thereof,

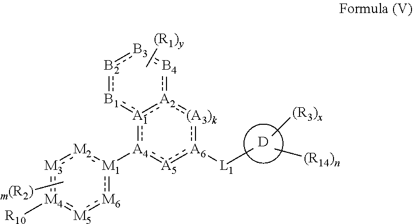

wherein,
[symbol] is a single bond or a double bond;
A₃ is CR₁₁ or NR₁₁;
A₁, A₂, A₄ and A₆ are independently selected from C or N;
A₅ is CR₁₅ or NR₁₅;
B₁, B₂, B₃ and B₄ are independently selected from C or N;
M₁, M₂, M₃, M₄, M₅ and M₆ are independently selected from C or N;
k is 0 or 1; and when k is 0, at least one of A₁, A₂, A₄, A₅ or A₆ is N;
R₁₁ is absent, H, oxo, hydroxyl, halogen, CN, —NH₂, —NO₂, ═NH, C₁₋₆ alkyl, C₁₋₄ haloalkyl, C₁₋₄ alkoxyl or C₁₋₄ haloalkoxyl;
R₁₅ is absent, H, oxo, hydroxyl, halogen, CN, —NO₂, —NH₂, ═NH, C₁₋₃ alkyl, C₂₋₄ alkenyl, C₂₋₄ alkynyl, C₁₋₃ haloalkyl, C₁₋₃ alkoxyl, C₁₋₃ haloalkoxyl, C₃₋₄ cycloalkyl or C₃₋₄ heterocycloalkyl;
R₁₀ is halogen, C₁₋₃ alkyl, C₂₋₆ alkenyl, C₂₋₆ alkynyl, C₃₋₆ cycloalkyl, C₃₋₆ halocycloalkyl, or C₅₋₆ aryl; the C₁₋₃ alkyl, C₂₋₆ alkenyl, C₂₋₆ alkynyl, C₃₋₆ cycloalkyl and C₅₋₆ aryl are each optionally substituted with halogen;
R₁₄ is —NRₐC(═O)R_{z}, —C₁₋₄ alkylene-NRₐC(═O)R_{z}, —C(═O)R₂, C₁₋₄ alkylene-C(═O)R_{z}, C₃₋₆ heterocycloalkyl-C(═O)R_{z}, C₃₋₆ heterocycloalkyl-NRₐC(═O)R_{z}, C₃₋₆cycloalkyl-NRₐC(═O)R_{z}, C₅₋₆ aryl-NRₐC(═O)R_{z} or C₅₋₆ heteroaryl-NRₐC(═O)R_{z} or C₃₋₆cycloalkyl-C(═O)R_{z}, wherein the C₅₋₆ heteroaryl and C₃₋₆ heterocycloalkyl optionally comprising 1, 2 or 3 hetero atoms independently selected from N, S, or O; the C₃₋₆ cycloalkyl, C₃₋₆ heterocycloalkyl, C₅₋₆ aryl and C₅₋₆ heteroaryl are each optionally substituted with one or more substitutents independently selected from oxo, hydroxyl, halogen, CN, —NO₂, C₁₋₆ alkyl, C₂₋₆ alkenyl, C₂₋₆ alkynyl, —C₁₋₄ alkylene-OH, C₁₋₄ haloalkyl, C₁₋₄ alkoxyl, —S(═O)₂R_{b}, —NRₐR_{b}, —C(═O)ORₐ, —C(═O)NRₐR_{b}, —C₁₋₄ alkylene-NRₐR_{b}, —C₁₋₄ alkylene-NRₐC(═O)R_{b}, C₁₋₄ alkylene-C(═O)NRₐR_{b}, C₃₋₆ cycloalkyl, C₃₋₆ heterocycloalkyl, C₅₋₆ aryl or C₅₋₆ heteroaryl;
R_{z} is C₂₋₆ alkenyl or C₂₋₆ alkynyl, the C₂₋₆ alkenyl and C₂₋₆ alkynyl are each optionally substituted with one or more substitutents independently selected from H, oxo, CN, halogen, —ORₐ, —NO₂, —NRₐR_{b}, —S(═O)R_{b}, —S(═O)₂R_{b}, —S(═O)NRₐR_{b}, —S(═O)₂NRₐR_{b}, C₁₋₆ haloalkyl, C₁₋₆ haloalkoxyl, C₃₋₆ cycloalkyl, C₃₋₆ heterocycloalkyl, C₅₋₆ aryl or C₅₋₆ heteroaryl;
t and n are each independently selected from 1, 2, 3 or 4;
y, m and x are each independently selected from 0, 1, 2, 3, 4 or 5; and
with the proviso that

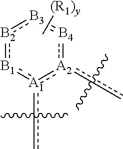

is not

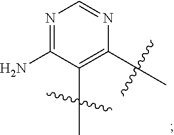 [SMILES: *c1ncnc(N)c1*]

each ring D, L₁, R₁, R₂, R₃ are as defined in embodiments and classes and subclasses herein.

In some embodiments, when A₃ is absent, at least one of A₁, A₂, A₄, A₅ and A₆ is N.

In some embodiments, R₁₄ is —NRₐC(═O)R_{z}, —C₁₋₄ alkylene-NRₐC(═O)R_{z}, —C(═O)R_{z} or C₃₋₆ heterocycloalkyl-C(═O)R_{z}, wherein the C₃₋₆ heterocycloalkyl optionally comprising 1, 2 or 3 hetero atoms independently selected from N, S, or O.

In some embodiments, R₁₅ is absent, H, oxo or ═NH.

In some embodiments, R_{z} is C₂₋₆ alkenyl or C₂₋₆ alkynyl, the C₂₋₆ alkenyl and C₂₋₆ alkynyl are each optionally substituted with one or more substitutents independently selected from H, CN, halogen, —ORₐ, C₃₋₆ cycloalkyl or —NRₐR_{b}.

In some embodiments, R_{z} is C₂₋₆ alkenyl or C₂₋₆ alkynyl, the C₂₋₆ alkenyl and C₂₋₆ alkynyl are each optionally substituted with one or more substitutents independently selected from H, halogen or —NRₐR_{b}.

In some embodiments, R_{z} is C₂₋₆ alkenyl or C₂₋₆ alkynyl, the C₂₋₆ alkenyl and C₂₋₆ alkynyl are each optionally substituted with —NRₐR_{b}.

In some embodiments, R₁₀ is C₁₋₃ haloalkyl.

In some embodiments, R₁₀ is —CF₃.

In some embodiments, R₁₁ is H, oxo, C₁₋₃alkyl.

In some embodiments, R₁₁ is H.

In some embodiments, the compound is of Formula (VI),

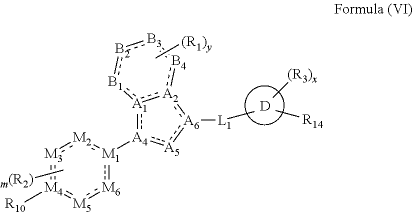

or a stereoisomer, tautomer, pharmaceutically acceptable salt, prodrug, chelate, non-covalent complex, or solvate thereof, wherein,
each A₁, A₂, A₄, A₅, A₆, B₁, B₂, B₃, B₄, M₁, M₂, M₃, M₄, M₅, M₆, ring D, L₁, R₁, R₂, R₃, R₁₀, R₁₄, m, y and x are as defined in embodiments and classes and subclasses herein.

In some embodiments, a compound disclosed herein is of the one of formulas:

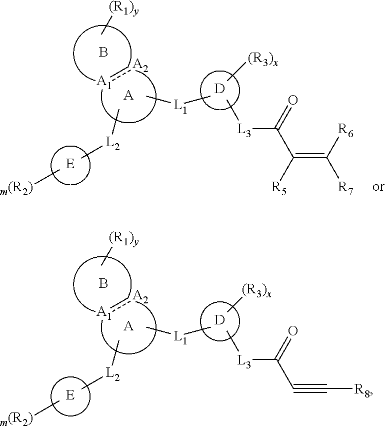

or a stereoisomer, tautomer, pharmaceutically acceptable salt, prodrug, chelate, non-covalent complex, or solvate thereof, wherein each ring A, ring B, ring E, ring D, L₁, L₂, L₃, R₁, R₂, R₃, R₅, R₆, R₇, R₈, m, y and x are as defined in embodiments and classes and subclasses herein.

In some embodiments, a compound disclosed herein is of the one of formulas:

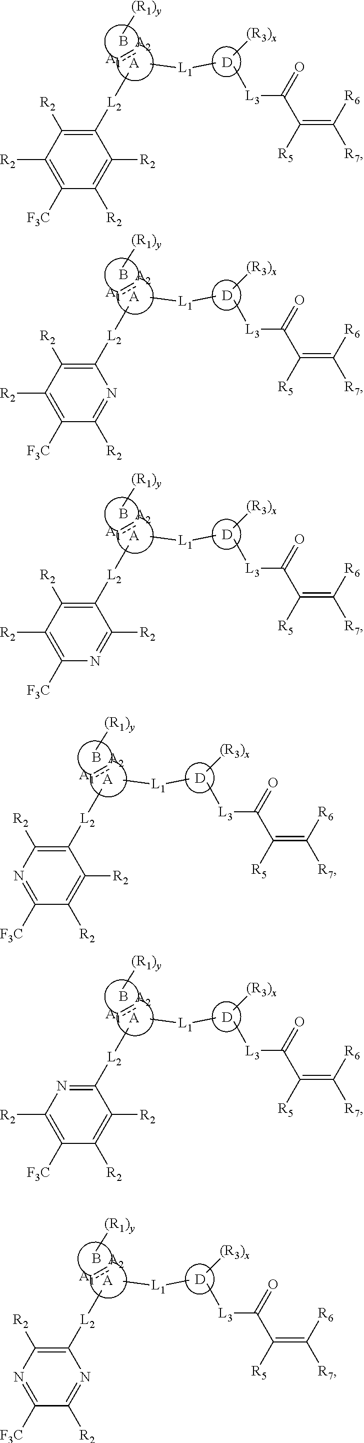

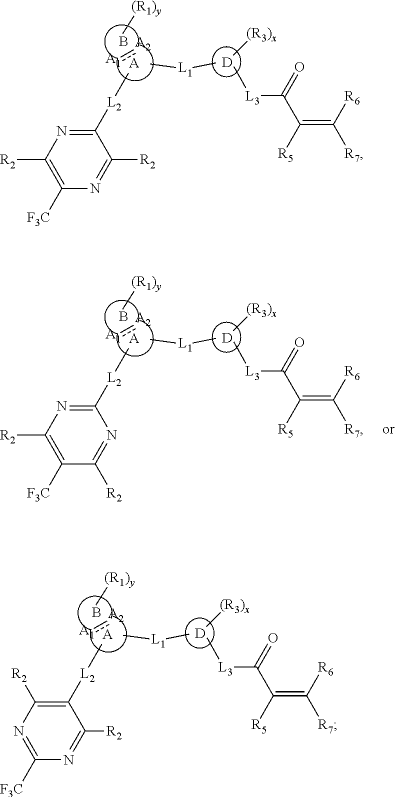

or a stereoisomer, tautomer, pharmaceutically acceptable salt, prodrug, chelate, non-covalent complex, or solvate thereof, wherein each ring A, ring B, ring D, L₁, L₂, L₃, R₁, R₂, R₃, R₅, R₆, R₇, y and x are as defined in embodiments and classes and subclasses herein.

In some embodiments, a compound disclosed herein is of the one of formulas:

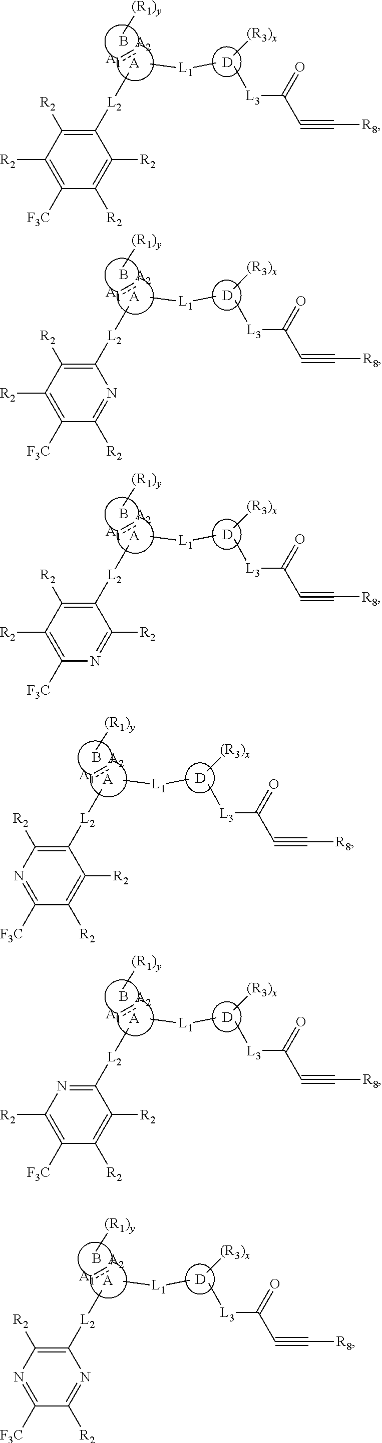

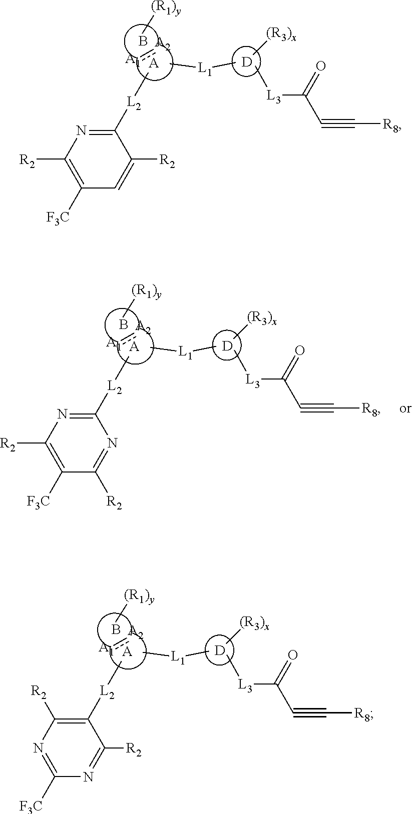

or a stereoisomer, tautomer, pharmaceutically acceptable salt, prodrug, chelate, non-covalent complex, or solvate thereof, wherein each ring A, ring B, ring D, L₁, L₂, L₃, R₁, R₂, R₃, R₈, y and x are as defined in embodiments and classes and subclasses herein.

In some embodiments, a compound disclosed herein is of the one of formulas:

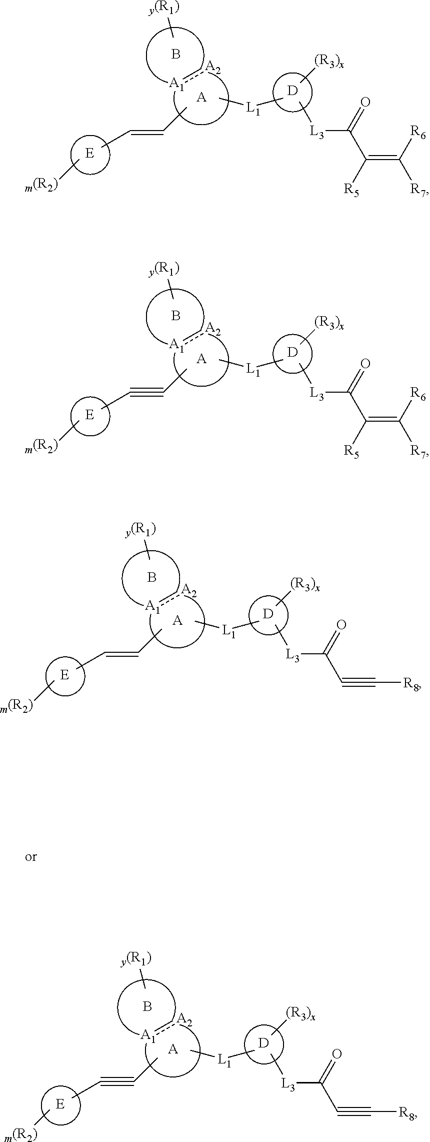

or a stereoisomer, tautomer, pharmaceutically acceptable salt, prodrug, chelate, non-covalent complex, or solvate thereof, wherein each ring A, ring B, ring E, ring D, L₁, L₃, R₁, R₂, R₃, R₅, R₆, R₇, R₈, m, y and x are as defined in embodiments and classes and subclasses herein.

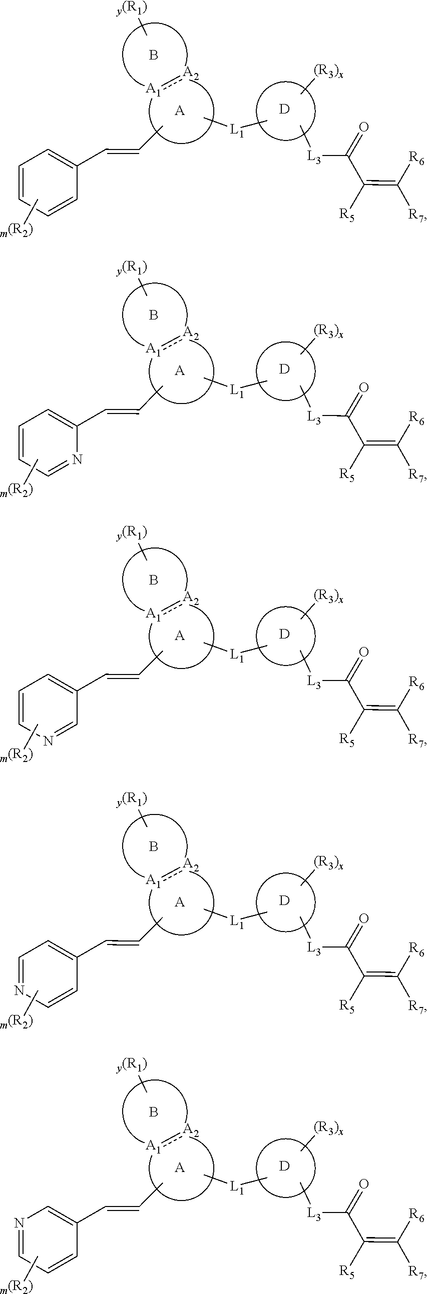

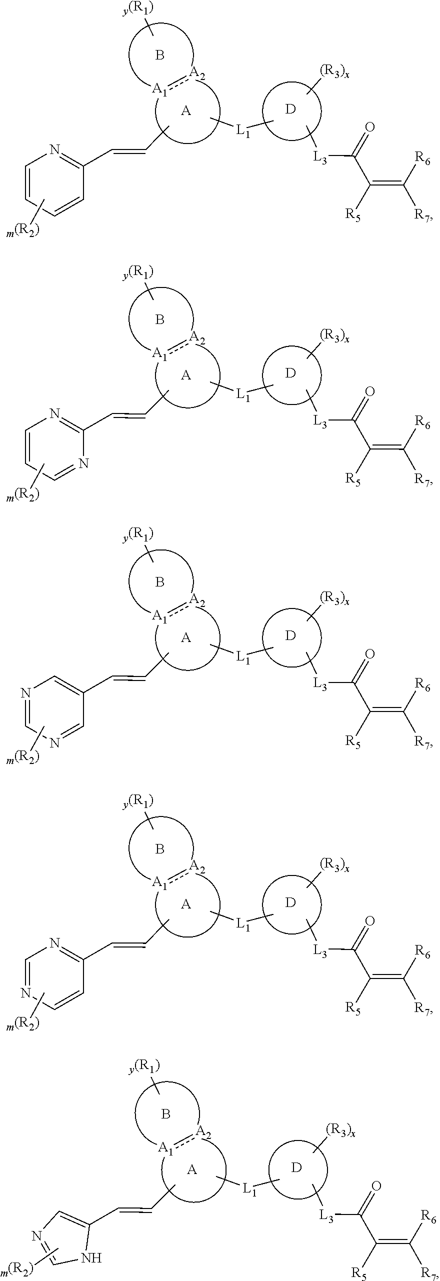

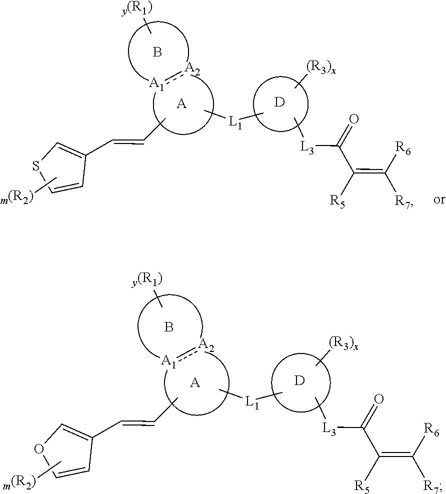

or a stereoisomer, tautomer, pharmaceutically acceptable salt, prodrug, chelate, non-covalent complex, or solvate thereof, wherein each ring A, ring B, ring D, L₁, L₃, R₁, R₂, R₃, R₅, R₆, R₇, R₈, m, y and x are as defined in embodiments and classes and subclasses herein.

In some embodiments, a compound disclosed herein is of the one of formulas:

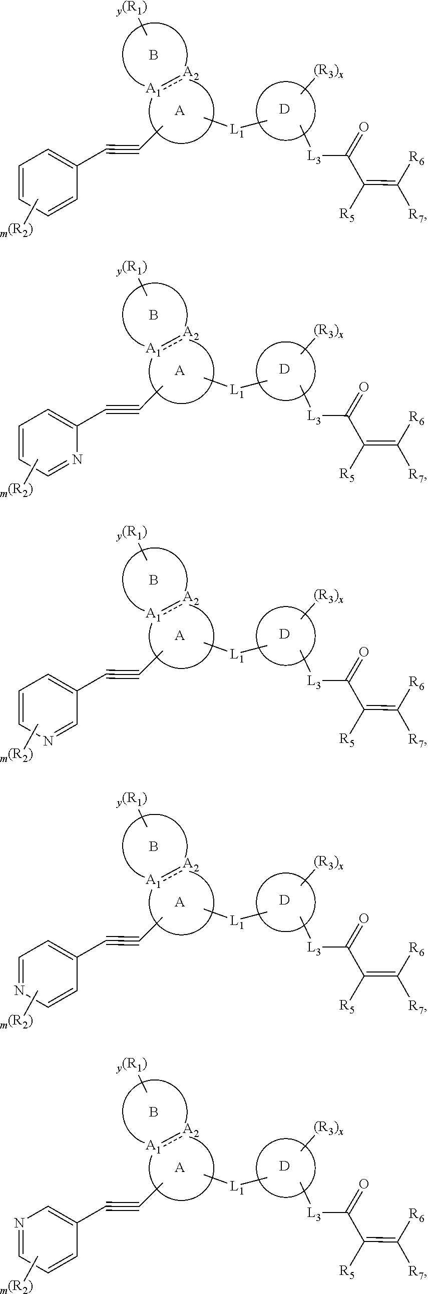

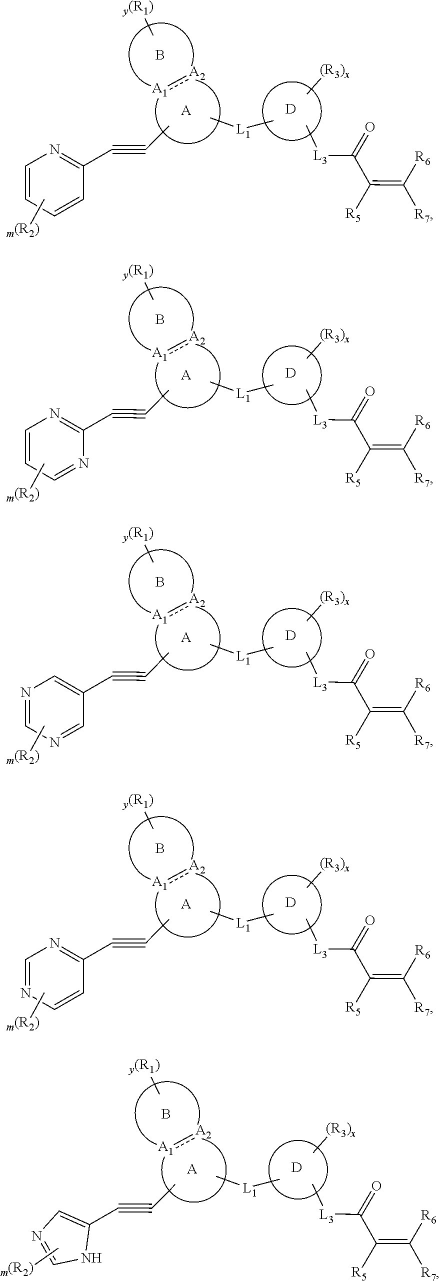

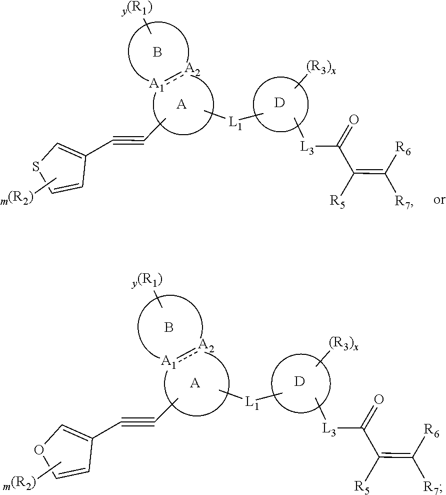

or a stereoisomer, tautomer, pharmaceutically acceptable salt, prodrug, chelate, non-covalent complex, or solvate thereof, wherein each ring A, ring B, ring D, L₁, L₃, R₁, R₂, R₃, R₅, R₆, R₇, R₈, m, y and x are as defined in embodiments and classes and subclasses herein.

In some embodiments, a compound disclosed herein is of the one of formulas:

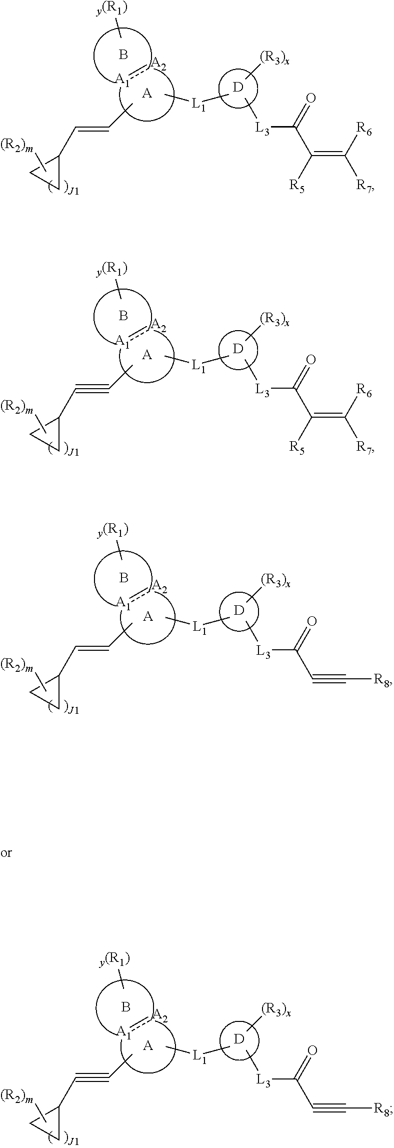

or a stereoisomer, tautomer, pharmaceutically acceptable salt, prodrug, chelate, non-covalent complex, or solvate thereof,
wherein J1 is 1, 2, 3 or 4, each ring A, ring B, ring D, L₁, L₃, R₁, R₂, R₃, R₅, R₆, R₇, R₈, m, y and x are as defined in embodiments and classes and subclasses herein.

In some embodiments, a compound disclosed herein is of the one of formulas:

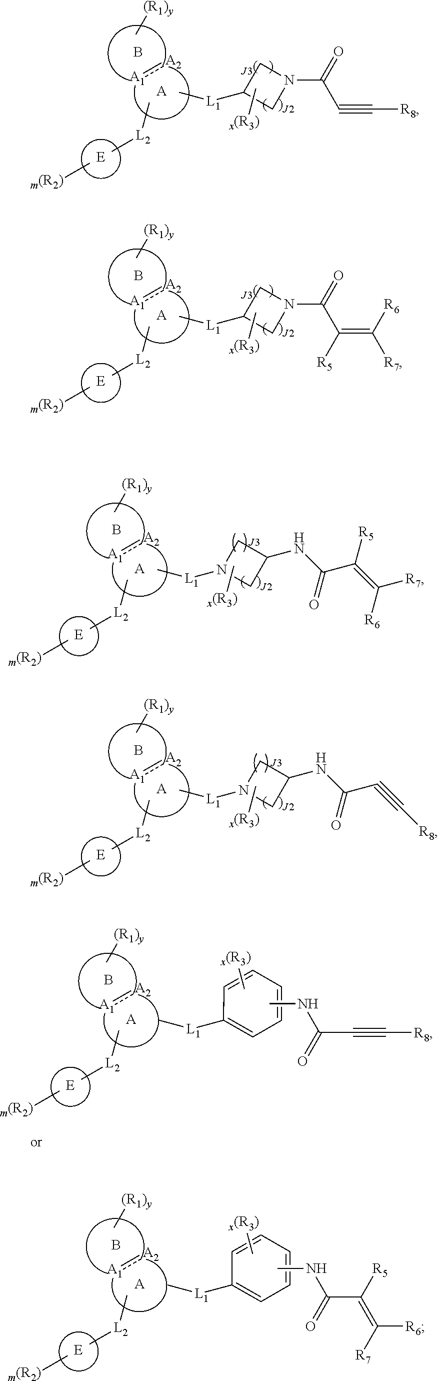

or a stereoisomer, tautomer, pharmaceutically acceptable salt, prodrug, chelate, non-covalent complex, or solvate thereof,
wherein J2 and J3 are independently 1 or 2, each ring A, ring B, ring E, L₁, L₂, R₁, R₂, R₃, R₅, R₆, R₇, R₈, m, y and x are as defined in embodiments and classes and subclasses herein.

In some embodiments, a compound disclosed herein is of the one of formulas:

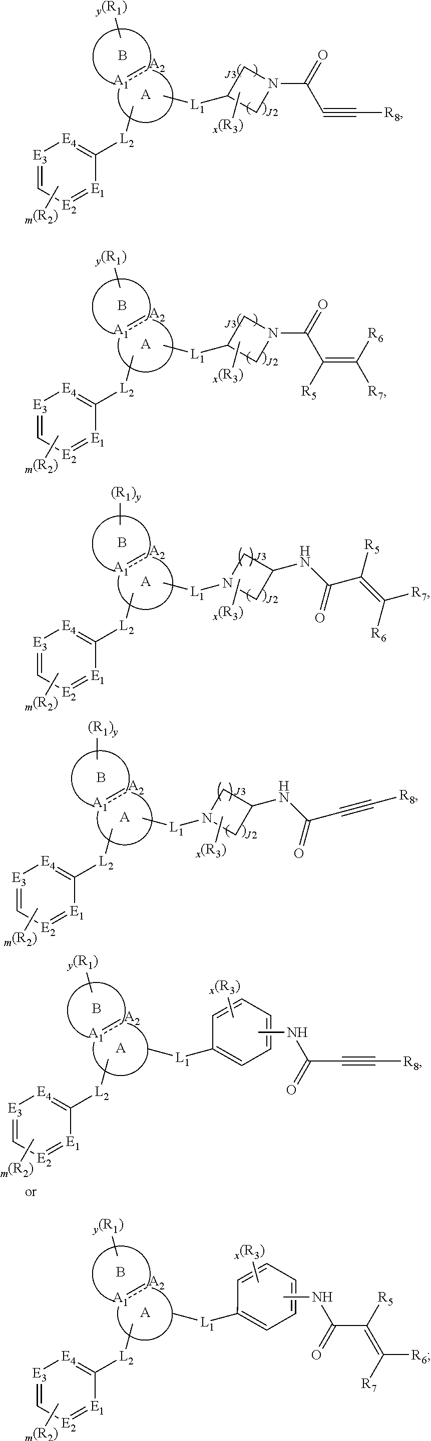

or a stereoisomer, tautomer, pharmaceutically acceptable salt, prodrug, chelate, non-covalent complex, or solvate thereof, wherein,
J2 and J3 are independently 1 or 2;
E₁, E₂, E₃ and E₄ are independently CH or N, and at most only one or two are N; each ring A, ring B, L₁, L₂, R₁, R₂, R₃, R₅, R₆, R₇, R₈, m, y and x are as defined in embodiments and classes and subclasses herein.

In some embodiments, a compound disclosed herein is of the one of formulas:

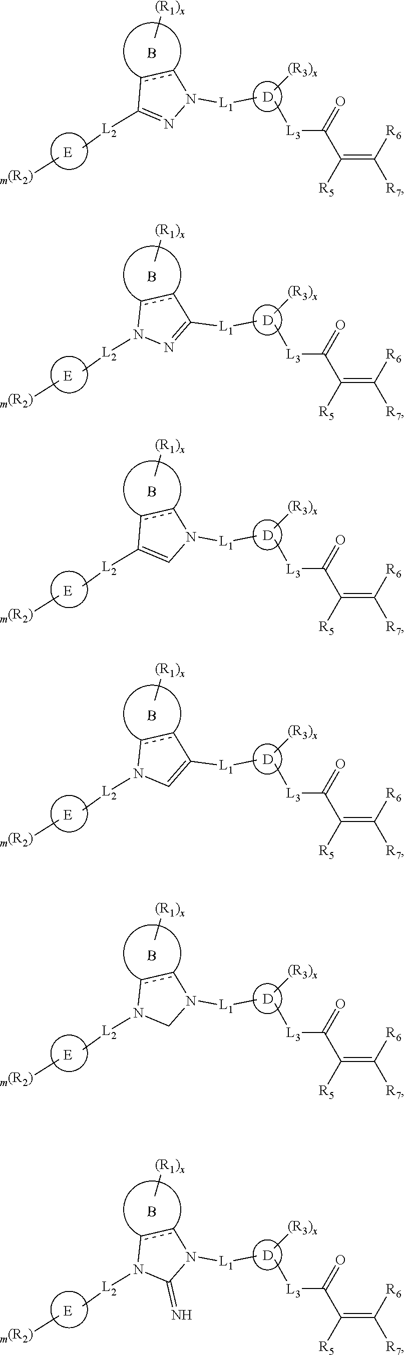

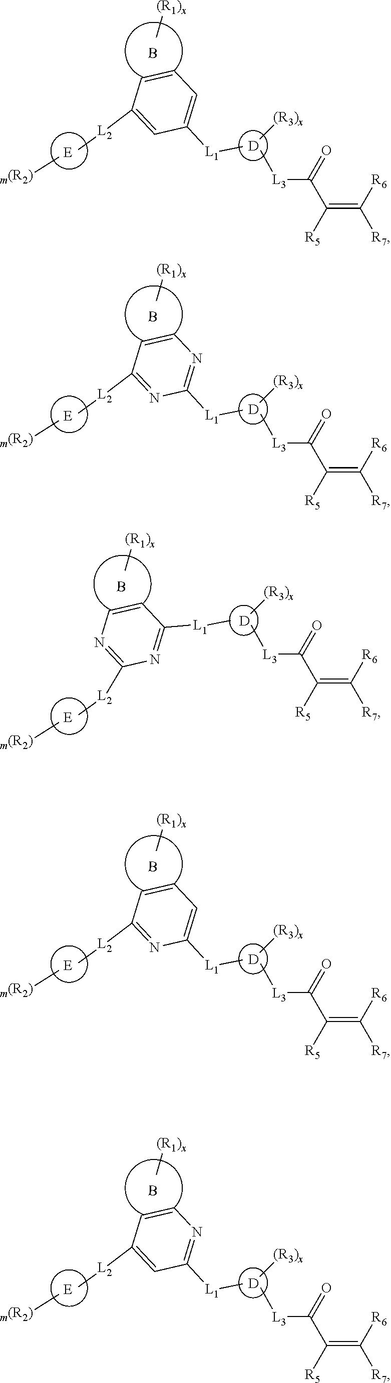

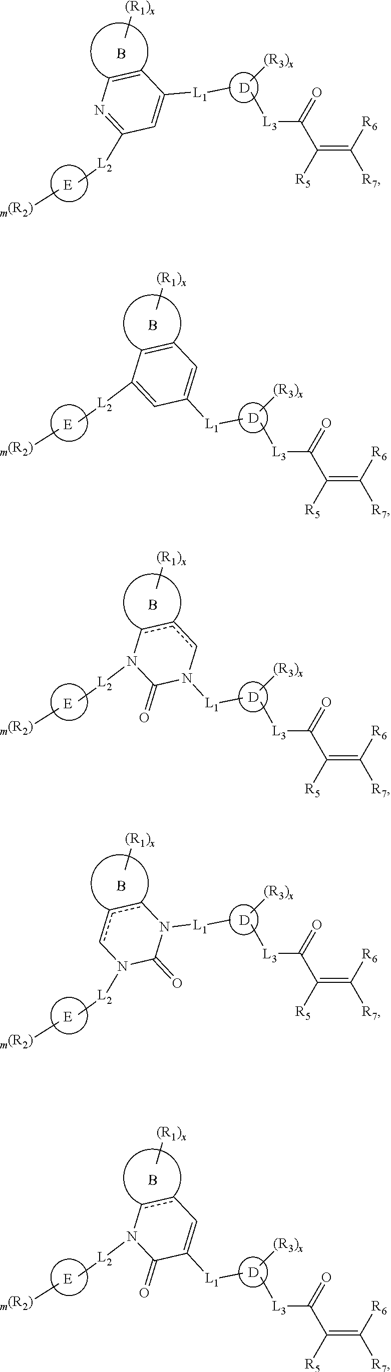

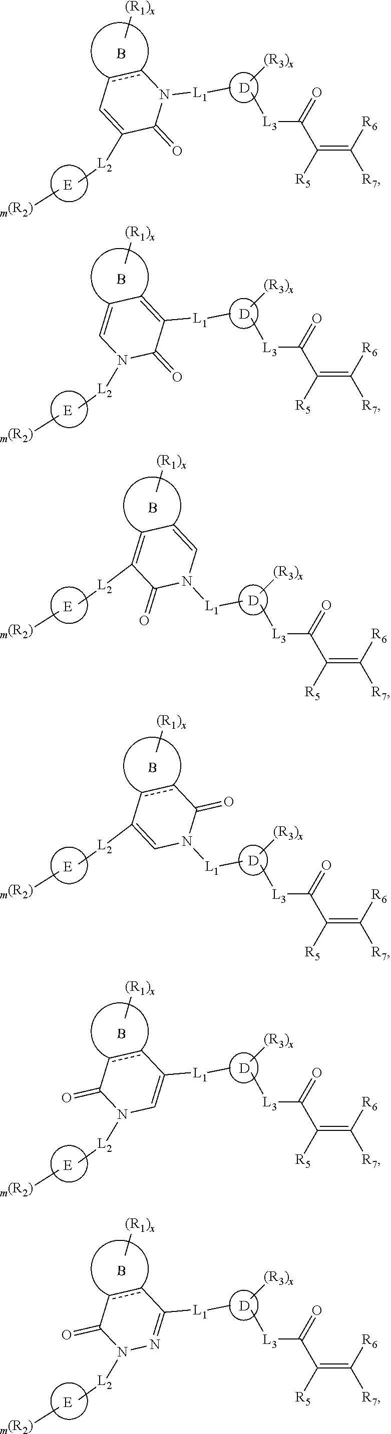

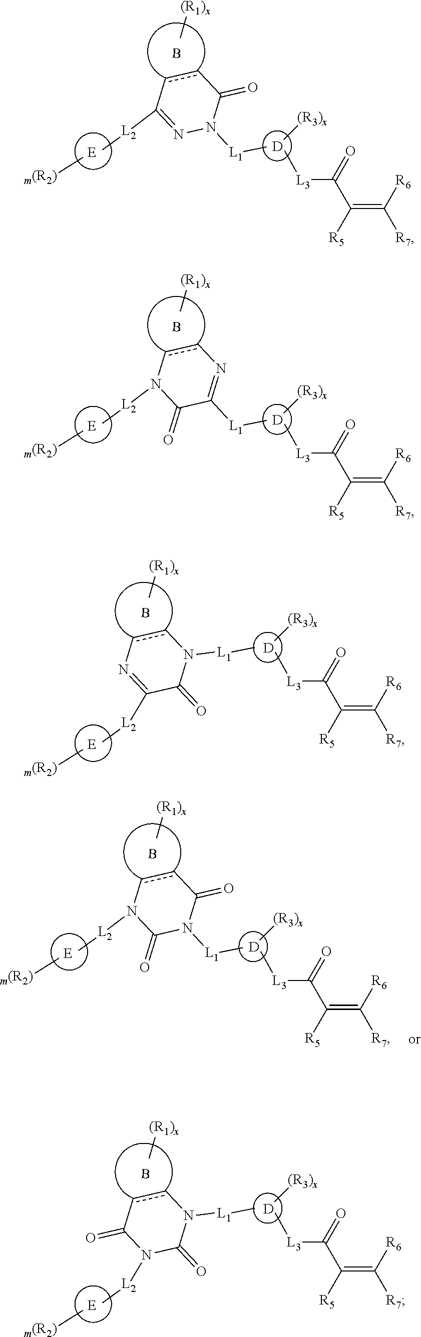

or a stereoisomer, tautomer, pharmaceutically acceptable salt, prodrug, chelate, non-covalent complex, or solvate thereof, wherein, each ring B, ring E, ring D, L₁, L₂, L₃, R₁, R₂, R₃, R₅, R₆, R₇, m, y and x are as defined in embodiments and classes and subclasses herein.

In some embodiments, a compound disclosed herein is of the one of formulas:

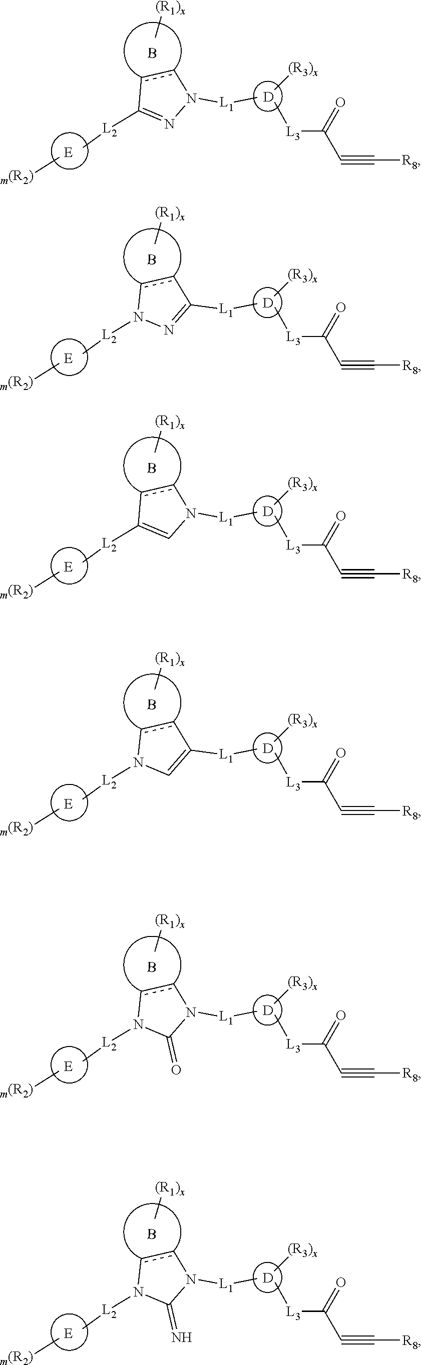

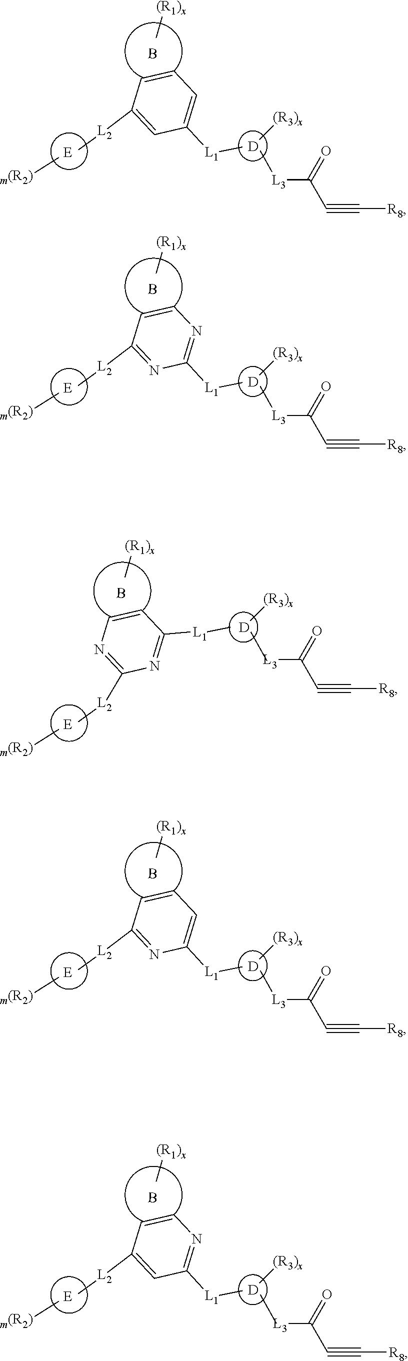

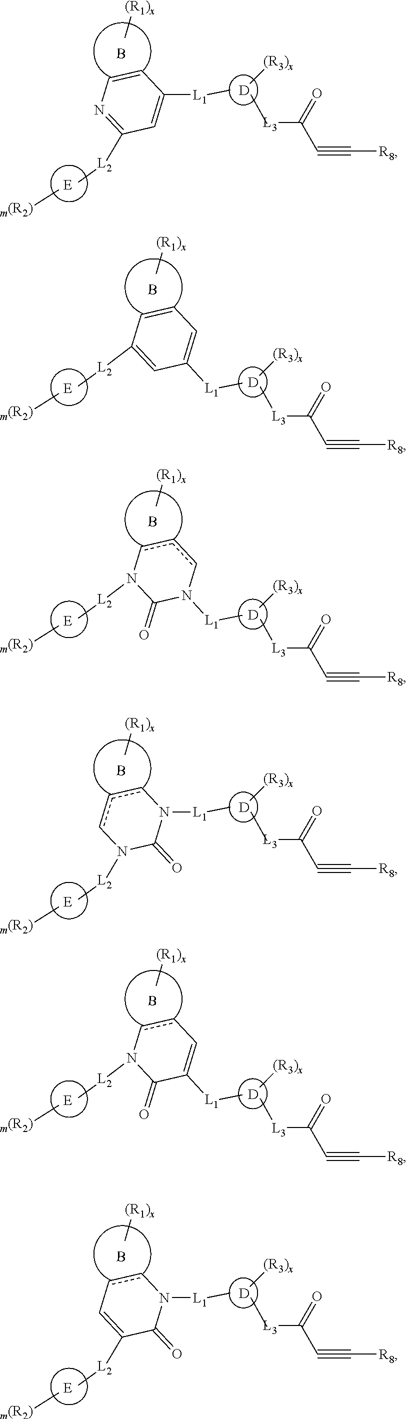

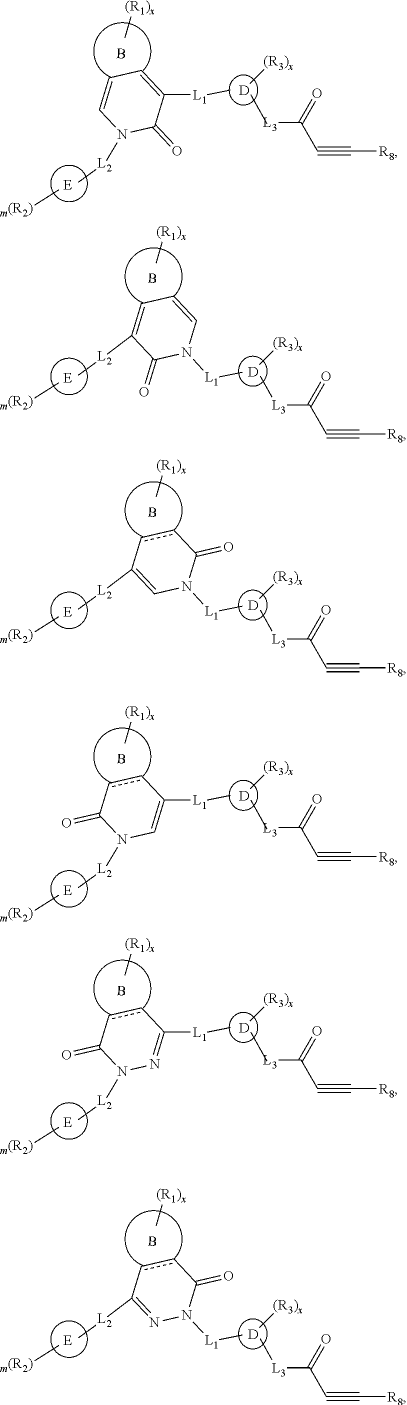

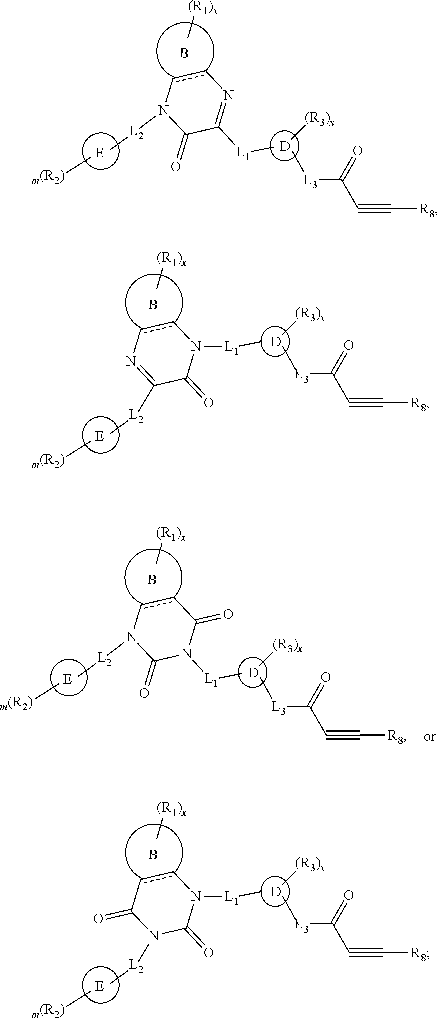

or a stereoisomer, tautomer, pharmaceutically acceptable salt, prodrug, chelate, non-covalent complex, or solvate thereof, wherein, each ring B, ring E, ring D, L₁, L₂, L₃, R₁, R₂, R₃, R₈, m, y and x are as defined in embodiments and classes and subclasses herein.

In some embodiments, a compound disclosed herein is of the one of formulas:

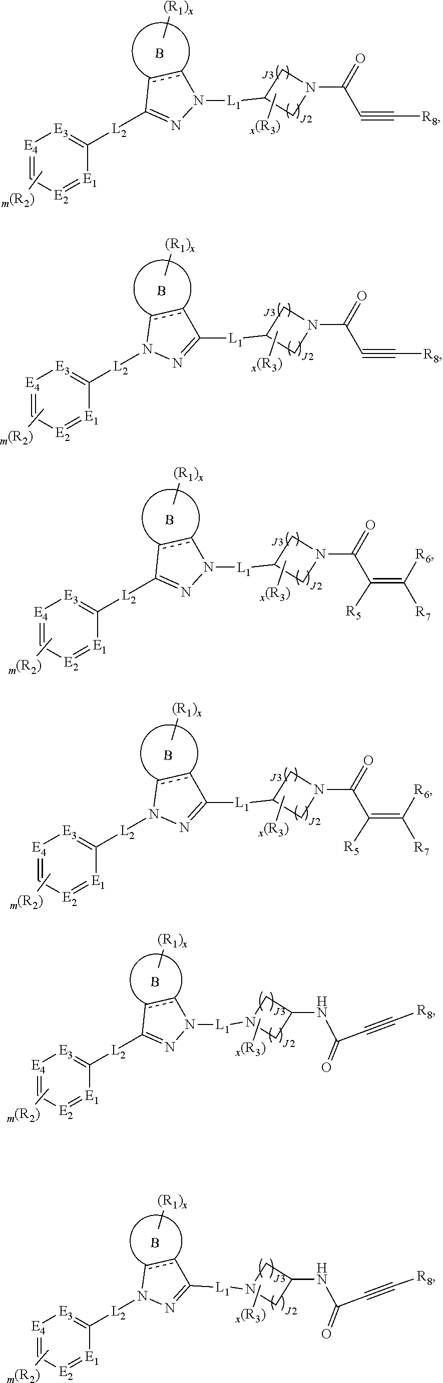

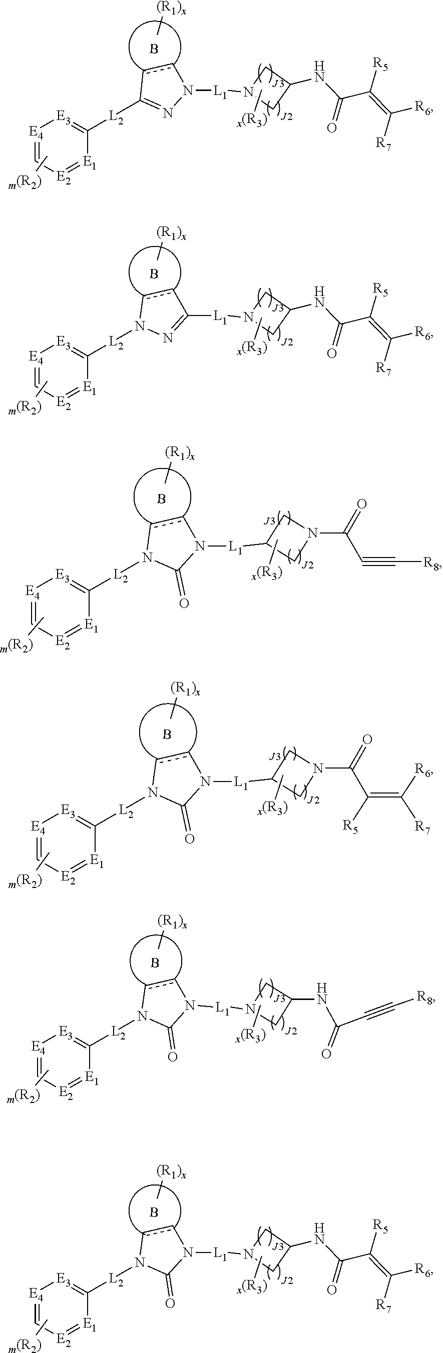

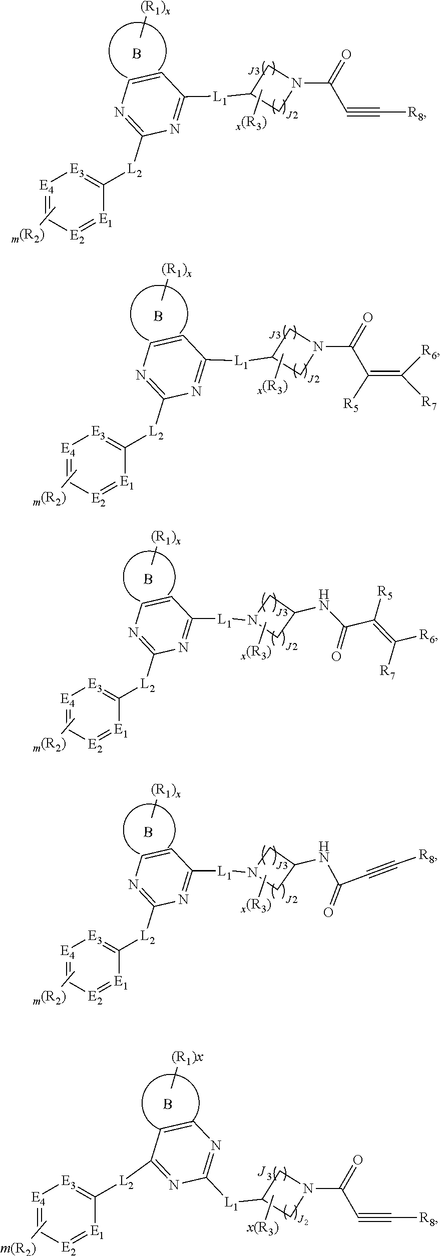

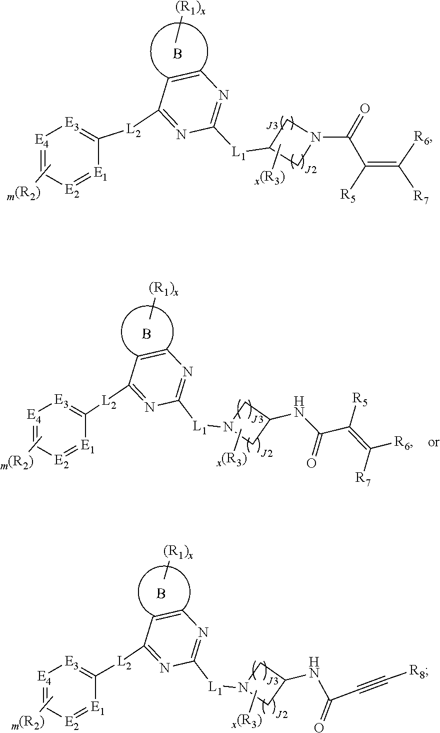

or a stereoisomer, tautomer, pharmaceutically acceptable salt, prodrug, chelate, non-covalent complex, or solvate thereof, wherein, each ring B, L₁, L₂, R₁, R₂, R₃, R₅, R₆, R₇, R₈, m, y and x are as defined in embodiments and classes and subclasses herein.

In some embodiments of Formula (I), wherein the compound is:
1) 1-(1-acryloylpyrrolidin-3-yl)-3-(4-cyclohexylphenyl)-1,6-dihydro-7H-pyrazolo[4,3-d]pyrimidin-7-one;
2) 1-(1-acryloylpyrrolidin-3-yl)-3-(4-(trifluoromethyl)phenyl)-1,6-dihydro-7H-pyrazolo[4,3-d]pyrimidin-7-one;
3) 1-(3-(3-(4-(trifluoromethyl)phenyl)-1H-indazol-1-yl)pyrrolidin-1-yl)prop-2-en-1-one;
4) 1-(3-(1-(4-(trifluoromethyl)phenyl)-1H-indazol-3-yl)pyrrolidin-1-yl)prop-2-en-1-one;
5) 2-fluoro-1-(3-(3-(4-(trifluoromethyl)phenyl)-1H-pyrazolo[3,4-b]pyridin-1-yl)azetidin-1-yl)prop-2-en-1-one;
6) 2-fluoro-1-(3-(3-(4-(trifluoromethyl)phenyl)-1H-pyrazolo[4,3-b]pyridin-1-yl)azetidin-1-yl)prop-2-en-1-one;
7) 1-(1-acryloylpyrrolidin-3-yl)-6-methyl-3-(4-(trifluoromethyl)phenyl)-1,6-dihydro-7H-pyrazolo[4,3-d]pyrimidin-7-one;
8) 2-fluoro-1-(3-methyl-3-(3-(4-(trifluoromethyl)phenyl)-1H-pyrazolo[3,4-b]pyridin-1-yl)azetidin-1-yl)prop-2-en-1-one;
9) 2-fluoro-1-(2-methyl-3-(3-(4-(trifluoromethyl)phenyl)-1H-pyrazolo[3,4-b]pyridin-1-yl)azetidin-1-yl)prop-2-en-1-one;
10) 2-fluoro-N-(2-methyl-5-(3-(4-(trifluoromethyl)phenyl)-1H-pyrazolo[3,4-b]pyridin-1-yl)phenyl)acrylamide;
11) 2-fluoro-1-(3-(6-methyl-3-((4-(trifluoromethyl)phenyl)amino)-1H-pyrazolo[3,4-b]pyridin-1-yl)azetidin-1-yl)prop-2-en-1-one;
12) 2-fluoro-1-(2-hydroxy-3-(3-(4-(trifluoromethyl)phenyl)-1H-pyrazolo[3,4-b]pyridin-1-yl)azetidin-1-yl)prop-2-en-1-one;
13) N-(1-(3-(4-(trifluoromethyl)phenyl)imidazo[1,5-a]pyridin-1-yl)azetidin-3-yl)acrylamide;
14) N-(1-(1-acryloylazetidin-3-yl)-3-(4-(trifluoromethyl) phenyl)-1H-indazol-7-yl) methanesulfonamide;
15) N-(1-(1-acryloylazetidin-3-yl)-3-(4-(trifluoromethyl)phenyl)-1H-indazol-7-yl)acetamide;
16) 1-(3-(4-amino-3-(4-cyclohexylphenyl)-1H-pyrazolo[3,4-d]pyrimidin-1-yl)pyrrolidin-1-yl)prop-2-en-1-one;
17) 1-(3-(3-(4-cyclohexylphenyl)-4-hydroxy-1H-pyrazolo[3,4-d]pyrimidin-1-yl)pyrrolidin-1-yl)prop-2-en-1-one;
18) 1-(3-(3-(4-(trifluoromethyl)phenyl)-1H-pyrazolo[3,4-b]pyrazin-1-yl)pyrrolidin-1-yl)prop-2-en-1-one;
19) 1-(3-(3-(4-(trifluoromethyl)phenyl)-1H-pyrazolo[3,4-d]pyrimidin-1-yl)pyrrolidin-1-yl)prop-2-en-1-one;
20) 1-(3-(6-chloro-3-(4-(trifluoromethyl)phenyl)-1H-pyrazolo[3,4-b]pyrazin-1-yl)pyrrolidin-1-yl)prop-2-en-1-one;
21) 1-(3-(3-(4-(trifluoromethyl)phenyl)-1H-pyrazolo[3,4-b]pyridin-1-yl)pyrrolidin-1-yl)prop-2-en-1-one;
22) 1-(3-(3-(4-(trifluoromethyl)phenyl)-1H-pyrazolo[3,4-c]pyridin-1-yl)pyrrolidin-1-yl)prop-2-en-1-one;
23) 1-(3-(3-(4-(trifluoromethyl)phenyl)-1H-pyrazolo[4,3-c]pyridin-1-yl)pyrrolidin-1-yl)prop-2-en-1-one;
24) 1-(3,3-difluoro-4-(3-(4-(trifluoromethyl)phenyl)-1H-pyrazolo[4,3-b]pyridin-1-yl)pyrrolidin-1-yl)prop-2-en-1-one;
25) 1-((3R,4S)-3-fluoro-4-(3-(4-(trifluoromethyl)phenyl)-1H-pyrazolo[4,3-b]pyridin-1-yl)pyrrolidin-1-yl)prop-2-en-1-one;
26) 1-(3-(3-(4-(trifluoromethyl)phenyl)-1H-pyrazolo[4,3-b]pyridin-1-yl)pyrrolidin-1-yl)prop-2-en-1-one;
27) 1-(3-(3-(5-(trifluoromethyl)pyridin-2-yl)-1H-pyrazolo[3,4-b]pyridin-1-yl)pyrrolidin-1-yl)prop-2-en-1-one;
28) N-(1-(1-(4-(trifluoromethyl)phenyl)-1H-pyrazolo[4,3-b]pyridin-3-yl)pyrrolidin-3-yl)but-2-ynamide;
29) 1-(3-(3-(2-fluoro-4-(trifluoromethyl)phenyl)-1H-pyrazolo[3,4-b]pyridin-1-yl)pyrrolidin-1-yl)prop-2-en-1-one;
30) 1-(3-(3-(4-(trifluoromethyl)phenyl)-1H-pyrazolo[3,4-b]pyridin-1-yl)azetidin-1-yl)prop-2-en-1-one;
31) 1-(3-(3-(4-(trifluoromethyl)phenoxy)-1H-pyrazolo[4,3-b]pyridin-1-yl)azetidin-1-yl)prop-2-en-1-one;
32) 1-(3-(3-(4-(trifluoromethyl)phenoxy)-1H-pyrazolo[3,4-b]pyridin-1-yl)azetidin-1-yl)prop-2-en-1-one;
33) 1-(3-(3-(4-(trifluoromethyl)phenyl)-1H-pyrazolo[3,4-b]pyridin-1-yl)piperidin-1-yl)prop-2-en-1-one;
34) 1-(3-((3-(4-(trifluoromethyl)phenyl)-1H-indazol-1-yl)methyl)pyrrolidin-1-yl)prop-2-en-1-one;
35) N-(1-(1-(4-(trifluoromethyl)phenyl)-1H-pyrazolo[4,3-b]pyridin-3-yl)pyrrolidin-3-yl)acrylamide;
36) (E)-N-(1-(1-(4-(trifluoromethyl)phenyl)-1H-pyrazolo[4,3-b]pyridin-3-yl)pyrrolidin-3-yl)but-2-enamide;
37) N-(3-(3-(4-(trifluoromethyl)phenyl)-1H-indazol-1-yl)cyclopentyl)acrylamide;
38) 1-(3-((3-(4-cyclohexylphenyl)-1H-indazol-1-yl)methyl)pyrrolidin-1-yl)prop-2-en-1-one;
39) 1-(3-(7-methyl-3-(4-(trifluoromethyl)phenyl)-1H-indazol-1-yl)pyrrolidin-1-yl)prop-2-en-1-one;
40) (E)-4-(dimethylamino)-1-(3-(3-(4-(trifluoromethyl)phenyl)-1H-indazol-1-yl)pyrrolidin-1-yl)but-2-en-1-one;
41) (E)-4-(dimethylamino)-N-(1-(1-(4-(trifluoromethyl)phenyl)-1H-pyrazolo[4,3-b]pyridin-3-yl)pyrrolidin-3-yl)but-2-enamide;
42) 1-(1-acryloylpyrrolidin-3-yl)-3-(4-(trifluoromethyl)phenyl)-1H-indazole-7-carboxamide;
43) 1-(4-(1-(4-(trifluoromethyl)phenyl)-1H-indazole-3-carbonyl)piperazin-1-yl)prop-2-en-1-one;
44) 1-(3-(7-methoxy-3-(4-(trifluoromethyl)phenyl)-1H-indazol-1-yl)pyrrolidin-1-yl)prop-2-en-1-one;
45) 1-(3-(7-chloro-3-(4-(trifluoromethyl)phenyl)-1H-indazol-1-yl)pyrrolidin-1-yl)prop-2-en-1-one;
46) 1-(3-(7-(trifluoromethyl)-3-(4-(trifluoromethyl)phenyl)-1H-indazol-1-yl)pyrrolidin-1-yl)prop-2-en-1-one;
47) 1-(3-(6-methyl-3-(4-(trifluoromethyl)phenyl)-1H-indazol-1-yl)pyrrolidin-1-yl)prop-2-en-1-one;
48) 1-(1-acryloylpyrrolidin-3-yl)-3-(4-(trifluoromethyl)phenyl)-1H-indazole-7-carbonitrile;
49) 1-(7-(3-(4-(trifluoromethyl)phenyl)-1H-pyrazolo[3,4-b]pyridin-1-yl)-2-azaspiro[4.4]nonan-2-yl)prop-2-en-1-one;
50) 1-(3-(6-fluoro-3-(4-(trifluoromethyl)phenyl)-1H-indazol-1-yl)pyrrolidin-1-yl)prop-2-en-1-one;
51) 1-(3-(5,6-difluoro-3-(4-(trifluoromethyl)phenyl)-1H-indazol-1-yl)pyrrolidin-1-yl)prop-2-en-1-one;
52) 1-(3-(1-(4-(trifluoromethyl)phenyl)-1H-pyrazolo[3,4-b]pyridin-3-yl)pyrrolidin-1-yl)prop-2-en-1-one;
53) N-(1-(1-(4-(trifluoromethyl)phenyl)-1H-pyrazolo[3,4-b]pyridin-3-yl)pyrrolidin-3-yl)acrylamide;
54) 1-(3-(6-methoxy-3-(4-(trifluoromethyl)phenyl)-1H-indazol-1-yl)pyrrolidin-1-yl)prop-2-en-1-one;
55) 1-(3-(3-(4-(trifluoromethyl)phenyl)-1H-indazol-1-yl)pyrrolidin-1-yl)but-2-yn-1-one;
56) (E)-1-(3-(3-(4-(trifluoromethyl)phenyl)-1H-indazol-1-yl)pyrrolidin-1-yl)but-2-en-1-one;
57) 1-(3-(3-(4-(trifluoromethyl)phenyl)-1H-indazol-1-yl)pyrrolidin-1-yl)prop-2-yn-1-one;
58) 1-(3-(3-(5-(trifluoromethyl)pyridin-2-yl)-1H-indazol-1-yl)pyrrolidin-1-yl)prop-2-en-1-one;
59) 1-(3-(3-(6-(trifluoromethyl)pyridin-3-yl)-1H-indazol-1-yl)pyrrolidin-1-yl)prop-2-en-1-one;
60) 1-(3-(3-(4-(trifluoromethyl)phenyl)-1H-indazol-1-yl)piperidin-1-yl)prop-2-en-1-one;
61) 1-(3-(3-(4-(trifluoromethyl)phenyl)-1H-indazol-1-yl)azetidin-1-yl)prop-2-en-1-one;
62) N-(4-(3-(4-(trifluoromethyl)phenyl)-1H-indazol-1-yl)tetrahydrofuran-3-yl)acrylamide;
63) N-((5-(3-(4-(trifluoromethyl)phenyl)-1H-indazol-1-yl)-1,3,4-oxadiazol-2-yl)methyl)acrylamide;
64) N-(1-(1-(4-(trifluoromethyl)phenyl)-1H-indazole-3-carbonyl)pyrrolidin-3-yl)acrylamide;
65) 1-(3-(1-(4-(trifluoromethyl)phenyl)-1H-pyrazolo[4,3-b]pyridin-3-yl)pyrrolidin-1-yl)prop-2-en-1-one;
66) N-(1-(5-methoxy-1-(4-(trifluoromethyl)phenyl)-1H-pyrazolo[4,3-b]pyridin-3-yl)pyrrolidin-3-yl)acrylamide;
67) 1-(3-(5-(3-(4-(trifluoromethyl)phenyl)-1H-indazol-1-yl)-1,3,4-oxadiazol-2-yl)pyrrolidin-1-yl)prop-2-en-1-one;
68) N-(4-(3-(4-(trifluoromethyl)phenyl)-1H-indazol-1-yl)tetrahydro-2H-pyran-3-yl)acrylamide;
69) N-(1-(5-cyano-1-(4-(trifluoromethyl)phenyl)-1H-pyrazolo[4,3-b]pyridin-3-yl)pyrrolidin-3-yl)acrylamide;
70) 1-(3-(7-fluoro-3-(4-(trifluoromethyl)phenyl)-1H-indazol-1-yl)pyrrolidin-1-yl)prop-2-en-1-one;
71) 2-fluoro-1-(3-(3-(4-(trifluoromethyl)phenyl)-1H-pyrazolo[4,3-b]pyridin-1-yl)pyrrolidin-1-yl)prop-2-en-1-one;
72) N-(1-(5-cyano-1-(4-(trifluoromethyl)phenyl)-1H-pyrazolo[3,4-b]pyridin-3-yl)pyrrolidin-3-yl)acrylamide;
73) 2-methyl-1-(3-(3-(4-(trifluoromethyl)phenyl)-1H-pyrazolo[3,4-b]pyridin-1-yl)azetidin-1-yl)prop-2-en-1-one;
74) N-(1-(5-methoxy-1-(4-(trifluoromethyl)phenyl)-1H-pyrazolo[3,4-b]pyridin-3-yl)pyrrolidin-3-yl)acrylamide;
75) N-(1-(5-methyl-1-(4-(trifluoromethyl)phenyl)-1H-pyrazolo[4,3-b]pyridin-3-yl)pyrrolidin-3-yl)acrylamide;
76) 5-methyl-2-(3-(3-(4-(trifluoromethyl)phenyl)-1H-pyrazolo[3,4-b]pyridin-1-yl)azetidine-1-carbonyl)hex-2-enenitrile;
77) 1-(1-(2-fluoroacryloyl)pyrrolidin-3-yl)-6-methyl-3-(4-(trifluoromethyl)phenyl)-1,6-dihydro-7H-pyrazolo[4,3-d]pyrimidin-7-one;
78) methyl 1-(1-acryloylpyrrolidin-3-yl)-3-(4-(trifluoromethyl)phenyl)-1H-indazole-7-carboxylate;
79) 1-(1-acryloylazetidin-3-yl)-6-methyl-3-(4-(trifluoromethyl)phenyl)-1,6-dihydro-7H-pyrazolo[4,3-d]pyrimidin-7-one;
80) 1-(1-(2-fluoroacryloyl)azetidin-3-yl)-6-methyl-3-(4-(trifluoromethyl)phenyl)-1,6-dihydro-7H-pyrazolo[4,3-d]pyrimidin-7-one;
81) N-(1-(6-methyl-1-(4-(trifluoromethyl)phenyl)-1H-pyrazolo[4,3-b]pyridin-3-yl)pyrrolidin-3-yl)acrylamide;
82) 1-(3-(3-(4-(trifluoromethyl)phenyl)-1H-pyrazolo[4,3-b]pyridin-1-yl)azetidin-1-yl)prop-2-en-1-one;
83) N-(1-(5-methyl-1-(4-(trifluoromethyl)phenyl)-1H-pyrazolo[3,4-b]pyridin-3-yl)pyrrolidin-3-yl)acrylamide;
84) N-(1-(5-chloro-1-(4-(trifluoromethyl)phenyl)-1H-pyrazolo[4,3-b]pyridin-3-yl)pyrrolidin-3-yl)acrylamide;
85) N-(1-(5-chloro-1-(4-(trifluoromethyl)phenyl)-1H-pyrazolo[3,4-b]pyridin-3-yl)pyrrolidin-3-yl)acrylamide;
86) N-(1-(6-chloro-1-(4-(trifluoromethyl)phenyl)-1H-pyrazolo[3,4-b]pyridin-3-yl)pyrrolidin-3-yl)acrylamide;
87) 2-methyl-1-(3-(3-(4-(trifluoromethyl)phenyl)-1H-pyrazolo[4,3-b]pyridin-1-yl)azetidin-1-yl)prop-2-en-1-one;
88) 4-methyl-4-morpholino-2-(3-(3-(4-(trifluoromethyl)phenyl)-1H-pyrazolo[4,3-b]pyridin-1-yl)azetidine-1-carbonyl)pent-2-enenitrile;
89) 1-(3-(5-methoxy-3-(4-(trifluoromethyl)phenyl)-1H-pyrazolo[4,3-b]pyridin-1-yl)pyrrolidin-1-yl)prop-2-en-1-one;
90) N-(1-(1-(4-(trifluoromethyl)phenyl)-1H-pyrazolo[3,4-b]pyridin-3-yl)pyrrolidin-3-yl)acrylamide;
91) N-(1-(1-(4-(trifluoromethyl)phenyl)-1H-pyrazolo[4,3-b]pyridin-3-yl)pyrrolidin-3-yl)propiolamide;
92) N-(1-(1-(4-(trifluoromethyl)phenyl)-1H-pyrazolo[3,4-b]pyridin-3-yl)pyrrolidin-3-yl)propiolamide;
93) 1-(1-acryloylpyrrolidin-3-yl)-3-(4-(trifluoromethyl)phenyl)-1H-pyrazolo[4,3-b]pyridine-5-carbonitrile;
94) 1-(3-(5-methoxy-3-(4-(trifluoromethyl)phenyl)-1H-pyrazolo[3,4-b]pyridin-1-yl)pyrrolidin-1-yl)prop-2-en-1-one;
95) 2-fluoro-1-(3-(5-methoxy-3-(4-(trifluoromethyl)phenyl)-1H-pyrazolo[3,4-b]pyridin-1-yl)pyrrolidin-1-yl)prop-2-en-1-one;
96) 1-(3-(5-methyl-3-(4-(trifluoromethyl)phenyl)-1H-pyrazolo[3,4-b]pyridin-1-yl)pyrrolidin-1-yl)prop-2-en-1-one;
97) N-(3-(1-(4-(trifluoromethyl)phenyl)-1H-pyrazolo[3,4-b]pyridin-3-yl)phenyl)acryl amide;
98) 1-(1-acryloylpyrrolidin-3-yl)-N-isopropyl-3-(6-(trifluoromethyl)pyridin-3-yl)-1H-indazole-7-carboxamide;
99) N-(1-(3-(4-(trifluoromethyl)phenyl)imidazo[1,5-a]pyridin-1-yl)pyrrolidin-3-yl)acrylamide;
100) 1-(1-acryloylpyrrolidin-3-yl)-N-cyclopropyl-3-(6-(trifluoromethyl)pyridin-3-yl)-1H-indazole-7-carboxamide;
101) 1-(1-acryloylpyrrolidin-3-yl)-N-(oxetan-3-yl)-3-(6-(trifluoromethyl)pyridin-3-yl)-1H-indazole-7-carboxamide;
102) 1-(1-acryloylpyrrolidin-3-yl)-N-methyl-3-(6-(trifluoromethyl)pyridin-3-yl)-1H-indazole-7-carboxamide;
103) 1-(1-acryloylpyrrolidin-3-yl)-N,N-dimethyl-3-(4-(trifluoromethyl)phenyl)-1H-indazole-7-carboxamide;
104) 1-(1-acryloylpyrrolidin-3-yl)-N-(3,3-difluorocyclobutyl)-3-(6-(trifluoromethyl)pyridin-3-yl)-1H-indazole-7-carboxamide;
105) 1-(3-(1-(4-(trifluoromethyl)phenyl)imidazo[1,5-a]pyridin-3-yl)pyrrolidin-1-yl)prop-2-en-1-one;
106) N-(1-(6-(4-(trifluoromethyl)phenyl)imidazo[1,5-a]pyrimidin-8-yl)pyrrolidin-3-yl)acrylamide;
107) 1-(1-acryloylpyrrolidin-3-yl)-N-phenyl-3-(4-(trifluoromethyl)phenyl)-1H-indazole-7-carboxamide;
108) 1-(3-(5-chloro-3-(4-(trifluoromethyl)phenyl)-1H-pyrazolo[3,4-b]pyridin-1-yl)pyrrolidin-1-yl)prop-2-en-1-one;
109) 1-(3-(8-(4-(trifluoromethyl)phenyl)imidazo[1,5-a]pyrimidin-6-yl)pyrrolidin-1-yl)prop-2-en-1-one;
110) 1-(3-(3-(4-(trifluoromethyl)phenyl)-7-(4-(trifluoromethyl)piperidine-1-carbonyl)-1H-indazol-1-yl)pyrrolidin-1-yl)prop-2-en-1-one;
111) 1-(1-acryloylpyrrolidin-3-yl)-N-(4,4-difluorocyclohexyl)-3-(4-(trifluoromethyl)phenyl)-1H-indazole-7-carboxamide;
112) 1-(3-(7-(3,3-difluoropyrrolidine-1-carbonyl)-3-(4-(trifluoromethyl)phenyl)-1H-indazol-1-yl)pyrrolidin-1-yl)prop-2-en-1-one;
113) 1-(1-acryloylpyrrolidin-3-yl)-N-(3,3-difluorocyclopentyl)-3-(4-(trifluoromethyl)phenyl)-1H-indazole-7-carboxamide;
114) 1-(1-acryloylpyrrolidin-3-yl)-N-(4-(trifluoromethyl)cyclohexyl)-3-(4-(trifluoromethyl)phenyl)-1H-indazole-7-carboxamide;
115) 1-(1-acryloylpyrrolidin-3-yl)-N-benzyl-3-(4-(trifluoromethyl)phenyl)-1H-indazole-7-carboxamide;
116) 1-(1-acryloylpyrrolidin-3-yl)-N-(tert-butyl)-3-(4-(trifluoromethyl)phenyl)-1H-indazole-7-carboxamide;
117) 1-(3-methyl-4-(3-(4-(trifluoromethyl)phenyl)-1H-pyrazolo[4,3-b]pyridin-1-yl)pyrrolidin-1-yl)prop-2-en-1-one;
118) 1-(7-(3-(4-(trifluoromethyl)phenyl)-1H-pyrazolo[4,3-b]pyridin-1-yl)-5-azaspiro[2.4]heptan-5-yl)prop-2-en-1-one;
119) N-(2-(3-(4-(trifluoromethyl)phenyl)-1H-pyrazolo[4,3-b]pyridin-1-yl)cyclopentyl)acrylamide;
120) 1-(3-(3-(4-cyclopropylphenyl)-1H-pyrazolo[4,3-b]pyridin-1-yl)azetidin-1-yl)prop-2-en-1-one;
121) 1-(3-(6-(dimethylamino)-3-(4-(trifluoromethyl)phenyl)-1H-pyrazolo[3,4-b]pyridin-1-yl)azetidin-1-yl)prop-2-en-1-one;
122) 1-(3-(6-(dimethylamino)-3-(4-(trifluoromethyl)phenyl)-1H-pyrazolo[3,4-b]pyridin-1-yl)azetidin-1-yl)-2-fluoroprop-2-en-1-one;
123) 1-(3-(3-(6-(trifluoromethyl)pyridin-3-yl)-1H-pyrazolo[3,4-b]pyridin-1-yl)azetidin-1-yl)prop-2-en-1-one;
124) 2-fluoro-1-(3-(3-(6-(trifluoromethyl)pyridin-3-yl)-1H-pyrazolo[3,4-b]pyridin-1-yl)azetidin-1-yl)prop-2-en-1-one;
125) 1-(7-(3-(4-(trifluoromethyl)phenyl)-1H-pyrazolo[4,3-b]pyridin-1-yl)-2-azaspiro[4.4]nonan-2-yl)prop-2-en-1-one;
126) 2-fluoro-1-(7-(3-(4-(trifluoromethyl)phenyl)-1H-pyrazolo[4,3-b]pyridin-1-yl)-2-azaspiro[4.4]nonan-2-yl)prop-2-en-1-one;
127) 1-(3-(3-(2-fluoro-4-(trifluoromethyl)phenyl)-1H-pyrazolo[3,4-b]pyridin-1-yl)azetidin-1-yl)prop-2-en-1-one;
128) 2-fluoro-1-(3-(3-(2-fluoro-4-(trifluoromethyl)phenyl)-1H-pyrazolo[3,4-b]pyridin-1-yl)azetidin-1-yl)prop-2-en-1-one;
129) 1-(3-(3-(2-fluoro-4-(trifluoromethyl)phenyl)-1H-pyrazolo[4,3-b]pyridin-1-yl)azetidin-1-yl)prop-2-en-1-one;
130) 2-fluoro-1-(3-(3-(2-fluoro-4-(trifluoromethyl)phenyl)-1H-pyrazolo[4,3-b]pyridin-1-yl)azetidin-1-yl)prop-2-en-1-one;
131) 1-(3-(3-(6-(trifluoromethyl)pyridin-3-yl)-1H-pyrazolo[4,3-b]pyridin-1-yl)azetidin-1-yl)prop-2-en-1-one;
132) 2-fluoro-1-(3-(3-(6-(trifluoromethyl)pyridin-3-yl)-1H-pyrazolo[4,3-b]pyridin-1-yl)azetidin-1-yl)prop-2-en-1-one;
133) N-(3-(4-(trifluoromethyl)phenyl)-1′H-[1,6′-biindazol]-4′-yl)acrylamide;
134) N-(6-(3-(4-(trifluoromethyl)phenyl)-1H-indazol-1-yl)-[1,2,4]triazolo[4,3-a]pyridin-8-yl)acrylamide;
135) N-(3-(3-(4-(trifluoromethyl)phenyl)-1H-indazol-1-yl)phenyl)acrylamide;
136) N-(3-methyl-5-(3-(4-(trifluoromethyl)phenyl)-1H-indazol-1-yl)phenyl)acrylamide;
137) N-(3-methoxy-5-(3-(4-(trifluoromethyl)phenyl)-1H-indazol-1-yl)phenyl)acryl amide;
138) N-(3-chloro-5-(3-(4-(trifluoromethyl)phenyl)-1H-indazol-1-yl)phenyl)acryl amide;
139) 1-(3-(3-(4-(trifluoromethyl)phenyl)-1H-pyrazolo[4,3-b]pyridin-1-yl)azetidin-1-yl)prop-2-yn-1-one;
140) 1-(3-(3-(4-(trifluoromethyl)phenyl)-1H-pyrazolo[3,4-b]pyridin-1-yl)azetidin-1-yl)prop-2-yn-1-one;
141) (E)-2-(3-(3-(4-(trifluoromethyl)phenyl)-1H-pyrazolo[4,3-b]pyridin-1-yl)azetidine-1-carbonyl)but-2-enenitrile;
142) 1-(1-acryloylpyrrolidin-3-yl)-3-(4-(trifluoromethyl)phenyl)-1H-pyrazolo[3,4-b]pyridine-5-carbonitrile;
143) 1-(3-(5-methyl-3-(4-(trifluoromethyl)phenyl)-1H-pyrazolo[4,3-b]pyridin-1-yl)pyrrolidin-1-yl)prop-2-en-1-one;
144) 1-(3-(6-methyl-3-(4-(trifluoromethyl)phenyl)-1H-pyrazolo[4,3-b]pyridin-1-yl)pyrrolidin-1-yl)prop-2-en-1-one;
145) 1-(1-acryloylpyrrolidin-3-yl)-N-(pyridin-2-yl)-3-(4-(trifluoromethyl)phenyl)-1H-indazole-7-carboxamide;
146) 1-(3-(5-chloro-3-(4-(trifluoromethyl)phenyl)-1H-pyrazolo[4,3-b]pyridin-1-yl)pyrrolidin-1-yl)prop-2-en-1-one;
147) 1-(3-(6-chloro-3-(4-(trifluoromethyl)phenyl)-1H-pyrazolo[3,4-b]pyridin-1-yl)pyrrolidin-1-yl)prop-2-en-1-one;
148) 1-(1-acryloylpyrrolidin-3-yl)-3-(4-(trifluoromethyl)phenyl)-N-(5-(trifluoromethyl)pyridin-2-yl)-1H-indazole-7-carboxamide;
149) 1-acryloyl-4-(3-(4-(trifluoromethyl)phenyl)-1H-pyrazolo[4,3-b]pyridin-1-yl)pyrrolidine-3-carbonitrile;
150) 1-(3-(5-methyl-1-(4-(trifluoromethyl)phenyl)-1H-pyrazolo[3,4-b]pyridin-3-yl)pyrrolidin-1-yl)prop-2-en-1-one;
151) N-(3-cyano-5-(3-(4-(trifluoromethyl)phenyl)-1H-indazol-1-yl)phenyl)acrylamide;
152) N-(3-cyano-5-(3-(4-(trifluoromethyl)phenyl)-1H-indazol-1-yl)phenyl)acrylamide;
153) N-(3-cyclopropyl-5-(3-(4-(trifluoromethyl)phenyl)-1H-indazol-1-yl)phenyl)acryl amide;
154) N-(3-(3,3-difluoroazetidin-1-yl)-5-(3-(4-(trifluoromethyl)phenyl)-1H-indazol-1-yl)phenyl)acrylamide;
155) N-(3-(3-methylpyridin-2-yl)-5-(3-(4-(trifluoromethyl)phenyl)-1H-indazol-1-yl)phenyl)acrylamide;
156) N-(3-(3-chloropyridin-2-yl)-5-(3-(4-(trifluoromethyl)phenyl)-1H-indazol-1-yl)phenyl)acrylamide;
157) N-(3-(1H-pyrazol-1-yl)-5-(3-(4-(trifluoromethyl)phenyl)-1H-indazol-1-yl)phenyl)acrylamide;
158) N-(3-morpholino-5-(3-(4-(trifluoromethyl)phenyl)-1H-indazol-1-yl)phenyl)acrylamide;
159) N-(3-(3-(4-(trifluoromethyl)phenyl)-1H-pyrazolo[3,4-b]pyridin-1-yl)phenyl)acrylamide;
160) 2-fluoro-1-(3-(3-(4-(trifluoromethyl)phenyl)-1H-pyrazolo[3,4-b]pyrazin-1-yl)azetidin-1-yl)prop-2-en-1-one;
161) 1-(3-(3-(4-(trifluoromethyl)phenyl)-1H-pyrazolo[3,4-b]pyrazin-1-yl)azetidin-1-yl)prop-2-en-1-one;
162) 2-fluoro-1-(3-fluoro-3-(3-(4-(trifluoromethyl)phenyl)-1H-pyrazolo[3,4-b]pyridin-1-yl)azetidin-1-yl)prop-2-en-1-one;
163) 2-fluoro-1-(2-fluoro-3-(3-(4-(trifluoromethyl)phenyl)-1H-pyrazolo[3,4-b]pyridin-1-yl)azetidin-1-yl)prop-2-en-1-one;
164) 2-fluoro-1-(3-hydroxy-3-(3-(4-(trifluoromethyl)phenyl)-1H-pyrazolo[3,4-b]pyridin-1-yl)azetidin-1-yl)prop-2-en-1-one;
165) 1-(1-(2-fluoroacryloyl)azetidin-3-yl)-3-(4-(trifluoromethyl)phenyl)-1H-pyrazolo[3,4-b]pyridine 7-oxide;
166) ethyl 2-(1-(2-fluoroacryloyl)-3-(3-(4-(trifluoromethyl)phenyl)-1H-pyrazolo[3,4-b]pyridin-1-yl)azetidin-3-yl)acetate;
167) 2-(1-(2-fluoroacryloyl)-3-(3-(4-(trifluoromethyl)phenyl)-1H-pyrazolo[3,4-b]pyridin-1-yl)azetidin-3-yl)acetonitrile;
168) 2-fluoro-1-(3-(fluoromethyl)-3-(3-(4-(trifluoromethyl)phenyl)-1H-pyrazolo[3,4-b]pyridin-1-yl)azetidin-1-yl)prop-2-en-1-one;
169) 2-(1-(2-fluoroacryloyl)-3-(3-(4-(trifluoromethyl)phenyl)-1H-pyrazolo[3,4-b]pyridin-1-yl)azetidin-3-yl) acetamide;
170) 1-(2-fluoroacryloyl)-3-(3-(4-(trifluoromethyl)phenyl)-1H-pyrazolo[3,4-b]pyridin-1-yl)azetidine-3-carbonitrile;
171) 2-fluoro-1-(3-(3-(4-isopropylphenyl)-1H-pyrazolo[3,4-b]pyridin-1-yl)azetidin-1-yl)prop-2-en-1-one;
172) 2-fluoro-1-(3-(3-(4-(trifluoromethoxy)phenyl)-1H-pyrazolo[3,4-b]pyridin-1-yl)azetidin-1-yl)prop-2-en-1-one;
173) 2-fluoro-1-(3-(3-(4-(pentafluoro-16-sulfanyl)phenyl)-1H-pyrazolo[3,4-b]pyridin-1-yl)azetidin-1-yl)prop-2-en-1-one;
174) 2-methyl-1-(3-(3-(4-(trifluoromethyl)phenyl)-1H-pyrazolo[3,4-b]pyridin-1-yl)azetidin-1-yl)prop-2-en-1-one;
175) (E)-1-(3-(3-(4-(trifluoromethyl)phenyl)-1H-pyrazolo[3,4-b]pyridin-1-yl)azetidin-1-yl)but-2-en-1-one;
176) 2-fluoro-1-(3-(3-(3-(trifluoromethyl)phenyl)-1H-pyrazolo[3,4-b]pyridin-1-yl)azetidin-1-yl)prop-2-en-1-one;
177) 5-(1-(1-(2-fluoroacryloyl)azetidin-3-yl)-1H-pyrazolo[3,4-b]pyridin-3-yl)-2-(trifluoromethyl)benzonitrile;
178) 4-(1-(1-(2-fluoroacryloyl)azetidin-3-yl)-1H-pyrazolo[3,4-b]pyridin-3-yl)-2-(trifluoromethyl)benzonitrile;
179) 2-fluoro-1-(3-(3-(6-(trifluoromethyl)pyridin-2-yl)-1H-pyrazolo[3,4-b]pyridin-1-yl)azetidin-1-yl)prop-2-en-1-one;
180) 2-fluoro-1-(3-(3-(2-(trifluoromethyl)pyridin-4-yl)-1H-pyrazolo[3,4-b]pyridin-1-yl)azetidin-1-yl)prop-2-en-1-one;
181) 2-fluoro-1-(3-(3-(5-methyl-6-(trifluoromethyl)pyridin-3-yl)-1H-pyrazolo[3,4-b]pyridin-1-yl)azetidin-1-yl)prop-2-en-1-one;
182) 2-fluoro-1-(3-(3-(2-methyl-6-(trifluoromethyl)pyridin-3-yl)-1H-pyrazolo[3,4-b]pyridin-1-yl)azetidin-1-yl)prop-2-en-1-one;
183) 2-fluoro-1-(3-(3-(2-(trifluoromethyl)pyrimidin-5-yl)-1H-pyrazolo[3,4-b]pyridin-1-yl)azetidin-1-yl)prop-2-en-1-one;
184) 2-fluoro-1-(3-(3-(5-fluoro-6-(trifluoromethyl)pyridin-3-yl)-1H-pyrazolo[3,4-b]pyridin-1-yl)azetidin-1-yl)prop-2-en-1-one;
185) 2-fluoro-1-(3-(3-(2-fluoro-6-(trifluoromethyl)pyridin-3-yl)-1H-pyrazolo[3,4-b]pyridin-1-yl)azetidin-1-yl)prop-2-en-1-one;
186) 2-fluoro-1-(3-(6-methyl-3-(4-(trifluoromethyl)phenyl)-1H-pyrazolo[3,4-b]pyridin-1-yl)azetidin-1-yl)prop-2-en-1-one;
187) 1-(3-(6-chloro-3-(4-(trifluoromethyl)phenyl)-1H-pyrazolo[3,4-b]pyridin-1-yl)azetidin-1-yl)-2-fluoroprop-2-en-1-one;
188) 2-fluoro-N-(2-methoxy-5-(3-(4-(trifluoromethyl)phenyl)-1H-pyrazolo[3,4-b]pyridin-1-yl)phenyl)acrylamide;
189) N-(2-chloro-5-(3-(4-(trifluoromethyl)phenyl)-1H-pyrazolo[3,4-b]pyridin-1-yl)phenyl)acrylamide;
190) N-(2,4-difluoro-5-(3-(4-(trifluoromethyl)phenyl)-1H-pyrazolo[3,4-b]pyridin-1-yl)phenyl)-2-fluoroacrylamide;
191) 2-fluoro-N-(4-fluoro-3-(3-(4-(trifluoromethyl)phenyl)-1H-pyrazolo[3,4-b]pyridin-1-yl)phenyl)acrylamide;
192) N-(2,4-dichloro-5-(3-(4-(trifluoromethyl)phenyl)-1H-pyrazolo[3,4-b]pyridin-1-yl)phenyl)-2-fluoroacrylamide;
193) 2-fluoro-1-(6-(3-(4-(trifluoromethyl)phenyl)-1H-pyrazolo[3,4-b]pyridin-1-yl)indolin-1-yl)prop-2-en-1-one;
194) N-(4-((dimethylamino)methyl)-3-(3-(4-(trifluoromethyl)phenyl)-1H-pyrazolo[3,4-b]pyridin-1-yl)phenyl)-2-fluoroacrylamide;
195) N-(2,4-difluoro-3-(3-(4-(trifluoromethyl)phenyl)-1H-pyrazolo[3,4-b]pyridin-1-yl)phenyl)-2-fluoroacrylamide;
196) 2-fluoro-1-(3-(3-(4-(trifluoromethyl)phenyl)-1H-indazol-1-yl)azetidin-1-yl)prop-2-en-1-one;
197) 2-fluoro-1-(3-(3-(6-(trifluoromethyl)pyridin-3-yl)-1H-indazol-1-yl)azetidin-1-yl)prop-2-en-1-one;
198) 2-fluoro-1-(3-(3-(6-(trifluoromethyl)pyridin-3-yl)-1H-pyrazolo[3,4-b]pyrazin-1-yl)azetidin-1-yl)prop-2-en-1-one;
199) 2-fluoro-1-(3-(6-methyl-3-(6-(trifluoromethyl)pyridin-3-yl)-1H-pyrazolo[3,4-b]pyridin-1-yl)azetidin-1-yl)prop-2-en-1-one;
200) 1-(1-(2-fluoroacryloyl)azetidin-3-yl)-3-(4-(trifluoromethyl)phenyl)-1H-pyrazolo[3,4-b]pyridine-6-carbonitrile;
201) 1-(1-(2-fluoroacryloyl)azetidin-3-yl)-N-methyl-3-(4-(trifluoromethyl)phenyl)-1H-indazole-7-sulfonamide;
202) 2-fluoro-1-(3-(5-fluoro-3-(4-(trifluoromethyl)phenyl)-1H-pyrazolo[3,4-b]pyridin-1-yl)azetidin-1-yl)prop-2-en-1-one;
203) 2-fluoro-1-(3-(6-fluoro-3-(4-(trifluoromethyl)phenyl)-1H-pyrazolo[3,4-b]pyridin-1-yl)azetidin-1-yl)prop-2-en-1-one;
204) 2-fluoro-1-(3-(6-fluoro-3-(4-(trifluoromethyl)phenyl)-1H-pyrazolo[4,3-b]pyridin-1-yl)azetidin-1-yl)prop-2-en-1-one;
205) 1-(3-(6-(difluoromethyl)-3-(4-(trifluoromethyl)phenyl)-1H-pyrazolo[3,4-b]pyridin-1-yl)azetidin-1-yl)-2-fluoroprop-2-en-1-one;
206) 1-(3-(6-chloro-3-(4-(trifluoromethyl)phenyl)-1H-pyrazolo[3,4-b]pyrazin-1-yl)azetidin-1-yl)-2-fluoroprop-2-en-1-one;
207) 2-fluoro-1-(3-(3-(4-(trifluoromethyl)phenyl)-4,5,6,7-tetrahydro-1H-indazol-1-yl)azetidin-1-yl)prop-2-en-1-one;
208) 2-fluoro-1-(3-(3-(4-(trifluoromethyl)phenyl)-6,7-dihydropyrano[4,3-c]pyrazol-1(4H)-yl)azetidin-1-yl)prop-2-en-1-one;
209) 2-fluoro-1-(3-(3-(4-(trifluoromethyl)phenyl)-6,7-dihydropyrano[4,3-c]pyrazol-2(4H)-yl)azetidin-1-yl)prop-2-en-1-one;
210) 1-(3-(4,4-difluoro-3-(4-(trifluoromethyl)phenyl)-4,5,6,7-tetrahydro-1H-indazol-1-yl)azetidin-1-yl)-2-fluoroprop-2-en-1-one;
211) 1-(3-(5-(difluoromethyl)-3-(4-(trifluoromethyl)phenyl)-1H-pyrazolo[3,4-b]pyridin-1-yl)azetidin-1-yl)-2-fluoroprop-2-en-1-one;
212) 1-(3-(5-(difluoromethyl)-3-(4-(trifluoromethyl)phenyl)-1H-pyrazolo[3,4-b]pyridin-1-yl)azetidin-1-yl)-2-fluoroprop-2-en-1-one;
213) 2-fluoro-1-(3-(5-methyl-3-(4-(trifluoromethyl)phenyl)-1H-pyrazolo[3,4-b]pyridin-1-yl)azetidin-1-yl)prop-2-en-1-one;
214) 2-fluoro-1-(3-(5-methoxy-3-(4-(trifluoromethyl)phenyl)-1H-pyrazolo[3,4-b]pyridin-1-yl)azetidin-1-yl)prop-2-en-1-one;
215) 2-fluoro-1-(3-(5-(trifluoromethyl)-3-(4-(trifluoromethyl)phenyl)-1H-pyrazolo[3,4-b]pyridin-1-yl)azetidin-1-yl)prop-2-en-1-one;
216) 2-fluoro-1-(3-(3-(4-(trifluoromethyl)phenyl)-1H-pyrazolo[4,3-d]pyrimidin-1-yl)azetidin-1-yl)prop-2-en-1-one;
217) 2-fluoro-1-(3-(3-(4-(trifluoromethyl)phenyl)-4,5,6,7-tetrahydro-1H-indazol-1-yl)azetidin-1-yl)prop-2-en-1-one;
218) 6-ethyl-1-(1-(2-fluoroacryloyl)azetidin-3-yl)-3-(4-(trifluoromethyl)phenyl)-1,6-dihydro-7H-pyrazolo[4,3-d]pyrimidin-7-one;
219) 1-(1-(2-fluoroacryloyl)azetidin-3-yl)-7-methyl-3-(4-(trifluoromethyl)phenyl)-1,7-dihydro-6H-pyrazolo[3,4-b]pyridin-6-one;
220) 1-(1-(2-fluoroacryloyl)azetidin-3-yl)-6-methyl-3-(4-(trifluoromethyl)phenyl)-1,6-dihydro-7H-pyrazolo[3,4-c]pyridin-7-one;
221) 2-fluoro-1-(3-(6-methoxy-3-(4-(trifluoromethyl)phenyl)-1H-pyrazolo[3,4-b]pyridin-1-yl)azetidin-1-yl)prop-2-en-1-one;
222) 2-fluoro-1-(3-(7-methoxy-3-(4-(trifluoromethyl)phenyl)-1H-pyrazolo[3,4-c]pyridin-1-yl) azetidin-1-yl)prop-2-en-1-one;
223) 2-fluoro-1-(3-(6-methoxy-3-(4-(trifluoromethyl)phenyl)-1H-pyrazolo[3,4-b]pyrazin-1-yl)azetidin-1-yl)prop-2-en-1-one;
224) 1-(1-(2-fluoroacryloyl)azetidin-3-yl)-7-methyl-3-(4-(trifluoromethyl)phenyl)-1,7-dihydro-6H-pyrazolo[3,4-b]pyrazin-6-one;
225) 1-(1-(2-fluoroacryloyl)azetidin-3-yl)-5,6-dimethyl-3-(4-(trifluoromethyl)phenyl)-1,6-dihydro-7H-pyrazolo[4,3-d]pyrimidin-7-one;
226) 2-fluoro-1-(3-(7-methoxy-5-methyl-3-(4-(trifluoromethyl)phenyl)-1H-pyrazolo[4,3-d]pyrimidin-1-yl)azetidin-1-yl)prop-2-en-1-one;
227) 2-fluoro-1-(3-(7-methoxy-3-(4-(trifluoromethyl)phenyl)-1H-pyrazolo[4,3-d]pyrimidin-1-yl)azetidin-1-yl)prop-2-en-1-one;
228) 1-(3-(5-bromo-3-(4-(trifluoromethyl)phenyl)-1H-pyrazolo[3,4-b]pyridin-1-yl)azetidin-1-yl)-2-fluoroprop-2-en-1-one;
229) 1-(1-(2-fluoroacryloyl)azetidin-3-yl)-3-(4-(trifluoromethyl)phenyl)-1,3-dihydro-2H-benzo[d]imidazol-2-one;
230) 2-fluoro-1-(3-(4-(4-(trifluoromethyl)phenyl)quinazolin-2-yl)azetidin-1-yl)prop-2-en-1-one;
231) 1-(3-(2-imino-3-(4-(trifluoromethyl)phenyl)-2,3-dihydro-1H-benzo[d]imidazol-1-yl)azetidin-1-yl)prop-2-en-1-one;
232) N-((5-(2-oxo-3-(4-(trifluoromethyl)phenyl)-2,3-dihydro-1H-benzo[d]imidazol-1-yl)-1,3,4-oxadiazol-2-yl)methyl)acrylamide;
233) 1-(1-acryloylpyrrolidin-3-yl)-3-(4-cyclohexylphenyl)-1,3-dihydro-2H-benzo[d]imidazol-2-one;
234) 1-(1-acryloylpyrrolidin-3-yl)-3-(4-(trifluoromethyl)phenyl)-1,3-dihydro-2H-benzo[d]imidazol-2-one;
235) 1-(3-(2-imino-3-(4-(trifluoromethyl)phenyl)-2,3-dihydro-1H-benzo[d]imidazol-1-yl)pyrrolidin-1-yl)prop-2-en-1-one;
236) 1-(1-acryloylazetidin-3-yl)-3-(4-(trifluoromethyl)phenyl)-1,3-dihydro-2H-benzo[d]imidazol-2-one;
237) 1-(1-(2-fluoroacryloyl)-3-methylazetidin-3-yl)-3-(4-(trifluoromethyl)phenyl)-1,3-dihydro-2H-benzo[d]imidazol-2-one;
238) 3-(1-(2-fluoroacryloyl)-3-methylazetidin-3-yl)-1-(4-(trifluoromethyl)phenyl)-1,3-dihydro-2H-imidazo[4,5-b]pyridin-2-one;
239) 1-(1-(2-fluoroacryloyl)azetidin-3-yl)-3-(4-(trifluoromethyl)phenyl)-1,3-dihydro-2H-imidazo[4,5-b]pyridin-2-one;
240) 1-(1-(2-fluoroacryloyl)azetidin-3-yl)-3-(4-(trifluoromethyl)phenyl)-1,3-dihydro-2H-imidazo[4,5-b]pyrazin-2-one;
241) 1-(1-(2-fluoroacryloyl)azetidin-3-yl)-3-(6-(trifluoromethyl)pyridin-3-yl)-1,3-dihydro-2H-imidazo[4,5-b]pyrazin-2-one;
242) 3-(1-(2-fluoroacryloyl)azetidin-3-yl)-5-methyl-1-(4-(trifluoromethyl)phenyl)-1,3-dihydro-2H-imidazo[4,5-b]pyridin-2-one;
243) 3-(1-(2-fluoroacryloyl)azetidin-3-yl)-5-methyl-1-(6-(trifluoromethyl)pyridin-3-yl)-1,3-dihydro-2H-imidazo[4,5-b]pyridin-2-one;
244) 3-(1-(2-fluoroacryloyl)azetidin-3-yl)-6-methyl-1-(4-(trifluoromethyl)phenyl)-1,3-dihydro-2H-imidazo[4,5-b]pyridin-2-one;
245) 3-(1-(2-fluoroacryloyl)azetidin-3-yl)-6-(trifluoromethyl)-1-(4-(trifluoromethyl)phenyl)-1,3-dihydro-2H-imidazo[4,5-b]pyridin-2-one;
246) 3-(1-(2-fluoroacryloyl)azetidin-3-yl)-6-methoxy-1-(4-(trifluoromethyl)phenyl)-1,3-dihydro-2H-imidazo[4,5-b]pyridin-2-one;
247) 1-(1-(2-fluoroacryloyl)azetidin-3-yl)-3-(4-(trifluoromethyl)phenyl)-1,3-dihydro-2H-imidazo[4,5-c]pyridin-2-one;
248) 3-(1-(2-fluoroacryloyl)azetidin-3-yl)-1-(4-(trifluoromethyl)phenyl)-1,3-dihydro-2H-imidazo[4,5-c]pyridin-2-one;
249) 6-chloro-3-(1-(2-fluoroacryloyl)azetidin-3-yl)-1-(4-(trifluoromethyl)phenyl)-1,3-dihydro-2H-imidazo[4,5-b]pyridin-2-one;
250) 9-(1-(2-fluoroacryloyl)azetidin-3-yl)-7-(4-(trifluoromethyl)phenyl)-7,9-dihydro-8H-purin-8-one;
251) 3-(1-(2-fluoroacryloyl)azetidin-3-yl)-5-methoxy-1-(4-(trifluoromethyl)phenyl)-1,3-dihydro-2H-imidazo[4,5-b]pyridin-2-one;
252) 5-chloro-3-(1-(2-fluoroacryloyl)azetidin-3-yl)-1-(4-(trifluoromethyl)phenyl)-1,3-dihydro-2H-imidazo[4,5-b]pyridin-2-one;
253) 6-fluoro-3-(1-(2-fluoroacryloyl)azetidin-3-yl)-1-(4-(trifluoromethyl)phenyl)-1,3-dihydro-2H-imidazo[4,5-b]pyridin-2-one;
254) N-(1-(2-(4-(trifluoromethyl)phenyl)quinazolin-4-yl)pyrrolidin-3-yl)acrylamide;
255) 2-fluoro-1-(3-((2-(4-(trifluoromethyl)phenyl)quinazolin-4-yl)amino)azetidin-1-yl)prop-2-en-1-one;
256) 2-fluoro-1-(3-(2-(4-(trifluoromethyl)phenyl)quinazolin-4-yl)azetidin-1-yl)prop-2-en-1-one;
257) 2-fluoro-1-(3-(4-(4-(trifluoromethyl)phenyl)quinazolin-2-yl)azetidin-1-yl)prop-2-en-1-one;
258) N-(1-(1-(4-(trifluoromethyl)phenyl)isoquinolin-3-yl)pyrrolidin-3-yl)acrylamide;
259) N-(3-(1-(4-(trifluoromethyl)phenyl)isoquinolin-3-yl)phenyl)acrylamide;
260) 1-(3-(4-(4-(trifluoromethyl)phenyl)quinolin-2-yl)pyrrolidin-1-yl)prop-2-en-1-one;
261) 1-(3-(4-(4-(trifluoromethyl)phenyl)quinazolin-2-yl)pyrrolidin-1-yl)prop-2-en-1-one;
262) 3-(1-acryloylpyrrolidin-3-yl)-1-(4-(trifluoromethyl)phenyl)quinazoline-2,4(1H,3H)-dione;
263) 2-(1-acryloylpyrrolidin-3-yl)-4-(4-(trifluoromethyl)phenyl)phthalazin-1(2H)-one;
264) 2-(1-acryloylpiperidin-3-yl)-4-(4-(trifluoromethyl)phenyl)phthalazin-1(2H)-one;
265) 3-(1-acryloylpyrrolidin-3-yl)-1-(4-(trifluoromethyl)phenyl)-3,4-dihydroquinazolin-2(1H)-one;
266) 3-(5-(1-acryloylpyrrolidin-3-yl)-1,3,4-oxadiazol-2-yl)-1-(4-(trifluoromethyl)phenyl)quinolin-2(1H)-one;
267) 1-(3-(5-(4-(4-(trifluoromethyl)phenyl)quinazolin-2-yl)-1,3,4-oxadiazol-2-yl)pyrrolidin-1-yl)prop-2-en-1-one;
268) N-(1-(2-(4-(trifluoromethyl)phenyl)quinazolin-4-yl)azetidin-3-yl)acrylamide;
269) 2-fluoro-N-(1-(2-(4-(trifluoromethyl)phenyl)quinazolin-4-yl)azetidin-3-yl)acrylamide;
270) 2-fluoro-N-(1-(2-(4-(trifluoromethyl)phenyl)quinazolin-4-yl)pyrrolidin-3-yl)acrylamide;
271) N-(1-(2-(4-(trifluoromethyl)phenyl)pyrido[2,3-d]pyrimidin-4-yl)pyrrolidin-3-yl)acrylamide;
272) N-(1-(2-(4-(trifluoromethyl)phenyl)pyrido[3,4-d]pyrimidin-4-yl)pyrrolidin-3-yl)acrylamide;
273) 1-(4-(2-(4-(trifluoromethyl)phenyl)pyrido[3,4-d]pyrimidin-4-yl)piperazin-1-yl)prop-2-en-1-one;
274) N-(5-methyl-1-(2-(4-(trifluoromethyl)phenyl)pyrido[3,4-d]pyrimidin-4-yl)pyrrolidin-3-yl)acrylamide;
275) N-(1-(2-(4-(trifluoromethyl)phenyl)pyrido[2,3-d]pyrimidin-4-yl)azetidin-3-yl)acrylamide;
276) 2-fluoro-N-(1-(2-(4-(trifluoromethyl)phenyl)pyrido[2,3-d]pyrimidin-4-yl)azetidin-3-yl)acrylamide;
277) 2-fluoro-N-(1-(2-(4-(trifluoromethyl)phenyl)pyrido[2,3-d]pyrimidin-4-yl)pyrrolidin-3-yl)acrylamide;
278) 2-fluoro-N-(1-(2-(4-(trifluoromethyl)phenyl)pyrido[3,4-d]pyrimidin-4-yl)pyrrolidin-3-yl)acrylamide;
279) N-(1-(2-(4-(trifluoromethyl)phenyl)pyrido[3,4-d]pyrimidin-4-yl)azetidin-3-yl)acrylamide;
280) 2-fluoro-N-(1-(2-(4-(trifluoromethyl)phenyl)pyrido[3,4-d]pyrimidin-4-yl)azetidin-3-yl)acrylamide;
281) N-(1-(2-(4-(trifluoromethyl)phenyl)pyrido[3,2-d]pyrimidin-4-yl)azetidin-3-yl)acrylamide;
282) 2-fluoro-N-(1-(2-(4-(trifluoromethyl)phenyl)pyrido[3,2-d]pyrimidin-4-yl)azetidin-3-yl)acrylamide;
283) 1-(3-((2-(4-(trifluoromethyl)phenyl)quinazolin-4-yl)amino)azetidin-1-yl)prop-2-en-1-one;
284) N-(3-(4-(4-(trifluoromethyl)phenoxy)naphthalen-2-yl)phenyl)acrylamide;
285) 1-(3-(2-(4-(trifluoromethyl)phenyl)quinolin-4-yl)pyrrolidin-1-yl)prop-2-en-1-one;
286) 1-(3-(6-(4-(trifluoromethyl)phenyl)quinolin-8-yl)pyrrolidin-1-yl)prop-2-en-1-one;
287) N-(1-(2-(4-(trifluoromethyl)phenyl)pyrido[3,2-d]pyrimidin-4-yl)pyrrolidin-3-yl)acrylamide;
288) 2-fluoro-N-(1-(2-(4-(trifluoromethyl)phenyl)pyrido[3,2-d]pyrimidin-4-yl)pyrrolidin-3-yl)acrylamide;
289) 3-(1-acryloylpyrrolidin-3-yl)-1-(4-(trifluoromethyl)phenyl)-4a,8a-dihydroquinolin-2(1H)-one;
290) 2-(1-acryloylpyrrolidin-3-yl)-4-(4-(trifluoromethyl)phenyl)-4a,8a-dihydroisoquinolin-1(2H)-one;
291) 2-(1-acryloylazetidin-3-yl)-4-(4-(trifluoromethyl)phenyl)phthalazin-1(2H)-one;
292) 3-(1-acryloylazetidin-3-yl)-1-(4-(trifluoromethyl)phenyl)quinazoline-2,4(1H,3H)-dione;
293) 2-(1-acryloylazetidin-3-yl)-4-(4-(trifluoromethyl)phenyl)isoquinolin-1(2H)-one;
294) 1-(3-(4-(4-(trifluoromethyl)phenyl)quinazolin-2-yl)azetidin-1-yl)prop-2-en-1-one;
295) 2-fluoro-N-(1-(3-(4-(trifluoromethyl)phenyl)naphthalen-1-yl)azetidin-3-yl)acrylamide;
296) 2-fluoro-N-(1-(6-(4-(trifluoromethyl)phenyl)quinolin-8-yl)azetidin-3-yl)acrylamide;
297) 1-(1-(2-fluoroacryloyl)azetidin-3-yl)-3-(4-(trifluoromethyl)phenyl)quinolin-2(1H)-one;
298) 1-(1-(2-fluoroacryloyl)azetidin-3-yl)-3-(4-(trifluoromethyl)phenyl)-1,8-naphthyridin-2(1H)-one;
299) 1-(1-(2-fluoroacryloyl)azetidin-3-yl)-3-(4-(trifluoromethyl)phenyl)quinoxalin-2(1H)-one;
300) 4-(1-(2-fluoroacryloyl)azetidin-3-yl)-2-(4-(trifluoromethyl)phenyl)pyrido[2,3-b]pyrazin-3(4H)-one;
301) 2-fluoro-N-(1-(2-(4-(trifluoromethyl)phenyl)pteridin-4-yl)azetidin-3-yl)acrylamide;
302) 2-fluoro-N-methyl-N-(1-(2-(4-(trifluoromethyl)phenyl)quinazolin-4-yl)azetidin-3-yl)acrylamide;
303) 2-fluoro-N-(1-(7-methoxy-2-(4-(trifluoromethyl)phenyl)quinazolin-4-yl)azetidin-3-yl)acrylamide;
304) 2-fluoro-N-(1-(5-(trifluoromethyl)-2-(4-(trifluoromethyl)phenyl)quinazolin-4-yl)azetidin-3-yl)acrylamide;
305) 2-fluoro-N-(1-(2-(4-(trifluoromethyl)phenyl)pyrido[4,3-d]pyrimidin-4-yl)azetidin-3-yl)acrylamide;
306) 2-fluoro-N-(1-(2-(6-(trifluoromethyl)pyridin-3-yl)pyrido[3,2-d]pyrimidin-4-yl)azetidin-3-yl)acrylamide;
307) 2-fluoro-1-(3-(3-(phenylethynyl)-1H-pyrazolo[3,4-b]pyridin-1-yl)azetidin-1-yl)prop-2-en-1-one;
308) (E)-2-fluoro-1-(3-(3-styryl-1H-pyrazolo[3,4-b]pyridin-1-yl)azetidin-1-yl)prop-2-en-1-one;
309) 1-(3-(3-((3,3-difluorocyclobutyl)ethynyl)-1H-pyrazolo[3,4-b]pyridin-1-yl)azetidin-1-yl)prop-2-en-1-one;
310) N-(3-(3-(4-(trifluoromethyl)phenyl)-1H-pyrazolo[3,4-b]pyridin-1-yl)phenyl)acrylamide;
311) 1-(3-(3-(cyclopentylethynyl)-1H-pyrazolo[3,4-b]pyridin-1-yl)azetidin-1-yl)prop-2-en-1-one;
312) 1-(3-(3-(cyclopentylethynyl)-1H-pyrazolo[3,4-b]pyridin-1-yl)azetidin-1-yl)-2-fluoroprop-2-en-1-one;
313) 1-(3-(3-(pyrimidin-2-ylethynyl)-1H-pyrazolo[3,4-b]pyridin-1-yl)azetidin-1-yl)prop-2-en-1-one;
314) 2-fluoro-1-(3-(3-(pyrimidin-2-ylethynyl)-1H-pyrazolo[3,4-b]pyridin-1-yl)azetidin-1-yl)prop-2-en-1-one;
315) 1-(3-(3-(cyclopropylethynyl)-1H-pyrazolo[3,4-b]pyridin-1-yl)azetidin-1-yl)prop-2-en-1-one;
316) 1-(3-(3-(thiophen-3-ylethynyl)-1H-pyrazolo[3,4-b]pyridin-1-yl)azetidin-1-yl)prop-2-en-1-one;
317) 2-fluoro-1-(3-(3-(thiophen-3-ylethynyl)-1H-pyrazolo[3,4-b]pyridin-1-yl)azetidin-1-yl)prop-2-en-1-one;
318) 1-(3-(3-((1-methyl-1H-imidazol-5-yl)ethynyl)-1H-pyrazolo[3,4-b]pyridin-1-yl)azetidin-1-yl)prop-2-en-1-one;
319) 1-(3-(3-(phenylethynyl)-1H-pyrazolo[3,4-b]pyridin-1-yl)azetidin-1-yl)prop-2-en-1-one;
320) 1-(3-(3-(cyclobutylethynyl)-1H-pyrazolo[3,4-b]pyridin-1-yl)azetidin-1-yl)prop-2-en-1-one;
321) 1-(3-(3-(cyclobutylethynyl)-1H-pyrazolo[3,4-b]pyridin-1-yl)azetidin-1-yl)-2-fluoroprop-2-en-1-one;
322) 1-(3-(3-(cyclopropylethynyl)-1H-pyrazolo[3,4-b]pyridin-1-yl)azetidin-1-yl)-2-fluoroprop-2-en-1-one;
323) 2-fluoro-1-(3-(3-((1-methyl-1H-imidazol-5-yl)ethynyl)-1H-pyrazolo[3,4-b]pyridin-1-yl)azetidin-1-yl)prop-2-en-1-one;
324) 1-(3-(3-(cyclohexylethynyl)-1H-pyrazolo[3,4-b]pyridin-1-yl)azetidin-1-yl)prop-2-en-1-one;
325) 1-(3-(3-(cyclohexylethynyl)-1H-pyrazolo[3,4-b]pyridin-1-yl)azetidin-1-yl)-2-fluoroprop-2-en-1-one;
326) 1-(3-(3-((3,3-difluorocyclobutyl)ethynyl)-1H-pyrazolo[3,4-b]pyridin-1-yl)azetidin-1-yl)-2-fluoroprop-2-en-1-one;
327) (E)-1-(3-(3-styryl-1H-pyrazolo[3,4-b]pyridin-1-yl)azetidin-1-yl)prop-2-en-1-one;
328) 1-(3-(3-((3,3-difluorocyclopentyl)ethynyl)-1H-pyrazolo[3,4-b]pyridin-1-yl)azetidin-1-yl)prop-2-en-1-one;
329) 1-(3-(3-((3,3-difluorocyclopentyl)ethynyl)-1H-pyrazolo[3,4-b]pyridin-1-yl)azetidin-1-yl)-2-fluoroprop-2-en-1-one;
330) (E)-1-(3-(3-(2-cyclohexylvinyl)-1H-pyrazolo[3,4-b]pyridin-1-yl)azetidin-1-yl)prop-2-en-1-one;
331) (E)-1-(3-(3-(2-cyclohexylvinyl)-1H-pyrazolo[3,4-b]pyridin-1-yl)azetidin-1-yl)-2-fluoroprop-2-en-1-one;
332) (E)-1-(3-(3-(2-cyclopropylvinyl)-1H-pyrazolo[3,4-b]pyridin-1-yl)azetidin-1-yl)prop-2-en-1-one;
333) (E)-1-(3-(3-(2-cyclopropylvinyl)-1H-pyrazolo[3,4-b]pyridin-1-yl)azetidin-1-yl)-2-fluoroprop-2-en-1-one;
334) 2-fluoro-1-(3-(3-((4-fluorophenyl)ethynyl)-1H-pyrazolo[3,4-b]pyridin-1-yl)azetidin-1-yl)prop-2-en-1-one;
335) 2-fluoro-1-(3-(3-((3-fluorophenyl)ethynyl)-1H-pyrazolo[3,4-b]pyridin-1-yl)azetidin-1-yl)prop-2-en-1-one;
336) 2-fluoro-1-(3-(3-((2-fluorophenyl)ethynyl)-1H-pyrazolo[3,4-b]pyridin-1-yl)azetidin-1-yl)prop-2-en-1-one;
337) 1-(3-(3-((3-chlorophenyl)ethynyl)-1H-pyrazolo[3,4-b]pyridin-1-yl)azetidin-1-yl)-2-fluoroprop-2-en-1-one;
338) 1-(3-(3-((3-((difluoro-13-methyl)-12-fluoranyl)phenyl)ethynyl)-1H-pyrazolo[3,4-b]pyridin-1-yl)azetidin-1-yl)-2-fluoroprop-2-en-1-one;
339) (E)-2-fluoro-1-(3-(3-(4-fluorostyryl)-1H-pyrazolo[3,4-b]pyridin-1-yl)azetidin-1-yl)prop-2-en-1-one;
340) (E)-2-fluoro-1-(3-(3-(3-fluorostyryl)-1H-pyrazolo[3,4-b]pyridin-1-yl)azetidin-1-yl)prop-2-en-1-one;
341) (E)-2-fluoro-1-(3-(3-(2-fluorostyryl)-1H-pyrazolo[3,4-b]pyridin-1-yl)azetidin-1-yl)prop-2-en-1-one;
342) (E)-1-(3-(3-(4-chlorostyryl)-1H-pyrazolo[3,4-b]pyridin-1-yl)azetidin-1-yl)-2-fluoroprop-2-en-1-one;
343) (E)-2-fluoro-1-(3-(3-(4-(trifluoromethyl)styryl)-1H-pyrazolo[3,4-b]pyridin-1-yl)azetidin-1-yl)prop-2-en-1-one;
344) (E)-4-(2-(1-(1-(2-fluoroacryloyl)azetidin-3-yl)-1H-pyrazolo[3,4-b]pyridin-3-yl)vinyl)benzonitrile;
345) (E)-2-fluoro-1-(3-(3-(3-(trifluoromethyl)styryl)-1H-pyrazolo[3,4-b]pyridin-1-yl)azetidin-1-yl)prop-2-en-1-one;
346) 1-(3-(3-((2,3-difluorophenyl)ethynyl)-1H-pyrazolo[3,4-b]pyridin-1-yl)azetidin-1-yl)-2-fluoroprop-2-en-1-one;
347) 2-fluoro-1-(3-(3-((2-fluorophenyl)ethynyl)-1H-pyrazolo[3,4-b]pyridin-1-yl)azetidin-1-yl)prop-2-en-1-one;
348) 1-(3-(3-((2-chlorophenyl)ethynyl)-1H-pyrazolo[3,4-b]pyridin-1-yl)azetidin-1-yl)-2-fluoroprop-2-en-1-one; or
349) 2-fluoro-1-(3-(3-((2-(trifluoromethyl)phenyl)ethynyl)-1H-pyrazolo[3,4-b]pyridin-1-yl)azetidin-1-yl)prop-2-en-1-one.

The present invention also provides a pharmaceutical composition comprising a compound of any of the present invention and a pharmaceutically acceptable excipient, such as hydroxypropyl methyl cellulose. In the composition, the said compound in a weight ratio to the said excipient within the range from about 0.0001 to about 10.

The present invention additionally provided a use of a pharmaceutical composition of Formula (I) for the preparation of a medicament for treating a disease in a subject.

The present invention further provides some preferred technical solutions with regard to above-mentioned uses.

In some embodiments, a medicament thus prepared can be used for the treatment or prevention of, or for delaying or preventing onset or progression in, cancer, cancer metastasis. The cancer is colon cancer, gastric cancer, thyroid cancer, lung cancer, leukemia, pancreatic cancer, melanoma, multiple melanoma, brain cancer, renal cancer, liver cancer, squamous cancer, gastrointestinal cancer, mesothelioma, prostate cancer, ovarian cancer or breast cancer.

The present invention provided a method for the therapeutic treatment of disease in a subject, the said method comprising administering to a subject in need thereof a therapeutically effective amount of a compound of the present invention, or a pharmaceutically acceptable salt or a stereoisomer thereof. Wherein the disease is colon cancer, gastric cancer, thyroid cancer, lung cancer, leukemia, pancreatic cancer, melanoma, multiple melanoma, brain cancer, renal cancer, liver cancer, squamous cancer, gastrointestinal cancer, mesothelioma, prostate cancer, ovarian cancer, or breast cancer.

The present invention also provides a use of the present compound or its pharmaceutical composition for the preparation of a medicament.

In some embodiments, the medicament is used for the treatment, prevention of cancer or hyperproliferative disorder.

In some embodiments, the cancer is colon cancer, gastric cancer, thyroid cancer, lung cancer, leukemia, pancreatic cancer, melanoma, multiple melanoma, brain cancer, renal cancer, liver cancer, squamous cancer, gastrointestinal cancer, mesothelioma, prostate cancer, ovarian cancer or breast cancer.

In some embodiments, the medicament is used as an inhibitor of YAP/TAZ-TEAD interaction.

The general chemical terms used in the formula above have their usual meanings. For example, the term “halogen”, as used herein, unless otherwise indicated, means fluoro, chloro, bromo or iodo. The preferred halogen groups include F, Cl and Br.

As used herein, unless otherwise indicated, alkyl includes saturated monovalent hydrocarbon radicals having straight, branched or cyclic moieties. For example, alkyl radicals include methyl, ethyl, propyl, isopropyl, cycicopropyl, n-butyl, isobutyl, sec-butyl, t-butyl, cycicobutyl, n-pentyl, 3-(2-methyl) butyl, 2-pentyl, 2-methylbutyl, neopentyl, cycicopentyl, n-hexyl, 2-hexyl, 2-methylpentyl and cyclohexyl. Similarly, C₁₋₄, as in C₁₋₄alkyl is defined to identify the group as having 1, 2, 3, or 4 carbon atoms in a linear or branched arrangement.

Alkenyl and alkynyl groups include straight, branched chain or cyclic alkenes and alkynes. Likewise, “C₂₋₈ alkenyl” and “C₂₋₈ alkynyl” means an alkenyl or alkynyl radicals having 2, 3, 4, 5, 6, 7 or 8 carbon atoms in a linear or branched arrangement.

Alkoxy radicals are oxygen ethers formed from the previously described straight, branched chain or cyclic alkyl groups.

The term “aryl”, as used herein, unless otherwise indicated, refers to an unsubstituted or substituted mono- or polycyclic ring system containing carbon ring atoms. The preferred aryls are mono cyclic or bicyclic 6-10 membered aromatic ring systems. Phenyl and naphthyl are preferred aryls. The most preferred aryl is phenyl.

The term “heterocyclyl”, as used herein, unless otherwise indicated, represents an unsubstituted or substituted stable three to ten membered saturated or partially unsaturated monocyclic, spirocyclic, bridged bicyclic or fused bicyclic ring system which consists of carbon atoms and one to three heteroatoms selected from N, O or S, and wherein the nitrogen or sulfur heteroatoms may optionally be oxidized, and the nitrogen heteroatom may optionally be quaternized. The heterocyclyl group may be attached at any heteroatom or carbon atom which results in the creation of a stable structure. Examples of such heterocyclyl groups include, but are not limited to azetidinyl, pyrrolidinyl, piperidinyl, piperazinyl, oxopiperazinyl, oxopiperidinyl, oxoazepinyl, azepinyl, tetrahydrofuranyl, dioxolanyl, tetrahydroimidazolyl, tetrahydrothiazolyl, tetrahydrooxazolyl, tetrahydropyranyl, morpholinyl, thiomorpholinyl, thiamorpholinyl sulfoxide, thiamorpholinyl sulfone and oxadiazolyl.

The term “heteroaryl”, as used herein, unless otherwise indicated, represents an unsubstituted or substituted stable five to six membered monocyclic aromatic ring system or an unsubstituted or substituted eight to ten membered fused heteroaromatic ring system or bicyclic heteroaromatic ring system which consists of carbon atoms and one to four heteroatoms selected from N, O or S, and wherein the nitrogen or sulfur heteroatoms may optionally be oxidized, and the nitrogen heteroatom may optionally be quaternized. The heteroaryl group may be attached at any heteroatom or carbon atom which results in the creation of a stable structure. Examples of heteroaryl groups include, but are not limited to thienyl, furanyl, imidazolyl, isoxazolyl, oxazolyl, pyrazolyl, pyrrolyl, thiazolyl, thiadiazolyl, triazolyl, pyridyl, pyridazinyl, indolyl, azaindolyl, indazolyl, benzimidazolyl, benzofuranyl, benzothienyl, benzisoxazolyl, benzoxazolyl, benzopyrazolyl, benzothiazolyl, benzothiadiazolyl, benzotriazolyl adeninyl, quinolinyl or isoquinolinyl.

The term “alkenyloxy” refers to the group —O-alkenyl, where alkenyl is defined as above.

The term “alknyloxy” refers to the group —O-alknyl, where alknyl is defined as above.

The term “cycloalkyl” refers to a cyclic saturated alkyl chain having from 3 to 12 carbon atoms, for example, cyclopropyl, cyclobutyl, cyclobutyl, cyclobutyl.

The term “heterocycloalkyl” refers to a cyclic saturated alkyl chain having carbon atoms and 1 to 3 heteroatoms selected from N, O or S, and wherein the nitrogen or sulfur heteroatoms may optionally be oxidized, and the nitrogen heteroatom may optionally be quaternized, for example, azetidinyl, pyrrolidinyl, piperidinyl, piperazinyl, oxopiperazinyl, oxopiperidinyl, oxoazapyridine.

The term “substituted” refers to a group in which one or more hydrogen atoms are each independently replaced with the same or different substituent(s). Typical substituents include, but are not limited to, halogen (F, Cl, Br or I), C₁₋₈ alkyl, C₃₋₁₂ cycloalkyl, —OR¹, SR¹, ═O, ═S, —C(O)R¹, —C(S)R¹, ═NR¹, —C(O)OR¹, —C(S)OR¹, —NR¹R², —C(O)NR¹R², cyano, nitro, —S(O)₂R¹, —OS(O₂)OR¹, —OS(O)₂R¹, —OP(O)(OR¹)(OR²); wherein R¹ and R² is independently selected from —H, lower alkyl, lower haloalkyl. In some embodiments, the substituent(s) is independently selected from the group consisting of —F, —Cl, —Br, —I, —OH, trifluromethoxy, ethoxy, propyloxy, iso-propyloxy, n-butyloxy, isobutyloxy, t-butyloxy, —SCH₃, —SC₂H₅, formaldehyde group, —C(OCH₃), cyano, nitro, CF₃,—OCF₃, amino, dimethylamino, methyl thio, sulfonyl and acetyl.

The “ [symbol] ” represent a site which is attached to L₁ or L₂.

The “ [symbol] ₐ” represent the site which is fused to ring A.

The term “composition”, as used herein, is intended to encompass a product comprising the specified ingredients in the specified amounts, as well as any product which results, directly or indirectly, from combinations of the specified ingredients in the specified amounts. Accordingly, pharmaceutical compositions containing the compounds of the present invention as the active ingredient as well as methods of preparing the instant compounds are also part of the present invention. Furthermore, some of the crystalline forms for the compounds may exist as polymorphs and as such are intended to be included in the present invention. In addition, some of the compounds may form solvates with water (i.e., hydrates) or common organic solvents and such solvates are also intended to be encompassed within the scope of this invention.

Examples of substituted alkyl group include, but not limited to, 2-aminoethyl, 2-hydroxyethyl, pentachloroethyl, trifluoromethyl, methoxymethyl, pentafluoroethyl and piperazinylmethyl.

Examples of substituted alkoxy groups include, but not limited to, aminomethoxy, thrifluoromethoxy, 2-diethylaminoethoxy, 2-ethoxycarbonylethoxy, 3-hydroxypropoxy.

The compounds of the present invention may also be present in the form of pharmaceutically acceptable salts. For use in medicine, the salts of the compounds of this invention refer to non-toxic “pharmaceutically acceptable salts”. The pharmaceutically acceptable salt forms include pharmaceutically acceptable acidic/anionic or basic/cationic salts. The pharmaceutically acceptable acidic/anionic salt generally takes a form in which the basic nitrogen is protonated with an inorganic or organic acid. Representative organic or inorganic acids include hydrochloric, hydrobromic, hydriodic, perchloric, sulfuric, nitric, phosphoric, acetic, propionic, glycolic, lactic, succinic, maleic, fumaric, malic, tartaric, citric, benzoic, mandelic, methanesulfonic, hydroxyethanesulfonic, benzenesulfonic, oxalic, pamoic, 2-naphthalenesulfonic, p-toluenesulfonic, cyclohexanesulfamic, salicylic, saccharinic or trifluoroacetic. Pharmaceutically acceptable basic/cationic salts include, and are not limited to aluminum, calcium, chloroprocaine, choline, diethanolamine, ethylenediamine, lithium, magnesium, potassium, sodium and zinc.

The present invention includes within its scope the prodrugs of the compounds of this invention. In general, such prodrugs will be functional derivatives of the compounds that are readily converted in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the subject. Conventional procedures for the selection and preparation of suitable prodrug derivatives are described, for example, in “Design of Prodrugs”, ed. H. Bundgaard, Elsevier, 1985.

It is intended that the definition of any substituent or variable at a particular location in a molecule be independent of its definitions elsewhere in that molecule. It is understood that substituents and substitution patterns on the compounds of this invention can be selected by one of ordinary skill in the art to provide compounds that are chemically stable and that can be readily synthesized by techniques know in the art as well as those methods set forth herein.

The present invention includes compounds described herein can contain one or more asymmetric centers and may thus give rise to diastereomers and optical isomers. The present invention includes all such possible diastereomers as well as their racemic mixtures, their substantially pure resolved enantiomers, all possible geometric isomers, and pharmaceutically acceptable salts thereof.

The above Formula I is showed without a definitive stereochemistry at certain positions. The present invention includes all stereoisomers of Formula I and pharmaceutically acceptable salts thereof. Further, mixtures of stereoisomers as well as isolated specific stereoisomers are also included. During the course of the synthetic procedures used to prepare such compounds, or in using racemization or epimerization procedures known to those skilled in the art, the products of such procedures can be a mixture of stereoisomers.

When a tautomer of the compound of Formula I exists, the present invention includes any possible tautomers and pharmaceutically acceptable salts thereof, and mixtures thereof, except where specifically stated otherwise.

When the compound of Formula I and pharmaceutically acceptable salts thereof exist in the form of solvates or polymorphic forms, the present invention includes any possible solvates and polymorphic forms. A type of a solvent that forms the solvate is not particularly limited so long as the solvent is pharmacologically acceptable. For example, water, ethanol, propanol, acetone or the like can be used.

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N′,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

When the compound of the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, formic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Preferred are citric, hydrobromic, formic, hydrochloric, maleic, phosphoric, sulfuric and tartaric acids, particularly preferred are formic and hydrochloric acid. Since the compounds of Formula I are intended for pharmaceutical use they are preferably provided in substantially pure form, for example at least 60% pure, more suitably at least 75% pure, especially at least 98% pure (% are on a weight for weight basis).

The pharmaceutical compositions of the present invention comprise a compound represented by Formula I (or a pharmaceutically acceptable salt thereof) as an active ingredient, a pharmaceutically acceptable carrier and optionally other therapeutic ingredients or adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the compounds represented by Formula I, or a prodrug, or a metabolite, or pharmaceutically acceptable salts thereof, of this invention can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as oil-in-water emulsion, or as a water-in-oil liquid emulsion. In addition to the common dosage forms set out above, the compound represented by Formula I, or a pharmaceutically acceptable salt thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention may include a pharmaceutically acceptable carrier and a compound, or a pharmaceutically acceptable salt, of Formula I. The compounds of Formula I, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include such as lactose, terra alba, sucrose, talc, gelatin, agar, pectin, acacia, magnesium stearate, and stearic acid. Examples of liquid carriers include such as sugar syrup, peanut oil, olive oil, and water. Examples of gaseous carriers include such as carbon dioxide and nitrogen. In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 5 g of the active ingredient and each cachet or capsule preferably containing from about 0.05 mg to about 5 g of the active ingredient. For example, a formulation intended for the oral administration to humans may contain from about 0.5 mg to about 5 g of active agent, compounded with an appropriate and convenient amount of carrier material which may vary from about 5 to about 95 percent of the total composition. Unit dosage forms will generally contain between from about 1 mg to about 2 g of the active ingredient, typically 25 mg, 50 mg, 100 mg, 200 mg, 300 mg, 400 mg, 500 mg, 600 mg, 800 mg, or 1000 mg.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared, utilizing a compound represented by Formula I of this invention, or a pharmaceutically acceptable salt thereof, via conventional processing methods. As an example, a cream or ointment is prepared by admixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including antioxidants) and the like. Furthermore, other adjuvants can be included to render the formulation isotonic with the blood of the intended recipient. Compositions containing a compound described by Formula I, or pharmaceutically acceptable salts thereof, may also be prepared in powder or liquid concentrate form.

Generally, dosage levels on the order of from about 0.01 mg/kg to about 150 mg/kg of body weight per day are useful in the treatment of the above-indicated conditions, or alternatively about 0.5 mg to about 7 g per patient per day. For example, colon cancer, rectal cancer, mantle cell lymphoma, multiple myeloma, breast cancer, prostate cancer, glioblastoma, squamous cell esophageal cancer, liposarcoma, T-cell lymphoma melanoma, pancreatic cancer, glioblastoma or lung cancer, may be effectively treated by the administration of from about 0.01 to 50 mg of the compound per kilogram of body weight per day, or alternatively about 0.5 mg to about 3.5 g per patient per day.

It is understood, however, that lower or higher doses than those recited above may be required. Specific dose level and treatment regimens for any particular subject will depend upon a variety of factors, including the activity of the specific compound employed, the age, body weight, general health, sex, diet, time of administration, route of administration, rate of excretion, drug combination, the severity and course of the particular disease undergoing therapy, the subject disposition to the disease, and the judgment of the treating physician.

These and other aspects will become apparent from the following written description of the invention.

The following Examples are provided to better illustrate the present invention. All parts and percentages are by weight and all temperatures are degrees Celsius, unless explicitly stated otherwise.

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results. The compounds of the Examples have been found to inhibit the transcription activity of YAP/TAZ-TEAD protein/protein interaction according to at least one assay described herein.

### DESCRIPTION OF THE DRAWING

FIG. 1: The inhibition curve of compound 5 in NCI-H226 cell line in Brdu assay.

FIG. 2: The inhibition curve of compound 6 in NCI-H226 cell line in Brdu assay.

FIG. 3: The inhibition curve of compound 30 in NCI-H226 cell line in Brdu assay.

FIG. 4: The inhibition curve of compound 73 in NCI-H226 cell line in Brdu assay.

FIG. 5: The inhibition curve of compound 80 in NCI-H226 cell line in Brdu assay.

FIG. 6: The inhibition curve of compound 124 in NCI-H226 cell line in Brdu assay.

FIG. 7: The inhibition curve of compound 132 in NCI-H226 cell line in Brdu assay.

FIG. 8: The tumor growth curves of different treatment groups of Balb/c nude mice bearing NCI-H226 tumor.

FIG. 9: Percentage change of the body weight of different treatment groups in in Balb/c nude mice bearing NCI-H226 tumor.

### EXAMPLES

The compounds described herein can be obtained from commercial sources or synthesized by conventional methods as shown below using commercially available starting materials and reagents. The following abbreviations have been used in the examples:
BOP: (tri(dimethylamino)benzotriazol-1-yloxyphosphonium hexafluorophosphate);
Cu(OAc)₂: cupric acetate;
DMA: Dimethylacetamide;
DMF: Dimethylformamide;
DIAD: Diisopropyl azodicarboxylate;
DEAD: diethyl azodicarboxylate;
DIEA or DIPEA: N,N-Diisopropylethylamine;
DMSO: Dimethyl sulfoxide;
DCM: Dichloromethane;
EA: Ethyl Acetate;
EDTA: Ethylenediaminetetraacetic acid;
HATU: 2-(7-Azabenzotriazol-1-yl)-N,N,N′,N′-tetramethyluronium hexafluorophosphate;
TMB: 3,3′,5,5′-Tetramethylbenzidine;
TBAF: Tetrabutylammonium fluoride;
TBDPSCl: tert-Butyldiphenylchlorosilane;
THF: Tetrahydrofuran;
TFA: Trifluoroacetic acid;
TEA: Triethylamine;
Mscl: Methanesulfonyl chloride;
NIS: N-iodosuccinimide;
NMP: N-Methylpyrrolidone;
PBS: phosphate buffered saline;
HRP: horseradish peroxidase;
h or hrs: hour or hours;
Hex: Hexane;
HATU: 1-[Bis(dimethylamino)methylene]-1H-1,2,3-triazolo[4,5-b]pyridinium 3-oxide hexafluorophosphate;
LCMS: Liquid Chromatography Mass Spectrometry;
MeOH: Methanol;
min: minute;
NIS: N-iodosuccinimide;
Pd/C: Palladium on carbon;
PE: Petroleum ether;
PPh₃: Triphenylphosphine;
Pd(PPh₃)₄: Tetrakis(triphenylphosphine)palladium;
Pd(dppf)Cl₂·CH₂Cl₂: [1,1′-Bis(diphenylphosphino)ferrocene]dichloropalladium(II), complex with dichloromethane;
Rt or r.t or RT: room temperature.

### Example 1 Synthesis of Compound 1 (1-(1-acryloylpyrrolidin-3 yl)-3-(4-cyclohexylphenyl)-1,6-dihydro-7H-pyrazolo[4,3-d]pyrimidin-7-one)

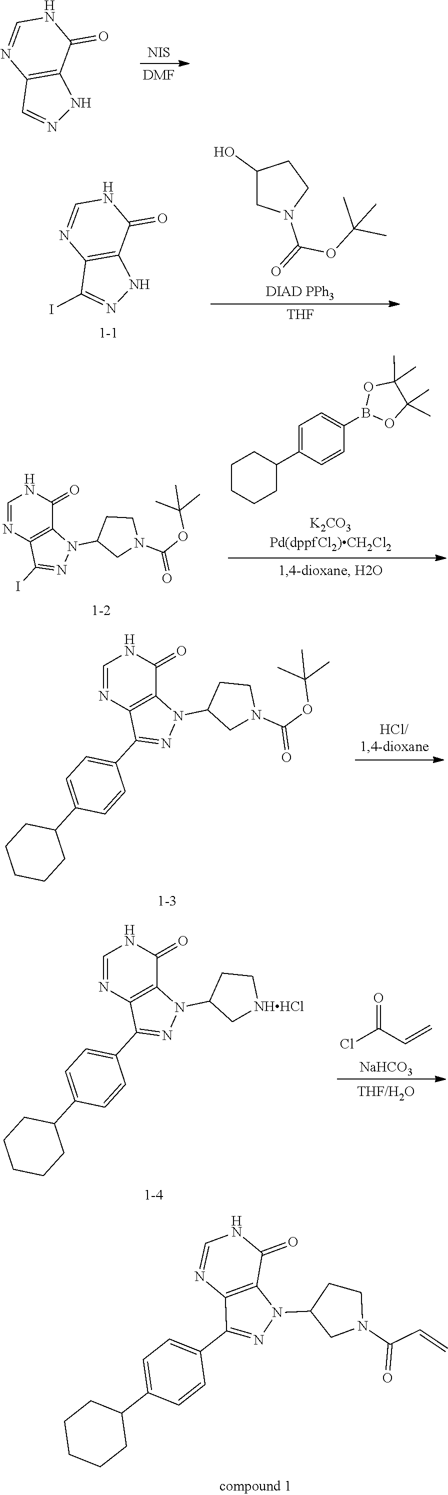

### Step 1: Preparation of 3-iodo-1,6-dihydro-7H-pyrazolo[4,3-d]pyrimidin-7-one

To a mixture of 1,6-dihydro-7H-pyrazolo[4,3-d]pyrimidin-7-one (1.00 g, 7.35 mmol) in DMF (20 mL) was added NIS (2.48 g, 11.02 mmol). The mixture was stirred at room temperature for 1 h and then stirred at 60° C. for 4 hs. After cooling to rt, the mixture was poured into ice water, stirred and filtered. The filter cake was suspended in toluene and concentrated under vacuum to afford the title compound 1-1 (1.90 g). LCMS [M+H]⁺=262.94.

### Step 2: Preparation of tert-butyl 3-(3-iodo-7-oxo-6,7-dihydro-1H-pyrazolo[4,3-d]pyrimidin-1 yl)pyrrolidine-1-carboxylate

PPh₃ (501 mg, 1.91 mmol) was added into a mixture of compound 1-1 (250 mg, 954.17 umol) and tert-butyl 3-hydroxypyrrolidine-1-carboxylate (250 mg, 1.34 mmol) in THE (5 mL), and then DIAD (386 mg, 1.91 mmol) was added dropwise at 0° C. The mixture was allowed to warm up to room temperature naturally and stirred for 16 hs. The mixture was concentrated under vacuum to get the residue, the residue was further purified by silica gel column (Hex:EA=0%-50%) to afford the tittle compound 1-2 (450 mg). LCMS [M+H]⁺=432.05.

### Step 3: Preparation of tert-butyl 3-(3-(4-cyclohexylphenyl)-7-oxo-6,7-dihydro-1H-pyrazolo[4,3-d]pyrimidin-1 yl)pyrrolidine-1-carboxylate

To a mixture of compound 1-2 (450 mg, 1.04 mmol) and 2-(4-cyclohexylphenyl)-4,4,5,5-tetramethyl-1,3,2-dioxaborolane (213 mg, 1.04 mmol) in 1,4-dioxane (10 mL) and H₂O (1 mL) was added Pd(dppf)Cl₂·CH₂Cl₂ (76 mg, 103 umol), K₂CO₃ (432 mg, 3.13 mmol). The mixture was stirred at 100° C. for 6 hs under nitrogen atmosphere. The reaction mixture was concentrated under vacuum to get the residue, the residue was further purified by silica gel column (Hex:EA=0%-50%) to afford the tittle compound1-3 (450 mg). LCMS [M+H]+=464.26.

### Step 4: Preparation of 3-(4-cyclohexylphenyl)-1-(pyrrolidin-3 yl)-1,6-dihydro-7H-pyrazolo[4,3-d]pyrimidin-7-one

A mixture of compound1-3 (340 mg, 733 mol) in HCl/1,4-dioxane (4.0N, 10 ml) was stirred at room temperature for 4 hs. The reaction mixture was concentrated under vacuum to afford the title compound1-4 (400 mg), which was used for the next step without any further purification. LCMS [M+H]+=400.18.

### Step 5: Preparation of 1-(1-acryloylpyrrolidin-3 yl)-3-(4-cyclohexylphenyl)-1,6-dihydro-7H-pyrazolo[4,3-d]pyrimidin-7-one (compound 1)

NaHCO₃ (139 mg, 1.65 mmol) was added into a mixture of compound 1-4 (200 mg, 550 umol) in THF/H₂O (v:v=1:1, 10 mL), and then acryloyl chloride (50 mg, 552 umol) was added dropwise at 0° C. under nitrogen atmosphere. The mixture was stirred at 0° C. for 0.5 h, then diluted with EA, washed with water, dried over anhydrous sodium sulfate and concentrated under vacuum to get the residue. The residue was purified by silica gel chromatography (DCM:MeOH=20:1) to afford the title compound 1 (31.9 mg). LCMS [M+H]+=418.22.

### Example 2 Synthesis of Compound 2 (1-(1-acryloylpyrrolidin-3 yl)-3-(4-(trifluoromethyl)phenyl)-1,6-dihydro-7H-pyrazolo[4,3-d]pyrimidin-7-one)

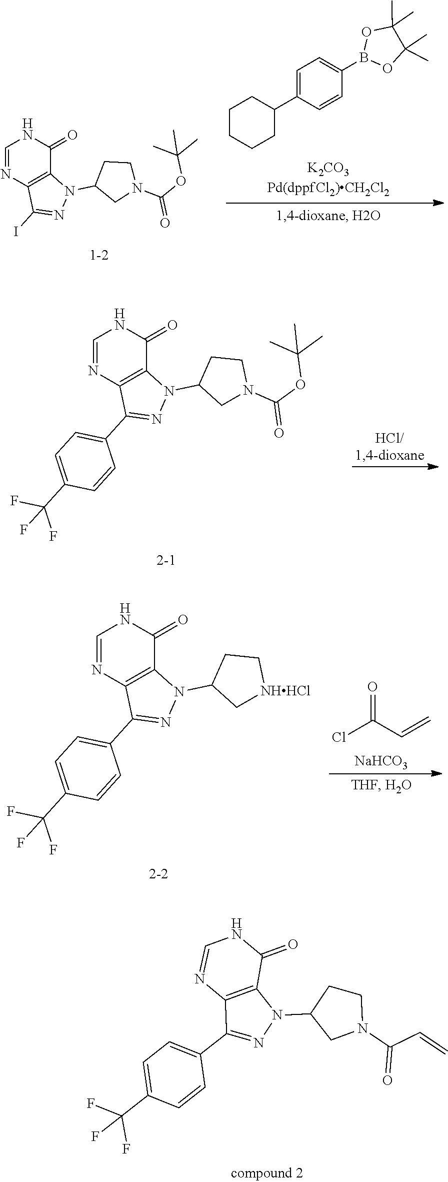

### Step 1: Preparation of tert-butyl 3-(7-oxo-3-(4-(trifluoromethyl)phenyl)-6,7-dihydro-1H-pyrazolo[4,3-d]pyrimidin-1 yl)pyrrolidine-1-carboxylate

Compound 1-2 (350 mg, 811 umol) and 4,4,5,5-tetramethyl-2-(4-(trifluoromethyl)phenyl)-1,3,2-dioxaborolane (231 mg, 1.22 mmol) were added into the mixture of Pd(dppf)Cl₂·CH₂Cl₂ (60 mg, 81 umol), K₂CO₃ (336 mg, 2.43 mmol) in 1,4-dioxane (10 mL) and H₂O (1 mL). the reaction mixture was stirred at 100° C. for 6 hs under N₂ atmosphere, concentrated under reduced pressure to get the residue. The residue was further purified by silica gel column (Hex:EA=0%-50%) to get compound 2-1 (350 mg). LCMS [M+4]⁺=450.17.

### Step 2: Preparation of 1-(pyrrolidin-3 yl)-3-(4-(trifluoromethyl)phenyl)-1,6-dihydro-7H-pyrazolo[4,3-d]pyrimidin-7-one

A mixture of compound 2-1 (350 mg, 733 umol) in HCl/1,4-dioxane (4.0N, 10 ml) was stirred overnight at room temperature, concentrated under reduced pressure to obtain compound 2-2 (400 mg). LCMS [M+H]+=400.18

### Step 3: Preparation of 1-(1-acryloylpyrrolidin-3-yl)-3-(4-(trifluoromethyl)phenyl)-1,6-dihydro-7H-pyrazolo[4,3-d]pyrimidin-7-one

NaHCO₃ (130 mg, 1.56 mmol) was added into a mixture of compound 2-2 (200 mg, 518 umol) in THF/H₂O (v:v=1:1, 10 mL), and then acryloyl chloride (47 mg, 518 umol) was added dropwises at 0° C. The mixture was stirred at 0° C. for 0.5 h, diluted with EA, washed with water, dried over MgSO₄ and concentrated in vacuo to get the residue. The residue was purified by Prep_TLC (DCM:MeOH=20:1) to afford compound 2 (10 mg). LCMS [M+H]+=404.13.

### Example 3 Synthesis of Compound 3(1-(3-(3-(4-(trifluoromethyl)phenyl)-1H-indazol-1 yl)pyrrolidin-1 yl)prop-2-en-1-one)

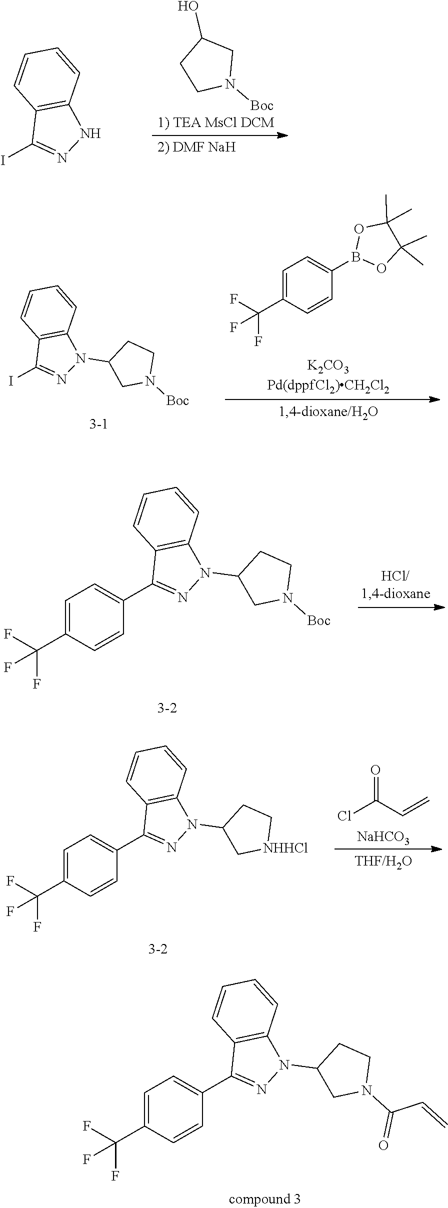

### Step 1 Preparation of tert-butyl 3-(3-iodo-1H-indazol-1 yl)pyrrolidine-1-carboxylate

Methanesulfonyl chloride (140 mg, 1.23 mmol) was added dropwise with stirring into a mixture of tert-butyl 3-hydroxypyrrolidine-1-carboxylate (184 mg, 0.98 mmol), TEA (248 mg, 0.34 mL), and DCM (5.00 mL) at 0° C. under nitrogen atmosphere. The reaction mixture was stirred at 0° C. for 0.5 h. The reaction mixture was then quenched by the addition of water, extracted with dichloromethane, washed with water, dried over anhydrous sodium sulfate, and filtered. The filtrate was concentrated under vacuum to afford intermediate 1 (200 mg).

A mixture of 3-iodo-1H-indazole (200 mg, 819 umol) in DMF (5.00 mL) was added NaH (19.67 mg) in portions at 0° C. under nitrogen atmosphere. After the mixture was stirred for 30 min at rt, the intermediate 1 obtained above was added into the reaction mixture and then the reaction mixture was stirred at 60° C. overnight. The reaction mixture was cooled down to room temperature and diluted with ethyl acetate, washed with water, dried, and concentrated under vacuum to get a residue. The residue was purified by silica gel chromatography (Hex:EA=1:1) to afford the tittle compound 3-1 (100 mg). LCMS [M+H]⁺=414.06.

### Step 2: Preparation of tert-butyl 3-(3-(4-(trifluoromethyl)phenyl)-1H-indazol-1-yl)pyrrolidine-1-carboxylate

A mixture of compound 3-1 (100 mg, 0.24 mmol), 4,4,5,5-tetramethyl-2-(4-(trifluoromethyl)phenyl)-1,3,2-dioxaborolane (45 mg, 0.24 mmol), potassium carbonate (100 mg, 0.73 mmol), Pd(dppf)Cl₂·CH₂Cl₂ (17 mg, 0.024 mmol), 1,4-dioxane (5 mL), and water (0.5 mL) was stirred for 6 h at 100° C. under nitrogen. The mixture was concentrated under vacuum to get the residue, the residue was further purified by silica gel column (Hex:EA=0%-50%) to afford the title compound 3-2 (120 mg). LCMS [M+H]⁺=432.18.

### Step 3: Preparation of 1-(pyrrolidin-3 yl)-3-(4-yl)-1H-indazole

A mixture of compound 3-2 (100 mg, 231 umol) in HCl/1,4-dioxane (4.0N, 10 ml) was stirred overnight at room temperature. The reaction mixture was concentrated under vacuum to afford the title compound 3-3 (100 mg), which was used for the next step without any further purification. LCMS [M+H]⁺=332.13.

### Step 4: Preparation of 1-(3-(3-(4-(trifluoromethyl)phenyl)-1H-indazol-1 yl)pyrrolidin-1-yl)prop-2-en-1-one

NaHCO₃ (130 mg, 1.56 mmol) was added into a mixture of compound 3-3 (300 mg, 905 umol) in THF/H₂O (v:v=1:1, 20 mL), and then acryloyl chloride (81 mg, 905 umol) was added dropwises at 0° C. under nitrogen atmosphere. The mixture was stirred at 0° C. for 10 min, and then diluted with EA, washed with water, dried over anhydrous sodium sulfate and concentrated under vacuum to get the residue. The residue was purified by silica gel column (DCM:MeOH=30:1) to afford the title compound 3 (44.1 mg).

LCMS [M+H]+=386.14.

¹H NMR (500 MHz, DMSO-d₆) δ 8.21 (d, J=3.2 Hz, 1H), 8.19 (d, J=3.2 Hz, 1H), 8.15 (d, J=8.2 Hz, 1H), 7.88 (d, J=3.4 Hz, 1H), 7.87 (s, 1H), 7.86 (d, J=3.7 Hz, 1H), 7.53 (dd, J=8.4, 6.8 Hz, 1H), 7.33 (t, J=7.5 Hz, 1H), 6.65 (ddd, J=38.9, 16.8, 10.3 Hz, 1H), 6.18 (ddd, J=16.7, 5.7, 2.4 Hz, 1H), 5.75-5.51 (m, 2H), 4.21-3.95 (m, 2H), 3.91-3.83 (m, 1H), 3.81-3.58 (m, 1H), 2.56 (dt, J=13.4, 6.5 Hz, 1H), 2.46 (q, J=6.9 Hz, 1H).

### Example 4 Synthesis of Compound 4 (1-(3-(1-(4-(trifluoromethyl)phenyl)-1H-indazol-3 yl)pyrrolidin-1 yl)prop-2-en-1-one)

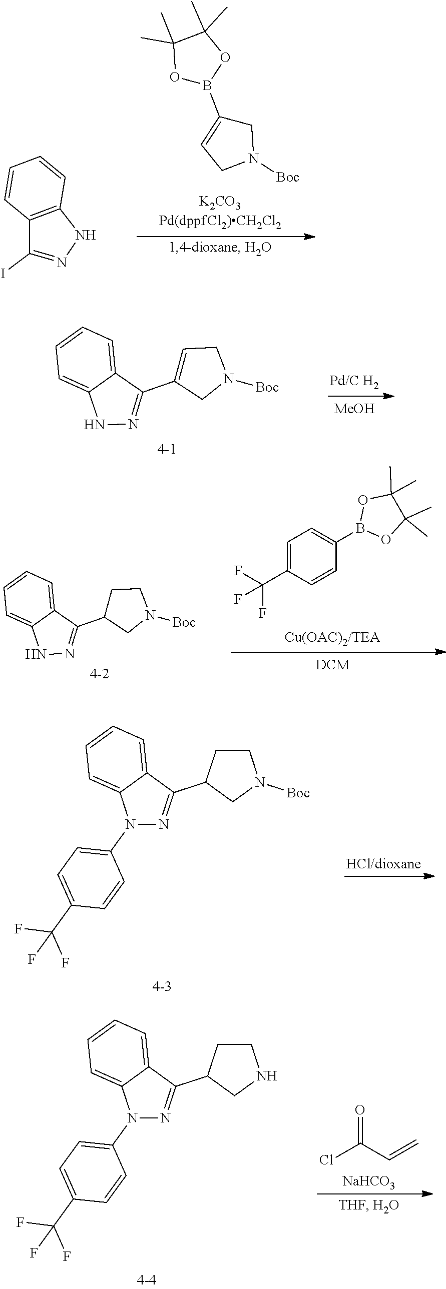

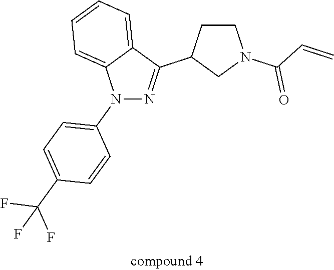 [SMILES: C=CC(=O)N1CCC(c2nn(-c3ccc(C(F)(F)F)cc3)c3ccccc23)C1]

### Step 1: Preparation of tert-butyl 3-(1H-indazol-3 yl)-2,5-dihydro-1H-pyrrole-1-carboxylate

A mixture of 3-iodo-1H-indazole (200 mg, 0.82 mmol), tert-butyl 3-(4,4,5,5-tetramethyl-1,3,2-dioxaborolan-2-yl)-2,5-dihydro-1H-pyrrole-1-carboxylate (290 mg, 0.98 mmol), Pd(dppf)Cl₂·CH₂Cl₂ (67 mg, 0.082 mmol), potassium carbonate (339 mg, 2.46 mmol), 1,4-dioxane (10 mL) and water (1 mL) was stirred for 6 h at 90° C. under nitrogen. The reaction was monitored by LCMS. The reaction mixture was cooled down to room temperature. The mixture was diluted with ethyl acetate, washed with water, dried over anhydrous sodium sulfate, and then filtered. The filtrate was concentrated under vacuum. The residue was purified by silica gel chromatography (DCM:MeOH=20:1) to afford the tittle compound 4-1 (210 mg). LCMS [M+H]⁺=286.15.

### Step 2: Preparation of tert-butyl 3-(1H-indazol-3-yl) pyrrolidine-1-carboxylate

A mixture of compound 4-1 (210 mg, 0.74 mmol), Pd/C (10 mg), and methanol (20 mL) was stirred for 6 h at room temperature under hydrogen atmosphere. The reaction was monitored by LCMS. The reaction mixture was filtered. The filtrate was concentrated under vacuum to afford the title compound 4-2 (200 mg). LCMS [M+H]⁺=288.16.

### Step 3: Preparation of tert-butyl 3-(1-(4-(trifluoromethyl)phenyl)-1H-indazol-3-yl)pyrrolidine-1-carboxylate

A mixture of compound 4-2 (200 mg, 0.70 mmol), 4,4,5,5-tetramethyl-2-(4-(trifluoromethyl)phenyl)-1,3,2-dioxaborolane (132 mg, 0.70 mmol), TEA (211 mg, 2.09 mmol), Cu(OAc)₂ (63 mg, 0.35 mmol) and dichloromethane (20 mL) was stirred for 14 h at room temperature. The reaction mixture was diluted with dichloromethane, washed with water, dried over anhydrous sodium sulfate, and filtered. The filtrate was concentrated under vacuum. The residue was purified by silica gel chromatography (DCM:MeOH=30:1) to afford the tittle compound 4-3 (210 mg). LCMS [M+H]⁺=432.18.

### Step 4: Preparation of 3-(pyrrolidin-3 yl)-1-(4-(trifluoromethyl)phenyl)-1H-indazole hydrochloride

A mixture of compound 4-3 (210 mg, 0.49 mmol) and hydrogen chloride in 1,4-dioxane (4.0 M, 10 mL) was stirred for 1.5 h at room temperature. The reaction mixture was concentrated under vacuum to afford the title compound 4-4 (200 mg), which was directly used for the next step without any further purification. LCMS [M+H]⁺=332.13.

### Step 5: Preparation of 1-(3-(1-(4-(trifluoromethyl)phenyl)-1H-indazol-3-yl)pyrrolidin-1-yl)prop-2-en-1-one

Acrylyl chloride (35 mg, 0.37 mmol) was added dropwise with stirring into a mixture of compound 4-4 (100 mg, 0.30 mmol), sodium bicarbonate (76 mg, 0.90 mmol), THE (10 mL), and water (5 mL) at 0° C. under nitrogen atmosphere. The mixture was stirred for 1 h at 0° C.

The reaction mixture was then diluted with ethyl acetate, washed with water, dried over anhydrous sodium sulfate, and then filtered. The filtrate was concentrated under vacuum. The residue was purified by silica gel chromatography (DCM: MeOH=20:1) to afford the tittle compound 4 (33 mg).

LCMS [M+H]⁺=386.14.

¹H NMR (500 MHz, DMSO-d₆) δ 8.04 (s, 1H), 8.04-7.96 (m, 3H), 7.93 (dd, J=8.8, 2.2 Hz, 2H), 7.66-7.54 (m, 1H), 7.34 (t, J=7.5 Hz, 1H), 6.66 (ddd, J=16.5, 10.3, 5.9 Hz, 1H), 6.17 (dt, J=16.8, 2.7 Hz, 1H), 5.69 (ddd, J=12.4, 10.4, 2.4 Hz, 1H), 4.24-3.98 (m, 2H), 3.98-3.75 (m, 2H), 3.72 (ddd, J=11.9, 8.5, 3.9 Hz, 1H), 2.48-2.20 (m, 2H).

### Example 5 Synthesis of Compound 5 (2 fluoro-1-(3-(3-(4-(trifluoromethyl)phenyl)-1H-pyrazolo[3,4-b]pyridin-1 yl)azetidin-1 yl)prop-2-en-1-one)

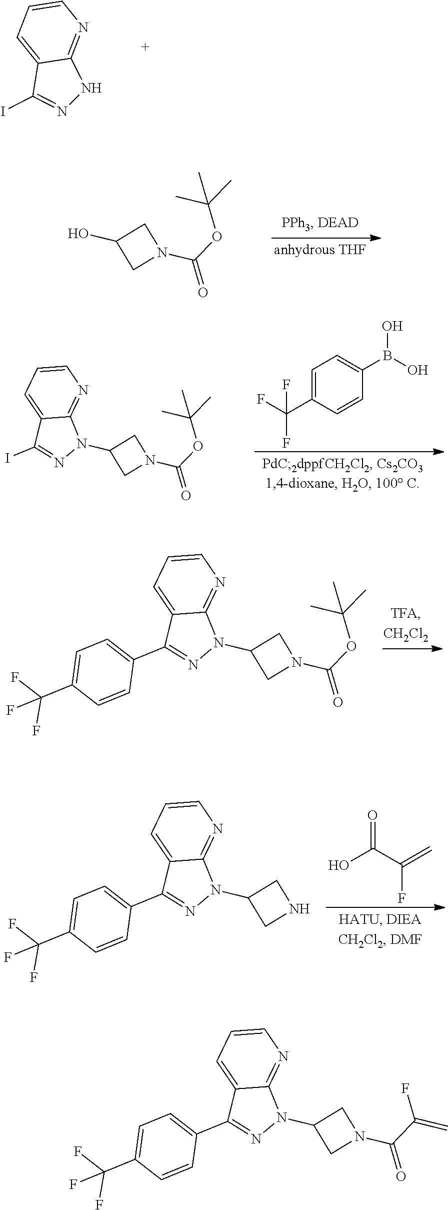

### Step 1: Preparation of tert-butyl 3-(3-iodo-1H-pyrazolo[3,4-b]pyridin-1 yl)azetidine-1-carboxylate

DEAD (8.53 g, 48.98 mmol) was added dropwise with stirring into a mixture of 3-iodo-1H-pyrazolo[3,4-b]pyridine (4.00 g, 16.33 mmol), tert-butyl 3-hydroxyazetidine-1-carboxylate (4.24 g, 24.49 mmol), triphenylphosphine (12.85 g, 48.98 mmol), and anhydrous THF (80 mL) at 0° C. The reaction mixture was stirred at 0° C. for 10 min, and then was allowed to warm up to room temperature. The reaction was stirred for overnight. The reaction was monitored by LCMS. The reaction mixture was concentrated under vacuum and the residue was purified by silica gel chromatography eluting with PE:EA=3:1 to afford the title compound (7.83 g). LCMS [M+H]⁺=401.04.

### Step 2: Preparation of tert-butyl 3-(3-(4-(trifluoromethyl)phenyl)-1H-pyrazolo[3,4-b]pyridin-1 yl)azetidine-1-carboxylate

A mixture of tert-butyl 3-(3-iodo-1H-pyrazolo[3,4-b]pyridin-1-yl)azetidine-1-carboxylate (4.00 g, 10 mmol), (4-(trifluoromethyl)phenyl)boronic acid (2.85 g, 15 mmol), Cs₂CO₃ (9.77 g, 30 mmol), Pd(dppf)Cl₂CH₂Cl₂ (0.82 g, 1 mmol), 1,4-dioxane (40 mL), and water (8 mL) was stirred for 6 h at 100° C. under nitrogen. The mixture was concentrated under vacuum. The residue was further purified by silica gel chromatograph eluting with DCM to afford the title compound (4.62 g). LCMS [M+4]⁺=419.16.

### Step 3: Preparation of 1-(azetidin-3-yl)-3-(4-(trifluoromethyl)phenyl)-1H-pyrazolo[3,4-b]pyridine

A mixture of tert-butyl 3-(3-(4-(trifluoromethyl)phenyl)-1H-pyrazolo[3,4-b]pyridin-1-yl)azetidine-1-carboxylate (4.62 g, 11.04 mmol), TFA (15 mL) and DCM (20 mL) was stirred for 1 h at room temperature. The reaction mixture was concentrated under vacuum to afford the title compound (3.18 g) as light yellow solid, which was used for the next step without any further purification. LCMS [M+H]⁺=319.11.

### Step 4: Preparation of 2 fluoro-1-(3-(3-(4-(trifluoromethyl)phenyl)-1H-pyrazolo[3,4-b]pyridin-1 yl)azetidin-1 yl)prop-2-en-1-one

A mixture of 1-(azetidin-3-yl)-3-(4-(trifluoromethyl)phenyl)-1H-pyrazolo[3,4-b]pyridine (3.18 g, 10 mmol), 2-fluoroacrylic acid (1.35 g, 15 mmol), HATU (7.60 g, 20 mmol), DIEA (8.07 mL, 50 mmol), DCM (100 mL) and DMF (2 mL) was stirred for 5 h at room temperature. The reaction mixture was washed with water and brine, dried over anhydrous sodium sulfate, and then filtered. The filtrate was concentrated under vacuum. The residue was further purified by silica gel chromatography eluting with Hex:EA=1.5:1 to afford the title compound (0.94 g) as light yellow solid.

LCMS [M+H]⁺=391.11.

¹H NMR (500 MHz, CDCl₃) δ 8.58 (d, J=3.4 Hz, 1H), 8.36 (dd, J=8.05, 0.9 Hz, 1H), 8.11 (d, J=8.1 Hz, 2H), 7.78 (d, J=8.2 Hz, 2H), 7.28 (dd, J=8.1, 4.5 Hz, 1H), 6.07-5.97 (m, 1H), 5.71 (dd, J=46.65, 3.0 Hz, 1H), 5.15 (dd, J=15.65, 3.0 Hz, 1H), 5.09-5.02 (m, 1H), 5.00-4.92 (m, 1H), 4.83-4.75 (m, 1H), 4.73-4.64 (m, 1H).

### Example 6 Synthesis of Compound 6 (2 fluoro-1-(3-(3-(4-(trifluoromethyl)phenyl)-1H-pyrazolo[4,3-b]pyridin-1 yl)azetidin-1 yl)prop-2-en-1-one)

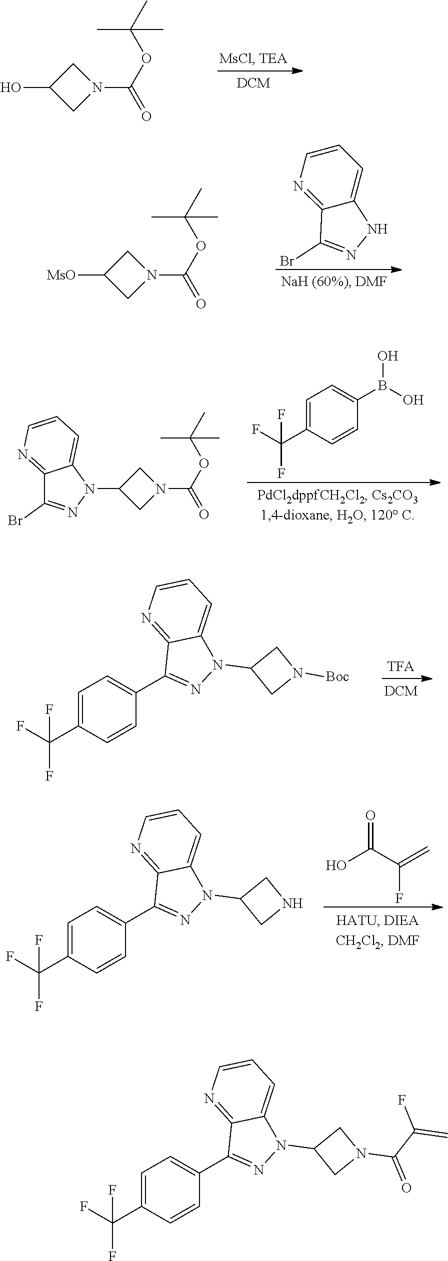

### Step 1: Preparation of tert-butyl 3-((methylsulfonyl)oxy)azetidine-1-carboxylate

Methanesulfonyl chloride (7.94 g, 69.28 mmol) was added dropwise with stirring into a mixture of tert-butyl 3-hydroxyazetidine-1-carboxylate (10.00 g, 57.73 mmol), TEA (16.05 mL, 115.47 mmol), and DCM (30 mL) at 0° C. The reaction mixture was stirred at 0° C. for 1.5 h. The reaction mixture was then quenched by the addition of water, extracted with dichloromethane, washed with water, dried over anhydrous sodium sulfate, and filtered. The filtrate was concentrated under vacuum to afford title compound (14.67 g), which was used for the next step without any further purification.

### Step 2: Preparation of tert-butyl 3-(3-bromo-1H-pyrazolo[4,3-b]pyridin-1 yl)azetidine-1-carboxylate

A mixture of 3-bromo-1H-pyrazolo[4,3-b]pyridine (1.60 g, 8.08 mmol) in DMF (20 mL) was added NaH (60% suspended in mineral oil, 0.97 g, 24.24 mmol) in portions at 0° C. under nitrogen. After the mixture was stirred for 30 min at room temperature, tert-butyl 3-((methylsulfonyl)oxy)azetidine-1-carboxylate (6.09 g, 24.24 mmol) was added into the reaction mixture. The reaction mixture was stirred at 90° C. overnight. The reaction mixture was cooled down to room temperature and diluted with DCM, washed with water, dried over anhydrous sodium sulfate, and filtered. The filtrate was concentrated under vacuum. The residue was purified by silica gel chromatography eluting with DCM:CH₃OH=30:1 to afford the tittle compound (3.17 g) as light yellow solid. LCMS [M+H]⁺=353.05.

### Step 3: Preparation of tert-butyl 3-(3-(4-(trifluoromethyl)phenyl)-1H-pyrazolo[4,3-b]pyridin-1 yl)azetidine-1-carboxylate

A mixture of tert-butyl 3-(3-bromo-1H-pyrazolo[4,3-b]pyridin-1-yl)azetidine-1-carboxylate (2.85 g, 8.07 mmol), (4-(trifluoromethyl)phenyl)boronic acid (2.30 g, 12.10 mmol), Cs₂CO₃ (7.89 g, 24.21 mmol), Pd(dppf)Cl₂CH₂Cl₂ (0.66 g, 0.81 mmol), 1,4-dioxane (30 mL), and water (6 mL) was stirred for 4 h at 120° C. under nitrogen. The mixture was concentrated under vacuum. The residue was further purified by silica gel chromatography eluting with DCM to afford the title compound (1.80 g) as light yellow solid. LCMS [M+H]⁺=419.16.

### Step 4: Preparation of 1-(azetidin-3-yl)-3-(4-(trifluoromethyl)phenyl)-1H-pyrazolo[4,3-b]pyridine

A mixture of tert-butyl 3-(3-(4-(trifluoromethyl)phenyl)-1H-pyrazolo[4,3-b]pyridin-1-yl)azetidine-1-carboxylate (1.80 g, 4.30 mmol), TFA (10 mL) and DCM (20 mL) was stirred for 1 h at room temperature. The reaction mixture was concentrated under vacuum to afford the title compound, which was used for the next step without any further purification. LCMS [M+H]⁺=319.11.

### Step 5: Preparation of 2 fluoro-1-(3-(3-(4-(trifluoromethyl)phenyl)-1H-pyrazolo[4,3-b]pyridin-1 yl)azetidin-1 yl)prop-2-en-1-one

A mixture of 1-(azetidin-3-yl)-3-(4-(trifluoromethyl)phenyl)-1H-pyrazolo[4,3-b]pyridine (1.22 g, 3.83 mmol), 2-fluoroacrylic acid (0.52 g, 5.75 mmol), HATU (2.91 g, 7.67 mmol), DIEA (3.16 mL, 19.15 mmol), DCM (100 mL) and DMF (2 mL) was stirred for 1 h at room temperature. The reaction mixture was washed with water and brine, dried over anhydrous sodium sulfate, and filtered. The filtrate was concentrated under vacuum. The residue was further purified by silica gel chromatography eluting with Hex:EA=1.5:1 to afford the title compound (0.60 g) as off white solid.

LCMS [M+H]⁺=391.11.

¹H NMR (500 MHz, CDCl₃) δ 8.73 (d, J=3.4 Hz, 1H), 8.70 (d, J=8.1 Hz, 2H), 7.81 (d, J=8.0 Hz, 1H), 7.76 (d, J=8.3 Hz, 2H), 7.39 (dd, J=8.6, 4.3 Hz, 1H), 5.73 (dd, J=46.7, 3.0 Hz, 1H), 5.58-5.47 (m, 1H), 5.18 (dd, J=15.6, 3.0 Hz, 1H), 5.10-5.01 (m, 1H), 5.00-4.90 (m, 1H), 4.84-4.75 (m, 1H), 4.73-4.65 (m, 1H).

### Example 7 Synthesis of Compound 7 (1-(1-acryloylpyrrolidin-3 yl)-6-methyl-3-(4-(trifluoromethyl)phenyl)-1,6-dihydro-7H-pyrazolo[4,3-d]pyrimidin-7-one)

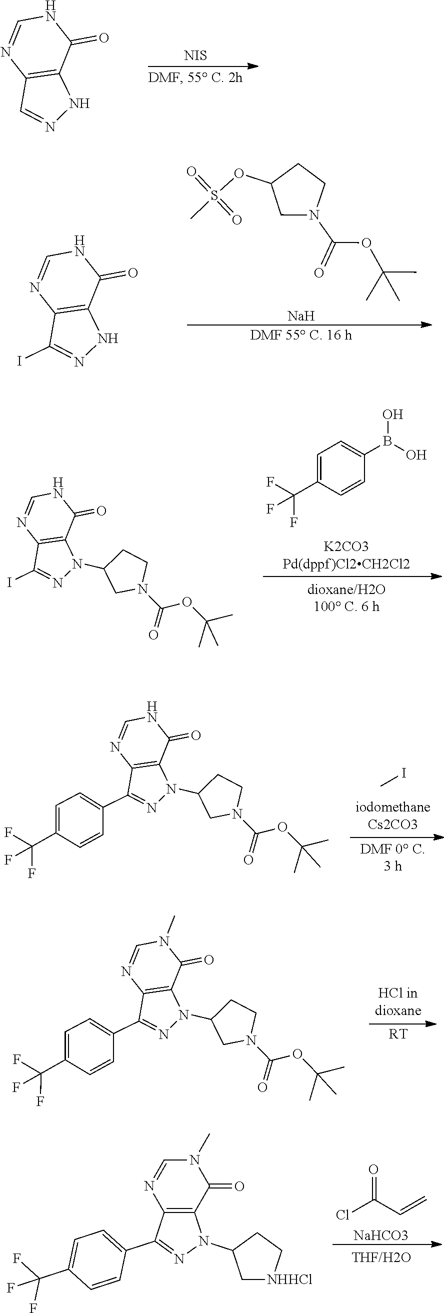

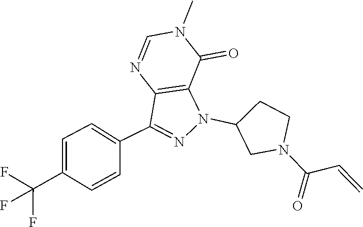 [SMILES: C=CC(=O)N1CCC(n2nc(-c3ccc(C(F)(F)F)cc3)c3ncn(C)c(=O)c32)C1]

### Step 1: Preparation of 3-iodo-1,6-dihydro-7H-pyrazolo[4,3-d]pyrimidin-7-one

A mixture of 1,6-dihydro-7H-pyrazolo[4,3-d]pyrimidin-7-one (2.00 g, 14.69 mmol), DMF (20 mL), and N-iodosuccinimide (4.96 g, 22.04 mmol) was stirred for 1 h at room temperature and then stirred for 4 h at 60° C. The reaction was monitored by LCMS. The reaction was cooled down to room temperature, poured into ice-water. The resulting mixture was filtered and washed with EA. The filter cake was suspended in toluene, and then concentrated under vacuum to afford the title compound (2.75 g) as off-white solid. LCMS [M+H]⁺=262.94.

### Step 2: Preparation of tert-butyl 3-(3-iodo-7-oxo-6,7-dihydro-1H-pyrazolo[4,3-d]pyrimidin-1 yl)pyrrolidine-1-carboxylate

Sodium hydride (60% suspended in mineral oil, 330 mg, 13.74 mmol) was added in portions into a mixture of 3-iodo-1,6-dihydro-7H-pyrazolo[4,3-d]pyrimidin-7-one (1.20 g, 4.58 mmol) and DMF (10 mL) at 0° C. under nitrogen. The reaction was allowed to warm up to room temperature naturally and stirred for 1 h. The reaction mixture was added tert-butyl 3-((methylsulfonyl)oxy)pyrrolidine-1-carboxylate (2.43 g, 9.16 mmol) at room temperature, and then stirred for 16 h at 60° C. The reaction was monitored by LCMS. The reaction was cooled down to room temperature, and then diluted with ethyl acetate, poured into ice-water. The organic layer was separated, dried over anhydrous sodium sulfate, and then filtered. The filtrate was concentrated under vacuum. The residue was washed with hexane (20 ml×3), and then then filtered. The filter cake was concentrated under vacuum to afford the title compound (1.52 g) as off-white solid. LCMS [M+H]⁺=376.23.

### Step 3: Preparation of tert-butyl 3-(7-oxo-3-(4-(trifluoromethyl)phenyl)-6,7-dihydro-1H-pyrazolo[4,3-d]pyrimidin-1 yl)pyrrolidine-1-carboxylate

A mixture of tert-butyl 3-(3-iodo-7-oxo-6,7-dihydro-1H-pyrazolo[4,3-d]pyrimidin-1-yl)pyrrolidine-1-carboxylate (1.20 g, 2.78 mmol), (4-(trifluoromethyl)phenyl)boronic acid (0.79 g, 4.17 mmol), Pd(dppf)Cl₂·CH₂Cl₂ (227 mg, 0.28 mmol), potassium carbonate (1.15 g, 8.35 mmol), 1,4-dioxane (20 mL), and water (2.0 mL) was stirred for 6 h at 100° C. under nitrogen. The reaction was monitored by LCMS. The reaction was cooled down to room temperature. The mixture was concentrated under vacuum. The residue was purified by silica gel chromatography eluting with Hex:EA=0%-40% to afford the tittle compound (1.52 g) as light yellow solid. LCMS [M+H]⁺=394.42.

### Step 4: Preparation of tert-butyl 3-(6-methyl-7-oxo-3-(4-(trifluoromethyl)phenyl)-6,7-dihydro-1H-pyrazolo[4,3-d]pyrimidin-1 yl)pyrrolidine-1-carboxylate

Iodomethane (0.24 g, 1.67 mmol) was added into a mixture of tert-butyl 3-(7-oxo-3-(4-(trifluoromethyl)phenyl)-6,7-dihydro-1H-pyrazolo[4,3-d]pyrimidin-1-yl)pyrrolidine-1-carboxylate (0.50 g, 1.11 mmol), cesium carbonate (1.09 g, 3.34 mmol), and DMF (5 mL) at 0° C. The reaction was allowed to warm up to room temperature naturally and stirred for 2 h. The reaction was monitored by LCMS. The reaction was diluted with ethyl acetate, poured into ice-water. The organic layer was separated, dried over anhydrous sodium sulfate, and then filtered. The filtrate was concentrated under vacuum to afford the title compound (0.42 g) as yellow solid, which was used for the next step without any further purification. LCMS [M+H]⁺=408.45.

### Step 5: Preparation of 6-methyl-1-(pyrrolidin-3 yl)-3-(4-(trifluoromethyl)phenyl)-1,6-dihydro-7H-pyrazolo[4,3-d]pyrimidin-7-one hydrochloride

A mixture of tert-butyl 3-(6-methyl-7-oxo-3-(4-(trifluoromethyl)phenyl)-6,7-dihydro-1H-pyrazolo[4,3-d]pyrimidin-1-yl)pyrrolidine-1-carboxylate (420 mg, 0.91 mmol) and hydrogen chloride in 1,4-dioxane (4.0 M, 10 mL) was stirred for 4 h at room temperature. The reaction was monitored by LCMS. The reaction mixture was concentrated under vacuum to afford the title compound (290 mg) as yellow oil, which was used for the next step without any further purification. LCMS [M+H]⁺=364.35.

### Step 6: Preparation of 1-(1-acryloylpyrrolidin-3-yl)-6-methyl-3-(4-(trifluoromethyl)phenyl)-1,6-dihydro-7H-pyrazolo[4,3-d]pyrimidin-7-one

Acrylyl chloride (99 mg, 1.09 mmol) was added dropwise with stirring into a mixture of 6-methyl-1-(pyrrolidin-3-yl)-3-(4-(trifluoromethyl)phenyl)-1,6-dihydro-7H-pyrazolo[4,3-d]pyrimidin-7-one hydrochloride (290 mg, 0.73 mmol), sodium bicarbonate (310 mg, 3.69 mmol), DCM (20 mL), and water (10 mL) at 0° C. under nitrogen atmosphere. The mixture was stirred for 30 min at 0° C. The reaction was monitored by LCMS. The reaction mixture was then diluted with dichloromethane, washed with water, dried over anhydrous sodium sulfate, and then filtered. The filtrate was concentrated under vacuum. The residue was purified by silica gel chromatography eluting with DCM:MeOH=95:5 to afford the tittle compound (148 mg) as white solid.

LCMS [M+H]⁺=418.22.

¹H NMR (500 MHz, DMSO-d₆) δ 8.48-8.41 (m, 2H), 8.37 (d, J=3.6 Hz, 1H), 7.87 (dd, J=8.5, 3.9 Hz, 2H), 6.63 (ddd, J=51.4, 16.7, 10.3 Hz, 1H), 6.17 (ddd, J=16.8, 7.9, 2.4 Hz, 1H), 5.90 (dp, J=30.5, 5.2, 4.5 Hz, 1H), 5.69 (ddd, J=27.3, 10.4, 2.4 Hz, 1H), 4.16-3.99 (m, 1H), 3.97-3.81 (m, 2H), 3.81-3.60 (m, 1H), 3.56 (s, 3H), 2.55 (d, J=5.6 Hz, 1H), 2.44 (dd, J=7.7, 5.7 Hz, 1H).

### Example 8 Synthesis of Compound 8 (2 fluoro-1-(3-methyl-3-(3-(4-(trifluoromethyl)phenyl)-1H-pyrazolo[3,4-b]pyridin-1 yl)azetidin-1 yl)prop-2-en-1-one)

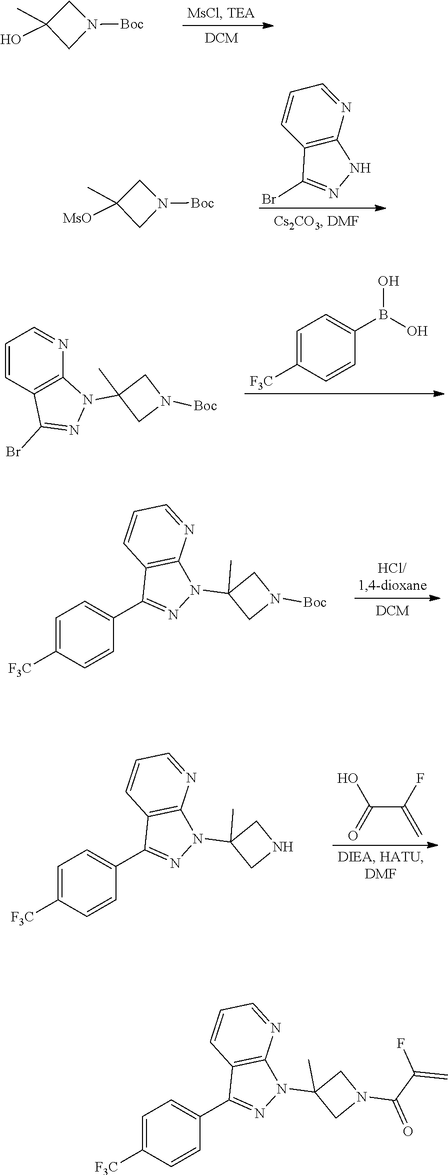

### Step 1: Preparation of tert-butyl 3-methyl-3-((methylsulfonyl)oxy)azetidine-1-carboxylate

Methanesulfonyl chloride (0.99 mL, 12.82 mmol) was added dropwise into a mixture of tert-butyl 3-hydroxy-3-methylazetidine-1-carboxylate (2.00 g, 10.68 mmol), TEA (2.97 mL, 21.36 mmol), and dichloromethane (25 mL) at 0° C. under nitrogen atmosphere. The reaction was allowed to warm up to room temperature naturally and stirred for 2 h. The mixture was quenched by the addition of water, and then extracted with dichloromethane. The organic phase was washed with brine, dried over anhydrous sodium sulfate, and filtered. The filtrate was concentrated under vacuum to afford the title compound (2.81 g) as yellow oil, which was used for the next step without any further purification.

### Step 2: Preparation of tert-butyl 3-(3-bromo-1H-pyrazolo[3,4-b]pyridin-1 yl)-3-methylazetidine-1-carboxylate

A mixture of tert-butyl 3-methyl-3-((methylsulfonyl)oxy)azetidine-1-carboxylate (2.81 g, 10.61 mmol), 3-bromo-1H-pyrazolo[3,4-b]pyridine (0.3 g, 1.51 mmol), cesium carbonate (2.47 g, 7.58 mmol), and DMF (20 mL) was stirred for 12 h at 125° C. The reaction mixture was cooled room temperature and then poured into water. The mixture was extracted with ethyl acetate, washed with brine, dried over anhydrous sodium sulfate and then filtered. The filtrate was concentrated under vacuum. The residue was purified by silica gel chromatography eluting with EA:Hex=0%-30% to afford the title compound (0.2 g) as light yellow solid. LCMS [M+H]⁺=367.07.

### Step 3: Preparation of tert-butyl 3-methyl-3-(3-(4-(trifluoromethyl)phenyl)-1H-pyrazolo[3,4-b]pyridin-1 yl)azetidine-1-carboxylate

A mixture of tert-butyl 3-(3-bromo-1H-pyrazolo[3,4-b]pyridin-1-yl)-3-methylazetidine-1-carboxylate (0.2 g, 0.54 mmol), (4-(trifluoromethyl)phenyl)boronic acid (0.15 g, 0.82 mmol), potassium carbonate (0.15 g, 1.09 mmol), [PdCl₂(dppf)]CH₂Cl₂ (0.04 g, 0.05 mmol), 1,4-dioxane (10 mL), and water (2 mL) was stirred for 4 h at 100° C. under nitrogen atmosphere. The reaction mixture was cooled down to room temperature and diluted with water. The mixture was extracted with ethyl acetate. The combined organic phase was washed with brine, dried over anhydrous sodium sulfate, and then filtered. The filtrate was concentrated under vacuum. The residue was purified by silica gel chromatography eluting with EA:Hex=0%-10% to afford the title compound (0.15 g) as off-white solid. LCMS [M+H]⁺=433.18.

### Step 4: Preparation of 1-(3-methylazetidin-3 yl)-3-(4-(trifluoromethyl)phenyl)-1H-pyrazolo[3,4-b]pyridine

A mixture of tert-butyl 3-methyl-3-(3-(4-(trifluoromethyl)phenyl)-1H-pyrazolo[3,4-b]pyridin-1-yl)azetidine-1-carboxylate (0.15 g, 0.35 mmol), dichloromethane (10 mL), and hydrogen chloride in 1,4-dioxane (1.77 mL, 4M, 7.08 mmol) was stirred for 1 h at room temperature. The mixture was concentrated under vacuum. The residue was suspended in water and the pH value was adjusted to 8-9 with saturated sodium bicarbonate. The mixture was extracted with ethyl acetate. The organic phase was washed with brine, dried over anhydrous sodium sulfate, and filtered. The filtrate was concentrated under vacuum to afford the tittle compound (0.10 g) as light yellow solid, which was used for the next step without any further purification. LCMS [M+H]⁺=333.12.

### Step 5: Preparation of 2 fluoro-1-(3-methyl-3-(3-(4-(trifluoromethyl)phenyl)-1H-pyrazolo[3,4-b]pyridin-1 yl)azetidin-1 yl)prop-2-en-1-one

A mixture of 2-fluoroacrylic acid (0.03 g, 0.37 mmol), DMF (5.00 mL), DIEA (0.15 mL, 0.93 mmol), HATU (0.17 g, 0.46 mmol), 1-(3-methylazetidin-3-yl)-3-(4-(trifluoromethyl)phenyl)-1H-pyrazolo[3,4-b]pyridine (0.1 g, 0.31 mmol) was stirred overnight at room temperature. The mixture was diluted with ethyl acetate, washed with brine, dried over anhydrous sodium sulfate, and filtered. The filtrate was concentrated under vacuum. The residue was purified by silica gel chromatography eluting with EA:Hex=0%-10% to afford the tittle compound (20 mg) as white solid.

LCMS [M+H]⁺=405.13.

¹H NMR (500 MHz, DMSO) (58.68 (dd, J=12.7, 6.0 Hz, 1H), 8.28 (d, J=7.9 Hz, 1H), 7.89 (d, J=8.0 Hz, 1H), 7.43 (dd, J=8.0, 4.5 Hz, 1H), 5.53 (dd, J=48.4, 3.2 Hz, 1H), 5.39-5.20 (m, 1H), 4.95 (d, J=10.6 Hz, 1H), 4.78 (d, J=6.8 Hz, 1H), 4.39 (d, J=10.5 Hz, 1H), 1.88 (s, 1H).

### Example 9 Synthesis of Compound 9 (2 fluoro-1-(2-methyl-3-(3-(4-(trifluoromethyl)phenyl)-1H-pyrazolo[3,4-b]pyridin-1 yl)azetidin-1 yl)prop-2-en-1-one)

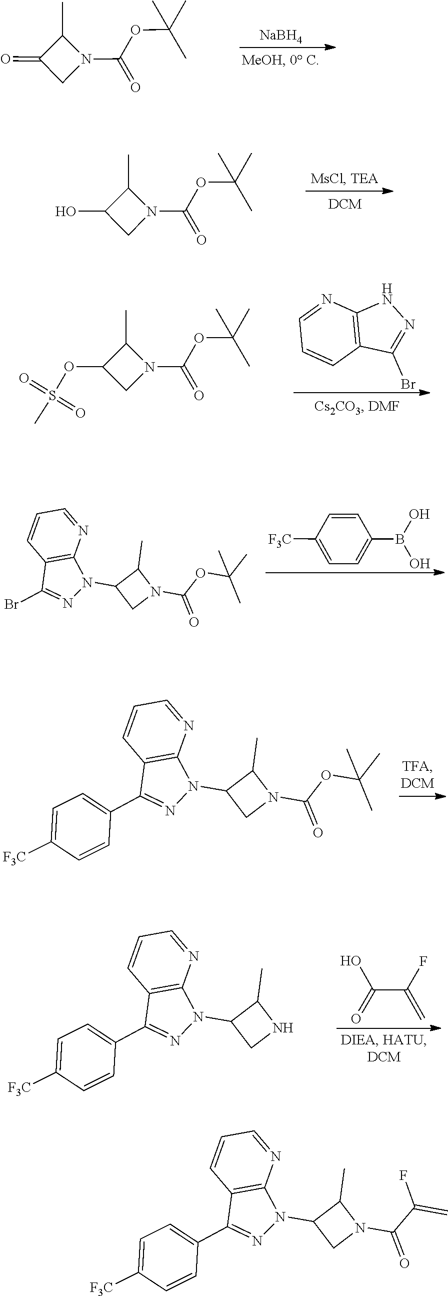

### Step 1: Preparation of tert-butyl 3-hydroxy-2-methylazetidine-1-carboxylate

Sodium borohydride (0.28 g, 7.29 mmol) was added in portions into a solution of tert-butyl 2-methyl-3-oxoazetidine-1-carboxylate (0.90 g, 4.86 mmol) and methanol (20 mL) at 0° C. under nitrogen atmosphere. The reaction was allowed to warm up to room temperature naturally and stirred for 3 h. The reaction mixture was quenched by the addition of water, and then concentrated under vacuum to remove the methanol. The residue was diluted with water, and extracted with ethyl acetate. The combined organic phase was washed with brine, dried over anhydrous sodium sulfate, and filtered. The filtrate was concentrated under vacuum to afford the tittle compound (0.80 g) as white solid, which was used for the next step without any further purification. LCMS [M+H]⁺=188.12.

### Step 2: Preparation of tert-butyl 2-methyl-3-((methylsulfonyl)oxy)azetidine-1-carboxylate

Methanesulfonyl chloride (0.73 g, 6.41 mmol) was added dropwise into a mixture of tert-butyl 3-hydroxy-2-methylazetidine-1-carboxylate (0.80 g, 4.27 mmol), TEA (1.2 mL), and dichloromethane (20 mL) at 0° C. under nitrogen atmosphere. The mixture was stirred for 2 h at the same temperature. The reaction was then quenched by the addition of water at 0° C., and then extracted with dichloromethane. The combined organic phase was washed with brine, dried over anhydrous sodium sulfate, and filtered. The filtrate was concentrated under vacuum to afford the tittle compound (1.00 g) as white solid, which was used for the next step without any further purification.

### Step 3: Preparation of tert-butyl 3-(3-bromo-1H-pyrazolo[3,4-b]pyridin-1 yl)-2-methylazetidine-1-carboxylate

A mixture of tert-butyl 2-methyl-3-((methylsulfonyl)oxy)azetidine-1-carboxylate (1.0 g, 3.78 mmol), 3-bromo-1H-pyrazolo[3,4-b]pyridine (0.60 g, 3.03 mmol), cesium carbonate (1.97 g, 6.06 mmol), and DMF (20 mL) was stirred for 3 h at 100° C. The reaction was then cooled down to room temperature, and then diluted with water. The mixture was extracted with ethyl acetate. The combined organic phase was washed with brine, dried over anhydrous sodium sulfate, and then filtered. The filtrate was concentrated under vacuum. The residue was purified by silica gel chromatography eluting with EA:Hex=0%-20% to afford the tittle compound (0.38 g) as yellow solid. LCMS [M+H]⁺=367.07.

### Step 4: Preparation of tert-butyl 2-methyl-3-(3-(4-(trifluoromethyl)phenyl)-1H-pyrazolo[3,4-b]pyridin-1 yl)azetidine-1-carboxylate

A mixture of tert-butyl 3-(3-bromo-1H-pyrazolo[3,4-b]pyridin-1-yl)-2-methylazetidine-1-carboxylate (0.36 g, 0.98 mmol), (4-(trifluoromethyl)phenyl)boronic acid (0.23 g, 1.17 mmol), potassium carbonate (0.41 g, 2.93 mmol), Pd(dppf)Cl₂CH₂Cl₂ (0.072 g, 0.10 mmol), 1,4-dioxane (20 mL), and water (4 mL) was stirred overnight at 90° C. under nitrogen atmosphere. The mixture was cooled down to room temperature, and then diluted with water. The mixture was then extracted with ethyl acetate. The combined organic phase was washed with brine, dried over anhydrous sodium sulfate, and then filtered. The filtrate was concentrated under vacuum. The residue was purified by silica gel chromatography eluting with EA:Hex=0%-50% to afford the tittle compound (0.35 g) as yellow solid. LCMS [M+H]+=433.18.

### Step 5: Preparation of 1-(2-methylazetidin-3-yl)-3-(4-(trifluoromethyl)phenyl)-1H-pyrazolo[3,4-b]pyridine

A mixture of tert-butyl 2-methyl-3-(3-(4-(trifluoromethyl)phenyl)-1H-pyrazolo[3,4-b]pyridin-1-yl)azetidine-1-carboxylate (0.35 g, 0.69 mmol), dichloromethane (20 mL), and trifluoroacetic acid (0.77 mL) was stirred for 2 h at room temperature. The pH value of the reaction mixture was adjusted with saturated sodium bicarbonate solution (50 mL). The mixture was extracted with ethyl acetate. The combined organic phase was washed with brine, dried over anhydrous sodium sulfate, and then filtered. The filtrate was concentrated under vacuum to afford the tittle compound (0.20 g) as yellow solid, which was used for the next step without any further purification. LCMS [M+H]⁺=333.12.

### Step 6: Preparation of 2 fluoro-1-(2-methyl-3-(3-(4-(trifluoromethyl)phenyl)-1H-pyrazolo[3,4-b]pyridin-1 yl)azetidin-1 yl)prop-2-en-1-one

A mixture of 2-fluoroacrylic acid (0.11 g, 1.26 mmol), DMF (5.00 mL), DIEA (0.42 mL, 2.52 mmol), HATU (0.48 g, 1.26 mmol), 1-(2-methylazetidin-3-yl)-3-(4-(trifluoromethyl)phenyl)-1H-pyrazolo[3,4-b]pyridine (0.20 g, 0.84 mmol) was stirred for 2 h at room temperature. The mixture was diluted with ethyl acetate, washed with brine, dried over anhydrous sodium sulfate, and filtered. The filtrate was concentrated under vacuum. The residue was purified by silica gel chromatography eluting with MeOH:DCM=0%-3% to afford the tittle compound (0.18 g) as white solid.

LCMS [M+H]⁺=405.13.

¹H NMR (500 MHz, CDCl₃-d₃) δ 8.59-8.58 (m, 1H), 8.37-8.35 (m, 1H), 8.12-8.10 (m, 2H), 7.78-7.77 (m, 2H), 7.28-7.26 (m, 1H), 5.75-5.65 (m, 1H), 5.53-5.52 (m, 1H), 5.16-5.13 (m, 2H), 2.81 (s, 2H), 1.73-1.72 (m, 3H).

### Example 10 Synthesis of Compound 10 (2 fluoro-N-(2-methyl-5-(3-(4-(trifluoromethyl)phenyl)-1H-pyrazolo[3,4-b]pyridin-1 yl)phenyl)acrylamide)

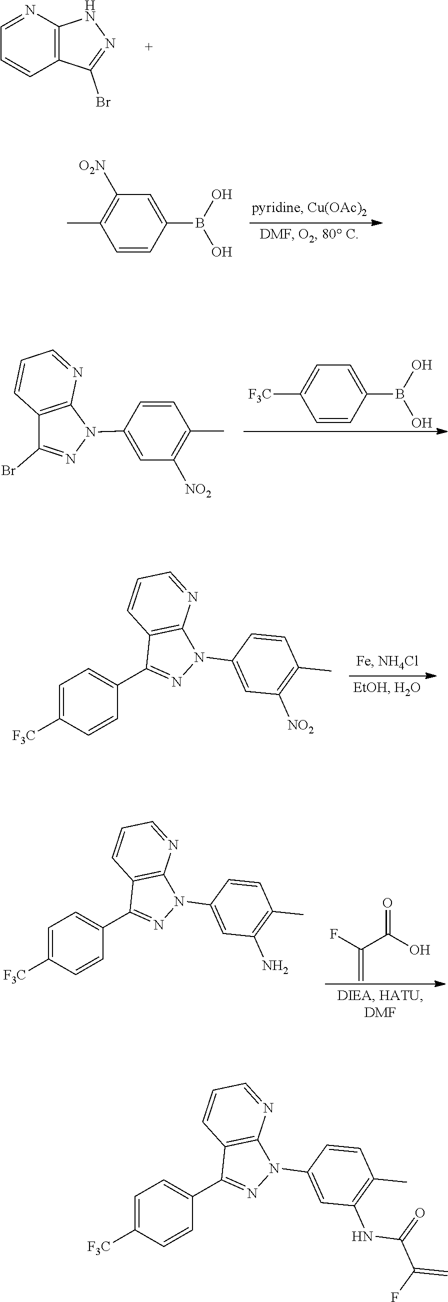

### Step 1: Preparation of 3-bromo-1-(4-methyl-3-nitrophenyl)-1H-pyrazolo[3,4-b]pyridine

A mixture of 3-bromo-1H-pyrazolo[3,4-b]pyridine (0.50 g, 2.52 mmol), (4-methyl-3-nitro-phenyl)boronic acid (685.36 mg, 3.79 mmol), pyridine (0.50 g, 2.52 mmol), Cu(OAc)₂ (0.92 g, 5.05 mmol), and DMF (20 mL) was stirred for 8 h at 80° C. under oxygen atmosphere. The mixture was diluted with ethyl acetate and then filtered through diatomaceous earth. The filtrate was washed with brine, dried over anhydrous sodium sulfate and then filtered. The filtrate was concentrated under vacuum. The residue was purified by silica gel chromatography eluting with EA:Hex=0%-20% to afford the tittle compound (0.65 g) as white solid. LCMS [M+H]⁺=332.99.

### Step 2: Preparation of 1-(4-methyl-3-nitrophenyl)-3-(4-(trifluoromethyl)phenyl)-1H-pyrazolo[3,4-b]pyridine

A mixture of 3-bromo-1-(4-methyl-3-nitrophenyl)-1H-pyrazolo[3,4-b]pyridine (0.35 g, 1.05 mmol), (4-(trifluoromethyl)phenyl)boronic acid (0.30 g, 1.58 mmol), potassium carbonate (0.44 g, 3.15 mmol), Pd(dppf)Cl₂CH₂Cl₂ (0.085 g, 0.11 mmol), 1,4-dioxane (20 mL), and water (4 mL) was stirred for 4 h at 90° C. under nitrogen atmosphere. The mixture was cooled down to room temperature, and then diluted with water. The mixture was then extracted with ethyl acetate. The combined organic phase was washed with brine, dried over anhydrous sodium sulfate, and then filtered. The filtrate was concentrated under vacuum. The residue was purified by silica gel chromatography eluting with EA:Hex=0%-50% to afford the tittle compound (0.26 g) as yellow solid. LCMS [M+H]⁺=399.10.

### Step 3: Preparation of 2-methyl-5-(3-(4-(trifluoromethyl)phenyl)-1H-pyrazolo[3,4-b]pyridin-1 yl)aniline

A mixture of 1-(4-methyl-3-nitrophenyl)-3-(4-(trifluoromethyl)phenyl)-1H-pyrazolo[3,4-b]pyridine (0.26 g, 0.88 mmol), EtOH (30 mL), H₂O (10 mL), ammonium chloride (0.47 g, 8.79 mmol), and iron powder (0.25 g, 4.39 mmol) ws stirred for 2 h at 75° C. The reaction mixture was cooled down to room temperature and then filtered through diatomaceous earth. The filtrate was concentrated under vacuum. The residue was diluted with ethyl acetate, washed with brine, dried over anhydrous sodium sulfate, and filtered. The filtrate was concentrated under vacuum to afford the tittle compound (0.20 g) as brown solid, which was used for the next step without any further purification. LCMS [M+H]⁺=369.12.

### Step 4: Preparation of 2 fluoro-N-(2-methyl-5-(3-(4-(trifluoromethyl)phenyl)-1H-pyrazolo[3,4-b]pyridin-1 yl)phenyl)acrylamide

A mixture of 2-fluoroacrylic acid (0.058 g, 0.65 mmol), DMF (20 mL), DIEA (0.27 mL, 1.63 mmol), HATU (0.27 g, 0.71 mmol), 2-methyl-5-(3-(4-(trifluoromethyl)phenyl)-1H-pyrazolo[3,4-b]pyridin-1-yl)aniline (0.20 g, 0.54 mmol) was stirred for 2 h at room temperature. The mixture was diluted with ethyl acetate, washed with brine, dried over anhydrous sodium sulfate, and filtered. The filtrate was concentrated under vacuum. The residue was purified by silica gel chromatography eluting with MeOH:DCM=0%-3% to afford the tittle compound (0.10 g) as white solid.

LCMS [M+H]⁺=441.13.

¹H NMR (500 MHz, CDCl₃-d₃) δ 8.99-8.98 (m, 1H), 8.71-8.70 (m, 1H), 8.42-8.40 (m, 1H), 8.18-8.17 (m, 2H), 8.13-8.11 (m, 1H), 7.97 (s, 1H), 7.80-7.78 (m, 2H), 7.41-7.39 (m, 1H), 7.33-7.31 (m, 1H), 5.93-5.83 (m, 1H), 5.32-5.28 (m, 1H), 2.38 (s, 3H).

### Example 11 Synthesis of Compound 11 (2 fluoro-1-(3-(6-methyl-3-((4-(trifluoromethyl)phenyl)amino)-1H-pyrazolo[3,4-b]pyridin-1 yl)azetidin-1 yl)prop-2-en-1-one)

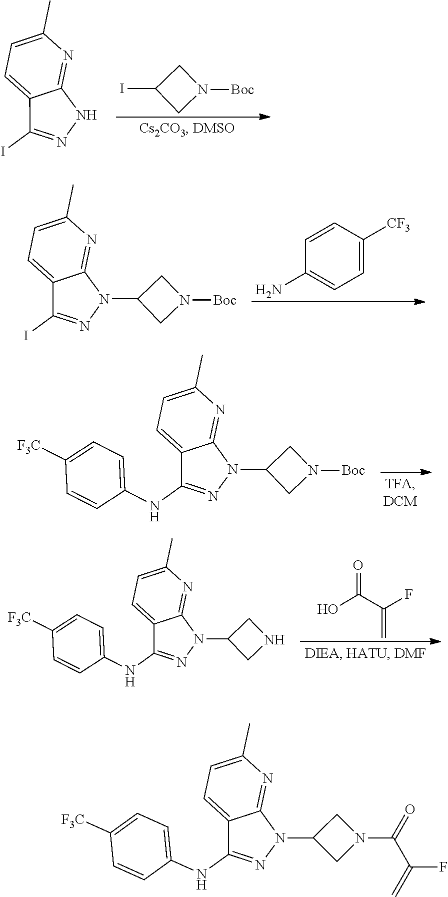

### Step 1: Preparation of tert-butyl 3-(3-iodo-6-methyl-1H-pyrazolo[3,4-b]pyridin-1-yl)azetidine-1-carboxylate

A mixture of 3-iodo-6-methyl-1H-pyrazolo[3,4-b]pyridine (0.4 g, 1.54 mmol), tert-butyl 3-iodoazetidine-1-carboxylate (0.7 g, 2.47 mmol), cesium carbonate (1.1 g, 3.4 mmol), and DMSO (20 ml) was stirred for 2 h at 80 s. The reaction mixture was cooled down to room temperature and then poured into water. The mixture was extracted with ethyl acetate. The combined organic phase was washed with water, dried over anhydrous sodium sulfate, and then filtered. The filtrate was concentrated under vacuum. The residue was purified by silica gel chromatography eluting with EA:Hex=0%-15% to afford the tittle compound (0.57 g) as white solid. LCMS [M+H]⁺=415.06.

### Step 2: Preparation of tert-butyl 3-(6-methyl-3-((4-(trifluoromethyl)phenyl)amino)-1H-pyrazolo[3,4-b]pyridin-1 yl)azetidine-1-carboxylate

A mixture of tert-butyl 3-(3-iodo-6-methyl-1H-pyrazolo[3,4-b]pyridin-1-yl)azetidine-1-carboxylate (0.25 g, 0.6 mmol), 4-(trifluoromethyl)aniline (0.29 g, 1.8 mmol), potassium phosphate (0.38 g, 1.8 mmol), Xphos (0.057 g, 0.12 mmol), Pd₂dba₃ (0.055 g, 0.06 mmol), and 1,4-dioxane (20 mL) was stirred for 8 h at 100° C. under nitrogen atmosphere. The reaction mixture was cooled down to room temperature and then concentrated under vacuum. The residue was purified by silica gel chromatography eluting with EA:Hex=0%-30% to afford the tittle compound (0.19 g) as white solid. LCMS [M+H]⁺=448.46.

### Step 3: Preparation of 1-(azetidin-3 yl)-6-methyl-N-(4-(trifluoromethyl)phenyl)-1H-pyrazolo[3,4-b]pyridin-3-amine

A mixture of tert-butyl 3-(6-methyl-3-((4-(trifluoromethyl)phenyl)amino)-1H-pyrazolo[3,4-b]pyridin-1-yl)azetidine-1-carboxylate (0.19 g, 0.43 mmol), dichloromethane (10 mL), and trifluoroacetic acid (0.3 mL, 4.29 mmol) was stirred for 2 h at room temperature. The reaction mixture was concentrated under vacuum. The residue was added saturated solution of sodium carbonate. The mixture was extracted with ethyl acetate. The combined organic phase was washed with brine, dried over anhydrous sodium sulfate, and then filtered. The filtrate was concentrated under vacuum to afford the tittle compound (0.15 g) as yellow solid, which was used for the next step without any further purification. LCMS [M+H]⁺=348.14.

### Step 4: Preparation oft fluoro-1-(3-(6-methyl-3-((4-(trifluoromethyl)phenyl)amino)-1H-pyrazolo[3,4-b]pyridin-1 yl)azetidin-1 yl)prop-2-en-1-one

A mixture of 2-fluoroacrylic acid (0.05 g, 0.56 mmol), DMF (10 mL), DIEA (0.21 mL, 1.3 mmol), HATU (0.24 g, 0.65 mmol), 1-(azetidin-3-yl)-6-methyl-N-(4-(trifluoromethyl)phenyl)-1H-pyrazolo[3,4-b]pyridin-3-amine (0.15 g, 0.43 mmol) was stirred for 2 h at room temperature. The mixture was diluted with ethyl acetate, washed with brine, dried over anhydrous sodium sulfate, and filtered. The filtrate was concentrated under vacuum. The residue was purified by silica gel chromatography eluting with EA:Hex=0%-20% to afford the tittle compound (10 mg) as white solid.

LCMS [M+H]⁺=420.14.

¹H NMR (500 MHz, DMSO) δ 9.75 (s, 1H), 8.29 (d, J=8.2 Hz, 1H), 7.83 (d, J=8.6 Hz, 2H), 7.63 (d, J=8.7 Hz, 2H), 7.10 (d, J=8.2 Hz, 1H), 5.80 (s, 1H), 5.56 (dd, J=48.4, 3.5 Hz, 1H), 5.37 (dd, J=16.5, 3.5 Hz, 1H), 4.89 (d, J=7.5 Hz, 1H), 4.76 (d, J=4.7 Hz, 1H), 4.54 (s, 1H), 4.45 (d, J=5.2 Hz, 1H), 2.59 (s, 3H).

### Example 12 Synthesis of Compound 12 (2 fluoro-1-(2-hydroxy-3-(3-(4-(trifluoromethyl)phenyl)-1H-pyrazolo[3,4-b]pyridin-1 yl)azetidin-1 yl)prop-2-en-1-one)

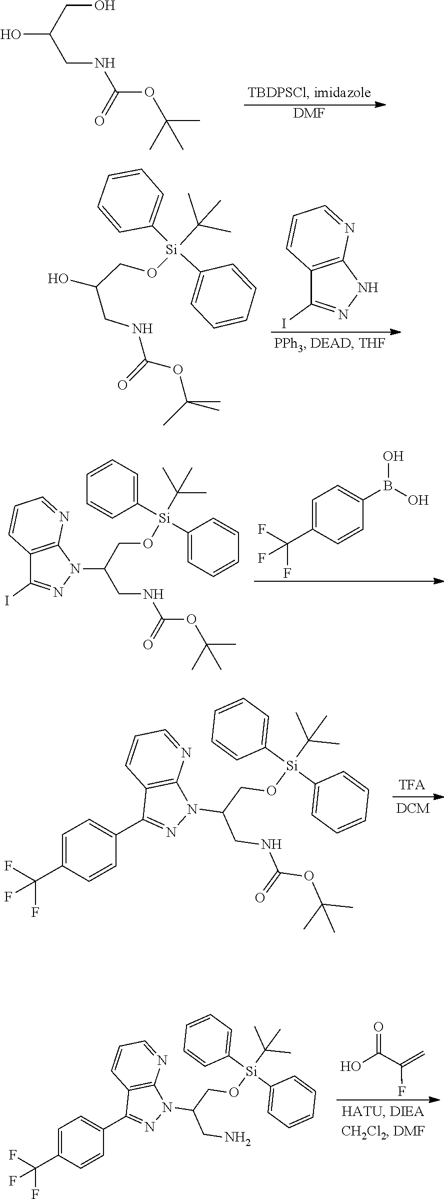

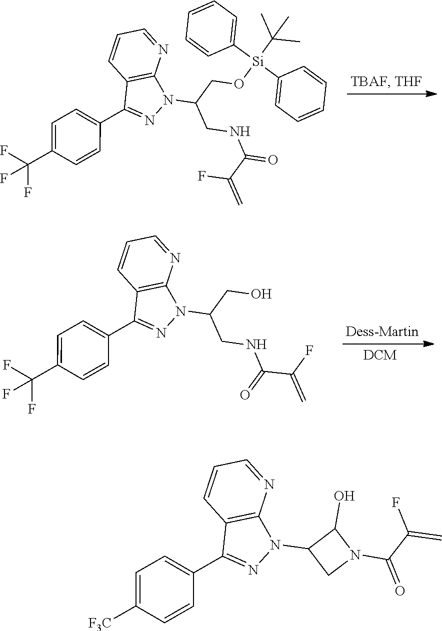 [SMILES: C=C(F)C(=O)NCC(CO[Si](c1ccccc1)(c1ccccc1)C(C)(C)C)n1nc(-c2ccc(C(F)(F)F)cc2)c2cccnc21 ; C=C(F)C(=O)NCC(CO)n1nc(-c2ccc(C(F)(F)F)cc2)c2cccnc21 ; C=C(F)C(=O)N1CC(n2nc(-c3ccc(C(F)(F)F)cc3)c3cccnc32)C1O]

### Step 1: Preparation of tert-butyl (3-((tert-butyldiphenylsilyl)oxy)-2-hydroxypropyl)carbamate

Tert-butylchlorodiphenylsilane (1.58 g, 5.75 mmol) was added into a mixture of tert-butyl (2,3-dihydroxypropyl)carbamate (1.00 g, 5.23 mmol) and imidazole (0.78 g, 11.50 mmol) in DMF (30 mL) at room temperature. The reaction was stirred at room temperature overnight. The reaction was monitored by LCMS. The reaction was quenched by the addition of water. The mixture was extracted with ethyl acetate. The combined organic phase was washed with brine, dried over anhydrous sodium sulfate, and then filtered. The filtrate was concentrated under vacuum. The residue was purified by silica gel chromatography eluted with (EA:Hex=0%-30%) to afford the title compound (2.1 g, 93%). LCMS [M+H]⁺=430.23.

### Step 2: Preparation of tert-butyl (3-((tert-butyldiphenylsilyl)oxy)-2-(3-iodo-1H-pyrazolo[3,4-b]pyridin-1 yl)propyl)carbamate

DEAD (2.56 g, 14.69 mmol) was added dropwise into a mixture of tert-butyl (3-((tert-butyldiphenylsilyl)oxy)-2-hydroxypropyl) carbamate (2.1 g, 4.9 mmol), PPh₃ (3.85 g, 14.69 mmol), 3-iodo-1H-pyrazolo[3,4-b]pyridine (1.20 g, 4.90 mmol), and THE (20 mL) at 0° C. under nitrogen atmosphere. The reaction mixture was allowed to warm up to room temperature and stirred for overnight. The reaction was monitored by LCMS. The reaction was poured into ice-water and extracted with ethyl acetate. The combined organic layer was washed with brine, dried over anhydrous sodium sulfate, and then filtered. The filtrate was concentrated under vacuum. The crude product was purified by silica gel chromatography eluting with (EA:Hex=0-30%) to afford the title compound (2.22 g, 69%) as a red oil. LCMS [M+H]⁺=657.17.

### Step 3: Preparation of tert-butyl (3-((tert-butyldiphenylsilyl)oxy)-2-(3-(4-(trifluoromethyl)phenyl)-1H-pyrazolo[3,4-b]pyridin-1 yl)propyl)carbamate

A mixture of tert-butyl (3-((tert-butyldiphenylsilyl)oxy)-2-(3-iodo-1H-pyrazolo[3,4-b]pyridin-1-yl)propyl)carbamate (1 g, 1.52 mmol), (4-(trifluoromethyl)phenyl)boronic acid (0.40 g, 2.13 mmol), K₂CO₃ (0.63 g, 4.57 mmol), Pd(dppf)Cl₂ (0.1 g, 0.23 mmol) in 1,4-dioxane (20 mL) and water (4 mL) was stirred at 90° C. for 6 h under nitrogen atmosphere. The mixture was concentrated under vacuum. The residue was further purified by silica gel chromatograph eluting with (EA:Hex=0-20%) to afford the title compound (0.65 g, 63%). LCMS [M+H]⁺=675.29.

### Step 4: Preparation of 3-((tert-butyldiphenylsilyl)oxy)-2-(3-(4-(trifluoromethyl) phenyl)-1H-pyrazolo[3,4-b]pyridin-1 yl)propan-1-amine

A mixture of tert-butyl (3-((tert-butyldiphenylsilyl)oxy)-2-(3-(4-(trifluoromethyl)phenyl)-1H-pyrazolo[3,4-b]pyridin-1-yl)propyl)carbamate (651 mg, 0.96 mmol), DCM (10 mL), and TFA (1 mL, 13.51 mmol) was stirred at room temperature for 4 h. The reaction was monitored by LCMS. The mixture was concentrated under vacuum. The residue was diluted with dichloromethane. The pH value of the solution was adjusted to 10 with potassium carbonate solution. The mixture was extracted with dichloromethane, washed with brine, dried over anhydrous sodium sulfate, and then filtered. The filtrate was concentrated under vacuum to afford title compound (542 mg, 98%), which was used for the next step without any further purification. LCMS [M+H]+=575.24.

### Step 5: Preparation of N-(3-((tert-butyldiphenylsilyl)oxy)-2-(3-(4-(trifluoromethyl)phenyl)-1H-pyrazolo[3,4-b]pyridin-1 yl)propyl)-2 fluoroacrylamide

A mixture of 2-fluoroacrylic acid (115 mg, 1.27 mmol), DIEA (0.42 mL, 2.55 mmol), HATU (387 mg, 1.02 mmol), DCM (20 mL), DMF (4 mL), and 3-((tert-butyldiphenylsilyl)oxy)-2-(3-(4-(trifluoromethyl)phenyl)-1H-pyrazolo[3,4-b]pyridin-1-yl)propan-1-amine (488 mg, 0.85 mmol) was stirred for 3 h at room temperature. The reaction mixture was quenched with water. The mixture was extracted with ethyl acetate, washed with brine, dried over anhydrous sodium sulfate, and then filtered. The filtrate was concentrated under vacuum. The residue was purified by silica gel chromatography eluting with EA:Hex=0-20% to afford the title compound (300 mg). LCMS [M+H]⁺=647.24.

### Step 6: Preparation of 2 fluoro-N-(3-hydroxy-2-(3-(4-(trifluoromethyl)phenyl)-1H-pyrazolo[3,4-b]pyridin-1 yl)propyl)acrylamide

To a stirred solution of N-(3-((tert-butyldiphenylsilyl)oxy)-2-(3-(4-(trifluoromethyl)phenyl)-1H-pyrazolo[3,4-b]pyridin-1-yl)propyl)-2-fluoroacrylamide (300 mg, 0.46 mmol) in THF (8 mL) was added TBAF (0.7 mL, 1 M in THF). The reaction was stirred at room temperature for 2 h. The reaction was monitored by LCMS. The reaction was poured into water and extracted with ethyl acetate. The organic layer was separated. The aqueous layer was extracted with ethyl acetate. The combined organic phase was washed with brine, dried over anhydrous sodium sulfate, and then filtered. The filtrate was concentrated under vacuum. The residue was purified by silica gel chromatography eluting with EA:Hex=0%-100% to afford the title compound (162 mg, 86%) as a white solid.

LCMS [M+H]⁺=409.12.

¹H NMR (500 MHz, DMSO) δ 8.70-8.58 (m, 3H), 8.27 (d, J=6.0 Hz, 2H), 7.89 (d, J=6.0 Hz, 2H), 7.35 (m, 1H), 5.49-5.33 (m, 1H), 5.32-5.23 (m, 1H), 5.15 (m, 1H), 5.04-4.91 (m, 1H), 4.05-3.97 (m, 1H), 3.96-3.88 (m, 1H), 3.84-3.76 (m, 1H), 3.71-3.62 (m, 1H).

### Step 7: Preparation of 2 fluoro-1-(2-hydroxy-3-(3-(4-(trifluoromethyl)phenyl)-1H-pyrazolo[3,4-b]pyridin-1 yl)azetidin-1 yl)prop-2-en-1-one

To a stirred solution of 2-fluoro-N-(3-hydroxy-2-(3-(4-(trifluoromethyl)phenyl)-1H-pyrazolo[3,4-b]pyridin-1-yl)propyl)acrylamide (40 mg, 0.10 mmol) in DCM (10 mL) was added Dess-Martin (54 mg, 0.13 mmol). The reaction was stirred at room temperature for 2 h. The reaction was monitored by LCMS. The reaction mixture was quenched by the addition of sodium bicarbonate solution and sodium thiosulfate solution. The mixture was extracted with dichloromethane, washed with brine, dried over anhydrous sodium sulfate, and then filtered. The filtrate was concentrated under vacuum. The residue was purified by Pre-TLC (Hex:EA=1:1) to afford the title compound (28 mg, 70%).

LCMS [M+H]⁺=407.11.

¹H NMR (500 MHz, DMSO) δ 8.65-8.54 (m, 2H), 8.39 (m, 1H), 8.26 (d, J=8.1 Hz, 2H), 7.90 (m, 2H), 7.34 (m, 1H), 5.37-5.24 (m, 1H), 5.16 (m, 1H), 5.07 (dd, J=15.7, 3.3 Hz, 1H), 4.09 (q, J=5.2 Hz, 1H), 3.96-3.84 (m, 2H).

### Example 13 Synthesis of Compound 13 (N-(1-(3-(4-(trifluoromethyl)phenyl)imidazo[1,5-a]pyridin-1 yl)azetidin-3 yl)acrylamide)

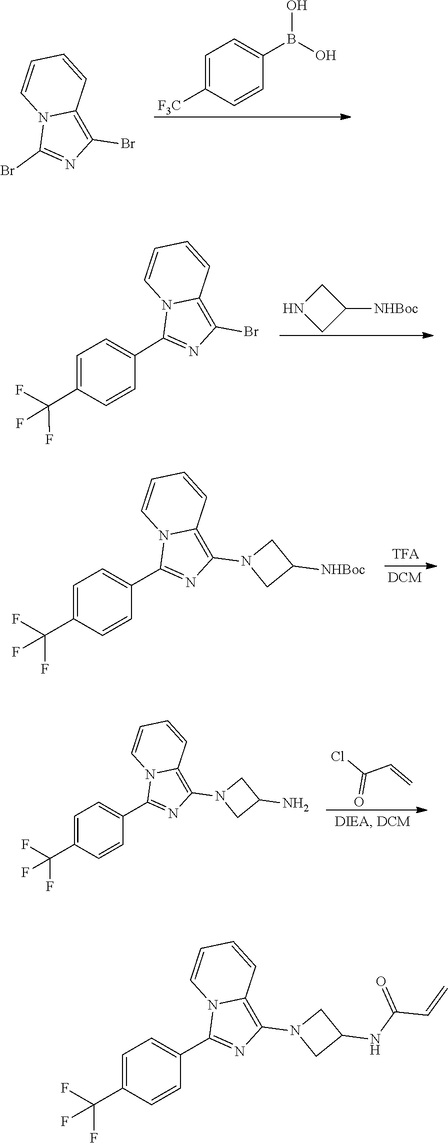

### Step 1: Preparation of 7-Bromo-9-[4-(trifluoromethyl) phenyl]-1,8-diazabicyclo [4.3.0]nona-2,4,6,8-tetraene

A mixture of 7,9-dibromo-1,8-diazabicyclo[4.3.0]nona-2,4,6,8-tetraene (0.70 g, 2.54 mmol), [4-(trifluoromethyl)phenyl]boronic acid (0.48 g, 2.54 mmol), Pd(PPh₃)₄ (0.15 g, 0.13 mmol), K₂CO₃ (0.70 g, 5.07 mmol), 1,4-dioxane (4 mL), and water (1 mL) was degassed with N₂ and stirred for 2 h at 85° C. The reaction was cooled to room temperature, diluted with water, extracted with ethyl acetate. The combined organic layers was washed with brine, dried over anhydrous sodium sulfate, and filtered. The filtrate was concentrated under vacuum. The residue was purified by silica gel chromatography eluting with hexane/ethyl acetate=5/1 to afford the title compound (0.50 g, 57.78%) as a brown solid. LCMS [M+H]⁺=342.13.

### Step 2: Preparation of tert-butyl N-[1-[9-[4-(trifluoromethyl)phenyl]-1,8-diazabicyclo[4.3.0]nona-2,4,6,8-tetraen-7 yl]azetidin-3 yl]carbamate

A mixture of 7-bromo-9-[4-(trifluoromethyl)phenyl]-1,8-diazabicyclo[4.3.0] nona-2,4,6,8-tetraene (0.50 g, 1.47 mmol), tert-butyl-N-(azetidin-3-yl) carbamate (0.25 g, 1.47 mmol), XantPhos (0.08 g, 0.15 mmol), Pd₂(dba)₃ (0.13 g, 0.15 mmol) and Cs₂CO₃ (0.96 g, 2.93 mmol) in 1,4-dioxane (5.00 mL) was degassed with N₂ and stirred overnight at 80° C. After completion, the reaction was cooled to room temperature, extracted with EA three times. The organic layers were concentrated under reduced pressure. The residue was purified by silica gel column (hexane/EA=10/1) to afford the title product (0.40 g, 63.11%) as a yellow solid. LCMS [M+H]⁺=433.45.

### Step 3: Preparation of 1-[9-[4-(Trifluoromethyl) phenyl]-1,8-diazabicyclo[4.3.0] nona-2,4,6,8-tetraen-7 yl]azetidin-3-amine

To a solution of tert-butyl-N-[1-[9-[4-(trifluoromethyl)phenyl]-1,8-diazabicyclo [4.3.0]nona-2,4,6,8-tetraen-7-yl]azetidin-3-yl]carbamate (0.20 g, 0.46 mmol) in DCM (2.00 mL) was added dropwise TFA (0.03 mL, 0.46 mmol) and stirred for 2 h at room temperature. After completion, the PH value of the reaction was adjusted to 7 and extracted with DCM, concentrated. The residue was directly used to next step without further purification. LCMS [M+H]⁺=333.33.

### Step 4: Preparation of N-[1-[9-[4-(Trifluoromethyl)phenyl]-1,8-diazabicyclo[4.3.0]nona-2,4,6,8-tetraen-7-yl]azetidin-3 yl]prop-2-enamide

To a solution of 1-[9-[4-(trifluoromethyl)phenyl]-1,8-diazabicyclo [4.3.0]nona-2,4,6,8-tetraen-7-yl]azetidin-3-amine (0.10 g, 0.30 mmol) and TEA (0.08 mL, 0.60 mmol) in DCM (3 mL) was added prop-2-enoyl chloride (0.03 g, 0.30 mmol) at 0° C. and stirred for 0.5 h at room temperature. After completion, the reaction was quenched by water, extracted with DCM. The combined organic layers was concentrated under reduced pressure. The residue was purified by Prep-TLC to afford the title product (10 mg, 8.6%) as an off white solid.

LCMS [M+H]⁺=387.38.

### Example 14 Synthesis of Compound 14 (N-(1-(1-acryloylazetidin-3 yl)-3-(4-(trifluoromethyl) phenyl)-1H-indazol-7-yl) methanesulfonamide)

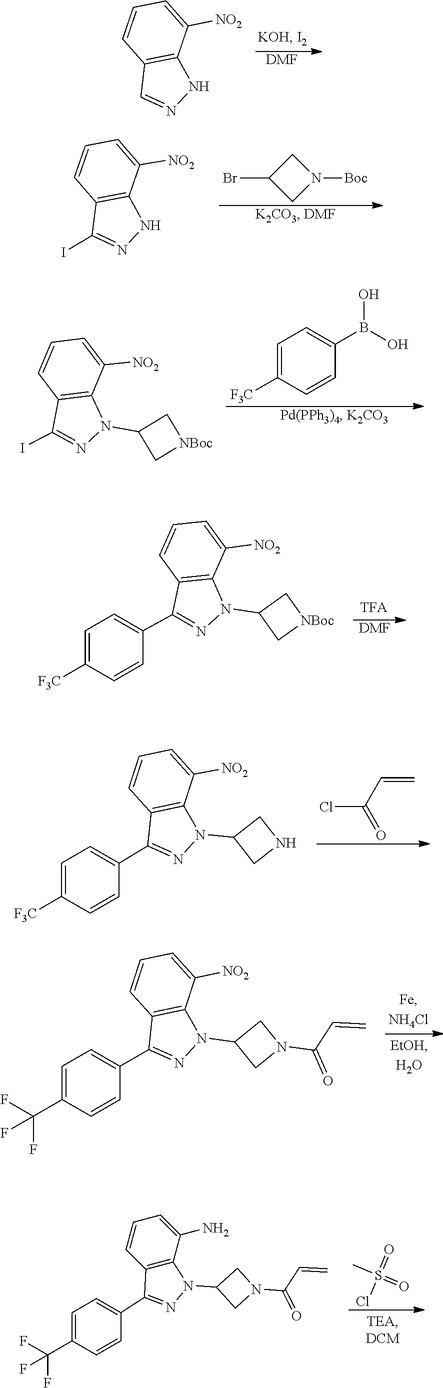

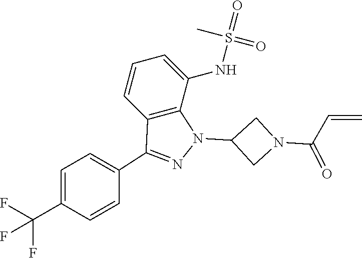 [SMILES: C=CC(=O)N1CC(n2nc(-c3ccc(C(F)(F)F)cc3)c3cccc(NS(C)(=O)=O)c32)C1]

### Step 1: Preparation of 3-Iodo-7-nitro-1H-indazole

A mixture of 7-nitro-1H-indazole (2.00 g, 12.26 mmol), KOH (2.75 g, 49.04 mmol) and I₂ (1.56 g, 12.26 mmol) in DMF (10.00 mL) was stirred overnight at room temperature. After completion, water was added to the reaction, extracted with EA three times, combined the organic layers and concentrated. The residue was purified by silica gel column to afford the title product (2.50 g, 70.55%) as a brown solid. LCMS [M+H]⁺=290.03.

### Step 2: Preparation of tert-butyl 3-(3-iodo-7-nitro-JH-indazol-1 yl)azetidine-1-carboxylate

A mixture of 3-iodo-7-nitro-1H-indazole (1.00 g, 3.46 mmol), tert-butyl 3-bromoazetidine-1-carboxylate (0.98 g, 4.15 mmol), Cs₂CO₃ (2.25 g, 6.92 mmol) in DMF (10.00 mL) was stirred for 3 h at 100° C. After completion, the reaction was cooled to room temperature; water was added to the reaction and extracted with EA three times. The organic layers were concentrated under reduced pressure. The residue was purified by silica gel column (hexane/EA=10/1) to afford the title product (1.00 g, 65.07%) as a brown solid. LCMS [M+H]⁺=445.23.

### Step 3 Preparation of tert-butyl 3-(7-nitro-3-(4-(trifluoromethyl)phenyl)-1H-indazol-1-yl)azetidine-1-carboxylate

A mixture of tert-butyl-3-(3-iodo-7-nitro-indazol-1-yl)azetidine-1-carboxylate (1.00 g, 2.25 mmol), [4-(trifluoromethyl)phenyl]boronic acid (0.43 g, 2.25 mmol), Pd(PPh₃)₄ (0.13 g, 0.11 mmol), K₂CO₃ (0.62 g, 4.50 mmol) in dioxane (5.00 mL) and H₂O (1.00 mL) was degassed with N₂ and stirred overnight at 80° C. After completion, the reaction was cooled to room temperature, extracted with EA three times. The organic layers were concentrated under reduced pressure. The residue was purified by silica gel column (hexane/EA=3/1) to afford the title product (0.40 g, 38.42%) as a yellow solid. LCMS [M+H]⁺=463.43.

### Step 4: Preparation of 1-(Azetidin-3 yl)-7-nitro-3-[4-(trifluoromethyl)phenyl] indazole

To a solution of tert-butyl-3-[7-nitro-3-[4-(trifluoromethyl) phenyl]indazol-1-yl] azetidine-1-carboxylate in DCM (4.00 mL) was added TFA (0.32 mL, 4.33 mmol) and stirred for 2 h at RT. After completion, the PH value of the reaction was adjusted to 7 and extracted with DCM, concentrated. The residue (0.25 g, 79.77%) was directly used to next step without further purification. LCMS [M+H]⁺=363.31.

### Step 5: Preparation of 1-[3-[7 Nitro-3-[4-(trifluoromethyl)phenyl]indazol-1 yl]azetidin-1-yl]prop-2-en-1-one

To a solution of 1-(azetidin-3-yl)-7-nitro-3-[4-(trifluoromethyl)phenyl]indazole (0.25 g, 0.69 mmol) and TEA (0.19 mL, 1.38 mmol) in DCM (4 mL) was added dropwise prop-2-enoyl chloride (0.07 g, 0.76 mmol) at 0° C. and stirred for 0.5 h at room temperature. After completion, the reaction was quenched by water, extracted with DCM. The combined organic layers were concentrated under reduced pressure. The residue was directly used to next step without further purification. LCMS [M+H]⁺=417.36.

### Step 6: Preparation of 1-[3-[7 Amino-3-[4-(trifluoromethyl) phenyl]indazol-1 yl]azetidin-1 yl]prop-2-en-1-one

A mixture of 1-[3-[7-Nitro-3-[4-(trifluoromethyl)phenyl]indazol-1-yl] azetidin-1-yl]prop-2-en-1-one (0.20 g, 0.48 mmol), Fe(0.08 g, 1.44 mmol), NH₄C₁ (0.03 g, 0.58 mmol) in EtOH/H₂O (4/1 mL) was stirred for 1 h at 80° C. After completion, the reaction was cooled to room temperature, filtered. The filtrate was concentrated under reduced pressure. The residue was purified by silica gel column (PE/EA=3/1) to afford the title product (0.17 g, 91.59%) as a brown solid. LCMS [M+H]⁺=387.38.

### Step 7: Preparation of N-(1-(1-acryloylazetidin-3-yl)-3-(4-(trifluoromethyl) phenyl)-1H-indazol-7-yl)methanesulfonamide

Methanesulfonyl chloride (0.02 g, 0.17 mmol) was added to a solution of 1-[3-[7-amino-3-[4-(trifluoromethyl)phenyl]indazol-1-yl]azetidin-1-yl]prop-2-en-1-one (0.05 g, 0.13 mmol) and TEA (0.06 g, 0.26 mmol) in DCM (3 mL) and was stirred for 1 h. After completion, water was added to the reaction, separated the organic layer and concentrated. The residue was purified by Prep-TLC (PE/EA=2/1) to afford the title product (5.8 mg, 9%) solid. LCMS [M+H]⁺=465.46.

### Example 15 Synthesis of Compound 15 (N-(1-(1-acryloylazetidin-3 yl)-3-(4-(trifluoromethyl)phenyl)-1H-indazol-7-yl)acetamide)

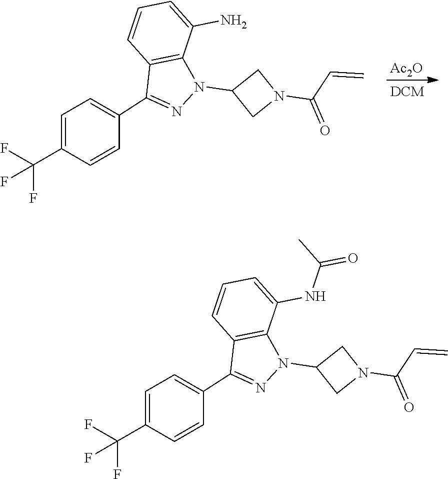 [SMILES: "Ac2O" ; C=CC(=O)N1CC(n2nc(-c3ccc(C(F)(F)F)cc3)c3cccc(N)c32)C1 ; C=CC(=O)N1CC(n2nc(-c3ccc(C(F)(F)F)cc3)c3cccc(NC(C)=O)c32)C1]

### Step 1: Preparation of N-(1-(I Acryloylazetidin-3 yl)-3-(4-(trifluoromethyl) phenyl)-1H-indazol-7-yl) acetamide

1-[3-[7-amino-3-[4-(trifluoromethyl)phenyl]indazol-1-yl]azetidin-1-yl]prop-2-en-1-one (170.00 mg, 0.44 mmol), Ac₂O (0.04 mL, 0.44 mmol) in DCM (2.00 mL) was stirred for 1 h at RT, water was added to the reaction, separated the organic layer and concentrated. The residue was purified by Prep-TLC to afford the title product (3.00 mg, 1.59%) as a white solid. LCMS [M+H]⁺=429.42. The compounds of table 1 were prepared in a similar manner to Examples 1-15 via different reaction starting materials and suitable reagents.

TABLE 1

| | | | Physical Data |
|---|---|---|---|
| EX | | | (LCMS) |
| No. | Structure | Chemical Name | (M + H)⁺ |
| 16 | 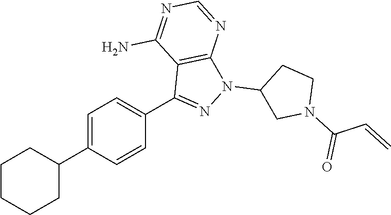 [SMILES: C=CC(=O)N1CCC(n2nc(-c3ccc(C4CCCCC4)cc3)c3c(N)ncnc32)C1] | 1-(3-(4-amino-3-(4-cyclohexyl phenyl)-1H-pyrazolo[3,4-d]pyrim- idin-1-yl)pyrrolidin-1-yl)prop-2- en-1-one | 417.2 |
| 17 | 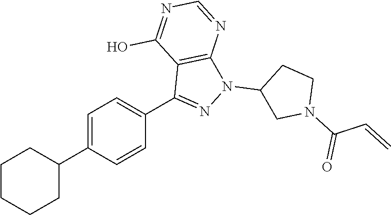 [SMILES: C=CC(=O)N1CCC(n2nc(-c3ccc(C4CCCCC4)cc3)c3c(O)ncnc32)C1] | 1-(3-(3-(4-cyclohexylphenyl)-4- hydroxy-1H-pyrazolo[3,4-d]- pyrimidin-1-yl)pyrrolidin-1-yl)- prop-2-en-1-one | 418.2 |
| 18 | 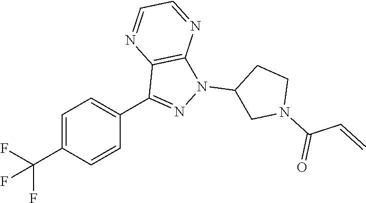 [SMILES: C=CC(=O)N1CCC(n2nc(-c3ccc(C(F)(F)F)cc3)c3nccnc32)C1] | 1-(3-(3-(4-(trifluoromethyl)phen- yl)-1H-pyrazolo[3,4-b]pyrazin-1- yl)pyrrolidin-1-yl)prop-2-en-1- one | 388.1 |
| 19 | 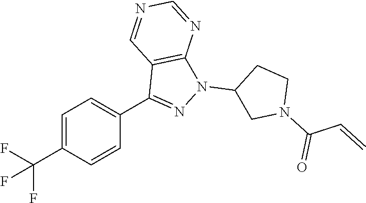 [SMILES: C=CC(=O)N1CCC(n2nc(-c3ccc(C(F)(F)F)cc3)c3cncnc32)C1] | 1-(3-(3-(4-(trifluoromethyl)phen- yl)-1H-pyrazolo[3,4-d]pyrimidin- 1-yl)pyrrolidin-1-yl)prop-2-en-1- one | 388.1 |
| 20 | 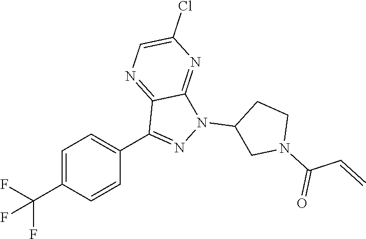 [SMILES: C=CC(=O)N1CCC(n2nc(-c3ccc(C(F)(F)F)cc3)c3ncc(Cl)nc32)C1] | 1-(3-(6-chloro-3-(4-(trifluoro- methyl)phenyl)-1H-pyrazolo[3,4- b]pyrazin-1-yl)pyrrolidin-1-yl)- prop-2-en-1-one | 422.1 |
| 21 | 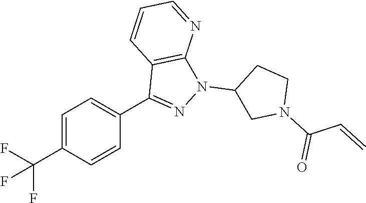 [SMILES: C=CC(=O)N1CCC(n2nc(-c3ccc(C(F)(F)F)cc3)c3cccnc32)C1] | 1-(3-(3-(4-(trifluoromethyl)phen- yl)-1H-pyrazolo[3,4-b]pyridin-1- yl)pyrrolidin-1-yl)prop-2-en-1- one | 387.1 |
| 22 | 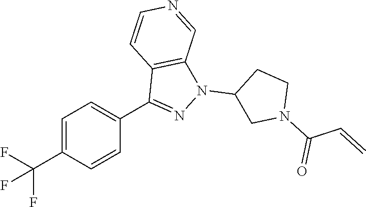 [SMILES: C=CC(=O)N1CCC(n2nc(-c3ccc(C(F)(F)F)cc3)c3ccncc32)C1] | 1-(3-(3-(4-(trifluoromethyl)phen- yl)-1H-pyrazolo[3,4-c]pyridin-1- yl)pyrrolidin-1-yl)prop-2-en-1- one | 387.1 |
| 23 | 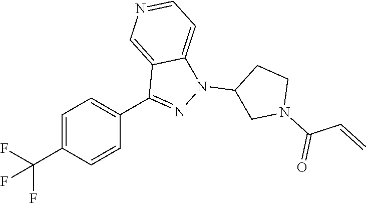 [SMILES: C=CC(=O)N1CCC(n2nc(-c3ccc(C(F)(F)F)cc3)c3cnccc32)C1] | 1-(3-(3-(4-(trifluoromethyl)phen- yl)-1H-pyrazolo[4,3-c]pyridin-1- yl)pyrrolidin-1-yl)prop-2-en-1- one | 387.1 |
| 24 | 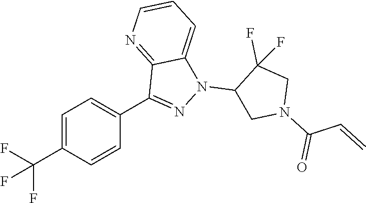 [SMILES: C=CC(=O)N1CC(n2nc(-c3ccc(C(F)(F)F)cc3)c3ncccc32)C(F)(F)C1] | 1-(3,3-difluoro-4-(3-(4-(trifluoro- methyl)phenyl)-1H-pyrazolo[4,3- b]pyridin-1-yl)pyrrolidin-1-yl)- prop-2-en-1-one | 423.1 |
| 25 | 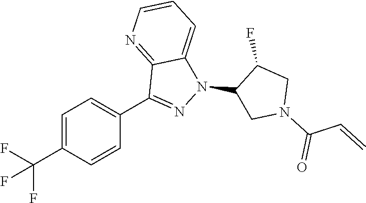 | 1-((3R,4S)-3-fluoro-4-(3-(4- (trifluoromethyl)phenyl)-1H- pyrazolo[4,3-b]pyridin-1-yl)- pyrrolidin-1-yl)prop-2-en-1-one | 405.1 |
| 26 | 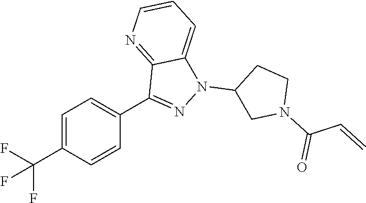 [SMILES: C=CC(=O)N1CCC(n2nc(-c3ccc(C(F)(F)F)cc3)c3ncccc32)C1] | 1-(3-(3-(4-(trifluoromethyl)phen- yl)-1H-pyrazolo[4,3-b]pyridin-1- yl)pyrrolidin-1-yl)prop-2-en-1- one | 387.1 |
| 27 | 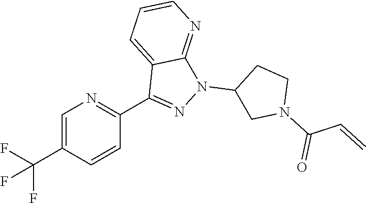 [SMILES: C=CC(=O)N1CCC(n2nc(-c3ccc(C(F)(F)F)cn3)c3cccnc32)C1] | 1-(3-(3-(5-(trifluoromethyl)- pyridin-2-yl)-1H-pyrazolo[3,4-b]- pyridin-1-yl)pyrrolidin-1-yl)prop- 2-en-1-one | 388.1 |
| 28 | 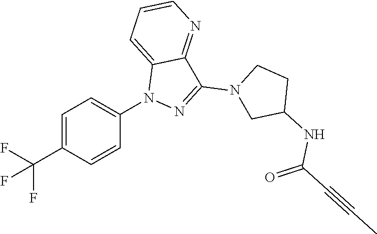 [SMILES: CC#CC(=O)NC1CCN(c2nn(-c3ccc(C(F)(F)F)cc3)c3cccnc23)C1] | N-(1-(1-(4-(trifluoromethyl)phen- yl)-1H-pyrazolo[4,3-b]pyridin-3- yl)pyrrolidin-3-yl)but-2-ynamide | 414.2 |
| 29 | 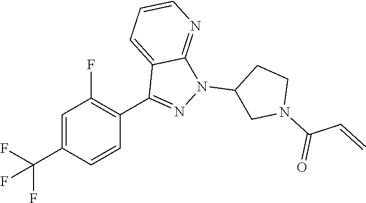 [SMILES: C=CC(=O)N1CCC(n2nc(-c3ccc(C(F)(F)F)cc3F)c3cccnc32)C1] | 1-(3-(3-(2-fluoro-4-(trifluoro- methyl)phenyl)-1H-pyrazolo[3,4- b]pyridin-1-yl)pyrrolidin-1-yl)- prop-2-en-1-one | 405.1 |
| 30 | 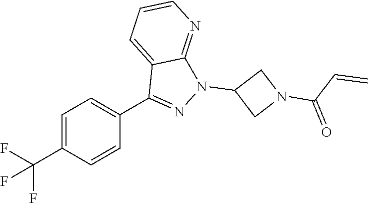 [SMILES: C=CC(=O)N1CC(n2nc(-c3ccc(C(F)(F)F)cc3)c3cccnc32)C1] | 1-(3-(3-(4-(trifluoromethyl)phen- yl)-1H-pyrazolo[3,4-b]pyridin-1- yl)azetidin-1-yl)prop-2-en-1-one | 373.1 |
| 31 | 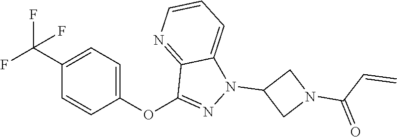 [SMILES: C=CC(=O)N1CC(n2nc(Oc3ccc(C(F)(F)F)cc3)c3ncccc32)C1] | 1-(3-(3-(4-(trifluoromethyl)phen- oxy)-1H-pyrazolo[4,3-b]pyridin- 1-yl)azetidin-1-yl)prop-2-en-1- one | 389.1 |
| 32 | 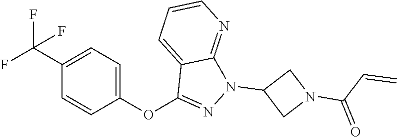 [SMILES: C=CC(=O)N1CC(n2nc(Oc3ccc(C(F)(F)F)cc3)c3cccnc32)C1] | 1-(3-(3-(4-(trifluoromethyl)phen- oxy)-1H-pyrazolo[3,4-b]pyridin- 1-yl)azetidin-1-yl)prop-2-en-1- one | 389.1 |
| 33 | 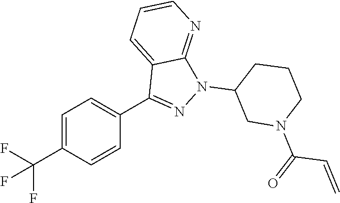 [SMILES: C=CC(=O)N1CCCC(n2nc(-c3ccc(C(F)(F)F)cc3)c3cccnc32)C1] | 1-(3-(3-(4-(trifluoromethyl)phen- yl)-1H-pyrazolo[3,4-b]pyridin-1- yl)piperidin-1-yl)prop-2-en-1-one | 401.1 |
| 34 | 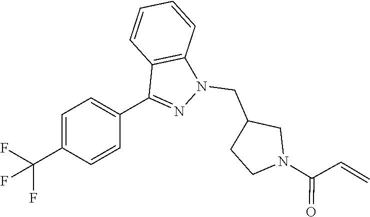 [SMILES: C=CC(=O)N1CCC(Cn2nc(-c3ccc(C(F)(F)F)cc3)c3ccccc32)C1] | 1-(3-((3-(4-(trifluoromethyl)phen- yl)-1H-indazol-1-yl)methyl)- pyrrolidin-1-yl)prop-2-en-1-one | 400.2 |
| 35 | 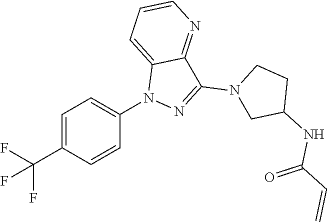 [SMILES: C=CC(=O)NC1CCN(c2nn(-c3ccc(C(F)(F)F)cc3)c3cccnc23)C1] | N-(1-(1-(4-(trifluoromethyl)phen- yl)-1H-pyrazolo[4,3-b]pyridin-3- yl)pyrrolidin-3-yl)acrylamide | 402.1 |
| 36 | 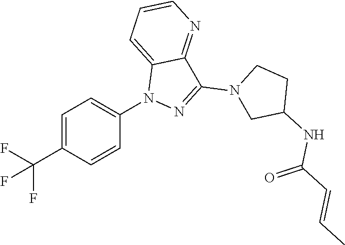 [SMILES: C/C=C/C(=O)NC1CCN(c2nn(-c3ccc(C(F)(F)F)cc3)c3cccnc23)C1] | (E)-N-(1-(1-(4-(trifluoromethyl)- phenyl)-1H-pyrazolo[4,3-b]- pyridin-3-yl)pyrrolidin-3-yl)but- 2-enamide | 416.2 |
| 37 | 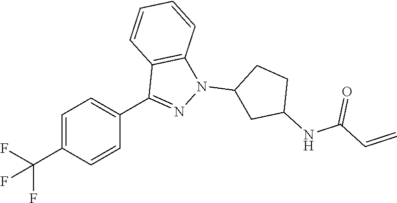 [SMILES: C=CC(=O)NC1CCC(n2nc(-c3ccc(C(F)(F)F)cc3)c3ccccc32)C1] | N-(3-(3-(4-(trifluoromethyl)phen- yl)-1H-indazol-1-yl)cyclopentyl)- acrylamide | 400.2 |
| 38 | 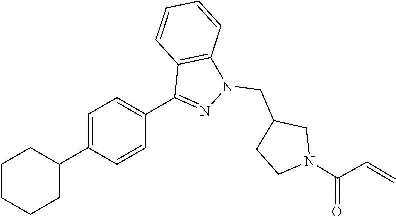 [SMILES: C=CC(=O)N1CCC(Cn2nc(-c3ccc(C4CCCCC4)cc3)c3ccccc32)C1] | 1-(3-((3-(4-cyclohexylphenyl)- 1H-indazol-1-yl)methyl)pyrrol- idin-1-yl)prop-2-en-1-one | 414.3 |
| 39 | 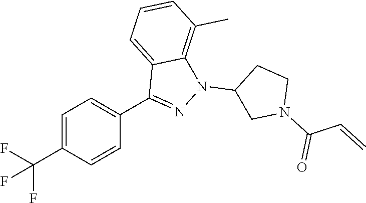 [SMILES: C=CC(=O)N1CCC(n2nc(-c3ccc(C(F)(F)F)cc3)c3cccc(C)c32)C1] | 1-(3-(7-methyl-3-(4-(trifluoro- methyl)phenyl)-1H-indazol-1- yl)pyrrolidin-1-yl)prop-2-en-1- one | 400.2 |
| 40 | 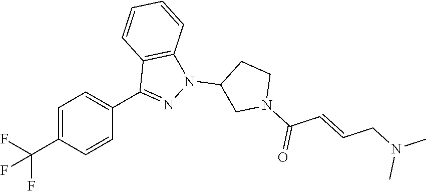 [SMILES: CN(C)C/C=C/C(=O)N1CCC(n2nc(-c3ccc(C(F)(F)F)cc3)c3ccccc32)C1] | (E)-4-(dimethylamino)-1-(3-(3- (4-(trifluoromethyl)phenyl)-1H- indazol-1-yl)pyrrolidin-1-yl)but- 2-en-1-one | 443.2 |
| 41 | 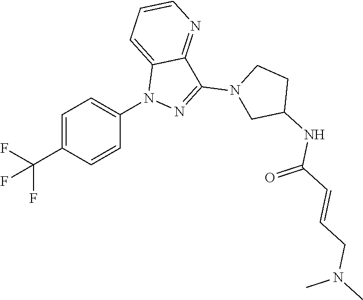 [SMILES: CN(C)C/C=C/C(=O)NC1CCN(c2nn(-c3ccc(C(F)(F)F)cc3)c3cccnc23)C1] | (E)-4-(dimethylamino)-N-(1-(1- (4-(trifluoromethyl)phenyl)-1H- pyrazolo[4,3-b]pyridin-3-yl)- pyrrolidin-3-yl)but-2-enamide | 459.2 |
| 42 | 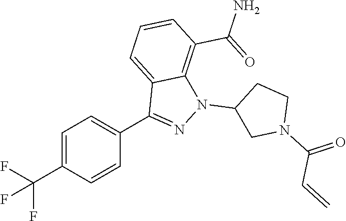 [SMILES: C=CC(=O)N1CCC(n2nc(-c3ccc(C(F)(F)F)cc3)c3cccc(C(N)=O)c32)C1] | 1-(1-acryloylpyrrolidin-3-yl)-3- (4-(trifluoromethyl)phenyl)-1H- indazole-7-carboxamide | 429.2 |
| 43 | 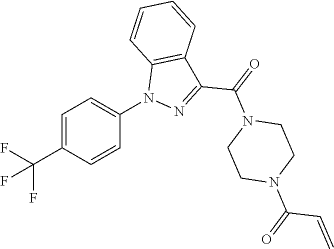 [SMILES: C=CC(=O)N1CCN(C(=O)c2nn(-c3ccc(C(F)(F)F)cc3)c3ccccc23)CC1] | 1-(4-(1-(4-(trifluoromethyl)phen- yl)-1H-indazole-3-carbonyl)- piperazin-1-yl)prop-2-en-1-one | 429.2 |
| 44 | 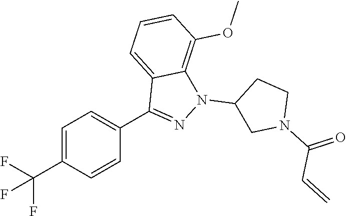 [SMILES: C=CC(=O)N1CCC(n2nc(-c3ccc(C(F)(F)F)cc3)c3cccc(OC)c32)C1] | 1-(3-(7-methoxy-3-(4-(trifluoro- methyl)phenyl)-1H-indazol-1-yl)- pyrrolidin-1-yl)prop-2-en-1-one | 416.2 |
| 45 | 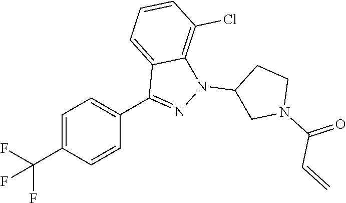 [SMILES: C=CC(=O)N1CCC(n2nc(-c3ccc(C(F)(F)F)cc3)c3cccc(Cl)c32)C1] | 1-(3-(7-chloro-3-(4-(trifluoro- methyl)phenyl)-1H-indazol-1-yl)- pyrrolidin-1-yl)prop-2-en-1-one | 420.1 |
| 46 | 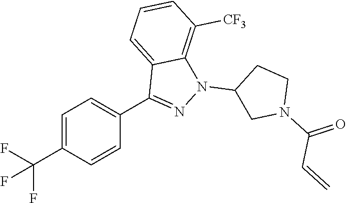 [SMILES: C=CC(=O)N1CCC(n2nc(-c3ccc(C(F)(F)F)cc3)c3cccc(C(F)(F)F)c32)C1] | 1-(3-(7-(trifluoromethyl)-3-(4- (trifluoromethyl)phenyl)-1H- indazol-1-yl)pyrrolidin-1-yl)- prop-2-en-1-one | 454.1 |
| 47 | 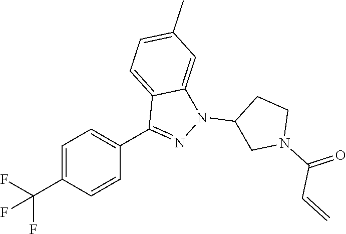 [SMILES: C=CC(=O)N1CCC(n2nc(-c3ccc(C(F)(F)F)cc3)c3ccc(C)cc32)C1] | 1-(3-(6-methyl-3-(4-(trifluoro- methyl)phenyl)-1H-indazol-1-yl)- pyrrolidin-1-yl)prop-2-en-1-one | 400.2 |
| 48 | 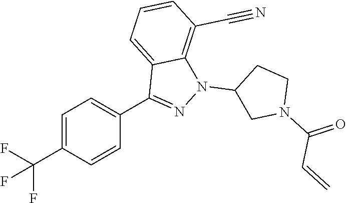 [SMILES: C=CC(=O)N1CCC(n2nc(-c3ccc(C(F)(F)F)cc3)c3cccc(C#N)c32)C1] | 1-(1-acryloylpyrrolidin-3-yl)-3-(4- (trifluoromethyl)phenyl)-1H- indazole-7-carbonitrile | 411.1 |
| 49 | 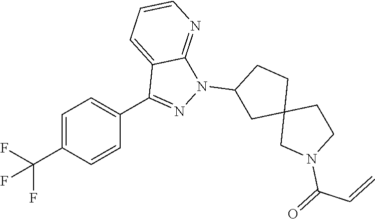 [SMILES: C=CC(=O)N1CCC2(CCC(n3nc(-c4ccc(C(F)(F)F)cc4)c4cccnc43)C2)C1] | 1-(7-(3-(4-(trifluoromethyl)phen- yl)-1H-pyrazolo[3,4-b]pyridin-1- yl)-2-azaspiro[4.4]nonan-2-yl)- prop-2-en-1-one | 441.2 |
| 50 | 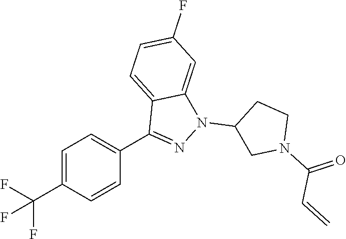 [SMILES: C=CC(=O)N1CCC(n2nc(-c3ccc(C(F)(F)F)cc3)c3ccc(F)cc32)C1] | 1-(3-(6-fluoro-3-(4-(trifluoro- methyl)phenyl)-1H-indazol-1-yl)- pyrrolidin-1-yl)prop-2-en-1-one | 404.1 |
| 51 | 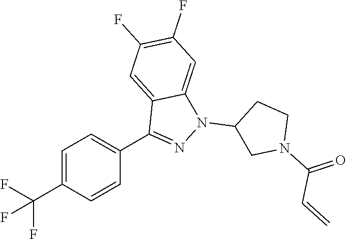 [SMILES: C=CC(=O)N1CCC(n2nc(-c3ccc(C(F)(F)F)cc3)c3cc(F)c(F)cc32)C1] | 1-(3-(5,6-difluoro-3-(4-(trifluoro- methyl)phenyl)-1H-indazol-1-yl)- pyrrolidin-1-yl)prop-2-en-1-one | 422.1 |
| 52 | 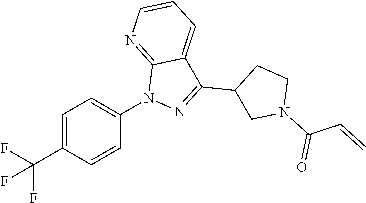 [SMILES: C=CC(=O)N1CCC(c2nn(-c3ccc(C(F)(F)F)cc3)c3ncccc23)C1] | 1-(3-(1-(4-(trifluoromethyl)phen- yl)-1H-pyrazolo[3,4-b]pyridin-3- yl)pyrrolidin-1-yl)prop-2-en-1-one | 387.1 |
| 53 | 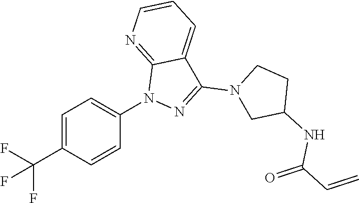 [SMILES: C=CC(=O)NC1CCN(c2nn(-c3ccc(C(F)(F)F)cc3)c3ncccc23)C1] | N-(1-(1-(4-(trifluoromethyl)phen- yl)-1H-pyrazolo[3,4-b]pyridin-3- yl)pyrrolidin-3-yl)acrylamide | 402.2 |
| 54 | 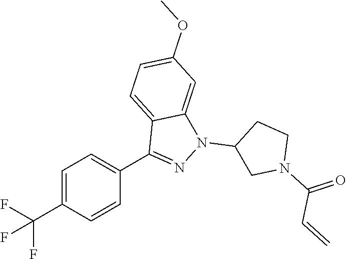 [SMILES: C=CC(=O)N1CCC(n2nc(-c3ccc(C(F)(F)F)cc3)c3ccc(OC)cc32)C1] | 1-(3-(6-methoxy-3-(4-(trifluoro- methyl)phenyl)-1H-indazol-1-yl)- pyrrolidin-1-yl)prop-2-en-1-one | 416.2 |
| 55 | 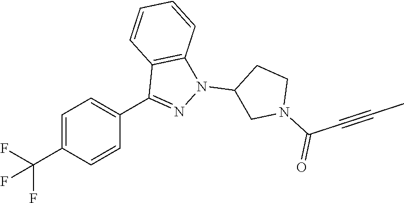 [SMILES: CC#CC(=O)N1CCC(n2nc(-c3ccc(C(F)(F)F)cc3)c3ccccc32)C1] | 1-(3-(3-(4-(trifluoromethyl)phen- yl)-1H-indazol-1-yl)pyrrolidin-1- yl)but-2-yn-1-one | 398.1 |
| 56 | 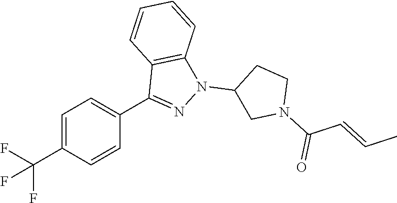 [SMILES: C/C=C/C(=O)N1CCC(n2nc(-c3ccc(C(F)(F)F)cc3)c3ccccc32)C1] | (E)-1-(3-(3-(4-(trifluoromethyl)- phenyl)-1H-indazol-1-yl)pyrrol- idin-1-yl)but-2-en-1-one | 400.2 |
| 57 | 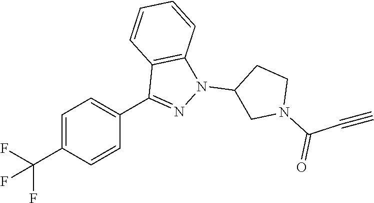 [SMILES: C#CC(=O)N1CCC(n2nc(-c3ccc(C(F)(F)F)cc3)c3ccccc32)C1] | 1-(3-(3-(4-(trifluoromethyl)phen- yl)-1H-indazol-1-yl)pyrrolidin-1- yl)prop-2-yn-1-one | 384.1 |
| 58 | 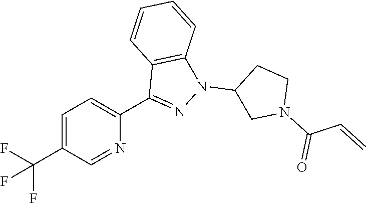 [SMILES: C=CC(=O)N1CCC(n2nc(-c3ccc(C(F)(F)F)cn3)c3ccccc32)C1] | 1-(3-(3-(5-(trifluoromethyl)pyr- idin-2-yl)-1H-indazol-1-yl)pyrrol- idin-1-yl)prop-2-en-1-one | 773.3 |
| 59 | 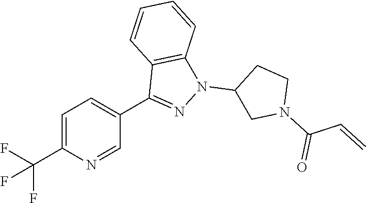 [SMILES: C=CC(=O)N1CCC(n2nc(-c3ccc(C(F)(F)F)nc3)c3ccccc32)C1] | 1-(3-(3-(6-(trifluoromethyl)pyr- idin-3-yl)-1H-indazol-1-yl)pyrrol- idin-1-yl)prop-2-en-1-one | 773.3 |
| 60 | 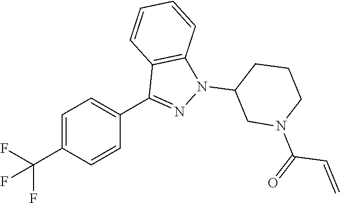 [SMILES: C=CC(=O)N1CCCC(n2nc(-c3ccc(C(F)(F)F)cc3)c3ccccc32)C1] | 1-(3-(3-(4-(trifluoromethyl)phen- yl)-1H-indazol-1-yl)piperidin-1- yl)prop-2-en-1-one | 400.2 |
| 61 | 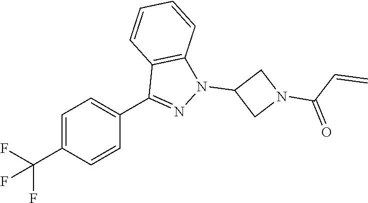 [SMILES: C=CC(=O)N1CC(n2nc(-c3ccc(C(F)(F)F)cc3)c3ccccc32)C1] | 1-(3-(3-(4-(trifluoromethyl)phen- yl)-1H-indazol-1-yl)azetidin-1-yl)- prop-2-en-1-one | 372.1 |
| 62 | 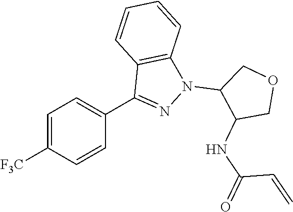 [SMILES: C=CC(=O)NC1COCC1n1nc(-c2ccc(C(F)(F)F)cc2)c2ccccc21] | N-(4-(3-(4-(trifluoromethyl)phen- yl)-1H-indazol-1-yl)tetrahydro- furan-3-yl)acrylamide | 402.1 |
| 63 | 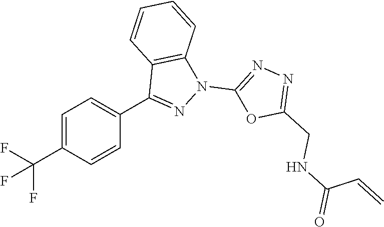 [SMILES: C=CC(=O)NCc1nnc(-n2nc(-c3ccc(C(F)(F)F)cc3)c3ccccc32)o1] | N-((5-(3-(4-(trifluoromethyl)phen- yl)-1H-indazol-1-yl)-1,3,4-oxa- diazol-2-yl)methyl)acrylamide | 414.1 |
| 64 | 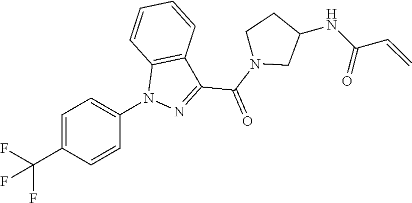 [SMILES: C=CC(=O)NC1CCN(C(=O)c2nn(-c3ccc(C(F)(F)F)cc3)c3ccccc23)C1] | N-(1-(1-(4-(trifluoromethyl)phen- yl)-1H-indazole-3-carbonyl)- pyrrolidin-3-yl)acrylamide | 429.1 |
| 65 | 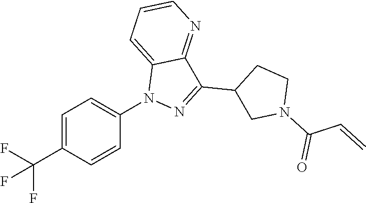 [SMILES: C=CC(=O)N1CCC(c2nn(-c3ccc(C(F)(F)F)cc3)c3cccnc23)C1] | 1-(3-(1-(4-(trifluoromethyl)phen- yl)-1H-pyrazolo[4,3-b]pyridin-3- yl)pyrrolidin-1-yl)prop-2-en-1-one | 387.1 |
| 66 | 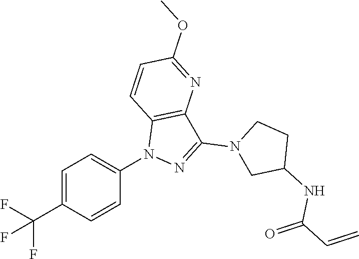 [SMILES: C=CC(=O)NC1CCN(c2nn(-c3ccc(C(F)(F)F)cc3)c3ccc(OC)nc23)C1] | N-(1-(5-methoxy-1-(4-(trifluoro- methyl)phenyl)-1H-pyrazolo[4,3- b]pyridin-3-yl)pyrrolidin-3-yl)- acrylamide | 432.2 |
| 67 | 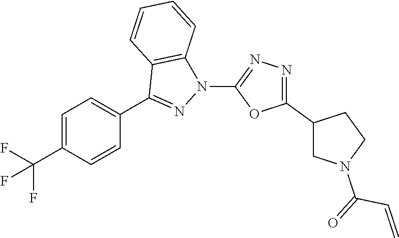 [SMILES: C=CC(=O)N1CCC(c2nnc(-n3nc(-c4ccc(C(F)(F)F)cc4)c4ccccc43)o2)C1] | 1-(3-(5-(3-(4-(trifluoromethyl)- phenyl)-1H-indazol-1-yl)-1,3,4- oxadiazol-2-yl)pyrrolidin-1-yl)- prop-2-en-1-one | 454.1 |
| 68 | 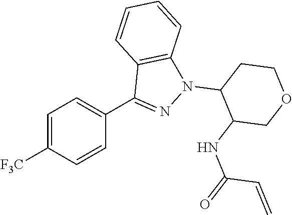 [SMILES: C=CC(=O)NC1COCCC1n1nc(-c2ccc(C(F)(F)F)cc2)c2ccccc21] | N-(4-(3-(4-(trifluoromethyl)phen- yl)-1H-indazol-1-yl)tetrahydro- 2H-pyran-3-yl)acrylamide | 416.2 |
| 69 | 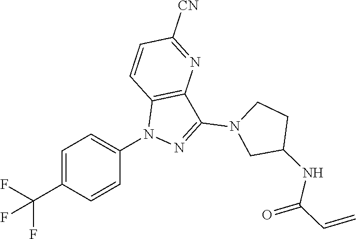 [SMILES: C=CC(=O)NC1CCN(c2nn(-c3ccc(C(F)(F)F)cc3)c3ccc(C#N)nc23)C1] | N-(1-(5-cyano-1-(4-(trifluoro- methyl)phenyl)-1H-pyrazolo[4,3- b]pyridin-3-yl)pyrrolidin-3-yl)- acrylamide | 427.1 |
| 70 | 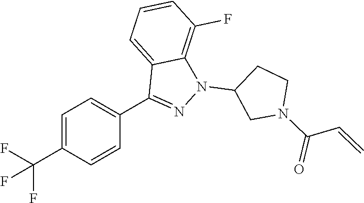 [SMILES: C=CC(=O)N1CCC(n2nc(-c3ccc(C(F)(F)F)cc3)c3cccc(F)c32)C1] | 1-(3-(7-fluoro-3-(4-(trifluoro- methyl)phenyl)-1H-indazol-1-yl)- pyrrolidin-1-yl)prop-2-en-1-one | 404.1 |
| 71 | 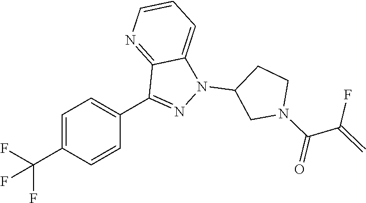 [SMILES: C=C(F)C(=O)N1CCC(n2nc(-c3ccc(C(F)(F)F)cc3)c3ncccc32)C1] | 2-fluoro-1-(3-(3-(4-(trifluoro- methyl)phenyl)-1H-pyrazolo[4,3- b]pyridin-1-yl)pyrrolidin-1-yl)- prop-2-en-1-one | 405.1 |
| 72 | 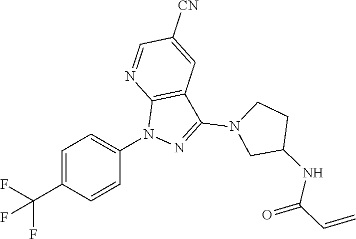 [SMILES: C=CC(=O)NC1CCN(c2nn(-c3ccc(C(F)(F)F)cc3)c3ncc(C#N)cc23)C1] | N-(1-(5-cyano-1-(4-(trifluoro- methyl)phenyl)-1H-pyrazolo[3,4- b]pyridin-3-yl)pyrrolidin-3-yl)- acrylamide | 427.1 |
| 73 | 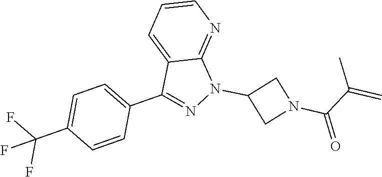 [SMILES: C=C(C)C(=O)N1CC(n2nc(-c3ccc(C(F)(F)F)cc3)c3cccnc32)C1] | 2-methyl-1-(3-(3-(4-(trifluoro- methyl)phenyl)-1H-pyrazolo[3,4- b]pyridin-1-yl)azetidin-1-yl)prop- 2-en-1-one | 387.1 |
| 74 | 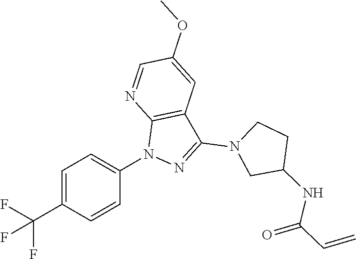 [SMILES: C=CC(=O)NC1CCN(c2nn(-c3ccc(C(F)(F)F)cc3)c3ncc(OC)cc23)C1] | N-(1-(5-methoxy-1-(4-(trifluoro- methyl)phenyl)-1H-pyrazolo[3,4- b]pyridin-3-yl)pyrrolidin-3-yl)- acrylamide | 432.2 |
| 75 | 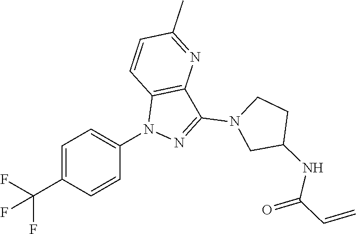 [SMILES: C=CC(=O)NC1CCN(c2nn(-c3ccc(C(F)(F)F)cc3)c3ccc(C)nc23)C1] | N-(1-(5-methyl-1-(4-(trifluoro- methyl)phenyl)-1H-pyrazolo[4,3- b]pyridin-3-yl)pyrrolidin-3-yl)- acrylamide | 416.2 |
| 76 | 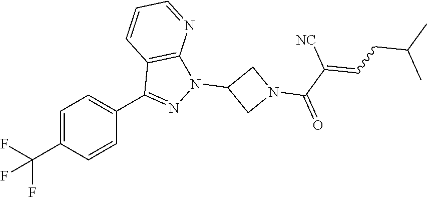 [SMILES: CC(C)CC=C(C#N)C(=O)N1CC(n2nc(-c3ccc(C(F)(F)F)cc3)c3cccnc32)C1] | 5-methyl-2-(3-(3-(4-(trifluoro- methyl)phenyl)-1H-pyrazolo[3,4- b]pyridin-1-yl)azetidine-1-carbon- yl)hex-2-enenitrile | 454.2 |
| 77 | 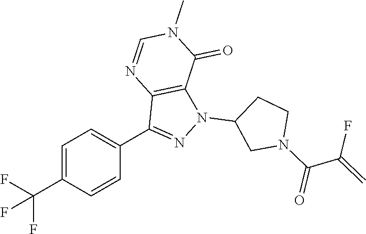 [SMILES: C=C(F)C(=O)N1CCC(n2nc(-c3ccc(C(F)(F)F)cc3)c3ncn(C)c(=O)c32)C1] | 1-(1-(2-fluoroacryloyl)pyrrolidin- 3-yl)-6-methyl-3-(4-(trifluoro- methyl)phenyl)-1,6-dihydro-7H- pyrazolo[4,3-d]pyrimidin-7-one | 435.38 |
| 78 | 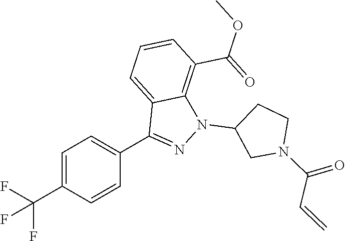 [SMILES: C=CC(=O)N1CCC(n2nc(-c3ccc(C(F)(F)F)cc3)c3cccc(C(=O)OC)c32)C1] | methyl 1-(1-acryloylpyrrolidin-3- yl)-3-(4-(trifluoromethyl)phenyl)- 1H-indazole-7-carboxylate | 444.1 |
| 79 | 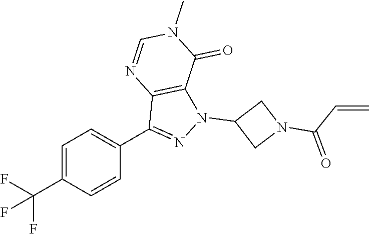 [SMILES: C=CC(=O)N1CC(n2nc(-c3ccc(C(F)(F)F)cc3)c3ncn(C)c(=O)c32)C1] | 1-(1-acryloylazetidin-3-yl)-6- methyl-3-(4-(trifluoromethyl)- phenyl)-1,6-dihydro-7H- pyrazolo[4,3-d]pyrimidin-7-one | 404.1 |
| 80 | 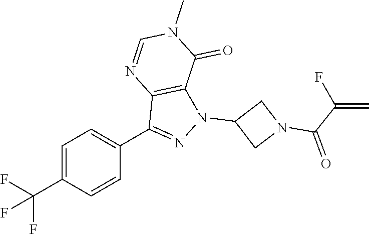 [SMILES: C=C(F)C(=O)N1CC(n2nc(-c3ccc(C(F)(F)F)cc3)c3ncn(C)c(=O)c32)C1] | 1-(1-(2-fluoroacryloyl)azetidin-3- yl)-6-methyl-3-(4-(trifluorometh- yl)phenyl)-1,6-dihydro-7H- pyrazolo[4,3-d]pyrimidin-7-one | 422.1 |
| 81 | 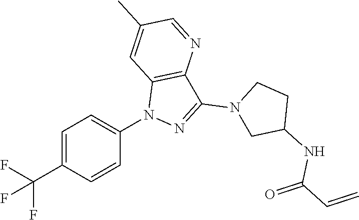 [SMILES: C=CC(=O)NC1CCN(c2nn(-c3ccc(C(F)(F)F)cc3)c3cc(C)cnc23)C1] | N-(1-(6-methyl-1-(4-(trifluoro- methyl)phenyl)-1H-pyrazolo[4,3- b]pyridin-3-yl)pyrrolidin-3-yl)- acrylamide | 416.2 |
| 82 | 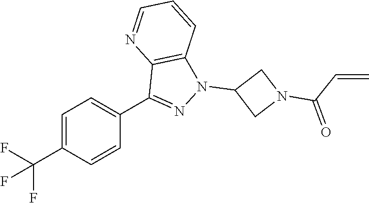 [SMILES: C=CC(=O)N1CC(n2nc(-c3ccc(C(F)(F)F)cc3)c3ncccc32)C1] | 1-(3-(3-(4-(trifluoromethyl)phen- yl)-1H-pyrazolo[4,3-b]pyridin-1- yl)azetidin-1-yl)prop-2-en-1-one | 373.1 |
| 83 | 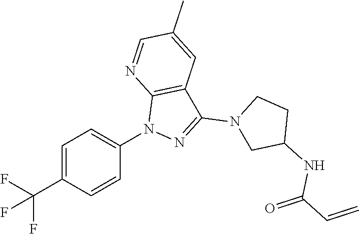 [SMILES: C=CC(=O)NC1CCN(c2nn(-c3ccc(C(F)(F)F)cc3)c3ncc(C)cc23)C1] | N-(1-(5-methyl-1-(4-(trifluoro- methyl)-phenyl)-1H-pyrazolo[3,4- b]pyridin-3-yl)pyrrolidin-3-yl)- acrylamide | 416.2 |
| 84 | 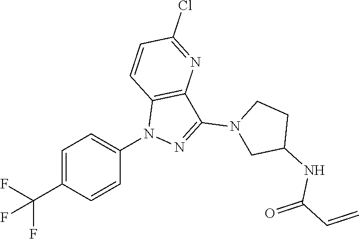 [SMILES: C=CC(=O)NC1CCN(c2nn(-c3ccc(C(F)(F)F)cc3)c3ccc(Cl)nc23)C1] | N-(1-(5-chloro-1-(4-(trifluoro- methyl)phenyl)-1H-pyrazolo[4,3- b]pyridin-3-yl)pyrrolidin-3-yl)- acrylamide | 436.1 |
| 85 | 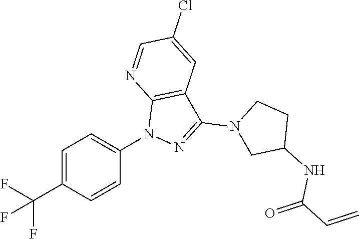 [SMILES: C=CC(=O)NC1CCN(c2nn(-c3ccc(C(F)(F)F)cc3)c3ncc(Cl)cc23)C1] | N-(1-(5-chloro-1-(4-(trifluoro- methyl)phenyl)-1H-pyrazolo[3,4- b]pyridin-3-yl)pyrrolidin-3-yl)- acrylamide | 436.1 |
| 86 | 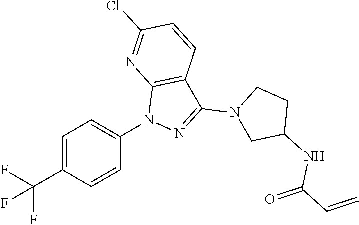 [SMILES: C=CC(=O)NC1CCN(c2nn(-c3ccc(C(F)(F)F)cc3)c3nc(Cl)ccc23)C1] | N-(1-(6-chloro-1-(4-(trifluoro- methyl)phenyl)-1H-pyrazolo[3,4- b]pyridin-3-yl)pyrrolidin-3-yl)- acrylamide | 436.1 |
| 87 | 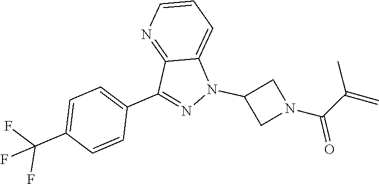 [SMILES: C=C(C)C(=O)N1CC(n2nc(-c3ccc(C(F)(F)F)cc3)c3ncccc32)C1] | 2-methyl-1-(3-(3-(4-(trifluoro- methyl)phenyl)-1H-pyrazolo[4,3- b]pyridin-1-yl)azetidin-1-yl)prop- 2-en-1-one | 387.1 |
| 88 | 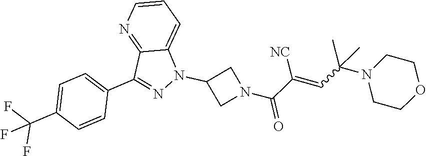 [SMILES: CC(C)(C=C(C#N)C(=O)N1CC(n2nc(-c3ccc(C(F)(F)F)cc3)c3ncccc32)C1)N1CCOCC1] | 4-methyl-4-morpholino-2-(3-(3- (4-(trifluoromethyl)phenyl)-1H- pyrazolo[4,3-b]pyridin-1-yl)- azetidine-1-carbonyl)pent-2- enenitrile | 525.2 |
| 89 | 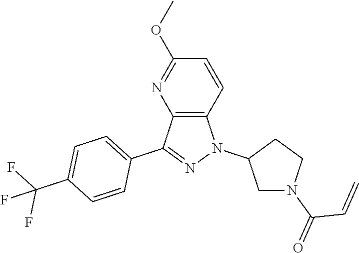 [SMILES: C=CC(=O)N1CCC(n2nc(-c3ccc(C(F)(F)F)cc3)c3nc(OC)ccc32)C1] | 1-(3-(5-methoxy-3-(4-(trifluoro- methyl)phenyl)-1H-pyrazolo[4,3- b]pyridin-1-yl)pyrrolidin-1-yl)- prop-2-en-1-one | 417.2 |
| 90 | 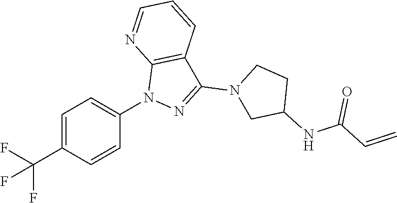 [SMILES: C=CC(=O)NC1CCN(c2nn(-c3ccc(C(F)(F)F)cc3)c3ncccc23)C1] | N-(1-(1-(4-(trifluoromethyl)phen- yl)-1H-pyrazolo[3,4-b]pyridin-3- yl)pyrrolidin-3-yl)acrylamide | 402.1 |
| 91 | 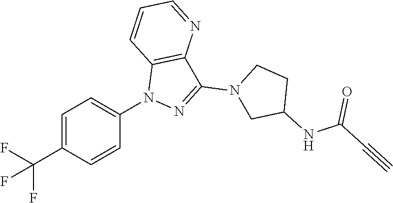 [SMILES: C#CC(=O)NC1CCN(c2nn(-c3ccc(C(F)(F)F)cc3)c3cccnc23)C1] | N-(1-(1-(4-(trifluoromethyl)phen- yl)-1H-pyrazolo[4,3-b]pyridin-3- yl)pyrrolidin-3-yl)propiolamide | 400.1 |
| 92 | 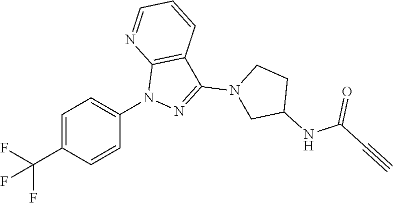 [SMILES: C#CC(=O)NC1CCN(c2nn(-c3ccc(C(F)(F)F)cc3)c3ncccc23)C1] | N-(1-(1-(4-(trifluoromethyl)phen- yl)-1H-pyrazolo[3,4-b]pyridin-3- yl)pyrrolidin-3-yl)propiolamide | 400.1 |
| 93 | 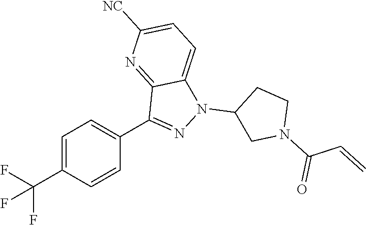 [SMILES: C=CC(=O)N1CCC(n2nc(-c3ccc(C(F)(F)F)cc3)c3nc(C#N)ccc32)C1] | 1-(1-acryloylpyrrolidin-3-yl)-3- (4-(trifluoromethyl)phenyl)-1H- pyrazolo[4,3-b]pyridine-5- carbonitrile | 412.1 |
| 94 | 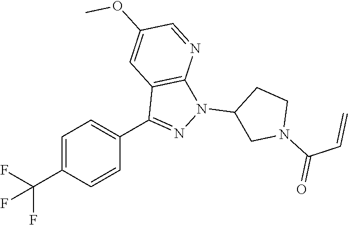 [SMILES: C=CC(=O)N1CCC(n2nc(-c3ccc(C(F)(F)F)cc3)c3cc(OC)cnc32)C1] | 1-(3-(5-methoxy-3-(4-(trifluoro- methyl)phenyl)-1H-pyrazolo[3,4- b]pyridin-1-yl)pyrrolidin-1-yl)- prop-2-en-1-one | 416.4 |
| 95 | 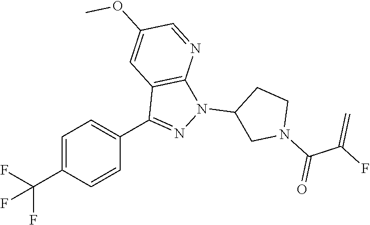 [SMILES: C=C(F)C(=O)N1CCC(n2nc(-c3ccc(C(F)(F)F)cc3)c3cc(OC)cnc32)C1] | 2-fluoro-1-(3-(5-methoxy-3-(4- (trifluoromethyl)phenyl)-1H- pyrazolo[3,4-b]pyridin-1-yl)- pyrrolidin-1-yl)prop-2-en-1-one | 435.1 |
| 96 | 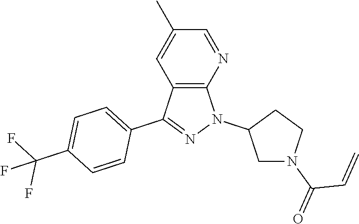 [SMILES: C=CC(=O)N1CCC(n2nc(-c3ccc(C(F)(F)F)cc3)c3cc(C)cnc32)C1] | 1-(3-(5-methyl-3-(4-(trifluoro- methyl)phenyl)-1H-pyrazolo[3,4- b]pyridin-1-yl)pyrrolidin-1-yl)- prop-2-en-1-one | 401.2 |
| 97 | 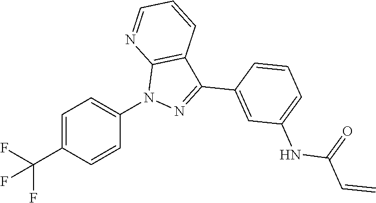 [SMILES: C=CC(=O)Nc1cccc(-c2nn(-c3ccc(C(F)(F)F)cc3)c3ncccc23)c1] | N-(3-(1-(4-(trifluoromethyl)phen- yl)-1H-pyrazolo[3,4-b]pyridin-3- yl)phenyl)acrylamide | 409.1 |
| 98 | 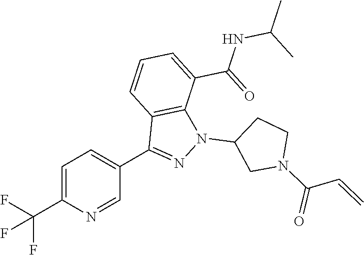 [SMILES: C=CC(=O)N1CCC(n2nc(-c3ccc(C(F)(F)F)nc3)c3cccc(C(=O)NC(C)C)c32)C1] | 1-(1-acryloylpyrrolidin-3-yl)-N- isopropyl-3-(6-(trifluoromethyl)- pyridin-3-yl)-1H-indazole-7- carboxamide | 472.2 |
| 99 | 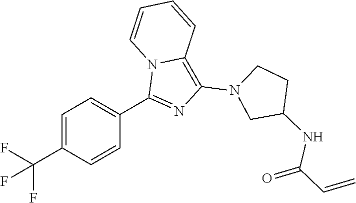 [SMILES: C=CC(=O)NC1CCN(c2nc(-c3ccc(C(F)(F)F)cc3)n3ccccc23)C1] | N-(1-(3-(4-(trifluoromethyl)phen- yl)imidazo[1,5-a]pyridin-1-yl)- pyrrolidin-3-yl)acrylamide | 401.2 |
| 100 | 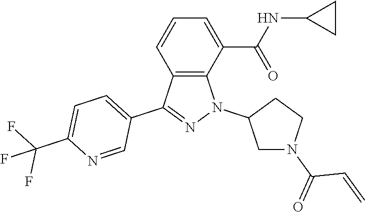 [SMILES: C=CC(=O)N1CCC(n2nc(-c3ccc(C(F)(F)F)nc3)c3cccc(C(=O)NC4CC4)c32)C1] | 1-(1-acryloylpyrrolidin-3-yl)-N- cyclopropyl-3-(6-(trifluorometh- yl)pyridin-3-yl)-1H-indazole-7- carboxamide | 470.2 |
| 101 | 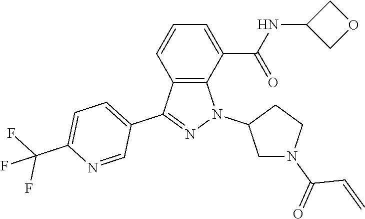 [SMILES: C=CC(=O)N1CCC(n2nc(-c3ccc(C(F)(F)F)nc3)c3cccc(C(=O)NC4COC4)c32)C1] | 1-(1-acryloylpyrrolidin-3-yl)-N- (oxetan-3-yl)-3-(6-(trifluorometh- yl)pyridin-3-yl)-1H-indazole-7- carboxamide | 486.2 |
| 102 | 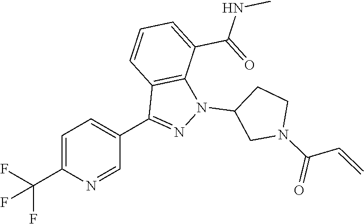 [SMILES: C=CC(=O)N1CCC(n2nc(-c3ccc(C(F)(F)F)nc3)c3cccc(C(=O)NC)c32)C1] | 1-(1-acryloylpyrrolidin-3-yl)-N- methyl-3-(6-(trifluoromethyl)- pyridin-3-yl)-1H-indazole-7- carboxamide | 444.2 |
| 103 | 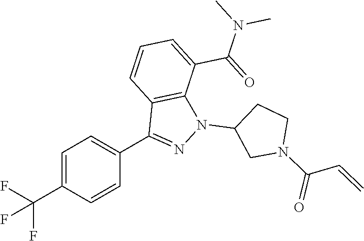 [SMILES: C=CC(=O)N1CCC(n2nc(-c3ccc(C(F)(F)F)cc3)c3cccc(C(=O)N(C)C)c32)C1] | 1-(1-acryloylpyrrolidin-3-yl)-N,N- dimethyl-3-(4-(trifluoromethyl)- phenyl)-1H-indazole-7-carbox- amide | 457.2 |
| 104 | 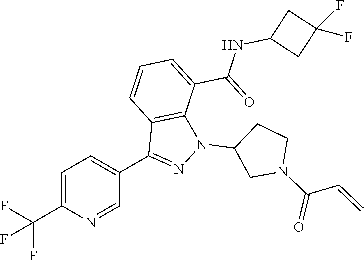 [SMILES: C=CC(=O)N1CCC(n2nc(-c3ccc(C(F)(F)F)nc3)c3cccc(C(=O)NC4CC(F)(F)C4)c32)C1] | 1-(1-acryloylpyrrolidin-3-yl)-N- (3,3-difluorocyclobutyl)-3-(6- (trifluoromethyl)pyridin-3-yl)- 1H-indazole-7-carboxamide | 520.2 |
| 105 | 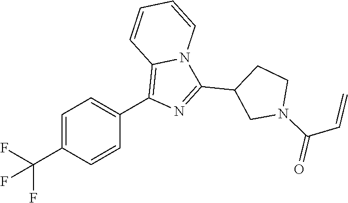 [SMILES: C=CC(=O)N1CCC(c2nc(-c3ccc(C(F)(F)F)cc3)c3ccccn23)C1] | 1-(3-(1-(4-(trifluoromethyl)phen- yl)imidazo[1,5-a]pyridin-3-yl)- pyrrolidin-1-yl)prop-2-en-1-one | 386.1 |
| 106 | 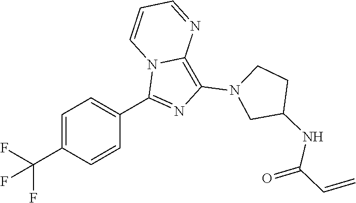 [SMILES: C=CC(=O)NC1CCN(c2nc(-c3ccc(C(F)(F)F)cc3)n3cccnc23)C1] | N-(1-(6-(4-(trifluoromethyl)phen- yl)imidazo[1,5-a]pyrimidin-8-yl)- pyrrolidin-3-yl)acrylamide | 402.2 |
| 107 | 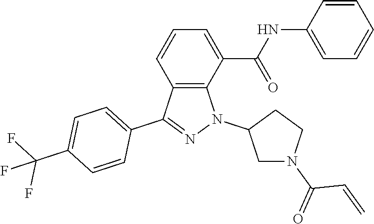 [SMILES: C=CC(=O)N1CCC(n2nc(-c3ccc(C(F)(F)F)cc3)c3cccc(C(=O)Nc4ccccc4)c32)C1] | 1-(1-acryloylpyrrolidin-3-yl)-N- phenyl-3-(4-(trifluoromethyl)- phenyl)-1H-indazole-7-carbox- amide | 505.2 |
| 108 | 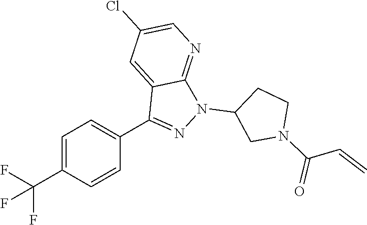 [SMILES: C=CC(=O)N1CCC(n2nc(-c3ccc(C(F)(F)F)cc3)c3cc(Cl)cnc32)C1] | 1-(3-(5-chloro-3-(4-(trifluoro- methyl)phenyl)-1H-pyrazolo[3,4- b]pyridin-1-yl)pyrrolidin-1-yl)- prop-2-en-1-one | 421.1 |
| 109 | 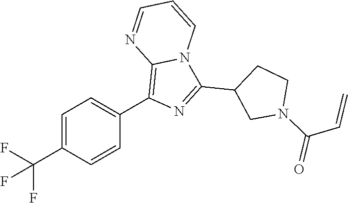 [SMILES: C=CC(=O)N1CCC(c2nc(-c3ccc(C(F)(F)F)cc3)c3ncccn23)C1] | 1-(3-(8-(4-(trifluoromethyl)phen- yl)imidazo[1,5-a]pyrimidin-6-yl)- pyrrolidin-1-yl)prop-2-en-1-one | 387.1 |
| 110 | 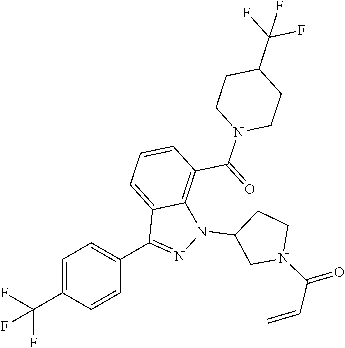 [SMILES: C=CC(=O)N1CCC(n2nc(-c3ccc(C(F)(F)F)cc3)c3cccc(C(=O)N4CCC(C(F)(F)F)CC4)c32)C1] | 1-(3-(3-(4-(trifluoromethyl)phen- yl)-7-(4-(trifluoromethyl)piperi- dine-1-carbonyl)-1H-indazol-1- yl)pyrrolidin-1-yl)prop-2-en-1-one | 565.2 |
| 111 | 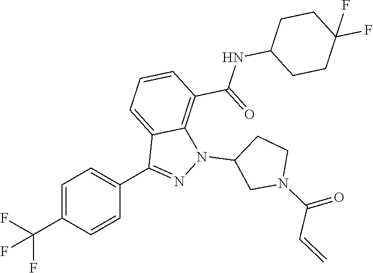 [SMILES: C=CC(=O)N1CCC(n2nc(-c3ccc(C(F)(F)F)cc3)c3cccc(C(=O)NC4CCC(F)(F)CC4)c32)C1] | 1-(1-acryloylpyrrolidin-3-yl)-N- (4,4-difluorocyclohexyl)-3-(4- (trifluoromethyl)phenyl)-1H- indazole-7-carboxamide | 547.2 |
| 112 | 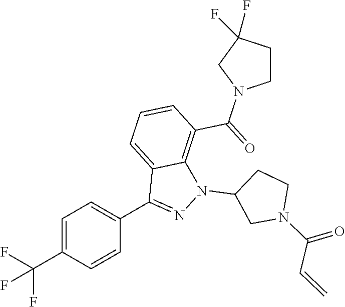 [SMILES: C=CC(=O)N1CCC(n2nc(-c3ccc(C(F)(F)F)cc3)c3cccc(C(=O)N4CCC(F)(F)C4)c32)C1] | 1-(3-(7-(3,3-difluoropyrrolidine- 1-carbonyl)-3-(4-(trifluoro- methyl)phenyl)-1H-indazol-1-yl)- pyrrolidin-1-yl)prop-2-en-1-one | 519.2 |
| 113 | 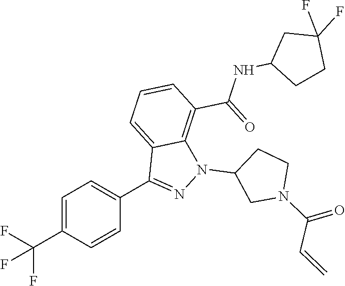 [SMILES: C=CC(=O)N1CCC(n2nc(-c3ccc(C(F)(F)F)cc3)c3cccc(C(=O)NC4CCC(F)(F)C4)c32)C1] | 1-(1-acryloylpyrrolidin-3-yl)-N- (3,3-difluorocyclopentyl)-3-(4- (trifluoromethyl)phenyl)-1H- indazole-7-carboxamide | 533.2 |
| 114 | 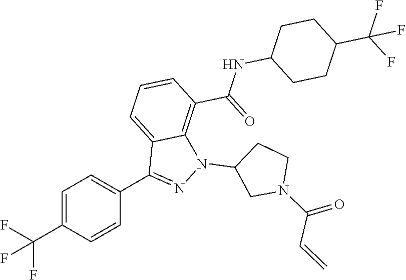 [SMILES: C=CC(=O)N1CCC(n2nc(-c3ccc(C(F)(F)F)cc3)c3cccc(C(=O)NC4CCC(C(F)(F)F)CC4)c32)C1] | 1-(1-acryloylpyrrolidin-3-yl)-N- (4-(trifluoromethyl)cyclohexyl)-3- (4-(trifluoromethyl)phenyl)-1H- indazole-7-carboxamide | 579.2 |
| 115 | 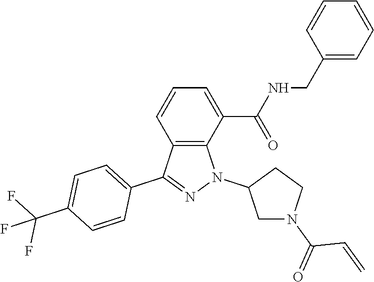 [SMILES: C=CC(=O)N1CCC(n2nc(-c3ccc(C(F)(F)F)cc3)c3cccc(C(=O)NCc4ccccc4)c32)C1] | 1-(1-acryloylpyrrolidin-3-yl)-N- benzyl-3-(4-(trifluoromethyl)- phenyl)-1H-indazole-7-carbox- amide | 519.2 |
| 116 | 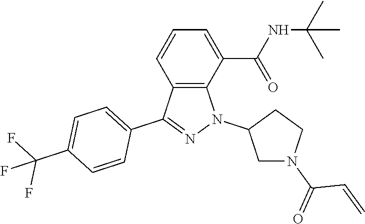 [SMILES: C=CC(=O)N1CCC(n2nc(-c3ccc(C(F)(F)F)cc3)c3cccc(C(=O)NC(C)(C)C)c32)C1] | 1-(1-acryloylpyrrolidin-3-yl)-N- (tert-butyl)-3-(4-(trifluoromethyl)- phenyl)-1H-indazole-7-carbox- amide | 485.2 |
| 117 | 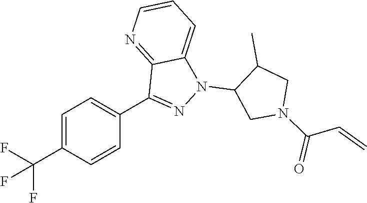 [SMILES: C=CC(=O)N1CC(C)C(n2nc(-c3ccc(C(F)(F)F)cc3)c3ncccc32)C1] | 1-(3-methyl-4-(3-(4-(trifluoro- methyl)phenyl)-1H-pyrazolo[4,3- b]pyridin-1-yl)pyrrolidin-1-yl)- prop-2-en-1-one | 401.2 |
| 118 | 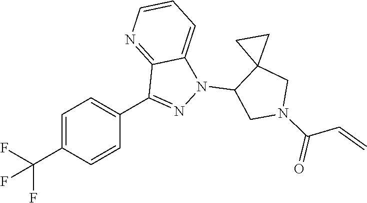 [SMILES: C=CC(=O)N1CC(n2nc(-c3ccc(C(F)(F)F)cc3)c3ncccc32)C2(CC2)C1] | 1-(7-(3-(4-(trifluoromethyl)phen- yl)-1H-pyrazolo[4,3-b]pyridin-1- yl)-5-azaspiro[2.4]heptan-5-yl)- prop-2-en-1-one | 413.2 |
| 119 | 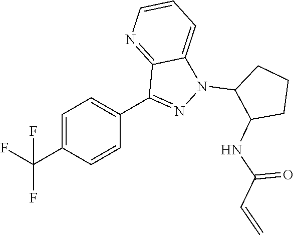 [SMILES: C=CC(=O)NC1CCCC1n1nc(-c2ccc(C(F)(F)F)cc2)c2ncccc21] | N-(2-(3-(4-(trifluoromethyl)phen- yl)-1H-pyrazolo[4,3-b]pyridin-1- yl)cyclopentyl)acrylamide | 401.2 |
| 120 | 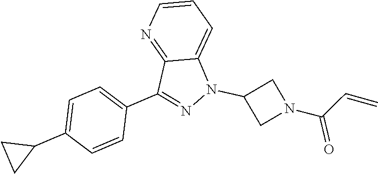 [SMILES: C=CC(=O)N1CC(n2nc(-c3ccc(C4CC4)cc3)c3ncccc32)C1] | 1-(3-(3-(4-cyclopropylphenyl)- 1H-pyrazolo[4,3-b]pyridin-1-yl)- azetidin-1-yl)prop-2-en-1-one | 345.2 |
| 121 | 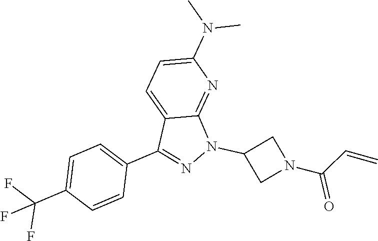 [SMILES: C=CC(=O)N1CC(n2nc(-c3ccc(C(F)(F)F)cc3)c3ccc(N(C)C)nc32)C1] | 1-(3-(6-(dimethylamino)-3-(4- (trifluoromethyl)phenyl)-1H- pyrazolo[3,4-b]pyridin-1-yl)- azetidin-1-yl)prop-2-en-1-one | 416.2 |
| 122 | 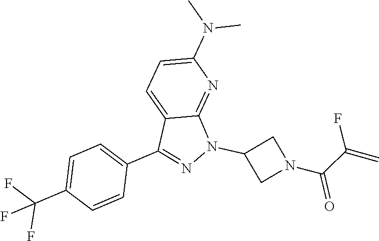 [SMILES: C=C(F)C(=O)N1CC(n2nc(-c3ccc(C(F)(F)F)cc3)c3ccc(N(C)C)nc32)C1] | 1-(3-(6-(dimethylamino)-3-(4- (trifluoromethyl)phenyl)-1H- pyrazolo[3,4-b]pyridin-1-yl)- azetidin-1-yl)-2-fluoroprop-2- en-1-one | 434.2 |
| 123 | 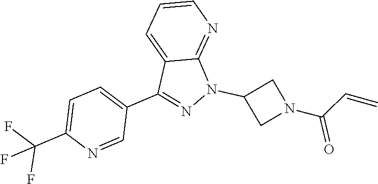 [SMILES: C=CC(=O)N1CC(n2nc(-c3ccc(C(F)(F)F)nc3)c3cccnc32)C1] | 1-(3-(3-(6-(trifluoromethyl)pyr- idin-3-yl)-1H-pyrazolo[3,4-b]- pyridin-1-yl)azetidin-1-yl)prop- 2-en-1-one | 374.1 |
| 124 | 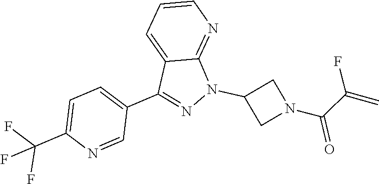 [SMILES: C=C(F)C(=O)N1CC(n2nc(-c3ccc(C(F)(F)F)nc3)c3cccnc32)C1] | 2-fluoro-1-(3-(3-(6-(trifluoro- methyl)pyridin-3-yl)-1H- pyrazolo[3,4-b]pyridin-1-yl)- azetidin-1-yl)prop-2-en-1-one | 392.1 |
| 125 | 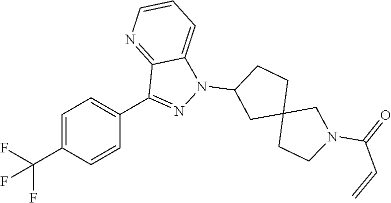 [SMILES: C=CC(=O)N1CCC2(CCC(n3nc(-c4ccc(C(F)(F)F)cc4)c4ncccc43)C2)C1] | 1-(7-(3-(4-(trifluoromethyl)phen- yl)-1H-pyrazolo[4,3-b]pyridin-1- yl)-2-azaspiro[4.4]nonan-2-yl)- prop-2-en-1-one | 441.2 |
| 126 | 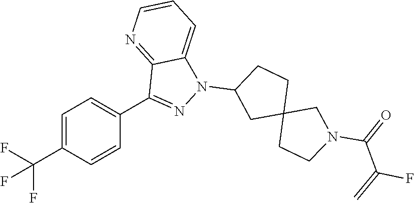 [SMILES: C=C(F)C(=O)N1CCC2(CCC(n3nc(-c4ccc(C(F)(F)F)cc4)c4ncccc43)C2)C1] | 2-fluoro-1-(7-(3-(4-(trifluoro- methyl)phenyl)-1H-pyrazolo[4,3- b]pyridin-1-yl)-2-azaspiro[4.4]- nonan-2-yl)prop-2-en-1-one | 459.2 |
| 127 | 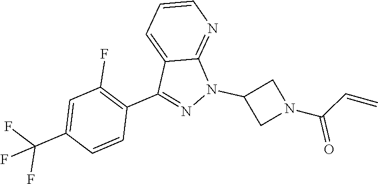 [SMILES: C=CC(=O)N1CC(n2nc(-c3ccc(C(F)(F)F)cc3F)c3cccnc32)C1] | 1-(3-(3-(2-fluoro-4-(trifluoro- methyl)phenyl)-1H-pyrazolo[3,4- b]pyridin-1-yl)azetidin-1-yl)prop- 2-en-1-one | 391.1 |
| 128 | 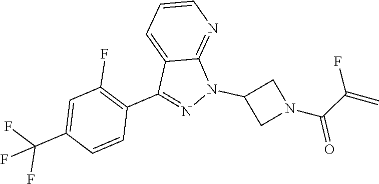 [SMILES: C=C(F)C(=O)N1CC(n2nc(-c3ccc(C(F)(F)F)cc3F)c3cccnc32)C1] | 2-fluoro-1-(3-(3-(2-fluoro-4- (trifluoromethyl)phenyl)-1H- pyrazolo[3,4-b]pyridin-1-yl)- azetidin-1-yl)prop-2-en-1-one | 409.1 |
| 129 | 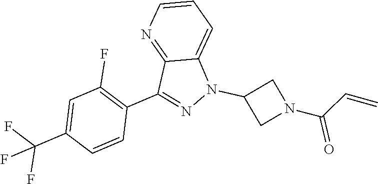 [SMILES: C=CC(=O)N1CC(n2nc(-c3ccc(C(F)(F)F)cc3F)c3ncccc32)C1] | 1-(3-(3-(2-fluoro-4-(trifluoro- methyl)phenyl)-1H-pyrazolo[4,3- b]pyridin-1-yl)azetidin-1-yl)prop- 2-en-1-one | 391.1 |
| 130 | 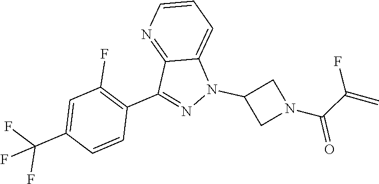 [SMILES: C=C(F)C(=O)N1CC(n2nc(-c3ccc(C(F)(F)F)cc3F)c3ncccc32)C1] | 2-fluoro-1-(3-(3-(2-fluoro-4- (trifluoromethyl)phenyl)-1H- pyrazolo[4,3-b]pyridin-1-yl)- azetidin-1-yl)prop-2-en-1-one | 409.1 |
| 131 | 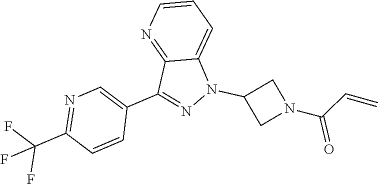 [SMILES: C=CC(=O)N1CC(n2nc(-c3ccc(C(F)(F)F)nc3)c3ncccc32)C1] | 1-(3-(3-(6-(trifluoromethyl)pyr- idin-3-yl)-1H-pyrazolo[4,3-b]- pyridin-1-yl)azetidin-1-yl)prop- 2-en-1-one | 374.1 |
| 132 | 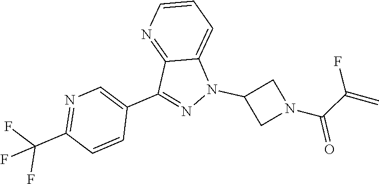 [SMILES: C=C(F)C(=O)N1CC(n2nc(-c3ccc(C(F)(F)F)nc3)c3ncccc32)C1] | 2-fluoro-1-(3-(3-(6-(trifluoro- methyl)pyridin-3-yl)-1H- pyrazolo[4,3-b]pyridin-1-yl)- azetidin-1-yl)prop-2-en-1-one | 392.1 |
| 133 | 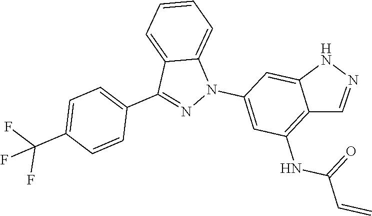 [SMILES: C=CC(=O)Nc1cc(-n2nc(-c3ccc(C(F)(F)F)cc3)c3ccccc32)cc2[nH]ncc12] | N-(3-(4-(trifluoromethyl)phenyl)-1′H-[1,6′-biindazol]-4′-yl)- acrylamide | 448.1 |
| 134 | 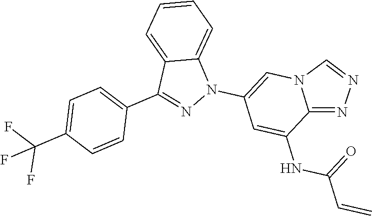 [SMILES: C=CC(=O)Nc1cc(-n2nc(-c3ccc(C(F)(F)F)cc3)c3ccccc32)cn2cnnc12] | N-(6-(3-(4-(trifluoromethyl)phen- yl)-1H-indazol-1-yl)-[1,2,4]- triazolo[4,3-a]pyridin-8-yl)- acrylamide | 449.1 |
| 135 | 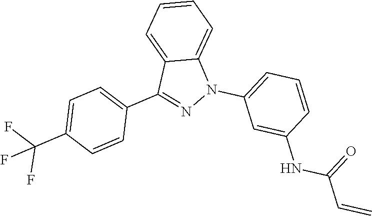 [SMILES: C=CC(=O)Nc1cccc(-n2nc(-c3ccc(C(F)(F)F)cc3)c3ccccc32)c1] | N-(3-(3-(4-(trifluoromethyl)phen- yl)-1H-indazol-1-yl)phenyl)- acrylamide | 408.1 |
| 136 | 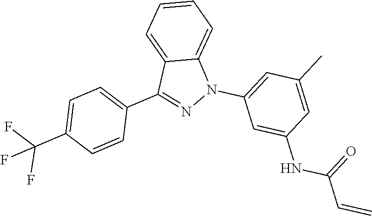 [SMILES: C=CC(=O)Nc1cc(C)cc(-n2nc(-c3ccc(C(F)(F)F)cc3)c3ccccc32)c1] | N-(3-methyl-5-(3-(4-(trifluoro- methyl)phenyl)-1H-indazol-1- yl)phenyl)acrylamide | 422.1 |
| 137 | 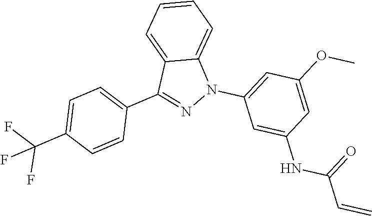 [SMILES: C=CC(=O)Nc1cc(OC)cc(-n2nc(-c3ccc(C(F)(F)F)cc3)c3ccccc32)c1] | N-(3-methoxy-5-(3-(4-(trifluoro- methyl)phenyl)-1H-indazol-1-yl)- phenyl)acrylamide | 438.1 |
| 138 | 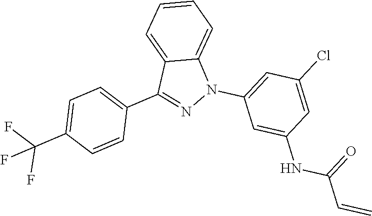 [SMILES: C=CC(=O)Nc1cc(Cl)cc(-n2nc(-c3ccc(C(F)(F)F)cc3)c3ccccc32)c1] | N-(3-chloro-5-(3-(4-(trifluoro- methyl)phenyl)-1H-indazol-1- yl)phenyl)acrylamide | 442.1 |
| 139 | 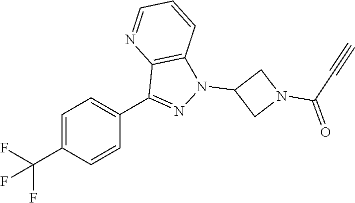 [SMILES: C#CC(=O)N1CC(n2nc(-c3ccc(C(F)(F)F)cc3)c3ncccc32)C1] | 1-(3-(3-(4-(trifluoromethyl)phen- yl)-1H-pyrazolo[4,3-b]pyridin-1- yl)azetidin-1-yl)prop-2-yn-1-one | 371.1 |
| 140 | 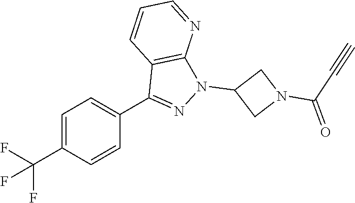 [SMILES: C#CC(=O)N1CC(n2nc(-c3ccc(C(F)(F)F)cc3)c3cccnc32)C1] | 1-(3-(3-(4-(trifluoromethyl)phen- yl)-1H-pyrazolo[3,4-b]pyridin-1- yl)azetidin-1-yl)prop-2-yn-1-one | 371.1 |
| 141 | 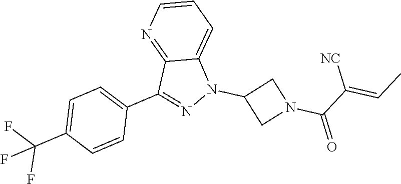 [SMILES: C/C=C(\C#N)C(=O)N1CC(n2nc(-c3ccc(C(F)(F)F)cc3)c3ncccc32)C1] | (E)-2-(3-(3-(4-(trifluoromethyl)- phenyl)-1H-pyrazolo[4,3-b]- pyridin-1-yl)azetidine-1-carbon- yl)-but-2-enenitrile | 412.1 |
| 142 | 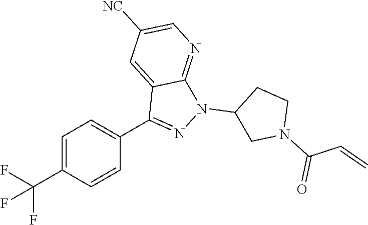 [SMILES: C=CC(=O)N1CCC(n2nc(-c3ccc(C(F)(F)F)cc3)c3cc(C#N)cnc32)C1] | 1-(1-acryloylpyrrolidin-3-yl)-3- (4-(trifluoromethyl)phenyl)-1H- pyrazolo[3,4-b]pyridine-5- carbonitrile | 412.1 |
| 143 | 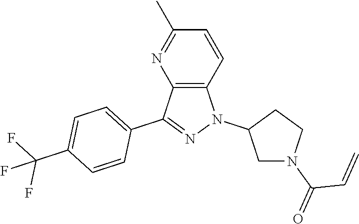 [SMILES: C=CC(=O)N1CCC(n2nc(-c3ccc(C(F)(F)F)cc3)c3nc(C)ccc32)C1] | 1-(3-(5-methyl-3-(4-(trifluoro- methyl)phenyl)-1H-pyrazolo[4,3- b]pyridin-1-yl)pyrrolidin-1-yl)- prop-2-en-1-one | 401.2 |
| 144 | 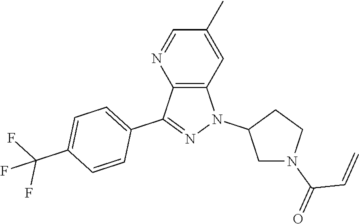 [SMILES: C=CC(=O)N1CCC(n2nc(-c3ccc(C(F)(F)F)cc3)c3ncc(C)cc32)C1] | 1-(3-(6-methyl-3-(4-(trifluoro- methyl)phenyl)-1H-pyrazolo[4,3- b]pyridin-1-yl)pyrrolidin-1-yl)- prop-2-en-1-one | 401.2 |
| 145 | 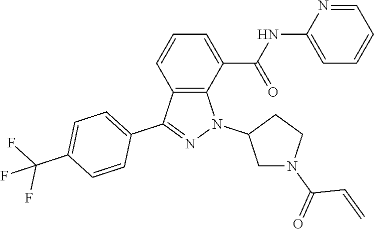 [SMILES: C=CC(=O)N1CCC(n2nc(-c3ccc(C(F)(F)F)cc3)c3cccc(C(=O)Nc4ccccn4)c32)C1] | 1-(1-acryloylpyrrolidin-3-yl)-N- (pyridin-2-yl)-3-(4-(trifluoro- methyl)phenyl)-1H-indazole-7- carboxamide | 506.2 |
| 146 | 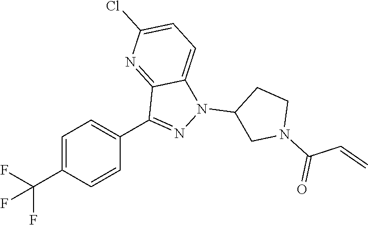 [SMILES: C=CC(=O)N1CCC(n2nc(-c3ccc(C(F)(F)F)cc3)c3nc(Cl)ccc32)C1] | 1-(3-(5-chloro-3-(4-(trifluoro- methyl)phenyl)-1H-pyrazolo[4,3- b]pyridin-1-yl)pyrrolidin-1-yl)- prop-2-en-1-one | 421.1 |
| 147 | 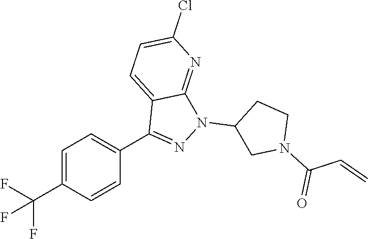 [SMILES: C=CC(=O)N1CCC(n2nc(-c3ccc(C(F)(F)F)cc3)c3ccc(Cl)nc32)C1] | 1-(3-(6-chloro-3-(4-(trifluoro- methyl)phenyl)-1H-pyrazolo[3,4- b]pyridin-1-yl)pyrrolidin-1-yl)- prop-2-en-1-one | 421.1 |
| 148 | 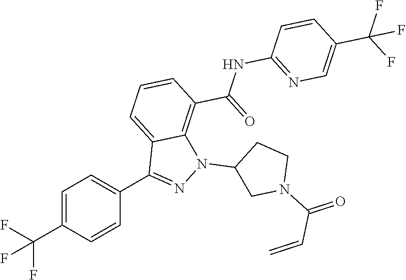 [SMILES: C=CC(=O)N1CCC(n2nc(-c3ccc(C(F)(F)F)cc3)c3cccc(C(=O)Nc4ccc(C(F)(F)F)cn4)c32)C1] | 1-(1-acryloylpyrrolidin-3-yl)-3- (4-(trifluoromethyl)phenyl)-N-(5- (trifluoromethyl)pyridin-2-yl)-1H- indazole-7-carboxamide | 574.2 |
| 149 | 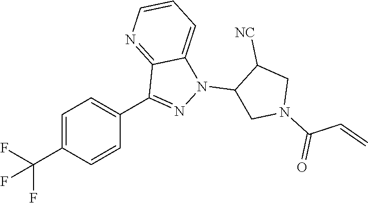 [SMILES: C=CC(=O)N1CC(C#N)C(n2nc(-c3ccc(C(F)(F)F)cc3)c3ncccc32)C1] | 1-acryloyl-4-(3-(4-(trifluoro- methyl)phenyl)-1H-pyrazolo[4,3- b]pyridin-1-yl)pyrrolidine-3- carbonitrile | 412.1 |
| 150 | 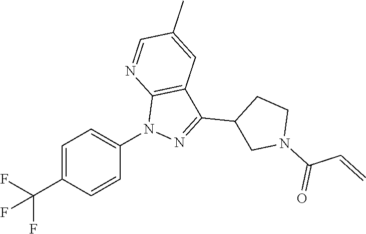 [SMILES: C=CC(=O)N1CCC(c2nn(-c3ccc(C(F)(F)F)cc3)c3ncc(C)cc23)C1] | 1-(3-(5-methyl-1-(4-(trifluoro- methyl)phenyl)-1H-pyrazolo[3,4- b]pyridin-3-yl)pyrrolidin-1-yl)- prop-2-en-1-one | 401.2 |
| 151 | 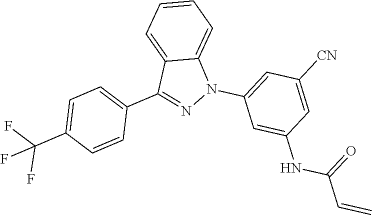 [SMILES: C=CC(=O)Nc1cc(C#N)cc(-n2nc(-c3ccc(C(F)(F)F)cc3)c3ccccc32)c1] | N-(3-cyano-5-(3-(4-(trifluoro- methyl)phenyl)-1H-indazol-1-yl)- phenyl)acrylamide | 433.1 |
| 152 | 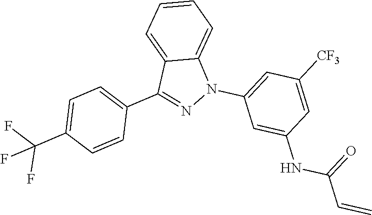 [SMILES: C=CC(=O)Nc1cc(-n2nc(-c3ccc(C(F)(F)F)cc3)c3ccccc32)cc(C(F)(F)F)c1] | N-(3-(trifluoromethyl)-5-(3-(4- (trifluoromethyl)phenyl)-1H- indazol-1-yl)phenyl)acrylamide | 476.1 |
| 153 | 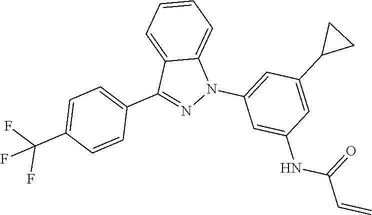 [SMILES: C=CC(=O)Nc1cc(C2CC2)cc(-n2nc(-c3ccc(C(F)(F)F)cc3)c3ccccc32)c1] | N-(3-cyclopropyl-5-(3-(4- (trifluoromethyl)phenyl)-1H- indazol-1-yl)phenyl)acrylamide | 448.2 |
| 154 | 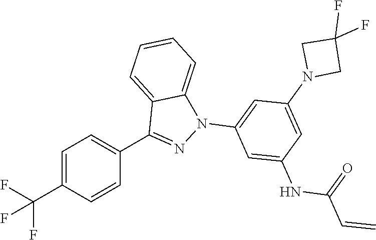 [SMILES: C=CC(=O)Nc1cc(N2CC(F)(F)C2)cc(-n2nc(-c3ccc(C(F)(F)F)cc3)c3ccccc32)c1] | N-(3-(3,3-difluoroazetidin-1-yl)- 5-(3-(4-(trifluoromethyl)phen- yl)-1H-indazol-1-yl)phenyl)- acrylamide | 499.2 |
| 155 | 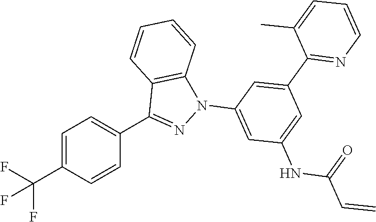 [SMILES: C=CC(=O)Nc1cc(-c2ncccc2C)cc(-n2nc(-c3ccc(C(F)(F)F)cc3)c3ccccc32)c1] | N-(3-(3-methylpyridin-2-yl)-5-(3- (4-(trifluoromethyl)phenyl)-1H- indazol-1-yl)phenyl)acrylamide | 499.2 |
| 156 | 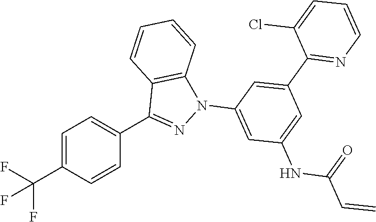 [SMILES: C=CC(=O)Nc1cc(-c2ncccc2Cl)cc(-n2nc(-c3ccc(C(F)(F)F)cc3)c3ccccc32)c1] | N-(3-(3-chloropyridin-2-yl)-5-(3- (4-(trifluoromethyl)phenyl)-1H- indazol-1-yl)phenyl)acrylamide | 519.1 |
| 157 | 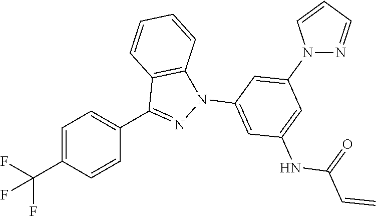 [SMILES: C=CC(=O)Nc1cc(-n2cccn2)cc(-n2nc(-c3ccc(C(F)(F)F)cc3)c3ccccc32)c1] | N-(3-(1H-pyrazol-1-yl)-5-(3-(4- (trifluoromethyl)phenyl)-1H- indazol-1-yl)phenyl)acrylamide | 474.2 |
| 158 | 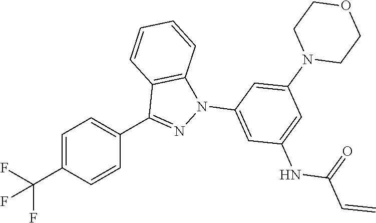 [SMILES: C=CC(=O)Nc1cc(N2CCOCC2)cc(-n2nc(-c3ccc(C(F)(F)F)cc3)c3ccccc32)c1] | N-(3-morpholino-5-(3-(4- (trifluoromethyl)phenyl)-1H- indazol-1-yl)phenyl)acrylamide | 493.2 |
| 159 | 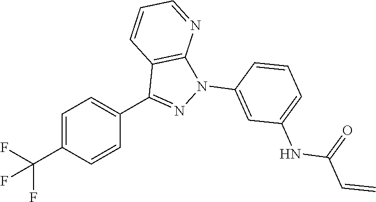 [SMILES: C=CC(=O)Nc1cccc(-n2nc(-c3ccc(C(F)(F)F)cc3)c3cccnc32)c1] | N-(3-(3-(4-(trifluoromethyl)phen- yl)-1H-pyrazolo[3,4-b]pyridin-1- yl)phenyl)acrylamide | 409.1 |
| 160 | 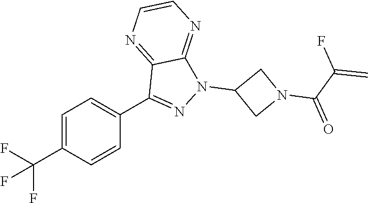 [SMILES: C=C(F)C(=O)N1CC(n2nc(-c3ccc(C(F)(F)F)cc3)c3nccnc32)C1] | 2-fluoro-1-(3-(3-(4-(trifluoro- methyl)phenyl)-1H-pyrazolo[3,4- b]pyrazin-1-yl)azetidin-1-yl)prop- 2-en-1-one | 392.1 |
| 161 | 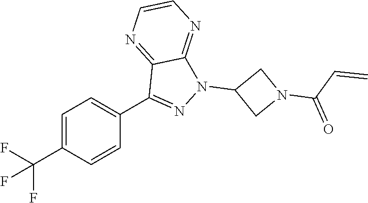 [SMILES: C=CC(=O)N1CC(n2nc(-c3ccc(C(F)(F)F)cc3)c3nccnc32)C1] | 1-(3-(3-(4-(trifluoromethyl)phen- yl)-1H-pyrazolo[3,4-b]pyrazin-1- yl)azetidin-1-yl)prop-2-en-1-one | 374.1 |
| 162 | 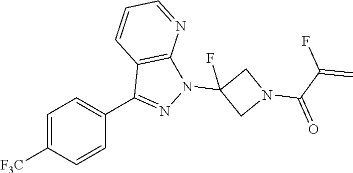 [SMILES: C=C(F)C(=O)N1CC(F)(n2nc(-c3ccc(C(F)(F)F)cc3)c3cccnc32)C1] | 2-fluoro-1-(3-fluoro-3-(3-(4- (trifluoromethyl)phenyl)-1H- pyrazolo[3,4-b]pyridin-1-yl)- azetidin-1-yl)prop-2-en-1-one | 409.1 |
| 163 | 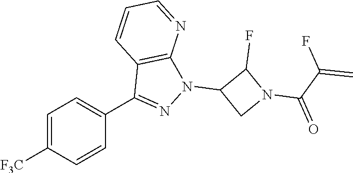 [SMILES: C=C(F)C(=O)N1CC(n2nc(-c3ccc(C(F)(F)F)cc3)c3cccnc32)C1F] | 2-fluoro-1-(2-fluoro-3-(3-(4- (trifluoromethyl)phenyl)-1H- pyrazolo[3,4-b]pyridin-1-yl)- azetidin-1-yl)prop-2-en-1-one | 409.1 |
| 164 | 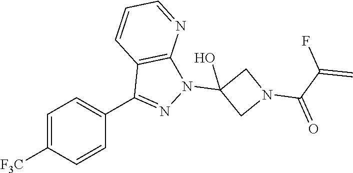 [SMILES: C=C(F)C(=O)N1CC(O)(n2nc(-c3ccc(C(F)(F)F)cc3)c3cccnc32)C1] | 2-fluoro-1-(3-hydroxy-3-(3-(4- (trifluoromethyl)phenyl)-1H- pyrazolo[3,4-b]pyridin-1-yl)- azetidin-1-yl)prop-2-en-1-one | 407.1 |
| 165 | 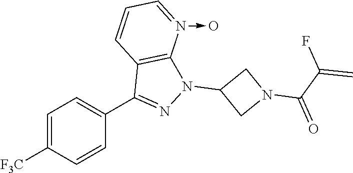 [SMILES: C=C(F)C(=O)N1CC(n2nc(-c3ccc(C(F)(F)F)cc3)c3ccc[n+]([O-])c32)C1] | 1-(1-(2-fluoroacryloyl)azetidin-3- yl)-3-(4-(trifluoromethyl)phenyl)- 1H-pyrazolo[3,4-b]pyridine 7- oxide | 407.1 |
| 166 | 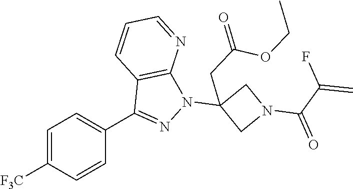 [SMILES: C=C(F)C(=O)N1CC(CC(=O)OCC)(n2nc(-c3ccc(C(F)(F)F)cc3)c3cccnc32)C1] | ethyl 2-(1-(2-fluoroacryloyl)-3-(3-(4-(trifluoromethyl)phenyl)-1H- pyrazolo[3,4-b]pyridin-1-yl)- azetidin-3-yl)acetate | 407.1 |
| 167 | 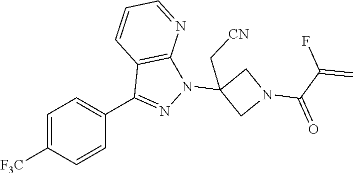 [SMILES: C=C(F)C(=O)N1CC(CC#N)(n2nc(-c3ccc(C(F)(F)F)cc3)c3cccnc32)C1] | 2-(1-(2-fluoroacryloyl)-3-(3-(4- (trifluoromethyl)phenyl)-1H- pyrazolo[3,4-b]pyridin-1-yl)- azetidin-3-yl)acetonitrile | 430.1 |
| 168 | 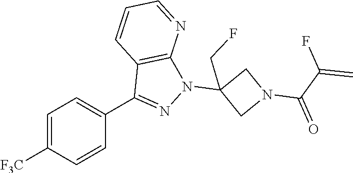 [SMILES: C=C(F)C(=O)N1CC(CF)(n2nc(-c3ccc(C(F)(F)F)cc3)c3cccnc32)C1] | 2-fluoro-1-(3-(fluoromethyl)-3-(3- (4-(trifluoromethyl)phenyl)-1H- pyrazolo[3,4-b]pyridin-1-yl)- azetidin-1-yl)prop-2-en-1-one | 423.1 |
| 169 | 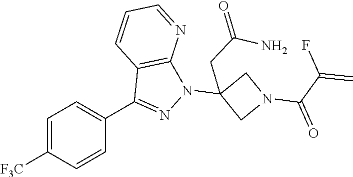 [SMILES: C=C(F)C(=O)N1CC(CC(N)=O)(n2nc(-c3ccc(C(F)(F)F)cc3)c3cccnc32)C1] | 2-(1-(2-fluoroacryloyl)-3-(3-(4- (trifluoromethyl)phenyl)-1H- pyrazolo[3,4-b]pyridin-1-yl)- azetidin-3-yl)acetamide | 448.1 |
| 170 | 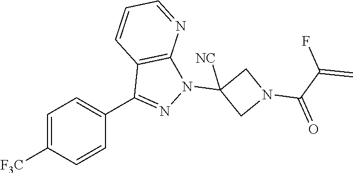 [SMILES: C=C(F)C(=O)N1CC(C#N)(n2nc(-c3ccc(C(F)(F)F)cc3)c3cccnc32)C1] | 1-(2-fluoroacryloyl)-3-(3-(4- (trifluoromethyl)phenyl)-1H- pyrazolo[3,4-b]pyridin-1-yl)- azetidine-3-carbonitrile | 416.1 |
| 171 | 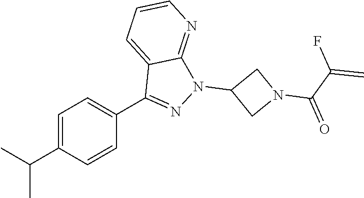 [SMILES: C=C(F)C(=O)N1CC(n2nc(-c3ccc(C(C)C)cc3)c3cccnc32)C1] | 2-fluoro-1-(3-(3-(4-isopropyl- phenyl)-1H-pyrazolo[3,4-b]- pyridin-1-yl)azetidin-1-yl)prop- 2-en-1-one | 365.2 |
| 172 | 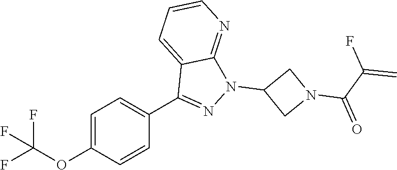 [SMILES: C=C(F)C(=O)N1CC(n2nc(-c3ccc(OC(F)(F)F)cc3)c3cccnc32)C1] | 2-fluoro-1-(3-(3-(4-(trifluoro- methoxy)phenyl)-1H-pyrazolo- [3,4-b]pyridin-1-yl)azetidin-1- yl)prop-2-en-1-one | 407.1 |
| 173 | 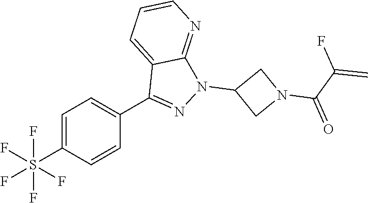 [SMILES: C=C(F)C(=O)N1CC(n2nc(-c3ccc(S(F)(F)(F)(F)F)cc3)c3cccnc32)C1] | 2-fluoro-1-(3-(3-(4-(pentafluoro- 16-sulfanyl)phenyl)-1H-pyrazolo- [3,4-b]pyridin-1-yl)azetidin-1- yl)prop-2-en-1-one | 449.1 |
| 174 | 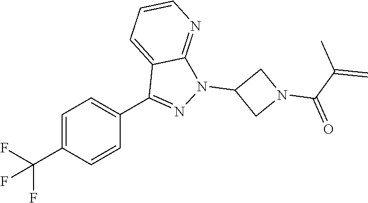 [SMILES: C=C(C)C(=O)N1CC(n2nc(-c3ccc(C(F)(F)F)cc3)c3cccnc32)C1] | 2-methyl-1-(3-(3-(4-(trifluoro- methyl)phenyl)-1H-pyrazolo[3,4- b]pyridin-1-yl)azetidin-1-yl)prop- 2-en-1-one | 387.1 |
| 175 | 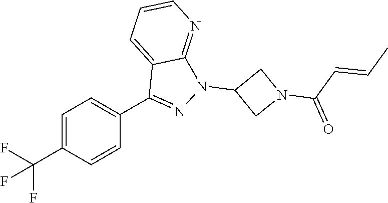 [SMILES: C/C=C/C(=O)N1CC(n2nc(-c3ccc(C(F)(F)F)cc3)c3cccnc32)C1] | (E)-1-(3-(3-(4-(trifluoromethyl)- phenyl)-1H-pyrazolo[3,4-b]pyr- idin-1-yl)azetidin-1-yl)but-2-en- 1-one | 387.1 |
| 176 | 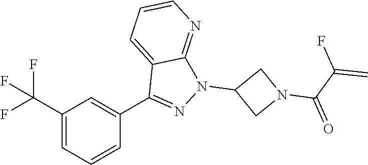 [SMILES: C=C(F)C(=O)N1CC(n2nc(-c3cccc(C(F)(F)F)c3)c3cccnc32)C1] | 2-fluoro-1-(3-(3-(3-(trifluoro- methyl)phenyl)-1H-pyrazolo[3,4- b]pyridin-1-yl)azetidin-1-yl)-prop- 2-en-1-one | 391.1 |
| 177 | 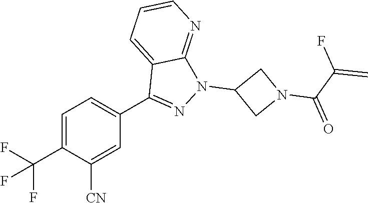 [SMILES: C=C(F)C(=O)N1CC(n2nc(-c3ccc(C(F)(F)F)c(C#N)c3)c3cccnc32)C1] | 5-(1-(1-(2-fluoroacryloyl)azetidin- 3-yl)-1H-pyrazolo[3,4-b]pyridin- 3-yl)-2-(trifluoromethyl)benzo- nitrile | 416.1 |
| 178 | 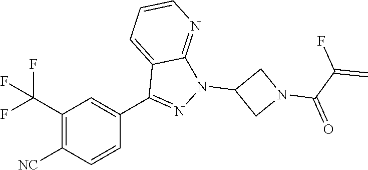 [SMILES: C=C(F)C(=O)N1CC(n2nc(-c3ccc(C#N)c(C(F)(F)F)c3)c3cccnc32)C1] | 4-(1-(1-(2-fluoroacryloyl)azetidin- 3-yl)-1H-pyrazolo[3,4-b]pyridin- 3-yl)-2-(trifluoromethyl)benzo- nitrile | 416.1 |
| 179 | 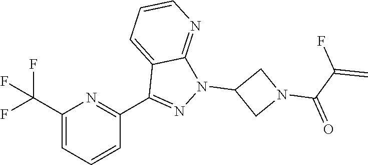 [SMILES: C=C(F)C(=O)N1CC(n2nc(-c3cccc(C(F)(F)F)n3)c3cccnc32)C1] | 2-fluoro-1-(3-(3-(6-(trifluorometh- yl)pyridin-2-yl)-1H-pyrazolo[3,4- b]pyridin-1-yl)azetidin-1-yl)prop- 2-en-1-one | 392.1 |
| 180 | 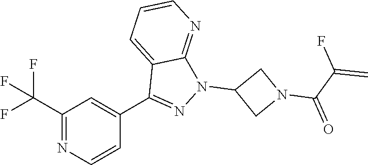 [SMILES: C=C(F)C(=O)N1CC(n2nc(-c3ccnc(C(F)(F)F)c3)c3cccnc32)C1] | 2-fluoro-1-(3-(3-(2-(trifluorometh- yl)pyridin-4-yl)-1H-pyrazolo[3,4- b]pyridin-1-yl)azetidin-1-yl)prop- 2-en-1-one | 392.1 |
| 181 | 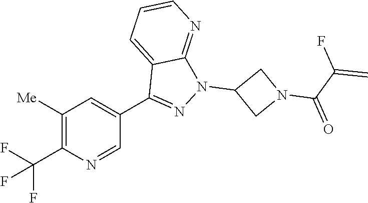 [SMILES: C=C(F)C(=O)N1CC(n2nc(-c3cnc(C(F)(F)F)c(C)c3)c3cccnc32)C1] | 2-fluoro-1-(3-(3-(5-methyl-6- (trifluoromethyl)pyridin-3-yl)- 1H-pyrazolo[3,4-b]pyridin-1-yl)- azetidin-1-yl)prop-2-en-1-one | 406.1 |
| 182 | 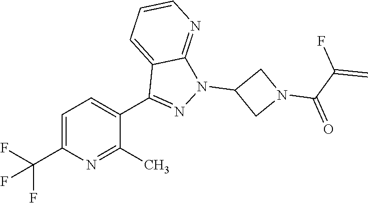 [SMILES: C=C(F)C(=O)N1CC(n2nc(-c3ccc(C(F)(F)F)nc3C)c3cccnc32)C1] | 2-fluoro-1-(3-(3-(2-methyl-6- (trifluoromethyl)pyridin-3-yl)- 1H-pyrazolo[3,4-b]pyridin-1- yl)azetidin-1-yl)prop-2-en-1- one | 406.1 |
| 183 | 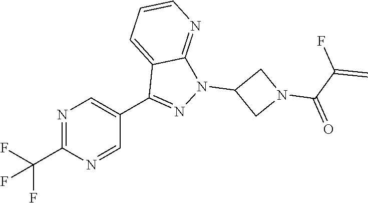 [SMILES: C=C(F)C(=O)N1CC(n2nc(-c3cnc(C(F)(F)F)nc3)c3cccnc32)C1] | 2-fluoro-1-(3-(3-(2-(trifluorometh- yl)pyrimidin-5-yl)-1H-pyrazolo- [3,4-b]pyridin-1-yl)azetidin-1-yl)- prop-2-en-1-one | 393.1 |
| 184 | 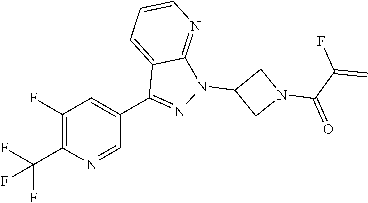 [SMILES: C=C(F)C(=O)N1CC(n2nc(-c3cnc(C(F)(F)F)c(F)c3)c3cccnc32)C1] | 2-fluoro-1-(3-(3-(5-fluoro-6- (trifluoromethyl)pyridin-3-yl)- 1H-pyrazolo[3,4-b]pyridin-1-yl)- azetidin-1-yl)prop-2-en-1-one | 410.1 |
| 185 | 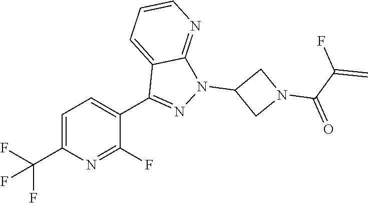 [SMILES: C=C(F)C(=O)N1CC(n2nc(-c3ccc(C(F)(F)F)nc3F)c3cccnc32)C1] | 2-fluoro-1-(3-(3-(2-fluoro-6- (trifluoromethyl)pyridin-3-yl)- 1H-pyrazolo[3,4-b]pyridin-1- yl)azetidin-1-yl)prop-2-en-1-one | 410.1 |
| 186 | 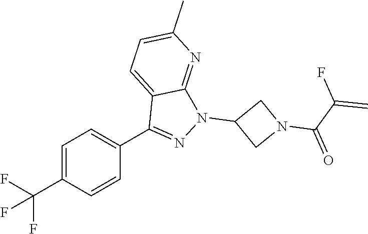 [SMILES: C=C(F)C(=O)N1CC(n2nc(-c3ccc(C(F)(F)F)cc3)c3ccc(C)nc32)C1] | 2-fluoro-1-(3-(6-methyl-3-(4- (trifluoromethyl)phenyl)-1H- pyrazolo[3,4-b]pyridin-1-yl)- azetidin-1-yl)prop-2-en-1-one | 405.1 |
| 187 | 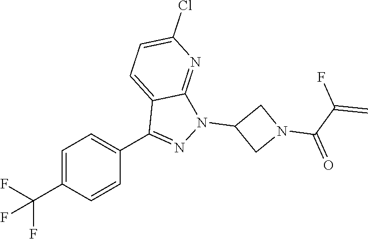 [SMILES: C=C(F)C(=O)N1CC(n2nc(-c3ccc(C(F)(F)F)cc3)c3ccc(Cl)nc32)C1] | 1-(3-(6-chloro-3-(4-(trifluoro- methyl)phenyl)-1H-pyrazolo[3,4- b]pyridin-1-yl)azetidin-1-yl)-2- fluoroprop-2-en-1-one | 425.1 |
| 188 | 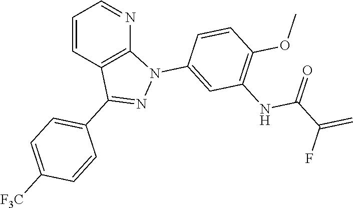 [SMILES: C=C(F)C(=O)Nc1cc(-n2nc(-c3ccc(C(F)(F)F)cc3)c3cccnc32)ccc1OC] | 2-fluoro-N-(2-methoxy-5-(3-(4- (trifluoromethyl)phenyl)-1H- pyrazolo[3,4-b]pyridin-1-yl)- phenyl)acrylamide | 457.1 |
| 189 | 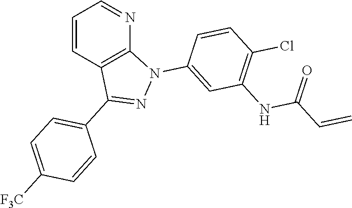 [SMILES: C=CC(=O)Nc1cc(-n2nc(-c3ccc(C(F)(F)F)cc3)c3cccnc32)ccc1Cl] | N-(2-chloro-5-(3-(4-(trifluoro- methyl)phenyl)-1H-pyrazolo[3,4- b]pyridin-1-yl)phenyl)acrylamide | 443.1 |
| 190 | 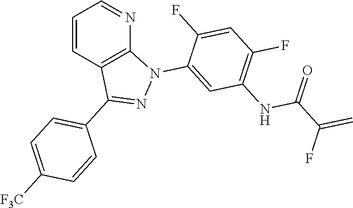 [SMILES: C=C(F)C(=O)Nc1cc(-n2nc(-c3ccc(C(F)(F)F)cc3)c3cccnc32)c(F)cc1F] | N-(2,4-difluoro-5-(3-(4-(trifluoro- methyl)phenyl)-1H-pyrazolo[3,4- b]pyridin-1-yl)phenyl)-2-fluoro- acrylamide | 463.1 |
| 191 | 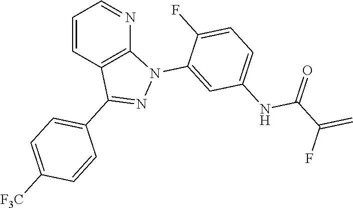 [SMILES: C=C(F)C(=O)Nc1ccc(F)c(-n2nc(-c3ccc(C(F)(F)F)cc3)c3cccnc32)c1] | 2-fluoro-N-(4-fluoro-3-(3-(4- (trifluoromethyl)phenyl)-1H- pyrazolo[3,4-b]pyridin-1-yl)- phenyl)acrylamide | 445.1 |
| 192 | 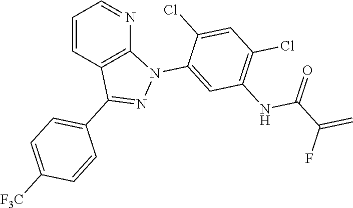 [SMILES: C=C(F)C(=O)Nc1cc(-n2nc(-c3ccc(C(F)(F)F)cc3)c3cccnc32)c(Cl)cc1Cl] | N-(2,4-dichloro-5-(3-(4-(trifluoro- methyl)phenyl)-1H-pyrazolo[3,4- b]pyridin-1-yl)phenyl)-2-fluoro- acrylamide | 495.0 |
| 193 | 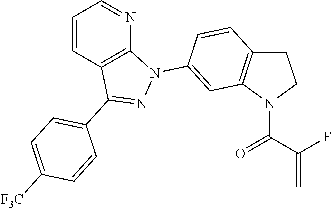 [SMILES: C=C(F)C(=O)N1CCc2ccc(-n3nc(-c4ccc(C(F)(F)F)cc4)c4cccnc43)cc21] | 2-fluoro-1-(6-(3-(4-(trifluoro- methyl)phenyl)-1H-pyrazolo[3,4- b]pyridin-1-yl)indolin-1-yl)prop- 2-en-1-one | 453.1 |
| 194 | 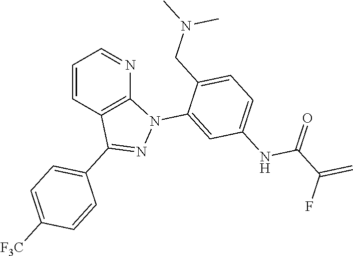 [SMILES: C=C(F)C(=O)Nc1ccc(CN(C)C)c(-n2nc(-c3ccc(C(F)(F)F)cc3)c3cccnc32)c1] | N-(4-((dimethylamino)methyl)-3- (3-(4-(trifluoromethyl)phenyl)- 1H-pyrazolo[3,4-b]pyridin-1-yl)- phenyl)-2-fluoroacrylamide | 484.2 |
| 195 | 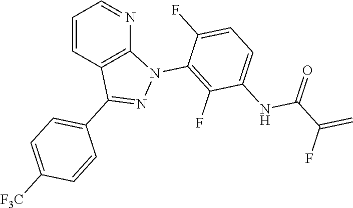 [SMILES: C=C(F)C(=O)Nc1ccc(F)c(-n2nc(-c3ccc(C(F)(F)F)cc3)c3cccnc32)c1F] | N-(2,4-difluoro-3-(3-(4-(trifluoro- methyl)phenyl)-1H-pyrazolo[3,4- b]pyridin-1-yl)phenyl)-2-fluoro- acrylamide | 463.1 |
| 196 | 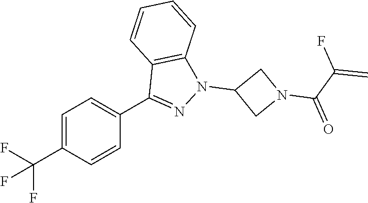 [SMILES: C=C(F)C(=O)N1CC(n2nc(-c3ccc(C(F)(F)F)cc3)c3ccccc32)C1] | 2-fluoro-1-(3-(3-(4-(trifluoro- methyl)phenyl)-1H-indazol-1- yl)azetidin-1-yl)prop-2-en-1-one | 390.1 |
| 197 | 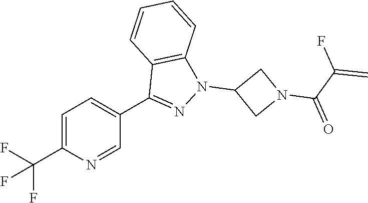 [SMILES: C=C(F)C(=O)N1CC(n2nc(-c3ccc(C(F)(F)F)nc3)c3ccccc32)C1] | 2-fluoro-1-(3-(3-(6-(trifluoro- methyl)pyridin-3-yl)-1H-indazol- 1-yl)azetidin-1-yl)prop-2-en-1- one | 391.1 |
| 198 | 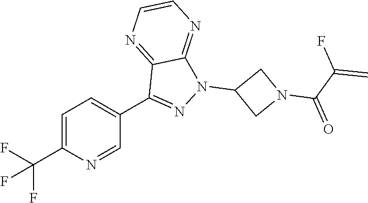 [SMILES: C=C(F)C(=O)N1CC(n2nc(-c3ccc(C(F)(F)F)nc3)c3nccnc32)C1] | 2-fluoro-1-(3-(3-(6-(trifluoro- methyl)pyridin-3-yl)-1H- pyrazolo[3,4-b]pyrazin-1-yl)- azetidin-1-yl)prop-2-en-1-one | 393.1 |
| 199 | 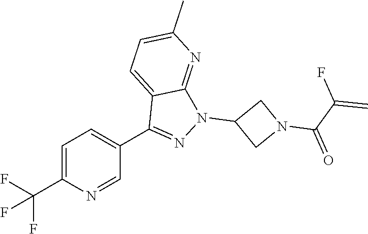 [SMILES: C=C(F)C(=O)N1CC(n2nc(-c3ccc(C(F)(F)F)nc3)c3ccc(C)nc32)C1] | 2-fluoro-1-(3-(6-methyl-3-(6- (trifluoromethyl)pyridin-3-yl)- 1H-pyrazolo[3,4-b]pyridin-1-yl)- azetidin-1-yl)prop-2-en-1-one | 406.1 |
| 200 | 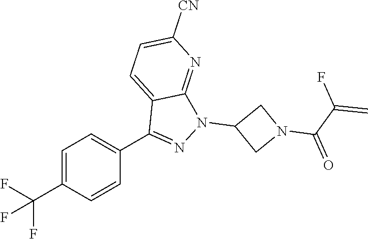 [SMILES: C=C(F)C(=O)N1CC(n2nc(-c3ccc(C(F)(F)F)cc3)c3ccc(C#N)nc32)C1] | 1-(1-(2-fluoroacryloyl)azetidin-3- yl)-3-(4-(trifluoromethyl)phenyl)- 1H-pyrazolo[3,4-b]pyridine-6- carbonitrile | 416.1 |
| 201 | 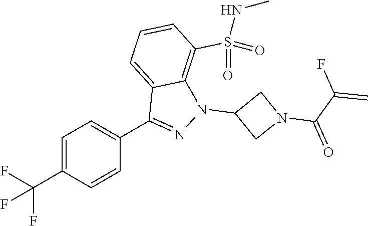 [SMILES: C=C(F)C(=O)N1CC(n2nc(-c3ccc(C(F)(F)F)cc3)c3cccc(S(=O)(=O)NC)c32)C1] | 1-(1-(2-fluoroacryloyl)azetidin-3- yl)-N-methyl-3-(4-(trifluorometh- yl)phenyl)-1H-indazole-7-sulfonamide | 483.1 |
| 202 | 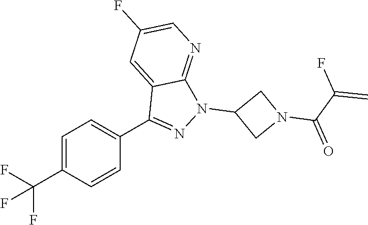 [SMILES: C=C(F)C(=O)N1CC(n2nc(-c3ccc(C(F)(F)F)cc3)c3cc(F)cnc32)C1] | 2-fluoro-1-(3-(5-fluoro-3-(4- (trifluoromethyl)phenyl)-1H- pyrazolo[3,4-b]pyridin-1-yl)- azetidin-1-yl)prop-2-en-1-one | 409.1 |
| 203 | 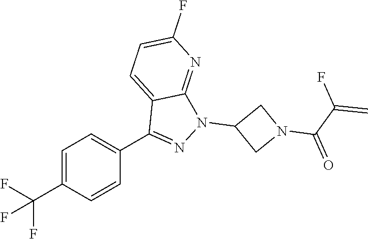 [SMILES: C=C(F)C(=O)N1CC(n2nc(-c3ccc(C(F)(F)F)cc3)c3ccc(F)nc32)C1] | 2-fluoro-1-(3-(6-fluoro-3-(4- (trifluoromethyl)phenyl)-1H- pyrazolo[3,4-b]pyridin-1-yl)- azetidin-1-yl)prop-2-en-1-one | 409.1 |
| 204 | 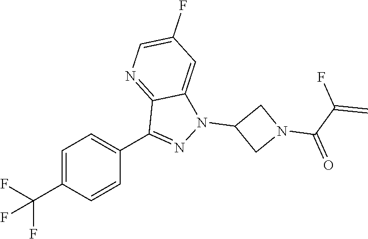 [SMILES: C=C(F)C(=O)N1CC(n2nc(-c3ccc(C(F)(F)F)cc3)c3ncc(F)cc32)C1] | 2-fluoro-1-(3-(6-fluoro-3-(4- (trifluoromethyl)phenyl)-1H- pyrazolo[4,3-b]pyridin-1-yl)- azetidin-1-yl)prop-2-en-1-one | 409.1 |
| 205 | 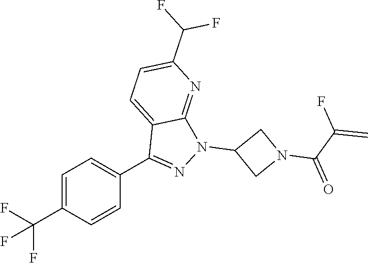 [SMILES: C=C(F)C(=O)N1CC(n2nc(-c3ccc(C(F)(F)F)cc3)c3ccc(C(F)F)nc32)C1] | 1-(3-(6-(difluoromethyl)-3-(4- (trifluoromethyl)phenyl)-1H- pyrazolo[3,4-b]pyridin-1-yl)- azetidin-1-yl)-2-fluoroprop-2- en-1-one | 441.1 |
| 206 | 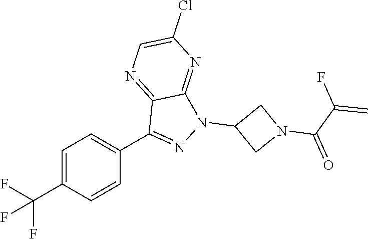 [SMILES: C=C(F)C(=O)N1CC(n2nc(-c3ccc(C(F)(F)F)cc3)c3ncc(Cl)nc32)C1] | 1-(3-(6-chloro-3-(4-(trifluoro- methyl)phenyl)-1H-pyrazolo[3,4- b]pyrazin-1-yl)azetidin-1-yl)-2- fluoroprop-2-en-1-one | 426.1 |
| 207 | 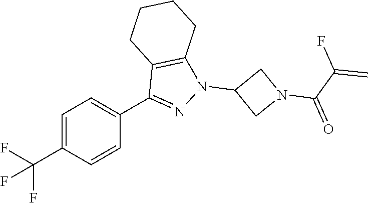 [SMILES: C=C(F)C(=O)N1CC(n2nc(-c3ccc(C(F)(F)F)cc3)c3c2CCCC3)C1] | 2-fluoro-1-(3-(3-(4-(trifluoro- methyl)phenyl)-4,5,6,7-tetra- hydro-1H-indazol-1-yl)azetidin- 1-yl)prop-2-en-1-one | 394.2 |
| 208 | 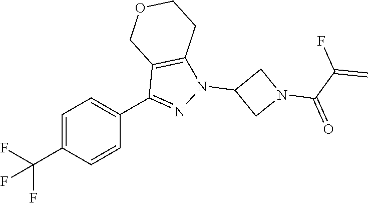 [SMILES: C=C(F)C(=O)N1CC(n2nc(-c3ccc(C(F)(F)F)cc3)c3c2CCOC3)C1] | 2-fluoro-1-(3-(3-(4-(trifluoro- methyl)phenyl)-6,7-dihydro- pyrano-[4,3-c]pyrazol-1(4H)-yl)- azetidin-1-yl)prop-2-en-1-one | 396.1 |
| 209 | 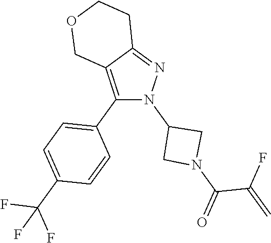 [SMILES: C=C(F)C(=O)N1CC(n2nc3c(c2-c2ccc(C(F)(F)F)cc2)COCC3)C1] | 2-fluoro-1-(3-(3-(4-(trifluoro- methyl)phenyl)-6,7-dihydro- pyrano[4,3-c]pyrazol-2(4H)- yl)azetidin-1-yl)prop-2-en-1-one | 396.1 |
| 210 | 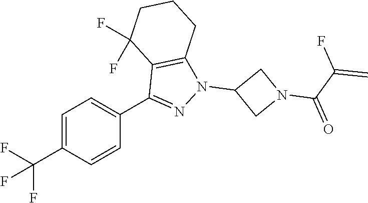 [SMILES: C=C(F)C(=O)N1CC(n2nc(-c3ccc(C(F)(F)F)cc3)c3c2CCCC3(F)F)C1] | 1-(3-(4,4-difluoro-3-(4-(trifluoro- methyl)phenyl)-4,5,6,7-tetra- hydro-1H-indazol-1-yl)azetidin-1- yl)-2-fluoroprop-2-en-1-one | 430.1 |
| 211 | 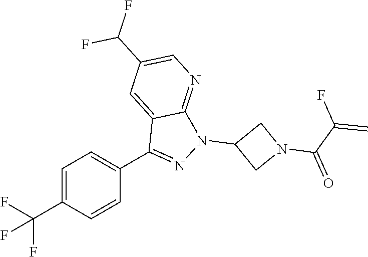 [SMILES: C=C(F)C(=O)N1CC(n2nc(-c3ccc(C(F)(F)F)cc3)c3cc(C(F)F)cnc32)C1] | 1-(3-(5-(difluoromethyl)-3-(4- (trifluoromethyl)phenyl)-1H- pyrazolo[3,4-b]pyridin-1-yl)- azetidin-1-yl)-2-fluoroprop-2- en-1-one | 441.1 |
| 212 | 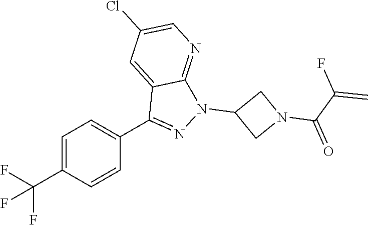 [SMILES: C=C(F)C(=O)N1CC(n2nc(-c3ccc(C(F)(F)F)cc3)c3cc(Cl)cnc32)C1] | 1-(3-(5-chloro-3-(4-(trifluoro- methyl)phenyl)-1H-pyrazolo[3,4- b]pyridin-1-yl)azetidin-1-yl)-2- fluoroprop-2-en-1-one | 425.1 |
| 213 | 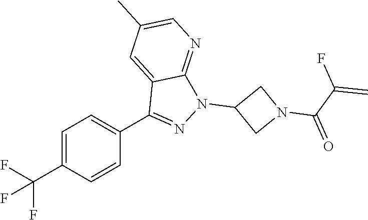 [SMILES: C=C(F)C(=O)N1CC(n2nc(-c3ccc(C(F)(F)F)cc3)c3cc(C)cnc32)C1] | 2-fluoro-1-(3-(5-methyl-3-(4- (trifluoromethyl)phenyl)-1H- pyrazolo[3,4-b]pyridin-1-yl)- azetidin-1-yl)prop-2-en-1-one | 405.1 |
| 214 | 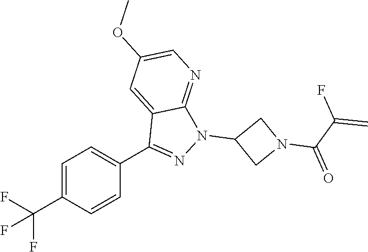 [SMILES: C=C(F)C(=O)N1CC(n2nc(-c3ccc(C(F)(F)F)cc3)c3cc(OC)cnc32)C1] | 2-fluoro-1-(3-(5-methoxy-3-(4- (trifluoromethyl)phenyl)-1H- pyrazolo[3,4-b]pyridin-1-yl)- azetidin-1-yl)prop-2-en-1-one | 421.1 |
| 215 | 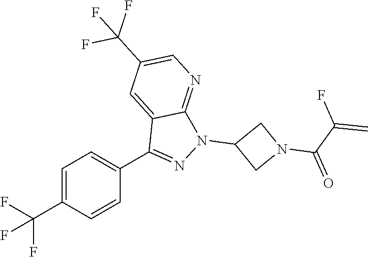 [SMILES: C=C(F)C(=O)N1CC(n2nc(-c3ccc(C(F)(F)F)cc3)c3cc(C(F)(F)F)cnc32)C1] | 2-fluoro-1-(3-(5-(trifluorometh- yl)-3-(4-(trifluoromethyl)phenyl)- 1H-pyrazolo[3,4-b]pyridin-1-yl)- azetidin-1-yl)prop-2-en-1-one | 459.1 |
| 216 | 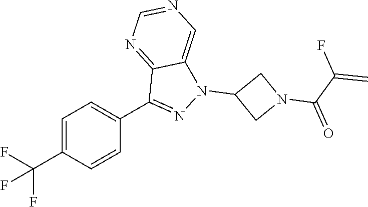 [SMILES: C=C(F)C(=O)N1CC(n2nc(-c3ccc(C(F)(F)F)cc3)c3ncncc32)C1] | 2-fluoro-1-(3-(3-(4-(trifluoro- methyl)phenyl)-1H-pyrazolo[4,3- d]pyrimidin-1-yl)azetidin-1-yl)- prop-2-en-1-one | 392.1 |
| 217 | 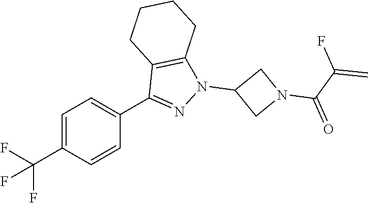 [SMILES: C=C(F)C(=O)N1CC(n2nc(-c3ccc(C(F)(F)F)cc3)c3c2CCCC3)C1] | 2-fluoro-1-(3-(3-(4-(trifluoro- methyl)phenyl)-4,5,6,7-tetra- hydro-1H-indazol-1-yl)azetidin- 1-yl)prop-2-en-1-one | 394.2 |
| 218 | 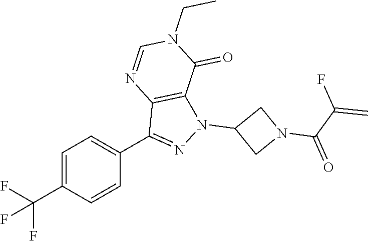 [SMILES: C=C(F)C(=O)N1CC(n2nc(-c3ccc(C(F)(F)F)cc3)c3ncn(CC)c(=O)c32)C1] | 6-ethyl-1-(1-(2-fluoroacryloyl)- azetidin-3-yl)-3-(4-(trifluoro- methyl)phenyl)-1,6-dihydro-7H- pyrazolo[4,3-d]pyrimidin-7-one | 436.1 |
| 219 | 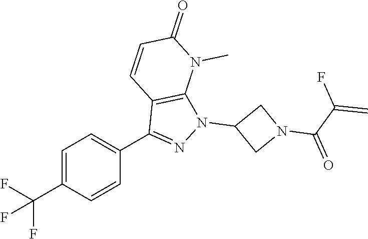 [SMILES: C=C(F)C(=O)N1CC(n2nc(-c3ccc(C(F)(F)F)cc3)c3ccc(=O)n(C)c32)C1] | 1-(1-(2-fluoroacryloyl)azetidin- 3-yl)-7-methyl-3-(4-(trifluoro- methyl)phenyl)-1,7-dihydro-6H- pyrazolo[3,4-b]pyridin-6-one | 421.1 |
| 220 | 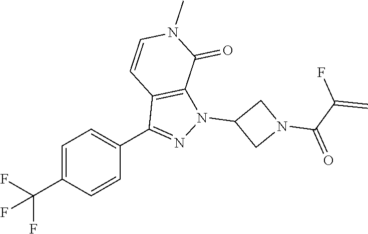 [SMILES: C=C(F)C(=O)N1CC(n2nc(-c3ccc(C(F)(F)F)cc3)c3ccn(C)c(=O)c32)C1] | 1-(1-(2-fluoroacryloyl)azetidin- 3-yl)-6-methyl-3-(4-(trifluoro- methyl)phenyl)-1,6-dihydro-7H- pyrazolo[3,4-c]pyridin-7-one | 421.1 |
| 221 | 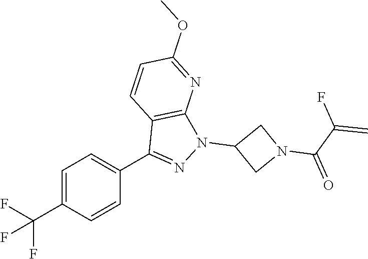 [SMILES: C=C(F)C(=O)N1CC(n2nc(-c3ccc(C(F)(F)F)cc3)c3ccc(OC)nc32)C1] | 2-fluoro-1-(3-(6-methoxy-3-(4- (trifluoromethyl)phenyl)-1H- pyrazolo[3,4-b]pyridin-1-yl)- azetidin-1-yl)prop-2-en-1-one | #REF! |
| 222 | 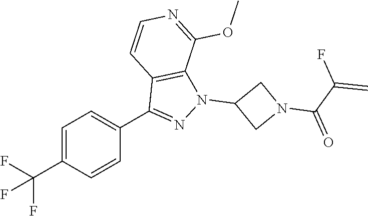 [SMILES: C=C(F)C(=O)N1CC(n2nc(-c3ccc(C(F)(F)F)cc3)c3ccnc(OC)c32)C1] | 2-fluoro-1-(3-(7-methoxy-3-(4- (trifluoromethyl)phenyl)-1H- pyrazolo[3,4-c]pyridin-1-yl)- azetidin-1-yl)prop-2-en-1-one | 421.1 |
| 223 | 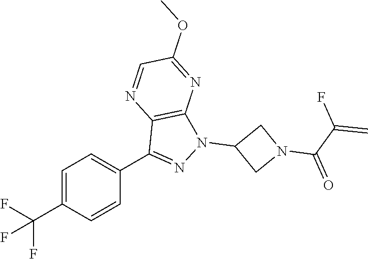 [SMILES: C=C(F)C(=O)N1CC(n2nc(-c3ccc(C(F)(F)F)cc3)c3ncc(OC)nc32)C1] | 2-fluoro-1-(3-(6-methoxy-3-(4- (trifluoromethyl)phenyl)-1H- pyrazolo[3,4-b]pyrazin-1-yl)- azetidin-1-yl)prop-2-en-1-one | 422.1 |
| 224 | 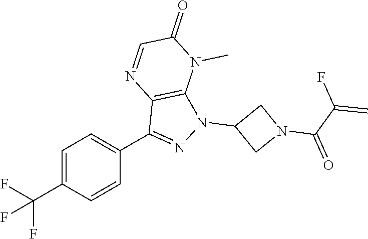 [SMILES: C=C(F)C(=O)N1CC(n2nc(-c3ccc(C(F)(F)F)cc3)c3ncc(=O)n(C)c32)C1] | 1-(1-(2-fluoroacryloyl)azetidin- 3-yl)-7-methyl-3-(4-(trifluoro- methyl)phenyl)-1,7-dihydro-6H-pyrazolo[3,4-b]pyrazin-6-one | 422.1 |
| 225 | 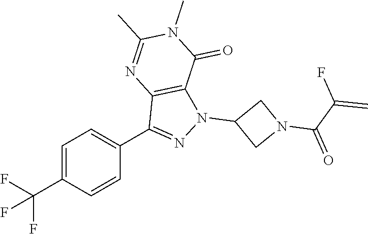 [SMILES: C=C(F)C(=O)N1CC(n2nc(-c3ccc(C(F)(F)F)cc3)c3nc(C)n(C)c(=O)c32)C1] | 1-(1-(2-fluoroacryloyl)azetidin-3- yl)-5,6-dimethyl-3-(4-(trifluoro- methyl)phenyl)-1,6-dihydro-7H- pyrazolo[4,3-d]pyrimidin-7-one | 436.1 |
| 226 | 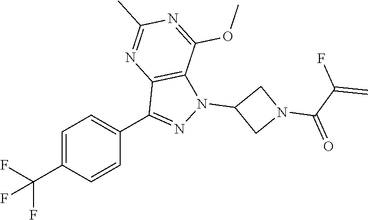 [SMILES: C=C(F)C(=O)N1CC(n2nc(-c3ccc(C(F)(F)F)cc3)c3nc(C)nc(OC)c32)C1] | 2-fluoro-1-(3-(7-methoxy-5- methyl-3-(4-(trifluoromethyl)- phenyl)-1H-pyrazolo[4,3-d]- pyrimidin-1-yl)azetidin-1-yl)- prop-2-en-1-one | 436.1 |
| 227 | 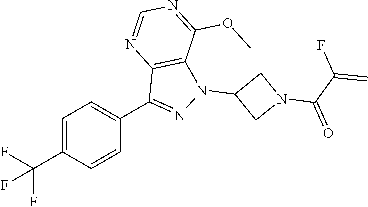 [SMILES: C=C(F)C(=O)N1CC(n2nc(-c3ccc(C(F)(F)F)cc3)c3ncnc(OC)c32)C1] | 2-fluoro-1-(3-(7-methoxy-3-(4- (trifluoromethyl)phenyl)-1H- pyrazolo[4,3-d]pyrimidin-1-yl)- azetidin-1-yl)prop-2-en-1-one | 422.1 |
| 228 | 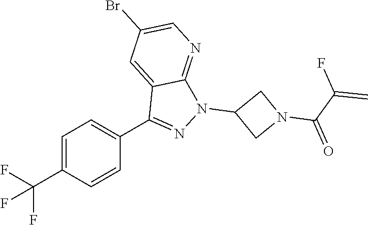 [SMILES: C=C(F)C(=O)N1CC(n2nc(-c3ccc(C(F)(F)F)cc3)c3cc(Br)cnc32)C1] | 1-(3-(5-bromo-3-(4-(trifluoro- methyl)phenyl)-1H-pyrazolo[3,4- b]pyridin-1-yl)azetidin-1-yl)-2- fluoroprop-2-en-1-one | 469.1 |

### Example 229 Synthesis of Compound 229 (1-(1-(2 fluoroacryloyl)azetidin-3 yl)-3-(4-(trifluoromethyl)phenyl)-1,3-dihydro-2H-benzo[d]imidazol-2-one)

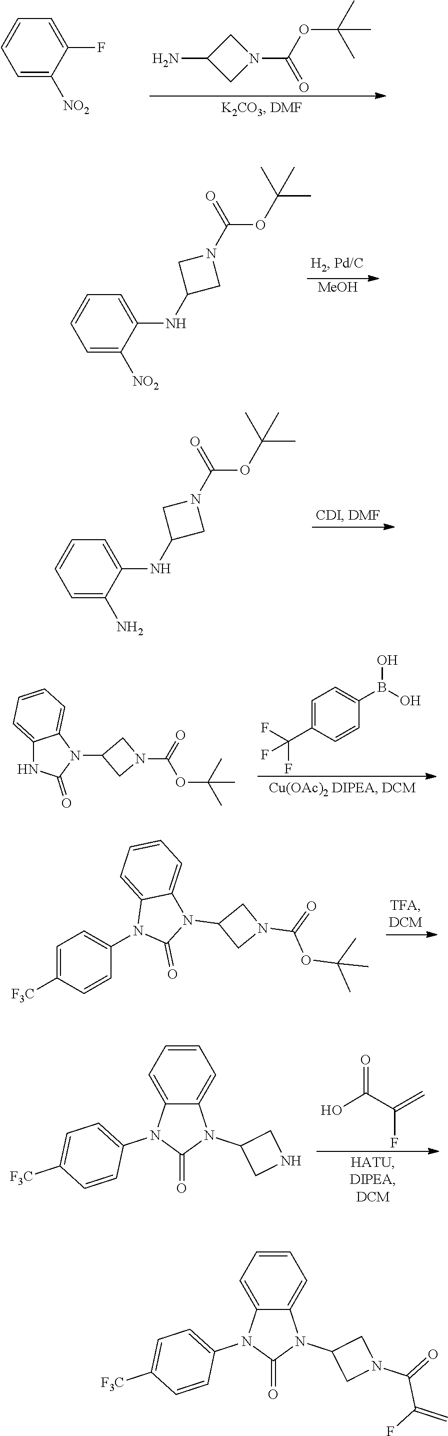

### Step 1: Preparation of tert-butyl 3-((2-nitrophenyl)amino)azetidine-1-carboxylate

A mixture of 1-fluoro-2-nitrobenzene (10.0 g, 70.87 mmol), tert-butyl 3-aminoazetidine-1-carboxylate (24.41 g, 141.74 mmol), potassium carbonate (29.39 g, 212.62 mmol), and DMF (200 mL) was stirred for 3 h at room temperature. The reaction was monitored by LCMS. The reaction was poured into ice-water. The mixture was filtered. The filter cake was washed with water and dried under vacuum to afford the title compound (18.12 g) as yellow solid, which was used for the next step without any further purification. LCMS [M+H]⁺=238.32.

### Step 2: Preparation of tert-butyl 3-((2-aminophenyl)amino)azetidine-1-carboxylate

A mixture of tert-butyl 3-((2-nitrophenyl)amino)azetidine-1-carboxylate (8.00 g, 27.27 mmol), Pd/C (1.0 g, 10%), and MeOH (100 mL) was stirred for overnight at room temperature under hydrogen atmosphere. The reaction was monitored by LCMS. The mixture was filtered. The filtrate was concentrated under vacuum to afford the tittle compound (10.50 g) as off white solid, which was used for the next step without any further purification. LCMS [M+H]⁺=264.42.

### Step 3: Preparation of tert-butyl 3-(2-oxo-2,3-dihydro-1H-benzo[d]imidazol-1-yl)azetidine-1-carboxylate

A mixture of tert-butyl 3-((2-aminophenyl)amino)azetidine-1-carboxylate (10.00 g, 37.97 mmol), DMF (50 mL), and 1,1′-carbonyldiimidazole (12.31 g, 75.95 mmol) was stirred for 2 h at 100° C. The reaction was monitored by LCMS. The reaction was cooled down to room temperature. The reaction mixture was then diluted with ethyl acetate, washed with water, dried over anhydrous sodium sulfate, and then filtered. The filtrate was concentrated under vacuum. The residue was purified by silica gel chromatography eluting with hexane:ethyl acetate=2:1 to afford the tittle compound (6.10 g) as off-white solid. LCMS [M+H]⁺=234.34.

### Step 4: Preparation of tert-butyl 3-(2-oxo-3-(4-(trifluoromethyl)phenyl)-2,3-dihydro-1H-benzo[d]imidazol-1 yl)azetidine-1-carboxylate

A mixture of tert-butyl 3-(2-oxo-2,3-dihydro-1H-benzo[d]imidazol-1-yl)azetidine-1-carboxylate (6.00 g, 20.74 mmol), (4-(trifluoromethyl)phenyl)boronic acid (5.91 g, 31.11 mmol), DIPEA (8.04 g, 62.21 mmol), Cu(OAc)₂ (3.77 g, 20.74 mmol), and DCM (60 mL) was stirred for 8 h at room tempter under oxygen atmosphere. The reaction was monitored by LCMS. The mixture was filtered through diatomaceous earth. The filtrate was washed with brine, dried over anhydrous sodium sulfate and then filtered. The filtrate was concentrated under vacuum. The residue was purified by silica gel chromatography eluting with hexane:ethyl acetate=3:1 to afford the tittle compound (7.65 g) as blue oil. LCMS [M+H]⁺=378.42.

### Step 5: Preparation of 1-(azetidin-3-yl)-3-(4-(trifluoromethyl)phenyl)-1,3-dihydro-2H-benzo[d]imidazol-2-one

A mixture of tert-butyl 3-(2-oxo-3-(4-(trifluoromethyl)phenyl)-2,3-dihydro-1H-benzo[d]imidazol-1-yl)azetidine-1-carboxylate (7.65 g, 17.65 mmol), TFA (38.0 mL), and DCM (75 mL) was stirred for 8 h at room temperature. The reaction was monitored by LCMS. The reaction mixture was concentrated under vacuum. The residue was diluted with dichloromethane. The pH value was adjusted to 10 with saturated sodium carbonate. The mixture was extracted with dichloromethane, dried over anhydrous sodium sulfate, and then filtered. The filtrate was concentrated under vacuum to afford the title compound (4.25 g) as off-white solid, which was used for the next step without any further purification. LCMS [M+H]⁺=334.31.

### Step 6: Preparation of 1-(1-(2 fluoroacryloyl)azetidin-3 yl)-3-(4-(trifluoromethyl)phenyl)-1,3-dihydro-2H-benzo[d]imidazol-2-one

A mixture of 1-(azetidin-3-yl)-3-(4-(trifluoromethyl)phenyl)-1,3-dihydro-2H-benzo[d]imidazol-2-one (2.80 g, 8.40 mmol), DIPEA 3.26 g, 25.20 mmol), DCM (20 mL), 2-fluoroacrylic acid (1.13 g, 12.60 mmol), and HATU (3.19 g, 8.40 mmol) was stirred for 2 h at room temperature. The reaction was monitored by LCMS. The reaction mixture was then diluted with dichloromethane, washed with water, dried over anhydrous sodium sulfate, and then filtered. The filtrate was concentrated under vacuum. The residue was purified by silica gel chromatography eluting with hexane:ethyl acetate=2:1 to afford the tittle compound (1.32 g) as white solid.

LCMS [M+H]⁺=406.46.

¹H NMR (500 MHz, DMSO-d₆) (57.96 (d, J=8.4 Hz, 2H), 7.84 (d, J=8.3 Hz, 2H), 7.39 (d, J=7.8 Hz, 1H), 7.27-7.18 (m, 2H), 7.14 (td, J=7.7, 1.1 Hz, 1H), 5.55 (dd, J=48.4, 3.5 Hz, 1H), 5.43 (tt, J=8.7, 5.7 Hz, 1H), 5.36 (dd, J=16.5, 3.5 Hz, 1H), 5.03-4.69 (m, 2H), 4.65-4.40 (m, 2H).

### Example 230 Synthesis of Compound 230 (2 fluoro-1-(3-(4-(4-(trifluoromethyl)phenyl)quinazolin-2 yl)azetidin-1 yl)prop-2-en-1-one)

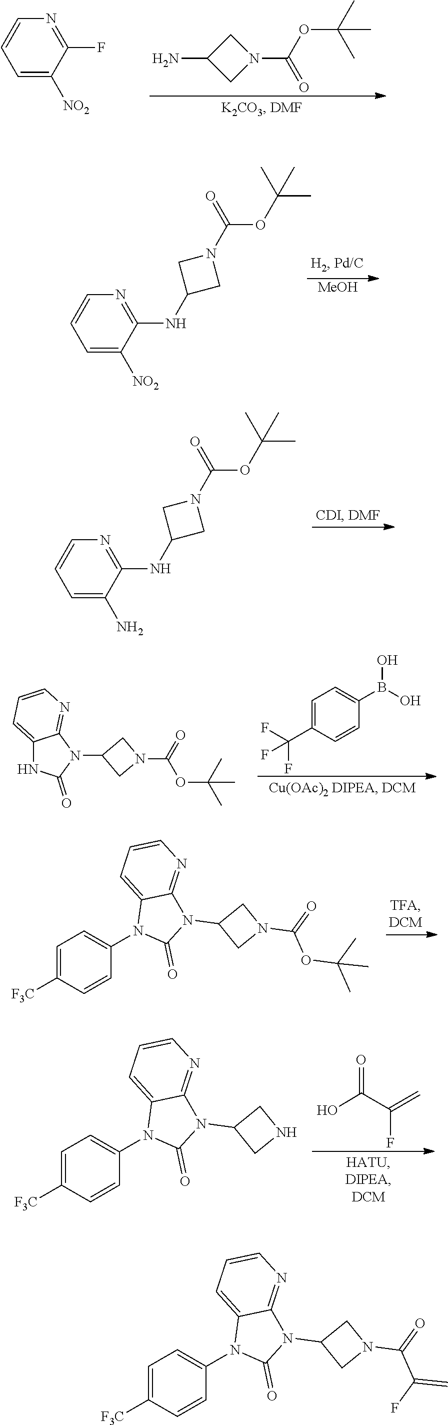

### Step 1: Preparation of tert-butyl 3-((3-nitropyridin-2 yl)amino)azetidine-1-carboxylate

A mixture of tert-butyl 3-aminoazetidine-1-carboxylate (5.45 g, 31.76 mmol), 2-fluoro-3-nitropyridine (3.0 g, 21.11 mmol), potassium carbonate (8.75 g, 63.34 mmol), and DMF (20 mL) was stirred for 2 h at 20° C. The reaction was monitored by LCMS. The reaction was diluted with ethyl acetate, washed with water, dried over anhydrous sodium sulfate, and then filtered. The filtrate was concentrated under vacuum to afford the title compound (9.25 g) as yellow oil, which was used for the next step without any further purification. LCMS [M+H]⁺=239.42.

### Step 2: Preparation of tert-butyl 3-((3-aminopyridin-2-yl)amino)azetidine-1-carboxylate

A mixture of tert-butyl 3-((3-nitropyridin-2-yl)amino)azetidine-1-carboxylate (9.25 g, 31.43 mmol), Pd/C (1.50 g, 10%), MeOH (100 mL) was stirred for overnight at room temperature under hydrogen atmosphere. The reaction was monitored by LCMS. The mixture was filtered and the filtrate was concentrated under vacuum to afford the tittle compound (6.50 g) as brown solid, which was used for the next step without any further purification. LCMS [M+H]⁺=265.42.

### Step 3: Preparation of tert-butyl 3-(2-oxo-1,2-dihydro-3H-imidazo[4,5-b]pyridin-3-yl)azetidine-1-carboxylate

A mixture of tert-butyl 3-((3-aminopyridin-2-yl)amino)azetidine-1-carboxylate (5.20 g, 19.67 mmol), DMF (25 mL), and 1,1′-carbonyldiimidazole (9.57 g, 59.02 mmol) was stirred for overnight at 65° C. The reaction was monitored by LCMS. The reaction was cooled down to room temperature. The reaction mixture was then diluted with ethyl acetate, washed with water, dried over anhydrous sodium sulfate, and then filtered. The filtrate was concentrated under vacuum. The residue was purified by silica gel chromatography eluting with hexane:ethyl acetate=2:1 to afford the tittle compound (4.25 g) as brown foam. LCMS [M+H]⁺=235.34

### Step 4: Preparation of tert-butyl 3-(2-oxo-1-(4-(trifluoromethyl)phenyl)-1,2-dihydro-3H-imidazo[4,5-b]pyridin-3 yl)azetidine-1-carboxylate

A mixture of tert-butyl 3-(2-oxo-1,2-dihydro-3H-imidazo[4,5-b]pyridin-3-yl)azetidine-1-carboxylate (3.52 g, 12.12 mmol), (4-(trifluoromethyl)phenyl)boronic acid (4.61 g, 24.25 mmol), TEA (6.13 g, 60.62 mmol), Cu(OAc)₂ (2.20 g, 12.12 mmol), 4A powder molecular sieve (7.0 g) and DCM (40 mL) was stirred for 8 h at room temperature under oxygen atmosphere. The reaction was monitored by LCMS. The mixture was filtered through diatomaceous earth, the filter cake was washed with DCM. The filtrate was washed with brine, dried over anhydrous sodium sulfate and then filtered. The filtrate was concentrated under vacuum. The residue was purified by silica gel chromatography eluting with hexane:ethyl acetate=2:1 to afford the tittle compound (5.50 g) as yellow solid. LCMS [M+H]⁺=379.42.

### Step 5: Preparation of 3-(azetidin-3 yl)-1-(4-(trifluoromethyl)phenyl)-1,3-dihydro-2H-imidazo[4,5-b]pyridin-2-one

A mixture of tert-butyl 3-(2-oxo-1-(4-(trifluoromethyl)phenyl)-1,2-dihydro-3H-imidazo[4,5-b]pyridin-3-yl)azetidine-1-carboxylate (5.50, 12.66 mmol), TFA (28.0 mL), and DCM (60 mL) was stirred for 8 h at room temperature. The reaction was monitored by LCMS. The reaction mixture was concentrated under vacuum. The residue was dissolved in dichloromethane. The pH value was adjusted to 10 with saturated sodium carbonate aqueous solution. The mixture was extracted with dichloromethane, washed with brine, dried over anhydrous sodium sulfate, and then filtered. The filtrate was concentrated under vacuum to afford the title compound (4.50 g) as yellow solid, which was used for the next step without any further purification. LCMS [M+H]⁺=335.31

### Step 6: Preparation of 1 3-(1-(2 fluoroacryloyl)azetidin-3 yl)-1-(4-(trifluoromethyl)phenyl)-1,3-dihydro-2H-imidazo[4,5-b]pyridin-2-one

A mixture of 2-fluoroacrylic acid (1.82 g, 20.19 mmol), DCM (45 mL), DIPEA (5.22 g, 40.38 mmol), HATU (4.50 g, 40.38 mmol), and 3-(azetidin-3-yl)-1-(4-(trifluoromethyl)phenyl)-1,3-dihydro-2H-imidazo[4,5-b]pyridin-2-one (4.50 g, 13.46 mmol) was stirred for 2 h at room temperature. The reaction was monitored by LCMS. The reaction mixture was then diluted with dichloromethane, washed with water, dried over anhydrous sodium sulfate, and then filtered. The filtrate was concentrated under vacuum. The residue was purified by silica gel chromatography eluting with hexane:ethyl acetate=2:1 to afford the tittle compound (3.40 g) as white solid.

LCMS [M+H]⁺=407.46.

¹H NMR (500 MHz, DMSO-d₆) δ 8.13 (dd, J=5.2, 1.4 Hz, 1H), 7.96 (d, J=8.7 Hz, 2H), 7.86 (d, J=8.3 Hz, 2H), 7.58 (dd, J=7.8, 1.4 Hz, 1H), 7.21-7.11 (m, 1H), 5.62-5.43 (m, 2H), 5.34 (dd, J=16.5, 3.4 Hz, 1H), 5.10-5.01 (m, 1H), 4.83-4.67 (m, 2H), 4.47-4.35 (m, 1H).

### Example 231 Synthesis of Compound 231 (1-(3-(2-imino-3-(4-(trifluoromethyl)phenyl)-2,3-dihydro-1H-benzo[d]imidazol-1 yl)azetidin-1 yl)prop-2-en-1-one)

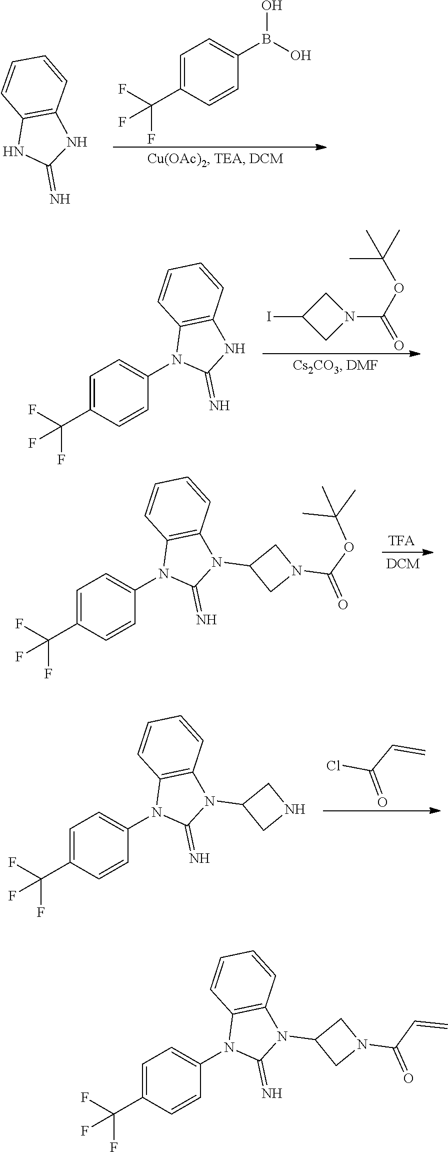

### Step 1: Preparation of 1-(4-(Trifluoromethyl)phenyl)-1,3-dihydro-2H-benzo [d]imidazol-2-imine

A mixture of 3H-benzoimidazol-2-amine (1.00 g, 7.51 mmol), 4-[4-(trifluoromethyl)phenyl]boronic acid (1.71 g, 9.01 mmol), Cu(OAc)₂ (0.27 g, 1.50 mmol), TEA (3.13 mL, 22.53 mmol) in DCM (10 mL) was stirred for 4 h at room temperature. The reaction mixture was filtered. The filtrate was concentrated under vacuum. The residue was purified by silica gel chromatography eluting with ethyl acetate in hexane from 0% to 50% to afford the tile product (1.50 g, 72%) as a brown solid. LCMS [M+H]⁺=278.25.

### Step 2: Preparation of Tert-butyl-3-(2-imino-3-(4-(trifluoromethyl)phenyl)-2,3-dihydro-1H-benzo[d]imidazol-1 yl)azetidine-1-carboxylate

A mixture of 1-(4-(trifluoromethyl)phenyl)-1,3-dihydro-2H-benzo[d]imidazol-2-imine (0.10 g, 0.36 mmol), tert-butyl-3-iodoazetidine-1-carboxylate (0.12 g, 0.43 mmol), Cs₂CO₃ (0.23 g, 0.72 mmol) in DMF (3 mL) was stirred for 4 h at 100° C. The reaction was cooled to room temperature and quenched by the addition of water. The mixture was extracted with ethyl acetate, washed with brine, dried over anhydrous sodium sulfate, and filtered. The filtrate was concentrated under vacuum. The residue was purified by silica gel chromatography eluting with ethyl acetate in hexane from 0% to 30% to afford the title compound (80 mg, 51%) as a brown solid. LCMS [M+H]⁺=433.45.

### Step 3: Preparation of 1-(Azetidin-3 yl)-3-(4-(trifluoromethyl)phenyl)-1,3-dihydro-2H-benzo[d]imidazol-2-imine

A mixture of tert-butyl-3-(2-imino-3-(4-(trifluoromethyl)phenyl)-2,3-dihydro-1H-benzo[d]imidazol-1-yl)azetidine-1-carboxylate (80 mg, 0.18 mmol), DCM (5 mL), and TFA (0.14 mL, 1.85 mmol) was stirred for 1 h at room temperature. The pH value of the reaction was adjusted to 10 with sodium carbonate aqueous solution, extracted with dichloromethane, washed with brine, dried over anhydrous sodium sulfate, and filtered. The filtrate was concentrated under vacuum to afford the title compound (50 mg) as yellow solid, which was used for the next step without any further purification. LCMS [M+H]⁺=333.33.

### Step 4: Preparation of 1-(3-(2 Imino-3-(4-(trifluoromethyl)phenyl)-2,3-dihydro-1H-benzo[d]imidazol-1 yl)azetidin-1 yl)prop-2-en-1-one

Acryloyl chloride (16.30 mg, 0.18 mmol) was added dropwise into a solution of 1-(azetidin-3-yl)-3-(4-(trifluoromethyl)phenyl)-1,3-dihydro-2H-benzo[d]imidazol-2-imine (50.00 mg, 0.15 mmol), TEA (0.04 mL, 0.30 mmol), and DCM (4 mL) at 0° C. under nitrogen. The reaction was stirred for 0.5 h at room temperature. The reaction was quenched by the addition of water. The mixture was extracted with dichloromethane. The combined organic layers was washed with brine, dried over anhydrous sodium sulfate, and filtered. The filtrate was concentrated under vacuum. The residue was purified by Prep-TLC (hexane/EA=2/1) to afford the title product (3.00 mg, 5%) as an off white solid. LCMS [M+H]⁺=387.38.

The compounds of table 2 were prepared in a similar manner to Examples 1-235 via different reaction starting materials and suitable reagents.

TABLE 2

| | | | Physical Data |
|---|---|---|---|
| EX | | | (LCMS) |
| No. | Structure | Chemical Name | (M + H)⁺ |
| 232 | 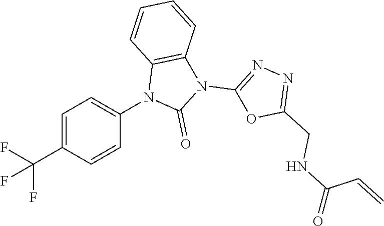 [SMILES: C=CC(=O)NCc1nnc(-n2c(=O)n(-c3ccc(C(F)(F)F)cc3)c3ccccc32)o1] | N-((5-(2-oxo-3-(4- (trifluoromethyl)phenyl)-2,3-dihydro- 1H-benzo[d]imidazol-1-yl)-1,3,4- oxadiazol-2-yl)methyl)acrylamide | 430.1 |
| 233 | 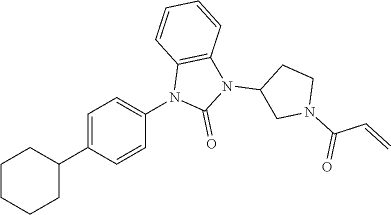 [SMILES: C=CC(=O)N1CCC(n2c(=O)n(-c3ccc(C4CCCCC4)cc3)c3ccccc32)C1] | 1-(1-acryloylpyrrolidin-3-yl)-3-(4- cyclohexylphenyl)-1,3-dihydro-2H- benzo[d]imidazol-2-one | 416.2 |
| 234 | 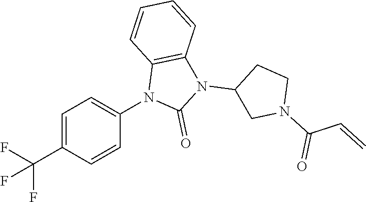 [SMILES: C=CC(=O)N1CCC(n2c(=O)n(-c3ccc(C(F)(F)F)cc3)c3ccccc32)C1] | 1-(1-acryloylpyrrolidin-3-yl)-3-(4- (trifluoromethyl)phenyl)-1,3-dihydro- 2H-benzo[d]imidazol-2-one | 402.1 |
| 235 | 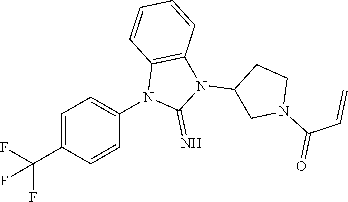 [SMILES: C=CC(=O)N1CCC(n2c(=N)n(-c3ccc(C(F)(F)F)cc3)c3ccccc32)C1] | 1-(3-(2-imino-3-(4- (trifluoromethyl)phenyl)-2,3-dihydro- 1H-benzo[d]imidazol-1-yl)pyrrolidin-1- yl)prop-2-en-1-one | 401.2 |
| 236 | 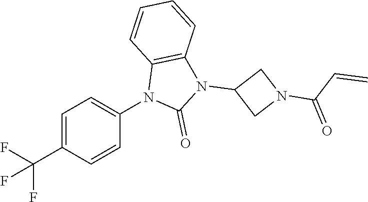 [SMILES: C=CC(=O)N1CC(n2c(=O)n(-c3ccc(C(F)(F)F)cc3)c3ccccc32)C1] | 1-(1-acryloylazetidin-3-yl)-3-(4- (trifluoromethyl)phenyl)-1,3-dihydro- 2H-benzo[d]imidazol-2-one | 388.1 |
| 237 | 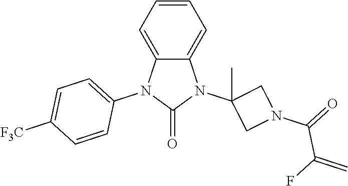 [SMILES: C=C(F)C(=O)N1CC(C)(n2c(=O)n(-c3ccc(C(F)(F)F)cc3)c3ccccc32)C1] | 1-(1-(2-fluoroacryloyl)-3- methylazetidin-3-yl)-3-(4- (trifluoromethyl)phenyl)-1,3-dihydro- 2H-benzo[d]imidazol-2-one | 420.1 |
| 238 | 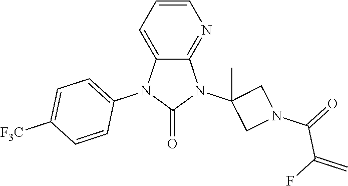 [SMILES: C=C(F)C(=O)N1CC(C)(n2c(=O)n(-c3ccc(C(F)(F)F)cc3)c3cccnc32)C1] | 3-(1-(2-fluoroacryloyl)-3- methylazetidin-3-yl)-1-(4- (trifluoromethyl)phenyl)-1,3-dihydro- 2H-imidazo[4,5-b]pyridin-2-one | 421.1 |
| 239 | 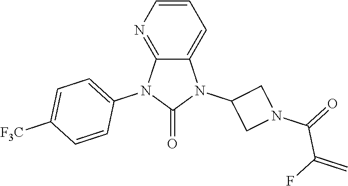 [SMILES: C=C(F)C(=O)N1CC(n2c(=O)n(-c3ccc(C(F)(F)F)cc3)c3ncccc32)C1] | 1-(1-(2-fluoroacryloyl)azetidin-3-yl)-3- (4-(trifluoromethyl)phenyl)-1,3- dihydro-2H-imidazo[4,5-b]pyridin-2- one | 407.1 |
| 240 | 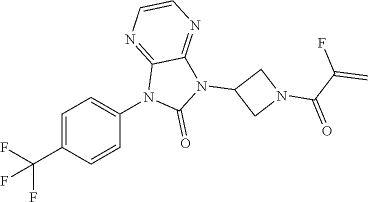 [SMILES: C=C(F)C(=O)N1CC(n2c(=O)n(-c3ccc(C(F)(F)F)cc3)c3nccnc32)C1] | 1-(1-(2-fluoroacryloyl)azetidin-3-yl)-3- (4-(trifluoromethyl)phenyl)-1,3- dihydro-2H-imidazo[4,5-b]pyrazin-2- one | 408.1 |
| 241 | 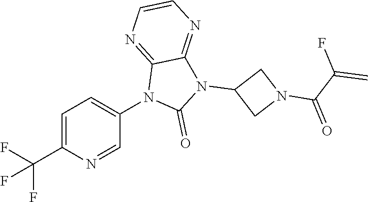 [SMILES: C=C(F)C(=O)N1CC(n2c(=O)n(-c3ccc(C(F)(F)F)nc3)c3nccnc32)C1] | 1-(1-(2-fluoroacryloyl)azetidin-3-yl)-3- (6-(trifluoromethyl)pyridin-3-yl)-1,3- dihydro-2H-imidazo[4,5-b]pyrazin-2- one | 409.1 |
| 242 | 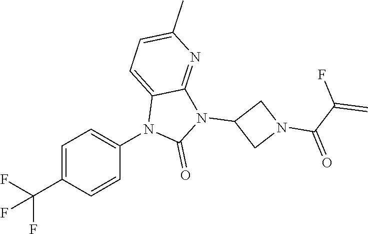 [SMILES: C=C(F)C(=O)N1CC(n2c(=O)n(-c3ccc(C(F)(F)F)cc3)c3ccc(C)nc32)C1] | 3-(1-(2-fluoroacryloyl)azetidin-3-yl)-5- methyl-1-(4-(trifluoromethyl)phenyl)- 1,3-dihydro-2H-imidazo[4,5-b]pyridin- 2-one | 421.1 |
| 243 | 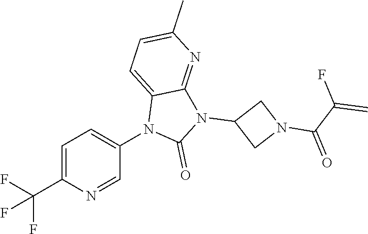 [SMILES: C=C(F)C(=O)N1CC(n2c(=O)n(-c3ccc(C(F)(F)F)nc3)c3ccc(C)nc32)C1] | 3-(1-(2-fluoroacryloyl)azetidin-3-yl)-5- methyl-1-(6-(trifluoromethyl)pyridin-3- yl)-1,3-dihydro-2H-imidazo[4,5- b]pyridin-2-one | 422.1 |
| 244 | 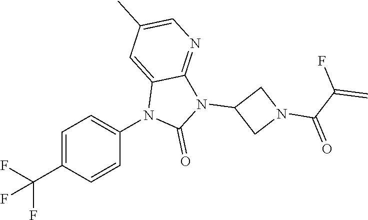 [SMILES: C=C(F)C(=O)N1CC(n2c(=O)n(-c3ccc(C(F)(F)F)cc3)c3cc(C)cnc32)C1] | 3-(1-(2-fluoroacryloyl)azetidin-3-yl)-6- methyl-1-(4-(trifluoromethyl)phenyl)- 1,3-dihydro-2H-imidazo[4,5-b]pyridin- 2-one | 421.1 |
| 245 | 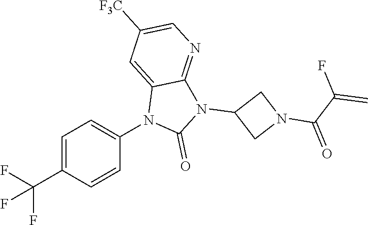 [SMILES: C=C(F)C(=O)N1CC(n2c(=O)n(-c3ccc(C(F)(F)F)cc3)c3cc(C(F)(F)F)cnc32)C1] | 3-(1-(2-fluoroacryloyl)azetidin-3-yl)-6- (trifluoromethyl)-1-(4- (trifluoromethyl)phenyl)-1,3-dihydro- 2H-imidazo[4,5-b]pyridin-2-one | 475.1 |
| 246 | 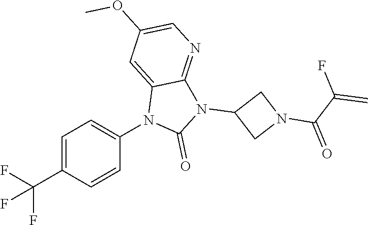 [SMILES: C=C(F)C(=O)N1CC(n2c(=O)n(-c3ccc(C(F)(F)F)cc3)c3cc(OC)cnc32)C1] | 3-(1-(2-fluoroacryloyl)azetidin-3-yl)-6- methoxy-1-(4-(trifluoromethyl)phenyl)- 1,3-dihydro-2H-imidazo[4,5-b]pyridin- 2-one | 437.1 |
| 247 | 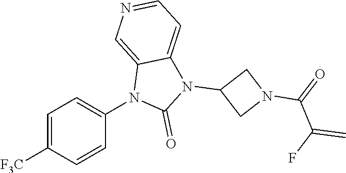 [SMILES: C=C(F)C(=O)N1CC(n2c(=O)n(-c3ccc(C(F)(F)F)cc3)c3cnccc32)C1] | 1-(1-(2-fluoroacryloyl)azetidin-3-yl)-3- (4-(trifluoromethyl)phenyl)-1,3- dihydro-2H-imidazo[4,5-c]pyridin-2- one | 407.3 |
| 248 | 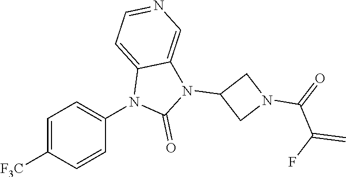 [SMILES: C=C(F)C(=O)N1CC(n2c(=O)n(-c3ccc(C(F)(F)F)cc3)c3ccncc32)C1] | 3-(1-(2-fluoroacryloyl)azetidin-3-yl)-1- (4-(trifluoromethyl)phenyl)-1,3- dihydro-2H-imidazo[4,5-c]pyridin-2- one | 407.1 |
| 249 | 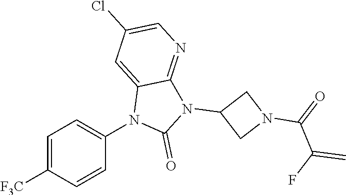 [SMILES: C=C(F)C(=O)N1CC(n2c(=O)n(-c3ccc(C(F)(F)F)cc3)c3cc(Cl)cnc32)C1] | 6-chloro-3-(1-(2- fluoroacryloyl)azetidin-3-yl)-1-(4- (trifluoromethyl)phenyl)-1,3-dihydro- 2H-imidazo[4,5-b]pyridin-2-one | 441.1 |
| 250 | 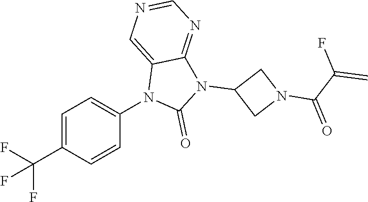 [SMILES: C=C(F)C(=O)N1CC(n2c(=O)n(-c3ccc(C(F)(F)F)cc3)c3cncnc32)C1] | 9-(1-(2-fluoroacryloyl)azetidin-3-yl)-7- (4-(trifluoromethyl)phenyl)-7,9- dihydro-8H-purin-8-one | 408.2 |
| 251 | 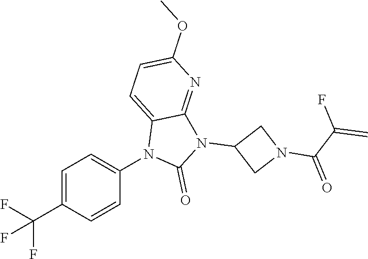 [SMILES: C=C(F)C(=O)N1CC(n2c(=O)n(-c3ccc(C(F)(F)F)cc3)c3ccc(OC)nc32)C1] | 3-(1-(2-fluoroacryloyl)azetidin-3-yl)-5- methoxy-1-(4-(trifluoromethyl)phenyl)- 1,3-dihydro-2H-imidazo[4,5-b]pyridin- 2-one | 437.1 |
| 252 | 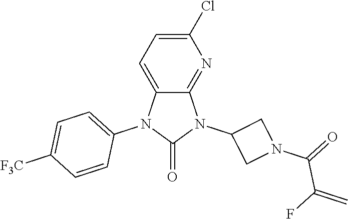 [SMILES: C=C(F)C(=O)N1CC(n2c(=O)n(-c3ccc(C(F)(F)F)cc3)c3ccc(Cl)nc32)C1] | 5-chloro-3-(1-(2- fluoroacryloyl)azetidin-3-yl)-1-(4- (trifluoromethyl)phenyl)-1,3-dihydro- 2H-imidazo[4,5-b]pyridin-2-one | 441.7 |
| 253 | 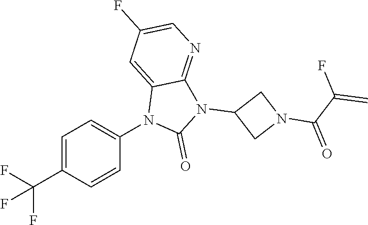 [SMILES: C=C(F)C(=O)N1CC(n2c(=O)n(-c3ccc(C(F)(F)F)cc3)c3cc(F)cnc32)C1] | 6-fluoro-3-(1-(2- fluoroacryloyl)azetidin-3-yl)-1-(4- (trifluoromethyl)phenyl)-1,3-dihydro- 2H-imidazo[4,5-b]pyridin-2-one | 425.1 |

### Example 254 Synthesis of Compound 254 (N-(1-(2-(4-(trifluoromethyl)phenyl)quinazolin-4 yl)pyrrolidin-3 yl)acrylamide)

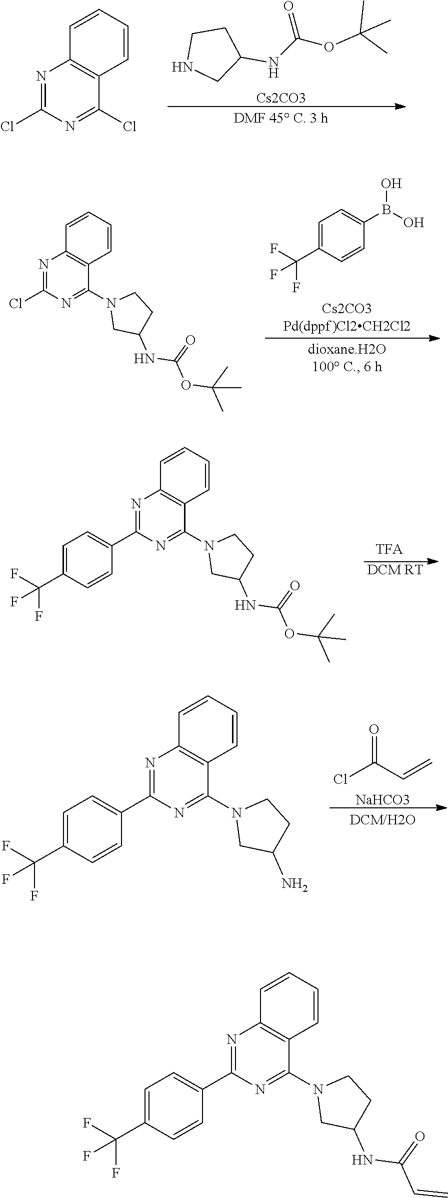

### Step 1: Preparation of tert-butyl (1-(2-chloroquinazolin-4 yl)pyrrolidin-3 yl)carbamate

A mixture of 2,4-dichloroquinazoline (1.00 g, 5.02 mmol) and tert-butyl 3-aminoazetidine-1-carboxylate (0.65 g, 3.77 mmol), carbonate (1.40 g, 7.54 mmol), and DMF (10 mL), was stirred for 3 h at 45° C. The reaction was monitored by LCMS. The reaction was diluted with ethyl acetate, poured into ice-water. The organic layer was separated, dried over anhydrous sodium sulfate, and then filtered. The filtrate was concentrated under vacuum to afford the title compound (1.30 g) as yellow solid. LCMS [M+H]⁺=293.12.

### Step 2: Preparation of tert-butyl (1-(2-(4-(trifluoromethyl)phenyl)quinazolin-4-yl)pyrrolidin-3 yl)carbamate

A mixture of tert-butyl (1-(2-chloroquinazolin-4-yl)pyrrolidin-3-yl)carbamate (1.30 g, 3.73 mmol), (4-(trifluoromethyl)phenyl)boronic acid (1.06 g, 5.59 mmol), Pd(dppf)Cl₂·CH₂Cl₂ (303 mg, 0.37 mmol), cesium carbonate (3.64 g, 11.18 mmol), 1,4-dioxane (20 mL), and water (2.0 mL) was stirred for 6 h at 100° C. under nitrogen. The reaction was monitored by LCMS. The reaction was cooled down to room temperature. The mixture was concentrated under vacuum. The residue was purified by silica gel chromatography eluting with Hex:EA=0%-30% to afford the tittle compound (1.30 g) as off white solid. LCMS [M+H]⁺=403.42.

### Step 3: Preparation of 1-(2-(4-(trifluoromethyl)phenyl)quinazolin-4 yl)pyrrolidin-3-amine

A mixture of tert-butyl (1-(2-(4-(trifluoromethyl)phenyl)quinazolin-4-yl)pyrrolidin-3-yl)carbamate (500 mg, 1.09 mmol), TFA (5 mL), and DCM (20 mL) was stirred for 4 h at room temperature. The reaction was monitored by LCMS. The reaction mixture was concentrated under vacuum to afford the title compound (600 mg) as off white solid, which was used for the next step without any further purification. LCMS [M+H]⁺=359.26.

### Step 4: Preparation of N-(1-(2-(4-(trifluoromethyl)phenyl)quinazolin-4 yl)pyrrolidin-3-yl)acrylamide

Acryloyl chloride (76 mg, 0.83 mmol) was added dropwise with stirring into a mixture of 1-(2-(4-(trifluoromethyl)phenyl)quinazolin-4-yl)pyrrolidin-3-amine (300 mg, 0.83 mmol), sodium bicarbonate (350 mg, 4.19 mmol), DCM (20 mL), and water (10 mL) at 0° C. under nitrogen atmosphere. The mixture was stirred for 30 min at 0° C. The reaction was monitored by LCMS. The reaction mixture was then diluted with dichloromethane, washed with water, dried over anhydrous sodium sulfate, and then filtered. The filtrate was concentrated under vacuum. The residue was purified by silica gel chromatography eluting with hexane:ethyl acetate=2:1 to afford the tittle compound (113 mg) as off white solid.

LCMS [M+H]⁺=413.33.

¹H NMR (500 MHz, DMSO-d₆) (58.68 (d, J=8.1 Hz, 2H), 8.50 (d, J=6.6 Hz, 1H), 8.31 (d, J=8.4 Hz, 1H), 7.90-7.84 (m, 3H), 7.81 (t, J=7.6 Hz, 1H), 7.51 (ddd, J=8.5, 6.7, 1.6 Hz, 1H), 6.23 (dd, J=17.1, 9.9 Hz, 1H), 6.13 (dd, J=17.1, 2.4 Hz, 1H), 5.62 (dd, J=10.0, 2.5 Hz, 1H), 4.52 (p, J=5.5 Hz, 1H), 4.26 (dd, J=11.8, 6.0 Hz, 1H), 4.15 (q, J=7.7, 5.8 Hz, 1H), 4.12-4.03 (m, 1H), 3.89 (dd, J=11.6, 4.0 Hz, 1H), 2.27 (pd, J=9.2, 8.4, 5.5 Hz, 1H), 2.06 (dq, J=11.8, 5.5 Hz, 1H).

### Example 255 Synthesis of Compound 255 (2 fluoro-1-(3-((2-(4-(trifluoromethyl)phenyl)quinazolin-4 yl)amino)azetidin-1 yl)prop-2-en-1-one)

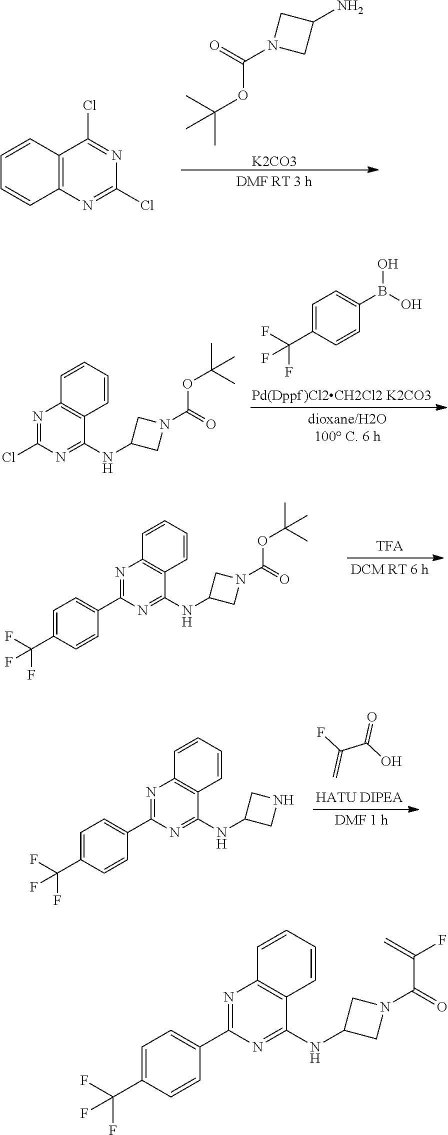

### Step 1: Preparation of tert-butyl 3-((2-chloroquinazolin-4 yl)amino)azetidine-1-carboxylate

A mixture of 2,4-dichloroquinazoline (500 mg, 2.51 mmol) and tert-butyl pyrrolidin-3-ylcarbamate (4.91 g, 15.07 mmol), potassium carbonate (1.04 g, 7.54 mmol), and DMF (10 mL) was stirred for 3 h at room temperature. The reaction was monitored by LCMS. The reaction was diluted with ethyl acetate, poured into ice-water. The organic layer was separated, dried over anhydrous sodium sulfate, and then filtered. The filtrate was concentrated under vacuum to afford the title compound (0.80 g) as yellow solid. LCMS [M+H]⁺=335.54.

### Step 2: Preparation of tert-butyl 3-((2-(4-(trifluoromethyl)phenyl)quinazolin-4-yl)amino)azetidine-1-carboxylate

A mixture of tert-butyl 3-((2-chloroquinazolin-4-yl)amino)azetidine-1-carboxylate (0.80 g, 2.39 mmol), (4-(trifluoromethyl)phenyl)boronic acid (0.68 g, 3.58 mmol), Pd(dppf)Cl₂·CH₂Cl₂ (0.20 mg, 0.24 mmol), potassium carbonate (0.99 g, 7.17 mmol), 1,4-dioxane (20 mL), and water (2.0 mL) was stirred for 6 h at 100° C. under nitrogen. The reaction was monitored by LCMS. The reaction was cooled down to room temperature. The mixture was concentrated under vacuum. The residue was purified by silica gel chromatography eluting with Hex:EA=0%-30% to afford the tittle compound (0.75 g) as off white solid. LCMS [M+H]⁺=445.43.

### Step 3: Preparation of N-(azetidin-3 yl)-2-(4-(trifluoromethyl)phenyl)quinazolin-4-amine

A mixture of tert-butyl 3-((2-(4-(trifluoromethyl)phenyl)quinazolin-4-yl)amino)azetidine-1-carboxylate (750 mg, 1.69 mmol), TFA (5 mL), and DCM (20 mL) was stirred for 4 h at room temperature. The reaction was monitored by LCMS. The reaction mixture was concentrated under vacuum to afford the title compound (600 mg) as yellow oil, which was used for the next step without any further purification. LCMS [M+H]⁺=345.35.

### Step 4: Preparation of 2 fluoro-1-(3-((2-(4-(trifluoromethyl)phenyl)quinazolin-4-yl)amino)azetidin-1 yl)prop-2-en-1-one

HATU (663 mg, 1.74 mmol) was added with stirring into a mixture of N-(azetidin-3-yl)-2-(4-(trifluoromethyl)phenyl)quinazolin-4-amine (300 mg, 0.87 mmol), N,N-diisopropylethylamine (563 mg, 4.36 mmol), 2-fluoroacrylic acid (118 mg, 1.31 mmol), and DMF (5 mL), was stirred for 2 h at room temperature. The reaction was monitored by LCMS. The reaction mixture was then diluted with ethyl acetate, washed with water, dried over anhydrous sodium sulfate, and then filtered. The filtrate was concentrated under vacuum. The residue was purified by silica gel chromatography eluting with hexane:ethyl acetate=1.5:1 to afford the tittle compound (57 mg) as off white solid.

LCMS [M+H]⁺=417.43.

¹H NMR (500 MHz, CDCl₃) (δ 8.62 (d, J=8.1 Hz, 2H), 7.98-7.96 (m, 1H), 7.92 (d, J=8.2 Hz, 1H), 7.82-7.79 (m, 1H), 7.74 (d, J=8.2 Hz, 2H), 7.52 (t, J=7.6 Hz, 1H), 6.62 (m, 1H), 5.71-5.41 (m, 1H), 5.22-5.10 (m, 1H), 5.00-4.86 (m, 1H), 4.69-4.45 (m, 2H), 4.31-3.97 (m, 1H), 3.67-3.31 (m, 1H).

### Example 256 Synthesis of Compound 256 (2 fluoro-1-(3-(2-(4-(trifluoromethyl)phenyl)quinazolin-4 yl)azetidin-1 yl)prop-2-en-1-one)

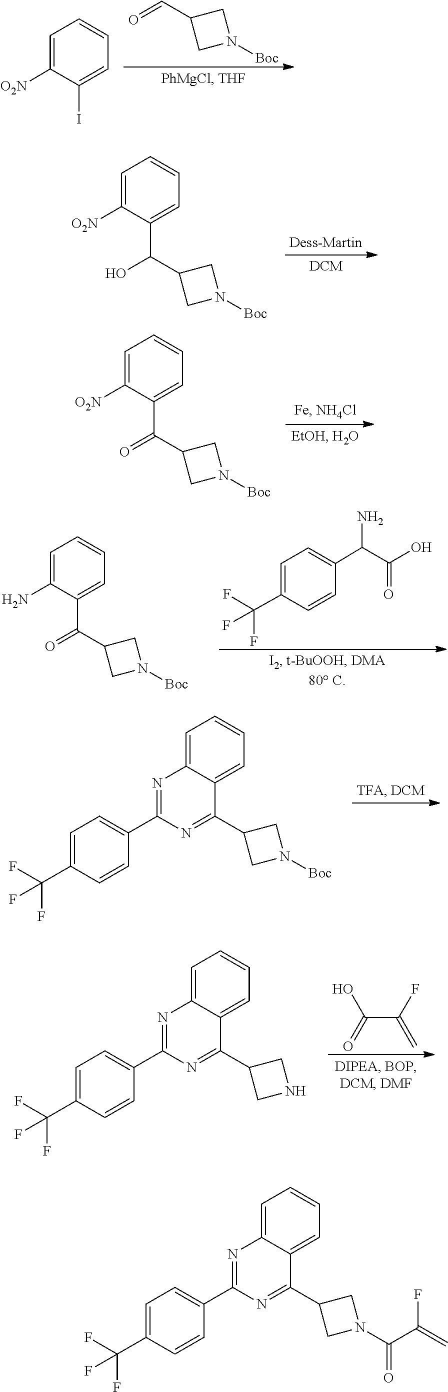

### Step 1: Preparation of tert-butyl 3-(hydroxy(2-nitrophenyl)methyl)azetidine-1-carboxylate

PhMgCl (7.23 mL, 2.00 mol/L, 14.46 mmol) was added dropwise into a solution of 1-iodo-2-nitrobenzene (3.00 g, 12.05 mmol) in THF (50 mL) at −60° C. under nitrogen atmosphere. The reaction was stirred for 30 minutes at −60° C. Then a solution of tert-butyl 3-formylazetidine-1-carboxylate (2.68 g, 14.46 mmol) in THF (6 mL) was added into the reaction. The mixture was warmed up to room temperature naturally and stirred for another 1 h. The reaction was monitored by LCMS. The reaction mixture was quenched by the addition of the saturated aqueous of NH₄C₁. The mixture was extracted with ethyl acetate, washed with brine, dried over anhydrous sodium sulfate, and then filtered. The filtrate was concentrated under vacuum. The residue was purified by silica gel chromatography eluting with EA:Hex=0-50% to afford the title compound (3.65 g, 98%) as a yellow solid. LCMS [M+H]⁺=309.14.

### Step 2: Preparation of tert-butyl 3-(2-nitrobenzoyl)azetidine-1-carboxylate

To a stirred solution of tert-butyl 3-(hydroxy(2-nitrophenyl)methyl)azetidine-1-carboxylate (3.7 g, 2.00 mmol) in DCM (50 mL) was added Dess-Martin (6.62 g, 15.60 mmol) in portions at 0° C. under nitrogen atmosphere. The reaction was stirred at room temperature for 2 h. The reaction was monitored by LCMS. The reaction mixture was quenched by the addition of sodium bicarbonate aqueous solution and sodium thiosulfate aqueous solution. The mixture was extracted with dichloroethane, washed with brine, dried over anhydrous sodium sulfate, and then filtered. The filtrate was concentrated under vacuum. The residue was purified by silica gel chromatography eluting with EA:Hex=0-30% to afford the title compound (3.4 g, 92.5%) as a yellow oil. LCMS [M+H]⁺=307.12.

### Step 3: Preparation of tert-butyl 3-(2-aminobenzoyl)azetidine-1-carboxylate

A mixture of tert-butyl 3-(2-nitrobenzoyl)azetidine-1-carboxylate (3.40 g, 11.10 mmol), iron powder (3.10 g, 55.50 mmol), and NH4Cl (2.97 g, 55.50 mmol) in EtOH (20 mL) and water (5 mL) was stirred for 3 h at 80° C. The reaction was monitored by LCMS. The reaction mixture was cooled down to room temperature and then filtered through a Celite pad. The filtrate was concentrated under vacuum. The residue was purified by silica gel column chromatograph eluting with EA:Hex=0-70% to afford the title compound (2.2 g, 72%) as a yellow oil. LCMS [M+H]⁺=277.15.

### Step 4: Preparation of tert-butyl 3-(2-(4-(trifluoromethyl)phenyl)quinazolin-4-yl)azetidine-1-carboxylate

A mixture of tert-butyl 3-(2-aminobenzoyl)azetidine-1-carboxylate (600 mg, 2.17 mmol), 2-amino-2-(4-(trifluoromethyl)phenyl)acetic acid (714 mg, 3.26 mmol), I2 (138 mg, 1.09 mmol), and 2-hydroperoxy-2-methylpropane (TBHP) (391 mg, 4.34 mmol) in DMA (15 mL) was stirred for 14 h at 80° C. The reaction was monitored by LCMS. The reaction was poured into water and extracted with ethyl acetate. The combined organic phase was washed with brine, dried over anhydrous sodium sulfate, and then filtered. The filtrate was concentrated under vacuum. The residue was purified by silica gel chromatography eluting with EA:Hex=0-30% to afford the title compound (610 mg, 65%) as a yellow oil. LCMS [M+H]⁺=430.17.

### Step 5: Preparation of 4-(azetidin-3 yl)-2-(4-(trifluoromethyl)phenyl)quinazoline

A mixture of tert-butyl 3-(2-(4-(trifluoromethyl)phenyl)quinazolin-4-yl)azetidine-1-carboxylate (610 mg, 1.42 mmol), dichloromethane (5 mL), and TFA (1 mL, 14.21 mmol) was stirred for 6 h at room temperature. The reaction was monitored by LCMS. The mixture was concentrated under vacuum. The residue was dissolved in dichloromethane. The pH value of the solution was adjusted to pH 10 with potassium carbonate aqueous solution. The mixture was extracted with dichloromethane, washed with brine, dried over anhydrous sodium sulfate, and filtered. The filtrate was concentrated under vacuum to afford the title compound (460 mg) as yellow solid, which was used for the next step without any further purification. LCMS [M+H]⁺=330.11.

### Step 6: Preparation of 2 fluoro-1-(3-(2-(4-(trifluoromethyl)phenyl)quinazolin-4-yl)azetidin-1 yl)prop-2-en-1-one

A mixture of 4-(azetidin-3-yl)-2-(4-(trifluoromethyl)phenyl) quinazoline (460 mg, 1.40 mmol), 2-fluoroacrylic acid (377 mg, 4.19 mmol), DIEA (1.39 mL, 8.38 mmol), dichloromethane (10 mL), DMF (2 mL), and 1H-benzotriazol-1-yloxytris(dimethylamino)phosphonium Hexafluorophosphate (BOP) (741 mg, 1.68 mmol) was stirred for 3 h at room temperature. The reaction was monitored by LCMS. The reaction was quenched by the addition of water and extracted with ethyl acetate. The combined organic phase was washed with brine, dried over anhydrous sodium sulfate, and then filtered. The filtrate was concentrated under vacuum. The residue was purified by silica gel chromatography eluting with EA:Hex=0-100% to afford the title compound (124 mg, 22%) as a white solid.

LCMS [M+H]⁺=402.12.

¹H NMR (500 MHz, DMSO-d₆) (58.80 (d, J=8.0 Hz, 2H), 8.15 (d, J=9.7 Hz, 2H), 8.07 (t, J=7.6 Hz, 1H), 7.97 (d, J=8.4 Hz, 2H), 7.79 (t, J=7.6 Hz, 1H), 5.53 (dd, J=48.5, 3.5 Hz, 1H), 5.34 (dd, J=16.6, 3.5 Hz, 1H), 5.03-4.95 (m, 2H), 4.87 (s, 1H), 4.68-4.51 (m, 2H).

### Example 257 Synthesis of Compound 257 (2 fluoro-1-(3-(4-(4-(trifluoromethyl)phenyl)quinazolin-2 yl)azetidin-1 yl)prop-2-en-1-one)

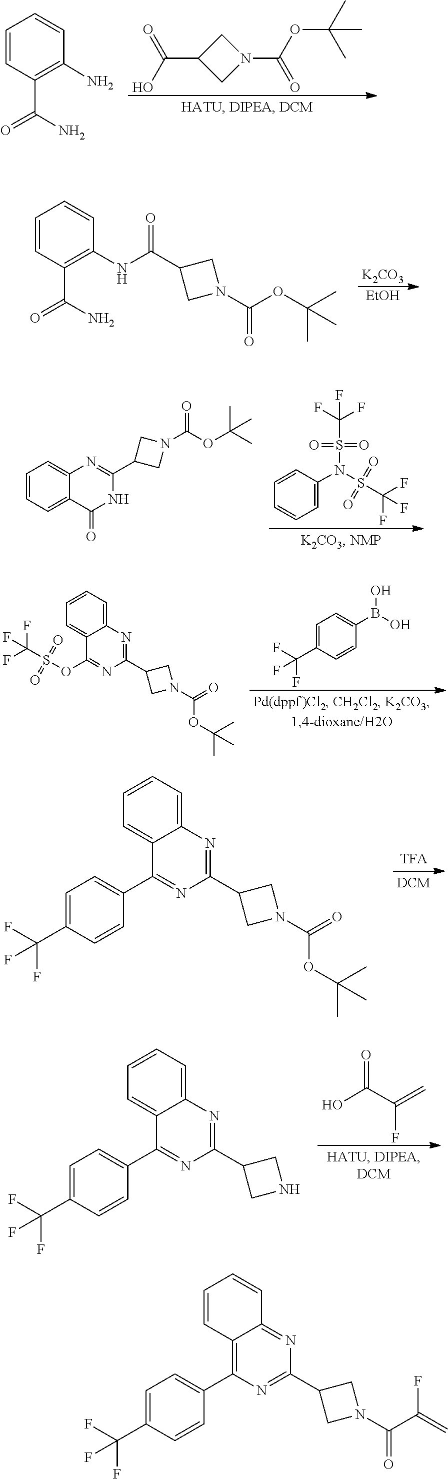

### Step 1: Preparation of tert-butyl 3-((2-carbamoylphenyl)carbamoyl)azetidine-1-carboxylate

HATU (12.57 g, 33.05 mmol) was added with stirring into a mixture of 2-aminobenzamide (4.05 g, 33.05 mmol), DIPEA (8.54 g, 66.10 mmol), 1-(tert-butoxycarbonyl)azetidine-3-carboxylic acid (4.66 g, 23.14 mmol), and DCM (20 mL) at 10° C. The mixture was stirred for 1 h at room temperature and the reaction was monitored by LCMS. The reaction was diluted with dichloromethane, poured into ice-water. The organic layer was separated, dried over anhydrous sodium sulfate, and then filtered. The filtrate was concentrated under vacuum. The residue was purified by silica gel chromatography eluting with hexane:EA=3:1 to afford the title compound (7.82 g) as white solid. LCMS [M+H]⁺=320.45

### Step 2: Preparation of tert-butyl 3-(4-oxo-3,4-dihydroquinazolin-2 yl)azetidine-1-carboxylate

A mixture of tert-butyl 3-((2-carbamoylphenyl)carbamoyl)azetidine-1-carboxylate (3.00 g, 9.39 mmol), potassium carbonate (12.98 g, 93.94 mmol), and EtOH (20 mL) was stirred for 6 h at 60° C. under nitrogen. The reaction was monitored by LCMS. The reaction was cooled down to room temperature. The mixture was concentrated under vacuum. The residue was added ice water and was neutralized with dilute hydrochloric acid to PH=4-5, and then filtered. The filter cake was washed with water and dried under vacuum to afford the title compound (2.32 g) as white solid, which was used for the next step without any further purification. LCMS [M+H]⁺=246.42.

### Step 3: Preparation of tert-butyl 3-(4-(((trifluoromethyl)sulfonyl)oxy)quinazolin-2-yl)azetidine-1-carboxylate

1,1,1-Trifluoro-N-phenyl-N-((trifluoromethyl)sulfonyl)methanesulfonamide (2.67 g, 7.74 mmol) was added with stirring into a mixture of tert-butyl 3-(4-oxo-3,4-dihydroquinazolin-2-yl)azetidine-1-carboxylate (1.50 g, 4.98 mmol), potassium carbonate (1.93 g, 14.93 mmol), and NMP (5.0 mL) at 0° C. under nitrogen atmosphere. The mixture was stirred for 2 h at room temperature. The reaction was monitored by LCMS. The reaction mixture was diluted with ethyl acetate, washed with water, dried over anhydrous sodium sulfate, and then filtered. The filtrate was concentrated under vacuum. The residue was purified by silica gel chromatography eluting with hexane:EA=4:1 to afford the title compound (1.60 g) as yellow oil. LCMS [M+H]⁺=378.26.

### Step 4: Preparation of tert-butyl 3-(4-(4-(trifluoromethyl)phenyl)quinazolin-2-yl)azetidine-1-carboxylate

A mixture of tert-butyl 3-(4-(((trifluoromethyl)sulfonyl)oxy)quinazolin-2-yl)azetidine-1-carboxylate (1.60 g, 3.69 mmol), (4-(trifluoromethyl)phenyl)boronic acid (1.05 g, 5.54 mmol), Pd(dppf)Cl₂·CH₂Cl₂ (300 mg, 0.37 mmol), potassium carbonate (1.53 g, 11.08 mmol), 1,4-dioxane (20 mL), and water (2.0 mL) was stirred for 6 h at 100° C. under nitrogen. The reaction was monitored by LCMS. The reaction was cooled down to room temperature. The mixture was concentrated under vacuum. The residue was purified by silica gel chromatography eluting with hexane:EA=3:1 to afford the title compound (1.40 g) as colorless oil. LCMS [M+H]⁺=374.44.

### Step 5: Preparation of 2-(azetidin-3 yl)-4-(4-(trifluoromethyl) phenyl)quinazoline

A mixture of tert-butyl 3-(4-(4-(trifluoromethyl) phenyl) quinazolin-2-yl)azetidine-1-carboxylate (1.40 g, 3.26 mmol), TFA (7.0 mL), and DCM (28 mL) was stirred for 8 h at room temperature. The reaction was monitored by LCMS. The reaction mixture was concentrated under vacuum. The residue was dissolved with dichloromethane. The pH value of the solution was adjusted to 10 with saturated sodium carbonate aqueous solution. The mixture was extracted with dichloromethane, washed with brine, dried over anhydrous sodium sulfate, and then filtered. The filtrate was concentrated under vacuum to afford the title compound (0.92 g) as colorless oil, which was used for the next step without any further purification. LCMS [M+H]⁺=330.23.

### Step 6: Preparation oft fluoro-1-(3-(4-(4-(trifluoromethyl)phenyl)quinazolin-2-yl)azetidin-1 yl)prop-2-en-1-one

A mixture of 2-(azetidin-3-yl)-4-(4-(trifluoromethyl) phenyl)quinazoline (900 mg, 2.73 mmol), DIPEA (1.06 g, 8.20 mmol), DCM (20 mL), 2-fluoroacrylic acid (370 mg, 4.10 mmol), and HATU (1.04 g, 2.73 mmol) was stirred for 1 h at room temperature. The reaction was monitored by LCMS. The reaction mixture was then diluted with ethyl acetate, washed with water, dried over anhydrous sodium sulfate, and then filtered. The filtrate was concentrated under vacuum. The residue was purified by silica gel chromatography eluting with Hex:EA=2:1 to afford the tittle compound (55 mg) as white solid.

LCMS [M+H]⁺=402.43.

¹H NMR (500 MHz, DMSO-d₆) δ 8.27-7.86 (m, 7H), 7.75 (ddd, J=8.3, 6.7, 1.4 Hz, 1H), 5.50 (dd, J=48.5, 3.5 Hz, 1H), 5.32 (dd, J=16.6, 3.5 Hz, 1H), 4.86 (td, J=8.9, 8.5, 4.0 Hz, 1H), 4.81-4.69 (m, 1H), 4.47 (t, J=9.3 Hz, 1H), 4.44-4.29 (m, 2H).

The compounds of table 3 were prepared in a similar manner to Examples 1-257 via different reaction starting materials and suitable reagents.

TABLE 3

| EX | | | Physical Data |
|---|---|---|---|
| No. | Structure | Chemical Name | (LCMS) (M + H)⁺ |
| 258 | 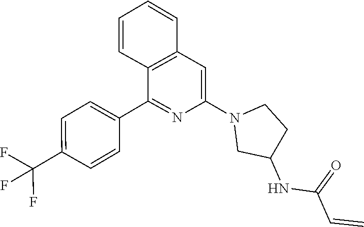 [SMILES: C=CC(=O)NC1CCN(c2cc3ccccc3c(-c3ccc(C(F)(F)F)cc3)n2)C1] | N-(1-(1-(4- (trifluoromethyl)phenyl) isoquinolin-3-yl)pyrrolidin-3- yl)acrylamide | 412.2 |
| 259 | 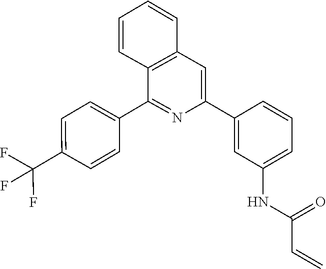 [SMILES: C=CC(=O)Nc1cccc(-c2cc3ccccc3c(-c3ccc(C(F)(F)F)cc3)n2)c1] | N-(3-(1-(4- (trifluoromethyl)phenyl) isoquinolin-3-yl)phenyl) acrylamide | 419.1 |
| 260 | 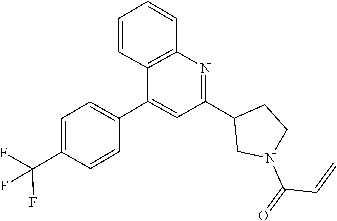 [SMILES: C=CC(=O)N1CCC(c2cc(-c3ccc(C(F)(F)F)cc3)c3ccccc3n2)C1] | 1-(3-(4-(4- (trifluoromethyl)phenyl)quinolin- 2-yl)pyrrolidin-1-yl)prop-2-en-1- one | 397.1 |
| 261 | 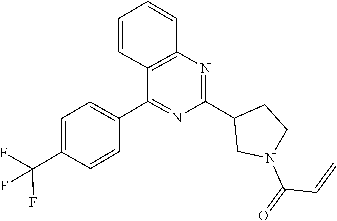 [SMILES: C=CC(=O)N1CCC(c2nc(-c3ccc(C(F)(F)F)cc3)c3ccccc3n2)C1] | 1-(3-(4-(4- (trifluoromethyl)phenyl) quinazolin-2-yl)pyrrolidin-1-yl) prop-2-en-1-one | 398.1 |
| 262 | 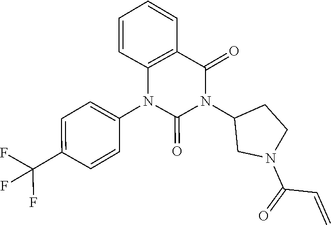 [SMILES: C=CC(=O)N1CCC(n2c(=O)c3ccccc3n(-c3ccc(C(F)(F)F)cc3)c2=O)C1] | 3-(1-acryloylpyrrolidin-3-yl)-1- (4- (trifluoromethyl)phenyl) quinazoline-2,4(1H,3H)-dione | 430.1 |
| 263 | 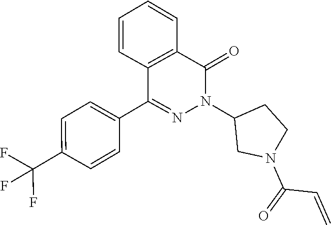 [SMILES: C=CC(=O)N1CCC(n2nc(-c3ccc(C(F)(F)F)cc3)c3ccccc3c2=O)C1] | 2-(1-acryloylpyrrolidin-3-yl)-4- (4- (trifluoromethyl)phenyl) phthalazin-1(2H)-one | 414.1 |
| 264 | 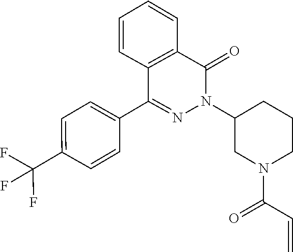 [SMILES: C=CC(=O)N1CCCC(n2nc(-c3ccc(C(F)(F)F)cc3)c3ccccc3c2=O)C1] | 2-(1-acryloylpiperidin-3-yl)-4-(4- (trifluoromethyl)phenyl) phthalazin-1(2H)-one | 428.2 |
| 265 | 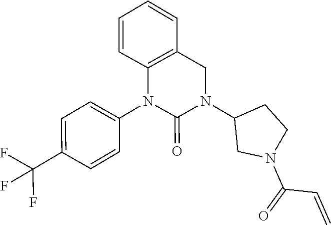 [SMILES: C=CC(=O)N1CCC(N2Cc3ccccc3N(c3ccc(C(F)(F)F)cc3)C2=O)C1] | 3-(1-acryloylpyrrolidin-3-yl)-1- (4-(trifluoromethyl)phenyl)-3,4- dihydroquinazolin-2(1H)-one | 416.2 |
| 266 | 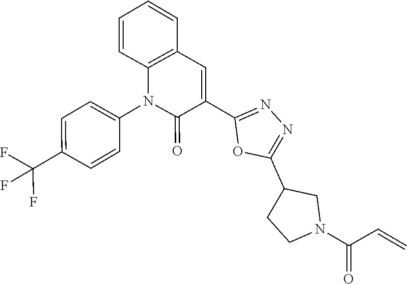 [SMILES: C=CC(=O)N1CCC(c2nnc(-c3cc4ccccc4n(-c4ccc(C(F)(F)F)cc4)c3=O)o2)C1] | 3-(5-(1-acryloylpyrrolidin-3-yl)- 1,3,4-oxadiazol-2-yl)-1-(4- (trifluoromethyl)phenyl)quinolin- 2(1H)-one | 481.1 |
| 267 | 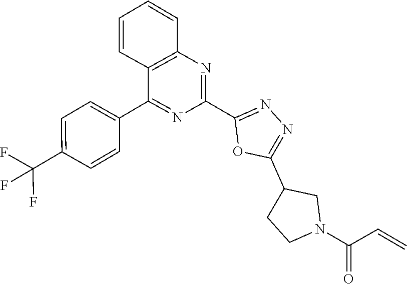 [SMILES: C=CC(=O)N1CCC(c2nnc(-c3nc(-c4ccc(C(F)(F)F)cc4)c4ccccc4n3)o2)C1] | 1-(3-(5-(4-(4- (trifluoromethyl)phenyl) quinazolin-2-yl)-1,3,4-oxadiazol- 2-yl)pyrrolidin-1-yl)prop-2-en-1- one | 466.1 |
| 268 | 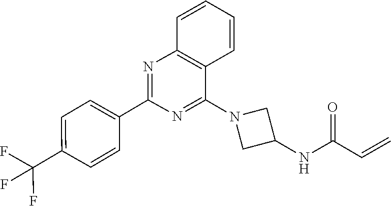 [SMILES: C=CC(=O)NC1CN(c2nc(-c3ccc(C(F)(F)F)cc3)nc3ccccc23)C1] | N-(1-(2-(4- (trifluoromethyl)phenyl) quinazolin- 4-yl)azetidin-3-yl)acrylamide | 399.1 |
| 269 | 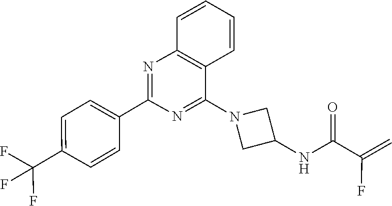 [SMILES: C=C(F)C(=O)NC1CN(c2nc(-c3ccc(C(F)(F)F)cc3)nc3ccccc23)C1] | 2-fluoro-N-(1-(2-(4- (trifluoromethyl)phenyl) quinazolin- 4-yl)azetidin-3-yl)acrylamide | 417.1 |
| 270 | 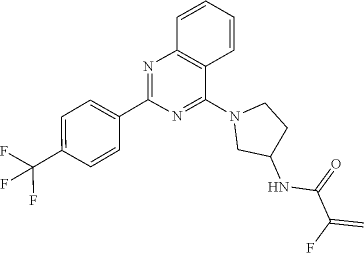 [SMILES: C=C(F)C(=O)NC1CCN(c2nc(-c3ccc(C(F)(F)F)cc3)nc3ccccc23)C1] | 2-fluoro-N-(1-(2-(4- (trifluoromethyl)phenyl) quinazolin- 4-yl)pyrrolidin-3-yl)acrylamide | 431.1 |
| 271 | 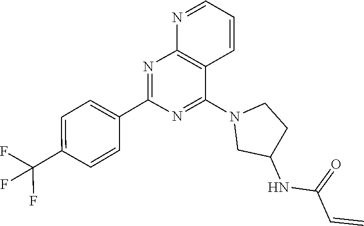 [SMILES: C=CC(=O)NC1CCN(c2nc(-c3ccc(C(F)(F)F)cc3)nc3ncccc23)C1] | N-(1-(2-(4- (trifluoromethyl)phenyl)pyrido[2, 3-d]pyrimidin-4-yl)pyrrolidin-3- yl)acrylamide | 414.2 |
| 272 | 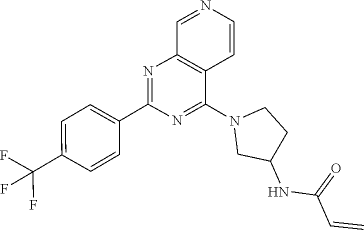 [SMILES: C=CC(=O)NC1CCN(c2nc(-c3ccc(C(F)(F)F)cc3)nc3cnccc23)C1] | N-(1-(2-(4- (trifluoromethyl)phenyl)pyrido[3, 4-d]pyrimidin-4-yl)pyrrolidin-3- yl)acrylamide | 414.2 |
| 273 | 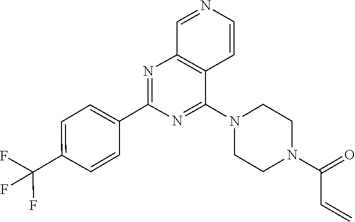 [SMILES: C=CC(=O)N1CCN(c2nc(-c3ccc(C(F)(F)F)cc3)nc3cnccc23)CC1] | 1-(4-(2-(4- (trifluoromethyl)phenyl)pyrido[3, 4-d]pyrimidin-4-yl)piperazin-1- yl)prop-2-en-1-one | 414.2 |
| 274 | 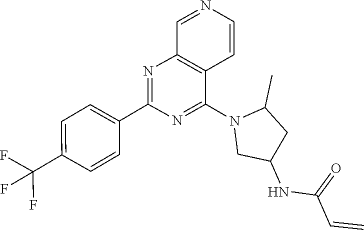 [SMILES: C=CC(=O)NC1CC(C)N(c2nc(-c3ccc(C(F)(F)F)cc3)nc3cnccc23)C1] | N-(5-methyl-1-(2-(4- (trifluoromethyl)phenyl)pyrido[3, 4-d]pyrimidin-4-yl)pyrrolidin-3- yl)acrylamide | 428.1 |
| 275 | 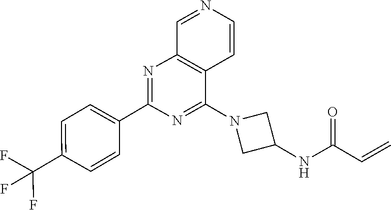 [SMILES: C=CC(=O)NC1CN(c2nc(-c3ccc(C(F)(F)F)cc3)nc3cnccc23)C1] | N-(1-(2-(4- (trifluoromethyl)phenyl)pyrido[2, 3-d]pyrimidin-4-yl)azetidin-3- yl)acrylamide | 400.1 |
| 276 | 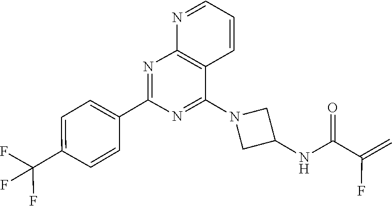 [SMILES: C=C(F)C(=O)NC1CN(c2nc(-c3ccc(C(F)(F)F)cc3)nc3ncccc23)C1] | 2-fluoro-N-(1-(2-(4- (trifluoromethyl)phenyl)pyrido[2, 3-d]pyrimidin-4-yl)azetidin-3- yl)acrylamide | 418.1 |
| 277 | 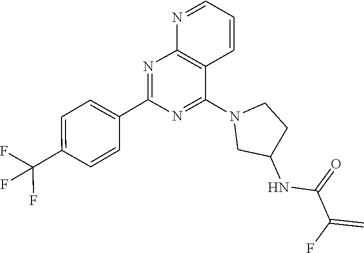 [SMILES: C=C(F)C(=O)NC1CCN(c2nc(-c3ccc(C(F)(F)F)cc3)nc3ncccc23)C1] | 2-fluoro-N-(1-(2-(4- (trifluoromethyl)phenyl)pyrido[2, 3-d]pyrimidin-4-yl)pyrrolidin-3- yl)acrylamide | 432.1 |
| 278 | 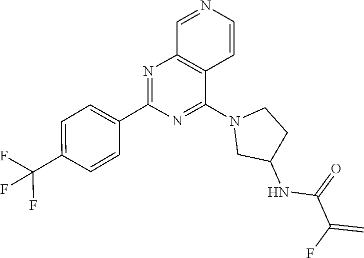 [SMILES: C=C(F)C(=O)NC1CCN(c2nc(-c3ccc(C(F)(F)F)cc3)nc3cnccc23)C1] | 2-fluoro-N-(1-(2-(4- (trifluoromethyl)phenyl)pyrido[3, 4-d]pyrimidin-4-yl)pyrrolidin-3- yl)acrylamide | 432.1 |
| 279 | 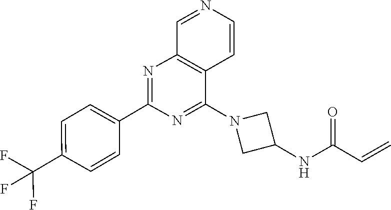 [SMILES: C=CC(=O)NC1CN(c2nc(-c3ccc(C(F)(F)F)cc3)nc3cnccc23)C1] | N-(1-(2-(4- (trifluoromethyl)phenyl)pyrido[3, 4-d]pyrimidin-4-yl)azetidin-3- yl)acrylamide | 400.1 |
| 280 | 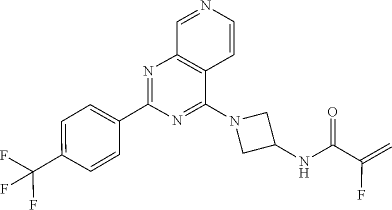 [SMILES: C=C(F)C(=O)NC1CN(c2nc(-c3ccc(C(F)(F)F)cc3)nc3cnccc23)C1] | 2-fluoro-N-(1-(2-(4- (trifluoromethyl)phenyl)pyrido[3, 4-d]pyrimidin-4-yl)azetidin-3- yl)acrylamide | 418.1 |
| 281 | 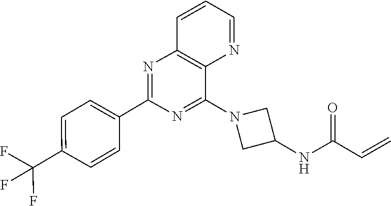 [SMILES: C=CC(=O)NC1CN(c2nc(-c3ccc(C(F)(F)F)cc3)nc3cccnc23)C1] | N-(1-(2-(4- (trifluoromethyl)phenyl)pyrido[3, 2-d]pyrimidin-4-yl)azetidin-3- yl)acrylamide | 400.1 |
| 282 | 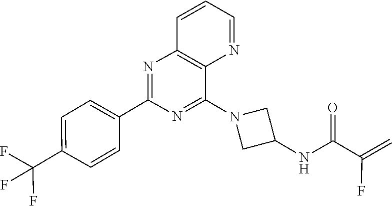 [SMILES: C=C(F)C(=O)NC1CN(c2nc(-c3ccc(C(F)(F)F)cc3)nc3cccnc23)C1] | 2-fluoro-N-(1-(2-(4- (trifluoromethyl)phenyl)pyrido[3, 2-d]pyrimidin-4-yl)azetidin-3- yl)acrylamide | 418.1 |
| 283 | 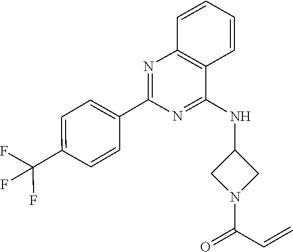 [SMILES: C=CC(=O)N1CC(Nc2nc(-c3ccc(C(F)(F)F)cc3)nc3ccccc23)C1] | 1-(3-((2-(4- (trifluoromethyl)phenyl) quinazolin-4-yl)amino)azetidin- 1-yl)prop-2-en-1-one | 399.1 |
| 284 | 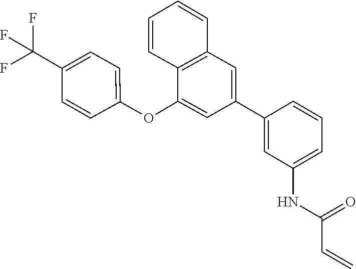 [SMILES: C=CC(=O)Nc1cccc(-c2cc(Oc3ccc(C(F)(F)F)cc3)c3ccccc3c2)c1] | N-(3-(4-(4- (trifluoromethyl)phenoxy) naphthalen-2-yl)phenyl) acrylamide | 434.1 |
| 285 | 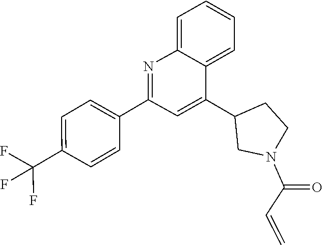 [SMILES: C=CC(=O)N1CCC(c2cc(-c3ccc(C(F)(F)F)cc3)nc3ccccc23)C1] | 1-(3-(2-(4- (trifluoromethyl)phenyl)quinolin- 4-yl)pyrrolidin-1-yl)prop-2-en-1- one | 397.1 |
| 286 | 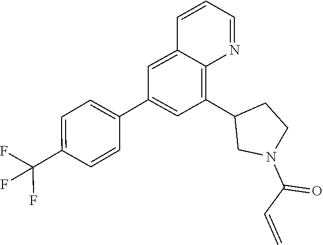 [SMILES: C=CC(=O)N1CCC(c2cc(-c3ccc(C(F)(F)F)cc3)cc3cccnc23)C1] | 1-(3-(6-(4- (trifluoromethyl)phenyl)quinolin- 8-yl)pyrrolidin-1-yl)prop-2-en-1- one | 397.1 |
| 287 | 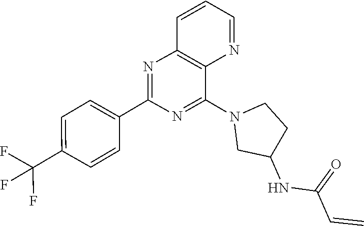 [SMILES: C=CC(=O)NC1CCN(c2nc(-c3ccc(C(F)(F)F)cc3)nc3cccnc23)C1] | N-(1-(2-(4- (trifluoromethyl)phenyl)pyrido[3, 2-d]pyrimidin-4-yl)pyrrolidin-3- yl)acrylamide | 414.2 |
| 288 | 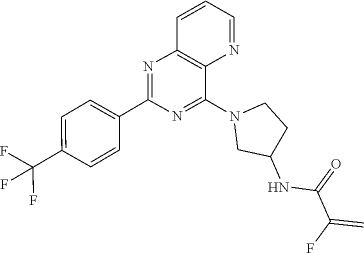 [SMILES: C=C(F)C(=O)NC1CCN(c2nc(-c3ccc(C(F)(F)F)cc3)nc3cccnc23)C1] | 2-fluoro-N-(1-(2-(4- (trifluoromethyl)phenyl)pyrido[3, 2-d]pyrimidin-4-yl)pyrrolidin-3- yl)acrylamide | 432.1 |
| 289 | 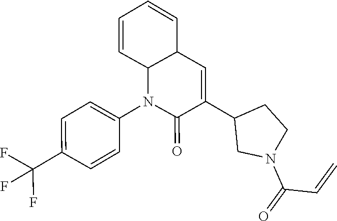 [SMILES: C=CC(=O)N1CCC(C2=CC3C=CC=CC3N(c3ccc(C(F)(F)F)cc3)C2=O)C1] | 3-(1-acryloylpyrrolidin-3-yl)-1- (4-(trifluoromethyl)phenyl)- 4a,8a-dihydroquinolin-2(1H)-one | 415.2 |
| 290 | 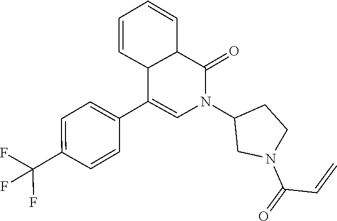 [SMILES: C=CC(=O)N1CCC(N2C=C(c3ccc(C(F)(F)F)cc3)C3C=CC=CC3C2=O)C1] | 2-(1-acryloylpyrrolidin-3-yl)-4- (4-(trifluoromethyl)phenyl)- 4a,8a-dihydroisoquinolin-1(2H)- one | 415.2 |
| 291 | 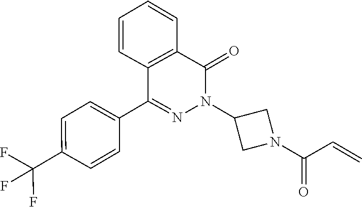 [SMILES: C=CC(=O)N1CC(n2nc(-c3ccc(C(F)(F)F)cc3)c3ccccc3c2=O)C1] | 2-(1-acryloylazetidin-3-yl)-4-(4- (trifluoromethyl)phenyl) phthalazin-1(2H)-one | 400.1 |
| 292 | 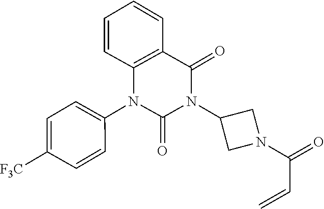 [SMILES: C=CC(=O)N1CC(n2c(=O)c3ccccc3n(-c3ccc(C(F)(F)F)cc3)c2=O)C1] | 3-(1-acryloylazetidin-3-yl)-1-(4- (trifluoromethyl)phenyl) quinazoline-2,4(1H,3H)-dione | 416.1 |
| 293 | 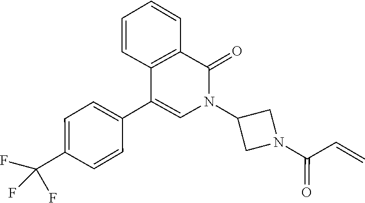 [SMILES: C=CC(=O)N1CC(n2cc(-c3ccc(C(F)(F)F)cc3)c3ccccc3c2=O)C1] | 2-(1-acryloylazetidin-3-yl)-4-(4- (trifluoromethyl)phenyl) isoquinolin-1(2H)-one | 399.1 |
| 294 | 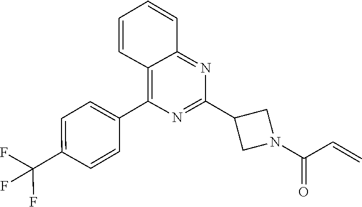 [SMILES: C=CC(=O)N1CC(c2nc(-c3ccc(C(F)(F)F)cc3)c3ccccc3n2)C1] | 1-(3-(4-(4- (trifluoromethyl)phenyl) quinazolin-2-yl)azetidin-1-yl) prop-2-en-1-one | 384.1 |
| 295 | 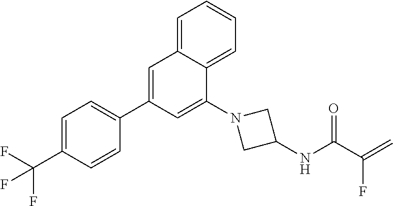 [SMILES: C=C(F)C(=O)NC1CN(c2cc(-c3ccc(C(F)(F)F)cc3)cc3ccccc23)C1] | 2-fluoro-N-(1-(3-(4- (trifluoromethyl)phenyl) naphthalen- 1-yl)azetidin-3-yl)acrylamide | 415.1 |
| 296 | 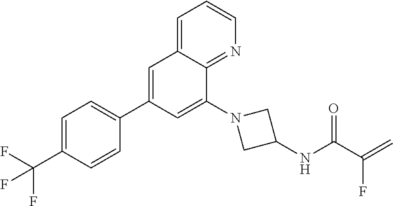 [SMILES: C=C(F)C(=O)NC1CN(c2cc(-c3ccc(C(F)(F)F)cc3)cc3cccnc23)C1] | 2-fluoro-N-(1-(6-(4- (trifluoromethyl)phenyl)quinolin- 8-yl)azetidin-3-yl)acrylamide | 416.1 |
| 297 | 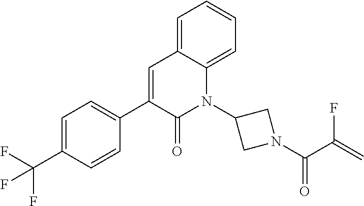 [SMILES: C=C(F)C(=O)N1CC(n2c(=O)c(-c3ccc(C(F)(F)F)cc3)cc3ccccc32)C1] | 1-(1-(2-fluoroacryloyl)azetidin-3- yl)-3-(4- (trifluoromethyl)phenyl)quinolin- 2(1H)-one | 417.1 |
| 298 | 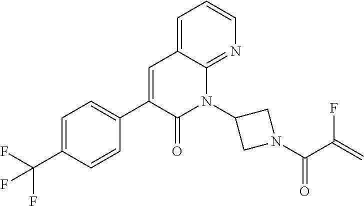 [SMILES: C=C(F)C(=O)N1CC(n2c(=O)c(-c3ccc(C(F)(F)F)cc3)cc3cccnc32)C1] | 1-(1-(2-fluoroacryloyl)azetidin-3- yl)-3-(4- (trifluoromethyl)phenyl)-1,8- naphthyridin-2(1H)-one | 418.1 |
| 299 | 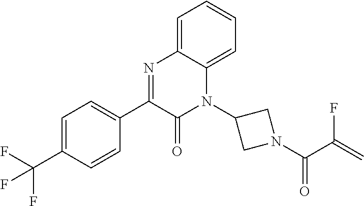 [SMILES: C=C(F)C(=O)N1CC(n2c(=O)c(-c3ccc(C(F)(F)F)cc3)nc3ccccc32)C1] | 1-(1-(2-fluoroacryloyl)azetidin-3- yl)-3-(4- (trifluoromethyl)phenyl) quinoxalin-2(1H)-one | 418.1 |
| 300 | 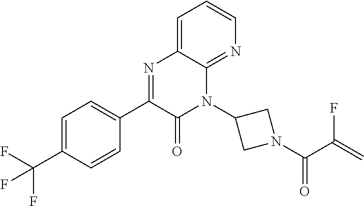 [SMILES: C=C(F)C(=O)N1CC(n2c(=O)c(-c3ccc(C(F)(F)F)cc3)nc3cccnc32)C1] | 4-(1-(2-fluoroacryloyl)azetidin-3- yl)-2-(4- (trifluoromethyl)phenyl)pyrido[2, 3-b]pyrazin-3(4H)-one | 419.1 |
| 301 | 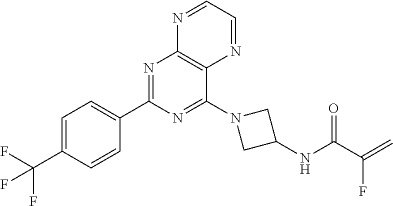 [SMILES: C=C(F)C(=O)NC1CN(c2nc(-c3ccc(C(F)(F)F)cc3)nc3nccnc23)C1] | 2-fluoro-N-(1-(2-(4- (trifluoromethyl)phenyl)pteridin- 4-yl)azetidin-3-yl)acrylamide | 419.1 |
| 302 | 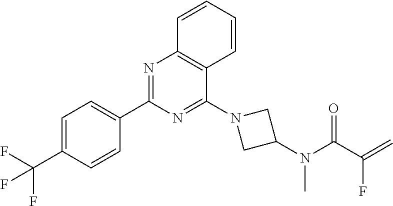 [SMILES: C=C(F)C(=O)N(C)C1CN(c2nc(-c3ccc(C(F)(F)F)cc3)nc3ccccc23)C1] | 2-fluoro-N-methyl-N-(1-(2-(4- (trifluoromethyl)phenyl) quinazolin- 4-yl)azetidin-3-yl)acrylamide | 431.1 |
| 303 | 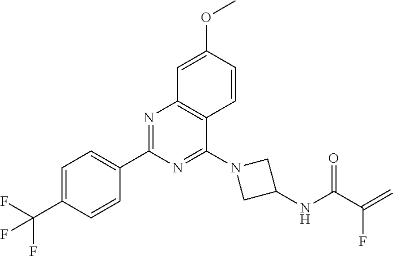 [SMILES: C=C(F)C(=O)NC1CN(c2nc(-c3ccc(C(F)(F)F)cc3)nc3cc(OC)ccc23)C1] | 2-fluoro-N-(1-(7-methoxy-2-(4- (trifluoromethyl)phenyl) quinazolin- 4-yl)azetidin-3-yl)acrylamide | 447.1 |
| 304 | 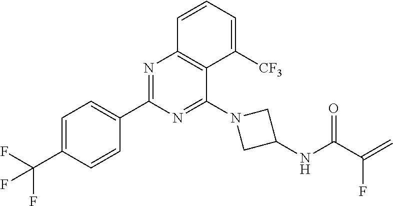 [SMILES: C=C(F)C(=O)NC1CN(c2nc(-c3ccc(C(F)(F)F)cc3)nc3cccc(C(F)(F)F)c23)C1] | 2-fluoro-N-(1-(5- (trifluoromethyl)-2-(4- (trifluoromethyl)phenyl) quinazolin- 4-yl)azetidin-3-yl)acrylamide | 485.1 |
| 305 | 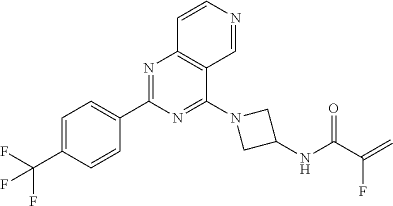 [SMILES: C=C(F)C(=O)NC1CN(c2nc(-c3ccc(C(F)(F)F)cc3)nc3ccncc23)C1] | 2-fluoro-N-(1-(2-(4- (trifluoromethyl)phenyl)pyrido[4, 3-d]pyrimidin-4-yl)azetidin-3- yl)acrylamide | 418.1 |
| 306 | 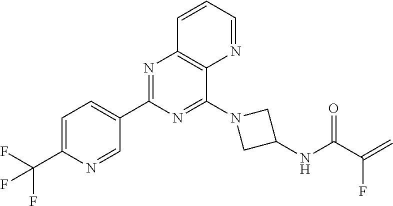 [SMILES: C=C(F)C(=O)NC1CN(c2nc(-c3ccc(C(F)(F)F)nc3)nc3cccnc23)C1] | 2-fluoro-N-(1-(2-(6- (trifluoromethyl)pyridin-3- yl)pyrido[3,2-d]pyrimidin-4- yl)azetidin-3-yl)acrylamide | 419.2 |

### Example 307 Synthesis of Compound 307 (2 fluoro-1-(3-(3-(phenylethynyl)-1H-pyrazolo[3,4-b]pyridin-1 yl)azetidin-1 yl)prop-2-en-1-one)

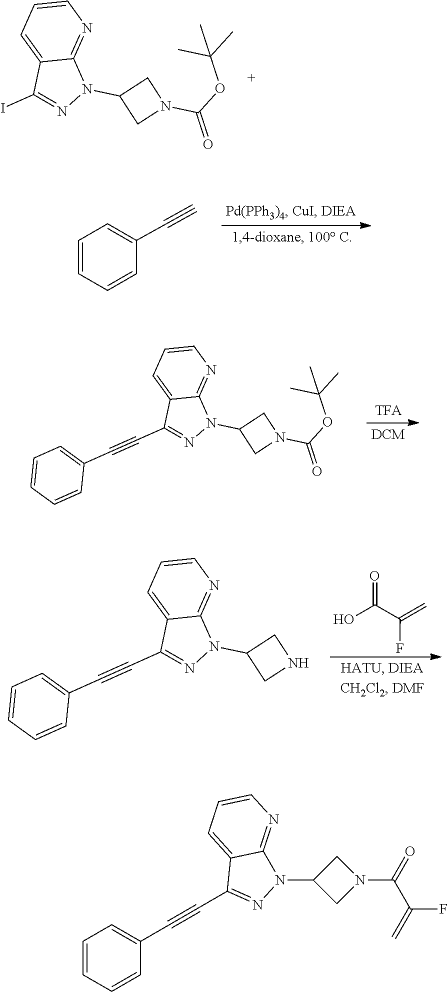

### Step 1: Preparation of tert-butyl 3-(3-(phenylethynyl)-1H-pyrazolo[3,4-b]pyridin-1-yl)azetidine-1-carboxylate

A mixture of tert-butyl 3-(3-iodo-1H-pyrazolo[3,4-b]pyridin-1-yl)azetidine-1-carboxylate (0.40 g, 1 mmol), ethynylbenzene (0.20 g, 2 mmol), DIEA (0.50 mL, 3 mmol), Pd(PPh₃)₄ (0.12 g, 0.1 mmol), CuI (0.08 g, 0.4 mmol), 1,4-dioxane (40 mL) was stirred for 4 h at 100° C. under nitrogen. The mixture was concentrated under vacuum to get the residue, the residue was washed with water for three times and brine for once, the organic phase was concentrated under vacuum. The residue was further purified by silica gel chromatography eluting with DCM:MeOH=30:1 to afford the title compound (0.50 g) as light yellow solid. LCMS [M+H]⁺=375.17.

### Step 2: Preparation of 1-(azetidin-3 yl)-3-(phenylethynyl)-1H-pyrazolo[3,4-b]pyridine

A mixture of tert-butyl 3-(3-(phenylethynyl)-1H-pyrazolo[3,4-b]pyridin-1-yl)azetidine-1-carboxylate (0.37 g, 1 mmol), TFA (5 mL) and DCM (10 mL) was stirred for 1 h at room temperature. The reaction mixture was concentrated under vacuum to afford the title compound, which was used for the next step without any further purification. LCMS [M+H]⁺=275.12.

### Step 3: Preparation of 2 fluoro-1-(3-(3-(phenylethynyl)-1H-pyrazolo[3,4-b]pyridin-1-yl)azetidin-1 yl)prop-2-en-1-one

A mixture of 1-(azetidin-3-yl)-3-(phenylethynyl)-1H-pyrazolo[3,4-b]pyridine (0.18 g, 0.66 mmol), 2-fluoroacrylic acid (0.09 g, 1 mmol), HATU (0.50 g, 1.32 mmol), DIEA (0.55 mL, 3.3 mmol), DCM (20 mL) and DMF (1 mL) was stirred for overnight at room temperature. The reaction mixture was washed with water and brine, dried over anhydrous sodium sulfate, and filtered. The filtrate was concentrated under vacuum. The residue was further purified by silica gel chromatography eluting with ethyl acetate to afford the title compound (0.08 g) as off white solid.

LCMS [M+H]⁺=347.12.

¹H NMR (500 MHz, CDCl₃) (δ 8.58 (dd, J=4.5, 1.5 Hz, 1H), 8.22 (dd, J=8.0, 1.5 Hz, 1H), 7.68-7.62 (m, 2H), 7.43-7.36 (m, 3H), 7.27 (dd, J=8.0, 4.5 Hz, 1H), 6.04-5.95 (m, 1H), 5.69 (dd, J=46.7, 3.1 Hz, 1H), 5.13 (dd, J=15.6, 3.1 Hz, 1H), 5.10-5.03 (m, 1H), 4.97-4.88 (m, 1H), 4.76-4.70 (m, 1H), 4.70-4.63 (m, 1H).

### Example 308 Synthesis of Compound 308 ((E)-2 fluoro-1-(3-(3-styryl-1H-pyrazolo[3,4-b]pyridin-1 yl)azetidin-1 yl)prop-2-en-1-one)

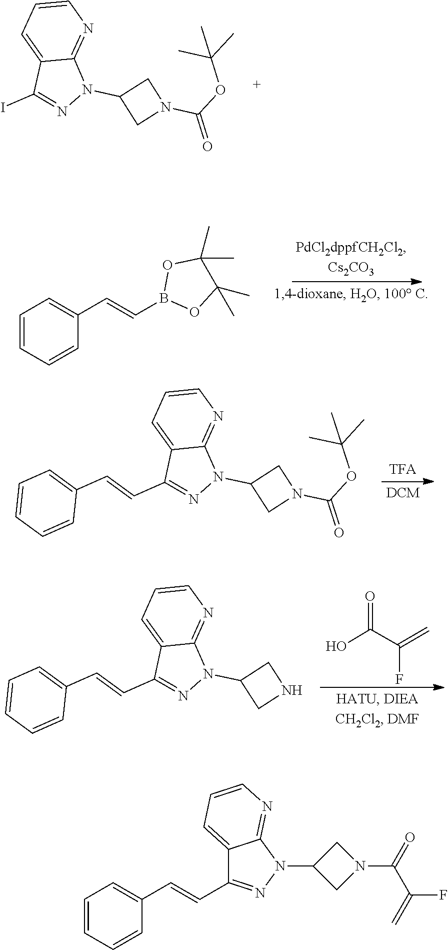

### Step 1: Preparation of tert-butyl (E)-3-(3-styryl-1H-pyrazolo[3,4-b]pyridin-1-yl)azetidine-1-carboxylate

A mixture of tert-butyl 3-(3-iodo-1H-pyrazolo[3,4-b]pyridin-1-yl)azetidine-1-carboxylate (0.50 g, 1.25 mmol), (E)-4,4,5,5-tetramethyl-2-styryl-1,3,2-dioxaborolane (0.43 g, 1.87 mmol), Cs₂CO₃ (1.22 g, 3.75 mmol), Pd(dppf)Cl₂CH₂Cl₂ (0.10 g, 0.12 mmol), 1,4-dioxane (20 mL), and water (4 mL) was stirred for overnight at 100° C. under nitrogen. The mixture was concentrated under vacuum. The residue was further purified by silica gel chromatography eluting with DCM:MeOH=30:1 to afford the title compound (0.50 g). LCMS [M+H]⁺=377.19.

### Step 2: Preparation of (E)-1-(azetidin-3 yl)-3-styryl-1H-pyrazolo[3,4-b]pyridine

A mixture of tert-butyl (E)-3-(3-styryl-1H-pyrazolo[3,4-b]pyridin-1-yl)azetidine-1-carboxylate (0.50 g, 1.33 mmol), TFA (5 mL) and DCM (10 mL) was stirred for 1 h at room temperature. The reaction mixture was concentrated under vacuum to afford the title compound, which was used for the next step without any further purification. LCMS [M+H]⁺=277.14.

### Step 3: Preparation of (E)-2 fluoro-1-(3-(3-styryl-1H-pyrazolo[3,4-b]pyridin-1-yl)azetidin-1 yl)prop-2-en-1-one

A mixture of (E)-1-(azetidin-3-yl)-3-styryl-1H-pyrazolo[3,4-b]pyridine (0.18 g, 0.66 mmol), 2-fluoroacrylic acid (0.09 g, 1 mmol), HATU (0.50 g, 1.32 mmol), DIEA (0.55 mL, 3.3 mmol), DCM (20 mL) and DMF (1 mL) was stirred for overnight at room temperature. The reaction mixture was washed with water and brine, dried over anhydrous sodium sulfate, and filtered. The filtrate was concentrated under vacuum. The residue was further purified by silica gel chromatography eluting with ethyl acetate to afford the title compound (0.12 g) as off white solid.

LCMS [M+H]⁺=349.14.

¹H NMR (500 MHz, CDCl₃) (δ 8.55 (dd, J=4.5, 1.4 Hz, 1H), 8.37 (dd, J=8.1, 1.4 Hz, 1H), 7.60 (d, J=7.3 Hz, 2H), 7.50 (d, J=16.7 Hz, 1H), 7.45-7.38 (m, 3H), 7.32 (t, J=7.3 Hz, 1H), 7.24 (dd, J=8.0, 4.5 Hz, 1H), 6.01-5.93 (m, 1H), 5.70 (dd, J=46.6, 3.0 Hz, 1H), 5.15 (dd, J=15.6, 3.0 Hz, 1H), 5.08-5.00 (m, 1H), 4.97-4.89 (m, 1H), 4.78-4.71 (m, 1H), 4.69-4.62 (m, 1H).

The compounds of table 4 were prepared in a similar manner to Examples 1-308 via different reaction starting materials and suitable reagents.

TABLE 4

| EX | | | Physical Data |
|---|---|---|---|
| No. | Structure | Chemical Name | (LCMS) (M + H)⁺ |
| 309 | 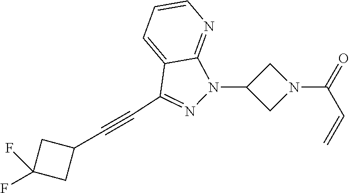 [SMILES: C=CC(=O)N1CC(n2nc(C#CC3CC(F)(F)C3)c3cccnc32)C1] | 1-(3-(3-((3,3- difluorocyclobutyl)ethynyl)-1H- pyrazolo[3,4-b]pyridin-1- yl)azetidin-1-yl)prop-2-en-1-one | 343.1 |
| 310 | 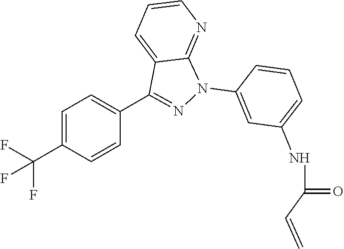 [SMILES: C=CC(=O)Nc1cccc(-n2nc(-c3ccc(C(F)(F)F)cc3)c3cccnc32)c1] | N-(3-(3-(4- (trifluoromethyl)phenyl)-1H- pyrazolo[3,4-b]pyridin-1- yl)phenyl)acrylamide | 409.1 |
| 311 | 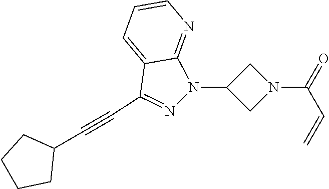 [SMILES: C=CC(=O)N1CC(n2nc(C#CC3CCCC3)c3cccnc32)C1] | 1-(3-(3-(cyclopentylethynyl)-1H- pyrazolo[3,4-b]pyridin-1- yl)azetidin-1-yl)prop-2-en-1-one | 321.2 |
| 312 | 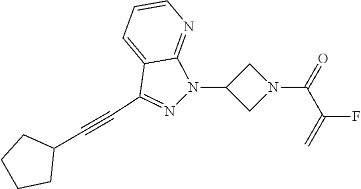 [SMILES: C=C(F)C(=O)N1CC(n2nc(C#CC3CCCC3)c3cccnc32)C1] | 1-(3-(3-(cyclopentylethynyl)-1H- pyrazolo[3,4-b]pyridin-1- yl)azetidin-1-yl)-2-fluoroprop-2- en-1-one | 339.2 |
| 313 | 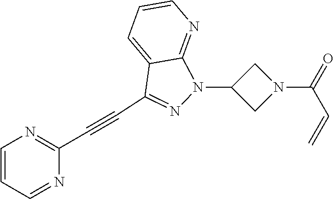 [SMILES: C=CC(=O)N1CC(n2nc(C#Cc3ncccn3)c3cccnc32)C1] | 1-(3-(3-(pyrimidin-2-ylethynyl)- 1H-pyrazolo[3,4-b]pyridin-1- yl)azetidin-1-yl)prop-2-en-1-one | 331.2 |
| 314 | 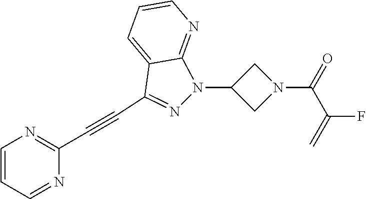 [SMILES: C=C(F)C(=O)N1CC(n2nc(C#Cc3ncccn3)c3cccnc32)C1] | 2-fluoro-1-(3-(3-(pyrimidin-2- ylethynyl)-1H-pyrazolo[3,4- b]pyridin-1-yl)azetidin-1- yl)prop-2-en-1-one | 349.1 |
| 315 | 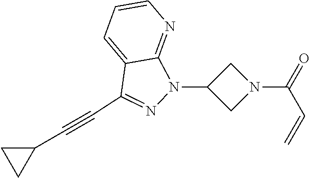 [SMILES: C=CC(=O)N1CC(n2nc(C#CC3CC3)c3cccnc32)C1] | 1-(3-(3-(cyclopropylethynyl)- 1H-pyrazolo[3,4-b]pyridin-1- yl)azetidin-1-yl)prop-2-en-1-one | 293.1 |
| 316 | 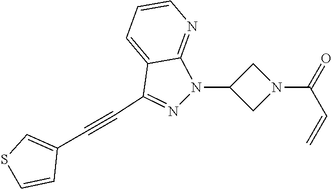 [SMILES: C=CC(=O)N1CC(n2nc(C#Cc3ccsc3)c3cccnc32)C1] | 1-(3-(3-(thiophen-3-ylethynyl)- 1H-pyrazolo[3,4-b]pyridin-1- yl)azetidin-1-yl)prop-2-en-1-one | 335.1 |
| 317 | 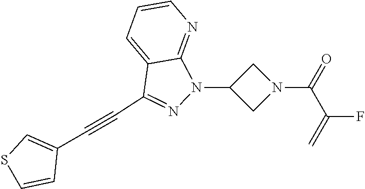 [SMILES: C=C(F)C(=O)N1CC(n2nc(C#Cc3ccsc3)c3cccnc32)C1] | 2-fluoro-1-(3-(3-(thiophen-3- ylethynyl)-1H-pyrazolo[3,4- b]pyridin-1-yl)azetidin-1- yl)prop-2-en-1-one | 353.1 |
| 318 | 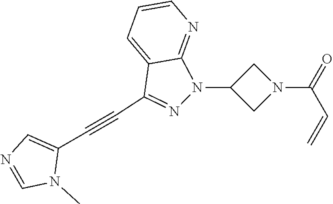 [SMILES: C=CC(=O)N1CC(n2nc(C#Cc3cncn3C)c3cccnc32)C1] | 1-(3-(3-((1-methyl-1H-imidazol- 5-yl)ethynyl)-1H-pyrazolo[3,4- b]pyridin-1-yl)azetidin-1- yl)prop-2-en-1-one | 333.1 |
| 319 | 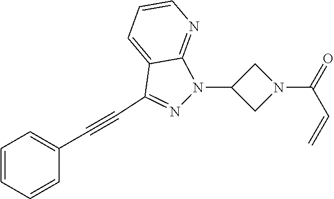 [SMILES: C=CC(=O)N1CC(n2nc(C#Cc3ccccc3)c3cccnc32)C1] | 1-(3-(3-(phenylethynyl)-1H- pyrazolo[3,4-b]pyridin-1- yl)azetidin-1-yl)prop-2-en-1-one | 329.1 |
| 320 | 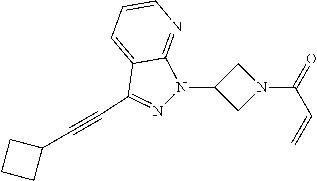 [SMILES: C=CC(=O)N1CC(n2nc(C#CC3CCC3)c3cccnc32)C1] | 1-(3-(3-(cyclobutylethynyl)-1H- pyrazolo[3,4-b]pyridin-1- yl)azetidin-1-yl)prop-2-en-1-one | 307.2 |
| 321 | 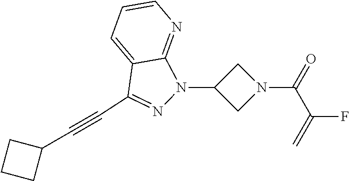 [SMILES: C=C(F)C(=O)N1CC(n2nc(C#CC3CCC3)c3cccnc32)C1] | 1-(3-(3-(cyclobutylethynyl)-1H- pyrazolo[3,4-b]pyridin-1- yl)azetidin-1-yl)-2-fluoroprop-2- en-1-one | 325.1 |
| 322 | 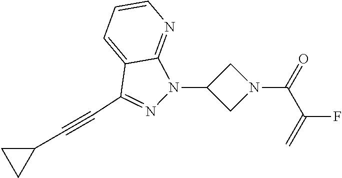 [SMILES: C=C(F)C(=O)N1CC(n2nc(C#CC3CC3)c3cccnc32)C1] | 1-(3-(3-(cyclopropylethynyl)- 1H-pyrazolo[3,4-b]pyridin-1- yl)azetidin-1-yl)-2-fluoroprop-2- en-1-one | 311.1 |
| 323 | 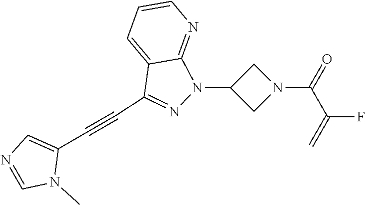 [SMILES: C=C(F)C(=O)N1CC(n2nc(C#Cc3cncn3C)c3cccnc32)C1] | 2-fluoro-1-(3-(3-((1-methy1-1H- imidazol-5-yl)ethynyl)-1H- pyrazolo[3,4-b]pyridin-1- yl)azetidin-1-yl)prop-2-en-1-one | 350.36 |
| 324 | 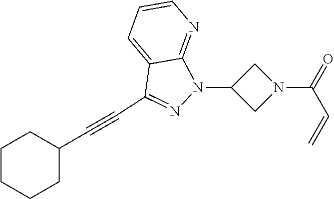 [SMILES: C=CC(=O)N1CC(n2nc(C#CC3CCCCC3)c3cccnc32)C1] | 1-(3-(3-(cyclohexylethynyl)-1H- pyrazolo[3,4-b]pyridin-1- yl)azetidin-1-yl)prop-2-en-1-one | 335.2 |
| 325 | 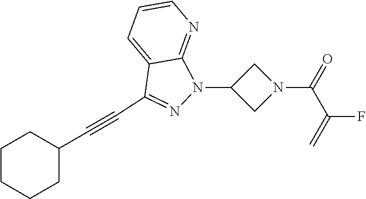 [SMILES: C=C(F)C(=O)N1CC(n2nc(C#CC3CCCCC3)c3cccnc32)C1] | 1-(3-(3-(cyclohexylethynyl)-1H- pyrazolo[3,4-b]pyridin-1- yl)azetidin-1-yl)-2-fluoroprop-2- en-1-one | 353.2 |
| 326 | 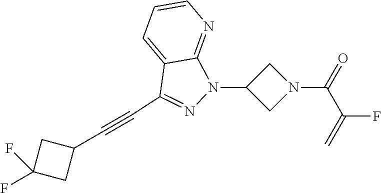 [SMILES: C=C(F)C(=O)N1CC(n2nc(C#CC3CC(F)(F)C3)c3cccnc32)C1] | 1-(3-(3-((3,3- difluorocyclobutyl)ethynyl)-1H- pyrazolo[3,4-b]pyridin-1- yl)azetidin-1-yl)-2-fluoroprop-2- en-1-one | 361.1 |
| 327 | 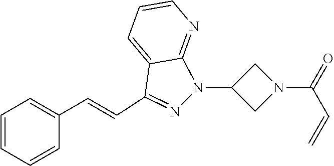 [SMILES: C=CC(=O)N1CC(n2nc(/C=C/c3ccccc3)c3cccnc32)C1] | (E)-1-(3-(3-styryl-1H- pyrazolo[3,4-b]pyridin-1- yl)azetidin-1-yl)prop-2-en-1-one | 331.2 |
| 328 | 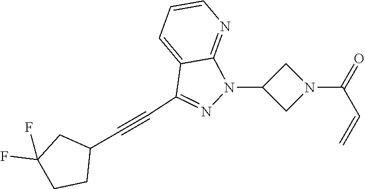 [SMILES: C=CC(=O)N1CC(n2nc(C#CC3CCC(F)(F)C3)c3cccnc32)C1] | 1-(3-(3-((3,3- difluorocyclopentyl)ethynyl)- 1H-pyrazolo[3,4-b]pyridin-1- yl)azetidin-1-yl)prop-2-en-1-one | 357.1 |
| 329 | 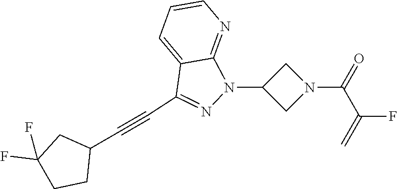 [SMILES: C=C(F)C(=O)N1CC(n2nc(C#CC3CCC(F)(F)C3)c3cccnc32)C1] | 1-(3-(3-((3,3- difluorocyclopentyl)ethynyl)- 1H-pyrazolo[3,4-b]pyridin-1- yl)azetidin-1-yl)-2-fluoroprop-2- en-1-one | 375.1 |
| 330 | 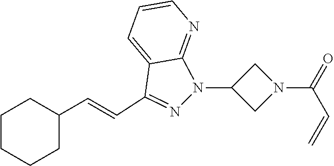 [SMILES: C=CC(=O)N1CC(n2nc(/C=C/C3CCCCC3)c3cccnc32)C1] | (E)-1-(3-(3-(2-cyclohexylvinyl)- 1H-pyrazolo[3,4-b]pyridin-1- yl)azetidin-1-yl)prop-2-en-1-one | 337.2 |
| 331 | 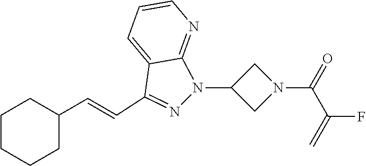 [SMILES: C=C(F)C(=O)N1CC(n2nc(/C=C/C3CCCCC3)c3cccnc32)C1] | (E)-1-(3-(3-(2-cyclohexylvinyl)- 1H-pyrazolo[3,4-b]pyridin-1- yl)azetidin-1-yl)-2-fluoroprop-2- en-1-one | 355.2 |
| 332 | 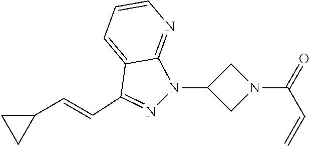 [SMILES: C=CC(=O)N1CC(n2nc(/C=C/C3CC3)c3cccnc32)C1] | (E)-1-(3-(3-(2- cyclopropylvinyl)-1H- pyrazolo[3,4-b]pyridin-1- yl)azetidin-1-yl)prop-2-en-1-one | 295.2 |
| 333 | 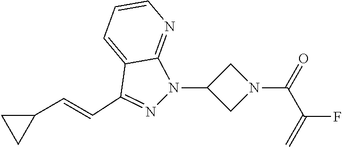 [SMILES: C=C(F)C(=O)N1CC(n2nc(/C=C/C3CC3)c3cccnc32)C1] | (E)-1-(3-(3-(2- cyclopropylvinyl)-1H- pyrazolo[3,4-b]pyridin-1- yl)azetidin-1-yl)-2-fluoroprop-2- en-1-one | 313.1 |
| 334 | 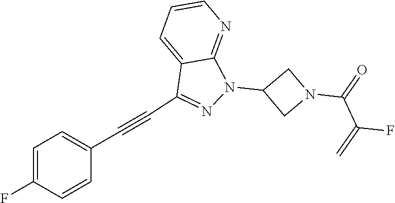 [SMILES: C=C(F)C(=O)N1CC(n2nc(C#Cc3ccc(F)cc3)c3cccnc32)C1] | 2-fluoro-1-(3-(3-((4- fluorophenyl)ethynyl)-1H- pyrazolo[3,4-b]pyridin-1- yl)azetidin-1-yl)prop-2-en-1-one | 365.1 |
| 335 | 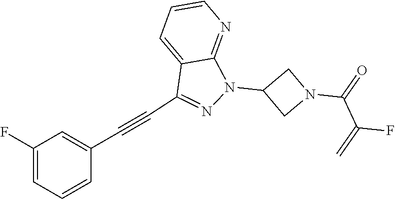 [SMILES: C=C(F)C(=O)N1CC(n2nc(C#Cc3cccc(F)c3)c3cccnc32)C1] | 2-fluoro-1-(3-(3-((3- fluorophenyl)ethynyl)-1H- pyrazolo[3,4-b]pyridin-1- yl)azetidin-1-yl)prop-2-en-1-one | 365.1 |
| 336 | 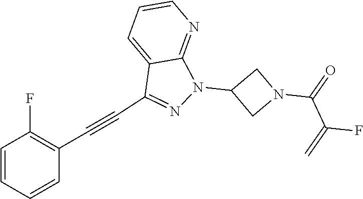 [SMILES: C=C(F)C(=O)N1CC(n2nc(C#Cc3ccccc3F)c3cccnc32)C1] | 2-fluoro-1-(3-(3-((2- fluorophenyl)ethynyl)-1H- pyrazolo[3,4-b]pyridin-1- yl)azetidin-1-yl)prop-2-en-1-one | 365.1 |
| 337 | 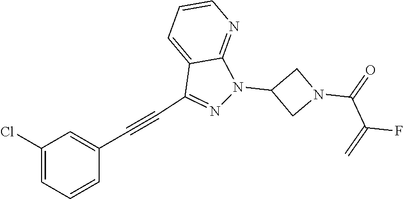 [SMILES: C=C(F)C(=O)N1CC(n2nc(C#Cc3cccc(Cl)c3)c3cccnc32)C1] | 1-(3-(3-((3- chlorophenyl)ethynyl)-1H- pyrazolo[3,4-b]pyridin-1- yl)azetidin-1-yl)-2-fluoroprop-2- en-1-one | 381.1 |
| 338 | 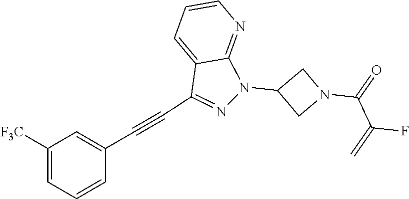 [SMILES: C=C(F)C(=O)N1CC(n2nc(C#Cc3cccc(C(F)(F)F)c3)c3cccnc32)C1] | 1-(3-(3-((3-((difluoro-13- methyl)-12- fluoranyl)phenyl)ethynyl)-1H- pyrazolo[3,4-b]pyridin-1- yl)azetidin-1-yl)-2-fluoroprop-2- en-1-one | 415.1 |
| 339 | 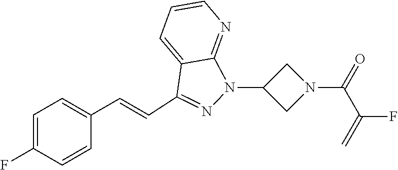 [SMILES: C=C(F)C(=O)N1CC(n2nc(/C=C/c3ccc(F)cc3)c3cccnc32)C1] | (E)-2-fluoro-1-(3-(3-(4- fluorostyryl)-1H-pyrazolo[3,4- b]pyridin-1-yl)azetidin-1- yl)prop-2-en-1-one | 367.1 |
| 340 | 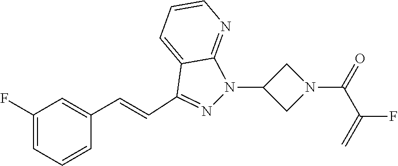 [SMILES: C=C(F)C(=O)N1CC(n2nc(/C=C/c3cccc(F)c3)c3cccnc32)C1] | (E)-2-fluoro-1-(3-(3-(3- fluorostyryl)-1H-pyrazolo[3,4- b]pyridin-1-yl)azetidin-1- yl)prop-2-en-1-one | 367.1 |
| 341 | 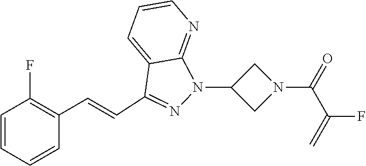 [SMILES: C=C(F)C(=O)N1CC(n2nc(/C=C/c3ccccc3F)c3cccnc32)C1] | (E)-2-fluoro-1-(3-(3-(2- fluorostyryl)-1H-pyrazolo[3,4- b]pyridin-1-yl)azetidin-1- yl)prop-2-en-1-one | 367.1 |
| 342 | 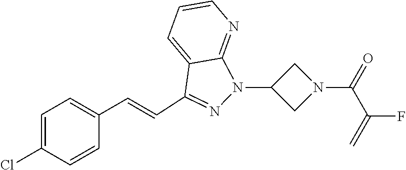 [SMILES: C=C(F)C(=O)N1CC(n2nc(/C=C/c3ccc(Cl)cc3)c3cccnc32)C1] | (E)-1-(3-(3-(4-chlorostyryl)-1H- pyrazolo[3,4-b]pyridin-1- yl)azetidin-1-yl)-2-fluoroprop-2- en-1-one | 383.1 |
| 343 | 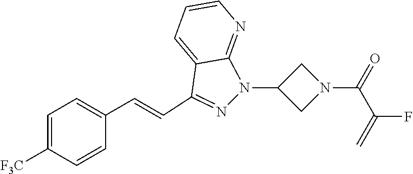 [SMILES: C=C(F)C(=O)N1CC(n2nc(/C=C/c3ccc(C(F)(F)F)cc3)c3cccnc32)C1] | (E)-2-fluoro-1-(3-(3-(4- (trifluoromethyl)styryl)-1H- pyrazolo[3,4-b]pyridin-1- yl)azetidin-1-yl)prop-2-en-1-one | 417.1 |
| 344 | 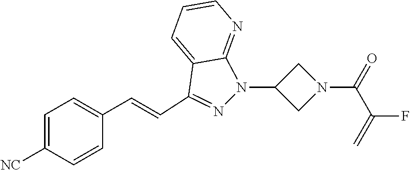 [SMILES: C=C(F)C(=O)N1CC(n2nc(/C=C/c3ccc(C#N)cc3)c3cccnc32)C1] | (E)-4-(2-(1-(1-(2- fluoroacryloyl)azetidin-3-yl)-1H- pyrazolo[3,4-b]pyridin-3- yl)vinyl)benzonitrile | 374.1 |
| 345 | 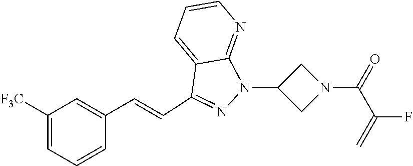 [SMILES: C=C(F)C(=O)N1CC(n2nc(/C=C/c3cccc(C(F)(F)F)c3)c3cccnc32)C1] | (E)-2-fluoro-1-(3-(3-(3- (trifluoromethyl)styryl)-1H- pyrazolo[3,4-b]pyridin-1- yl)azetidin-1-yl)prop-2-en-1-one | 417.1 |
| 346 | 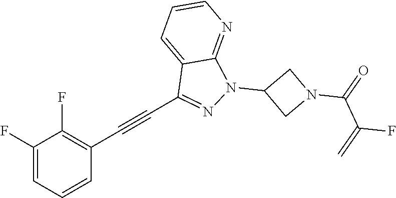 [SMILES: C=C(F)C(=O)N1CC(n2nc(C#Cc3cccc(F)c3F)c3cccnc32)C1] | 1-(3-(3-((2,3- difluorophenyl)ethynyl)-1H- pyrazolo[3,4-b]pyridin-1- yl)azetidin-1-yl)-2-fluoroprop-2- en-1-one | 383.1 |
| 347 | 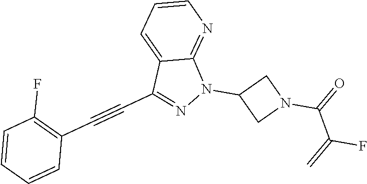 [SMILES: C=C(F)C(=O)N1CC(n2nc(C#Cc3ccccc3F)c3cccnc32)C1] | 2-fluoro-1-(3-(3-((2- fluorophenyl)ethynyl)-1H- pyrazolo[3,4-b]pyridin-1- yl)azetidin-1-yl)prop-2-en-1-one | 365.1 |
| 348 | 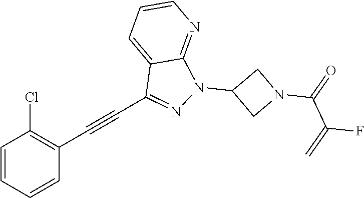 [SMILES: C=C(F)C(=O)N1CC(n2nc(C#Cc3ccccc3Cl)c3cccnc32)C1] | 1-(3-(3-((2- chlorophenyl)ethynyl)-1H- pyrazolo[3,4-b]pyridin-1- yl)azetidin-1-yl)-2-fluoroprop-2- en-1-one | 381.1 |
| 349 | 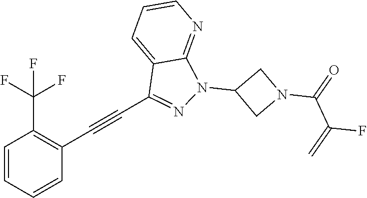 [SMILES: C=C(F)C(=O)N1CC(n2nc(C#Cc3ccccc3C(F)(F)F)c3cccnc32)C1] | 2-fluoro-1-(3-(3-((2- (trifluoromethyl)phenyl)ethynyl)- 1H-pyrazolo[3,4-b]pyridin-1- yl)azetidin-1-yl)prop-2-en-1-one | 415.1 |

The ¹HNMR data of compounds are as follows:

1H NMR (500 MHz, CDCl₃) δ 8.59 (dd, J=4.5, 1.3 Hz, 1H), 8.37 (dd, J=8.1, 1.3 Hz, 1H), 8.11 (d, J=8.1 Hz, 2H), 7.78 (d, J=8.1 Hz, 2H), 7.28 (dd, J=8.1, 4.5 Hz, 1H), 6.43 (dd, J=17.0, 1.7 Hz, 1H), 6.30 (dd, J=17.0, 10.3 Hz, 1H), 6.06-5.98 (m, 1H), 5.76 (dd, J=10.3, 1.7 Hz, 1H), 4.96-4.88 (m, 1H), 4.83-4.75 (m, 2H), 4.72-4.65 (m, 1H). (Compound 30)

¹H NMR (500 MHz, DMSO-d₆) δ 8.33 (d, J=9.2 Hz, 1H), 8.23 (dt, J=8.2, 1.4 Hz, 1H), 8.17 (d, J=8.1 Hz, 2H), 8.00-7.80 (m, 3H), 7.60 (dd, J=7.1, 2.8 Hz, 1H), 7.35 (ddd, J=8.5, 7.0, 1.6 Hz, 1H), 6.62 (ddd, J=41.2, 16.8, 10.3 Hz, 1H), 6.16 (dt, J=16.8, 2.7 Hz, 1H), 5.71-5.65 (m, 1H), 4.17-4.03 (m, 1H), 3.98-3.76 (m, 2H), 3.74-3.51 (m, 1H), 3.21-2.83 (m, 1H), 2.61 (dd, J=8.5, 4.2 Hz, 1H), 2.45-2.34 (m, 1H). (Compound 42)

¹H NMR (500 MHz, DMSO-d₆) δ 8.16 (dd, J=8.4, 4.1 Hz, 2H), 7.99 (dd, J=8.9, 1.4 Hz, 1H), 7.84 (dd, J=8.4, 3.8 Hz, 2H), 7.35 (dd, J=12.3, 2.2 Hz, 1H), 6.93 (dd, J=8.9, 2.2 Hz, 1H), 6.18 (ddd, J=16.7, 5.3, 2.5 Hz, 1H), 5.70 (ddd, J=28.7, 10.3, 2.5 Hz, 1H), 5.57 (dq, J=38.1, 6.2 Hz, 1H), 4.20-3.99 (m, 1H), 3.99-3.93 (m, 1H), 3.90 (s, 3H), 3.86-3.78 (m, 2H), 3.75-3.54 (m, 1H), 2.54 (d, J=7.1 Hz, 1H), 2.45 (qd, J=6.7, 1.9 Hz, 1H). (Compound 54)

¹H NMR (500 MHz, CDCl₃) δ 8.58 (dd, J=4.5, 1.4 Hz, 1H), 8.36 (dd, J=8.1, 1.4 Hz, 1H), 8.12 (d, J=8.1 Hz, 2H), 7.78 (d, J=8.2 Hz, 2H), 7.27 (dd, J=8.1, 4.5 Hz, 1H), 6.00-5.92 (m, 1H), 5.49-5.42 (m, 2H), 4.95-4.59 (m, 4H), 2.01 (s, 3H). (Compound 73) 1H NMR (500 MHz, DMSO) δ 8.52 (d, J=8.1 Hz, 2H), 8.38 (s, 1H), 7.89 (d, J=8.3 Hz, 2H), 6.08 (m, 1H), 5.55 (dd, J=48.4, 3.5 Hz, 1H), 5.36 (dd, J=16.6, 3.5 Hz, 1H), 4.97-4.76 (m, 2H), 4.60-4.43 (m, 2H), 3.55 (s, 3H). (Compound 80)

¹H NMR (500 MHz, CDCl₃) δ 8.73 (dd, J=4.3, 1.2 Hz, 1H), 8.69 (d, J=8.1 Hz, 2H), 7.82 (dd, J=8.6, 1.2 Hz, 1H), 7.76 (d, J=8.2 Hz, 2H), 7.39 (dd, J=8.6, 4.3 Hz, 1H), 6.45 (dd, J=17.0, 1.8 Hz, 1H), 6.30 (dd, J=17.0, 10.3 Hz, 1H), 5.78 (dd, J=10.3, 1.8 Hz, 1H), 5.56-5.49 (m, 1H), 4.96-4.86 (m, 1H), 4.83-4.72 (m, 2H), 4.72-4.64 (m, 1H). (Compound 82)

¹H NMR (500 MHz, CDCl₃) δ 8.73 (dd, J=4.3, 1.1 Hz, 1H), 8.69 (d, J=8.1 Hz, 2H), 7.82 (dd, J=8.6, 1.0 Hz, 1H), 7.77 (d, J=8.2 Hz, 2H), 7.39 (dd, J=8.6, 4.3 Hz, 1H), 5.53-5.44 (m, 3H), 4.97-4.83 (m, 1H), 4.82-4.60 (m, 3H), 2.02 (s, 3H). (Compound 87)

¹H NMR (500 MHz, DMSO-d₆) δ 9.13 (dd, J=13.9, 7.0 Hz, 1H), 8.26 (dd, J=8.3, 2.3 Hz, 1H), 8.22-8.05 (m, 2H), 7.88 (dd, J=8.5, 2.6 Hz, 2H), 7.56 (dd, J=7.0, 3.8 Hz, 1H), 7.37 (ddd, J=8.5, 7.1, 1.8 Hz, 1H), 6.61 (ddd, J=42.4, 16.8, 10.3 Hz, 1H), 6.15 (dt, J=16.8, 3.0 Hz, 1H), 5.68 (ddd, J=12.4, 10.2, 2.4 Hz, 1H), 5.58-5.40 (m, 1H), 4.25-4.02 (m, 3H), 3.96-3.75 (m, 2H), 3.75-3.52 (m, 2H), 2.59 (td, J=16.9, 14.5, 7.5 Hz, 2H), 2.43-2.31 (m, 2H), 2.26-2.15 (m, 2H). (Compound 113)

¹H NMR (500 MHz, CDCl₃) δ 8.67 (dd, J=4.3, 1.3 Hz, 1H), 8.65 (d, J=8.1 Hz, 2H), 8.17 (dd, J=8.6, 1.3 Hz, 1H), 7.73 (d, J=8.2 Hz, 2H), 7.34 (dd, J=8.6, 4.3 Hz, 1H), 6.29 (dd, J=16.9, 1.2 Hz, 1H), 6.05 (dd, J=16.9, 10.3 Hz, 1H), 5.66 (dd, J=10.3, 1.2 Hz, 1H), 5.62 (d, J=5.4 Hz, 1H), 5.34-5.27 (m, 1H), 4.45-4.35 (m, 1H), 2.64-2.53 (m, 1H), 2.40-2.20 (m, 3H), 2.08-1.96 (m, 1H), 1.95-1.83 (m, 1H). (Compound 119)

¹H NMR (500 MHz, CDCl₃) δ 8.69 (dd, J=4.3, 1.3 Hz, 1H), 8.40 (d, J=8.3 Hz, 2H), 7.77 (dd, J=8.6, 1.3 Hz, 1H), 7.34 (dd, J=8.5, 4.4 Hz, 1H), 7.21 (d, J=8.3 Hz, 2H), 6.44 (dd, J=17.0, 1.8 Hz, 1H), 6.29 (dd, J=17.0, 10.3 Hz, 1H), 5.76 (dd, J=10.3, 1.8 Hz, 1H), 5.53-5.45 (m, 1H), 4.98-4.87 (m, 1H), 4.80-4.71 (m, 2H), 4.69-4.62 (m, 1H), 2.00-1.93 (m, 1H), 1.04-0.98 (m, 2H), 0.80-0.74 (m, 2H). (Compound 120)

¹H NMR (500 MHz, CDCl₃) δ 8.04 (d, J=8.1 Hz, 2H), 8.00 (d, J=9.0 Hz, 1H), 7.72 (d, J=8.2 Hz, 2H), 6.58 (d, J=9.0 Hz, 1H), 6.40 (dd, J=17.0, 2.0 Hz, 1H), 6.29 (dd, J=17.0, 10.2 Hz, 1H), 5.79-5.73 (m, 1H), 5.72 (dd, J=10.2, 1.9 Hz, 1H), 4.99-4.92 (m, 1H), 4.85-4.78 (m, 1H), 4.74-4.67 (m, 1H), 4.64-4.57 (m, 1H), 3.19 (s, 6H). (Compound 121)

¹H NMR (500 MHz, CDCl₃) δ 9.34 (d, J=1.3 Hz, 1H), 8.63 (dd, J=4.5, 1.4 Hz, 1H), 8.52 (dd, J=8.1, 1.5 Hz, 1H), 8.36 (dd, J=8.2, 1.4 Hz, 1H), 7.84 (d, J=8.2 Hz, 1H), 7.33 (dd, J=8.2, 4.5 Hz, 1H), 6.44 (dd, J=17.0, 1.8 Hz, 1H), 6.30 (dd, J=17.0, 10.3 Hz, 1H), 6.07-5.99 (m, 1H), 5.76 (dd, J=10.3, 1.8 Hz, 1H), 4.95-4.85 (m, 1H), 4.84-4.73 (m, 2H), 4.72-4.65 (m, 1H). (Compound 123)

¹H NMR (500 MHz, CDCl₃) δ 8.58 (dd, J=4.5, 1.4 Hz, 1H), 8.26 (ddd, J=8.2, 3.6, 1.5 Hz, 1H), 8.10 (t, J=7.6 Hz, 1H), 7.57 (d, J=8.1 Hz, 1H), 7.52 (d, J=10.5 Hz, 1H), 7.28-7.24 (m, 1H), 6.42 (dd, J=17.0, 1.9 Hz, 1H), 6.29 (dd, J=17.0, 10.3 Hz, 1H), 6.03 (dq, J=8.2, 5.7 Hz, 1H), 5.74 (dd, J=10.3, 1.9 Hz, 1H), 4.93-4.87 (m, 1H), 4.82-4.73 (m, 2H), 4.71-4.64 (m, 1H). (Compound 127)

¹H NMR (500 MHz, CDCl₃) δ 8.73 (dd, J=4.3, 1.1 Hz, 1H), 8.41 (t, J=7.5 Hz, 1H), 7.87 (dd, J=8.6, 1.1 Hz, 1H), 7.59 (d, J=8.1 Hz, 1H), 7.52 (d, J=10.1 Hz, 1H), 7.41 (dd, J=8.6, 4.3 Hz, 1H), 6.43 (dd, J=17.0, 1.8 Hz, 1H), 6.29 (dd, J=17.0, 10.3 Hz, 1H), 5.77 (dd, J=10.3, 1.8 Hz, 1H), 5.61-5.53 (m, 1H), 5.00-4.89 (m, 1H), 4.84-4.76 (m, 1H), 4.75-4.65 (m, 2H). (Compound 129)

¹H NMR (500 MHz, CDCl₃) δ 8.72 (d, J=4.0 Hz, 1H), 8.40 (t, J=7.5 Hz, 1H), 7.86 (d, J=8.4 Hz, 1H), 7.59 (d, J=8.0 Hz, 1H), 7.52 (d, J=10.1 Hz, 1H), 7.42 (dd, J=8.5, 4.3 Hz, 1H), 5.71 (dd, J=46.7, 3.1 Hz, 1H), 5.62-5.52 (m, 1H), 5.17 (dd, J=15.6, 3.1 Hz, 1H), 5.12-5.04 (m, 1H), 5.03-4.93 (m, 1H), 4.81-4.65 (m, 2H). (Compound 130).

¹H NMR (500 MHz, CDCl₃) δ 9.85 (d, J=1.6 Hz, 1H), 9.06 (dd, J=8.2, 1.5 Hz, 1H), 8.74 (dd, J=4.3, 1.0 Hz, 1H), 7.85 (dd, J=8.6, 0.9 Hz, 1H), 7.81 (d, J=8.1 Hz, 1H), 7.42 (dd, J=8.6, 4.3 Hz, 1H), 6.45 (dd, J=17.0, 1.8 Hz, 1H), 6.30 (dd, J=17.0, 10.3 Hz, 1H), 5.79 (dd, J=10.3, 1.8 Hz, 1H), 5.59-5.50 (m, 1H), 4.97-4.87 (m, 1H), 4.86-4.77 (m, 1H), 4.75-4.65 (m, 2H). (Compound 131).

¹H NMR (500 MHz, CDCl₃) δ 9.86 (d, J=1.4 Hz, 1H), 9.07 (dd, J=8.2, 1.4 Hz, 1H), 8.74 (dd, J=4.3, 0.9 Hz, 1H), 7.87-7.78 (m, 2H), 7.42 (dd, J=8.6, 4.3 Hz, 1H), 5.74 (dd, J=46.7, 3.1 Hz, 1H), 5.61-5.50 (m, 1H), 5.19 (dd, J=15.6, 3.1 Hz, 1H), 5.10-4.94 (m, 2H), 4.81-4.66 (m, 2H). (Compound 132).

¹H NMR (500 MHz, DMSO-d₆) δ 8.84 (d, J=2.2 Hz, 1H), 8.74 (d, J=2.2 Hz, 1H), 8.65 (d, J=8.0 Hz, 2H), 7.93 (d, J=8.1 Hz, 2H), 6.45 (dd, J=16.9, 10.3 Hz, 1H), 6.21 (dd, J=17.0, 2.3 Hz, 1H), 5.96 (ddd, J=13.6, 8.2, 5.5 Hz, 1H), 5.76 (dd, J=10.3, 2.3 Hz, 1H), 5.00-4.65 (m, 2H), 4.64-4.37 (m, 2H). (Compound 161)

1H NMR (500 MHz, DMSO-d₆) δ 8.78-8.56 (m, 2H), 8.27 (d, J=8.1 Hz, 2H), 7.89 (d, J=8.2 Hz, 2H), 7.43 (dd, J=8.2, 4.4 Hz, 2H), 6.88 (d, J=2.3 Hz, 1H), 5.51 (dd, J=48.5, 3.6 Hz, 1H), 5.36-5.21 (m, 2H), 5.11-4.83 (m, 2H), 4.63 (d, J=11.0 Hz, 1H), 3.20 (d, J=3.9 Hz, 2H). (Compound 169)

¹H NMR (500 MHz, DMSO-d₆) δ 8.67-8.55 (m, 2H), 8.00-7.91 (m, 2H), 7.45-7.39 (m, 2H), 7.36 (dd, J=8.1, 4.4 Hz, 1H), 5.99 (tt, J=8.2, 5.5 Hz, 1H), 5.57 (dd, J=48.4, 3.5 Hz, 1H), 5.37 (dd, J=16.6, 3.5 Hz, 1H), 5.02-4.80 (m, 2H), 4.72-4.41 (m, 2H), 3.05-2.81 (m, 1H), 1.26 (d, J=6.9 Hz, 6H). (Compound 171)

1H NMR (500 MHz, DMSO-d₆) δ 8.98 (s, 1H), 8.72-8.55 (m, 2H), 8.22-8.17 (m, 1H), 7.63-7.46 (m, 2H), 7.40 (dd, J=8.0, 4.6 Hz, 1H), 6.01 (tt, J=8.3, 5.5 Hz, 1H), 5.57 (dd, J=48.4, 3.5 Hz, 1H), 5.37 (dd, J=16.6, 3.5 Hz, 1H), 5.02-4.81 (m, 2H), 4.70-4.43 (m, 2H). (Compound 172)

1H NMR (500 MHz, DMSO-d₆) δ 8.75-8.59 (m, 2H), 8.31 (d, J=8.4 Hz, 2H), 8.20-8.00 (m, 2H), 7.45 (dd, J=8.0, 4.5 Hz, 1H), 6.07-6.00 (m, 1H), 5.57 (dd, J=48.4, 3.5 Hz, 1H), 5.37 (dd, J=16.6, 3.6 Hz, 1H), 4.93 (d, J=49.3 Hz, 2H), 4.68-4.47 (m, 2H). (Compound 173)

1H NMR (500 MHz, DMSO-d₆) δ 8.69-8.53 (m, 2H), 8.43-8.24 (m, 2H), 7.90-7.69 (m, 2H), 7.41 (dd, J=8.1, 4.5 Hz, 1H), 4.70-4.32 (m, 4H), 4.07 (dq, J=26.8, 7.1 Hz, 3H). (Compound 176)

¹H NMR (500 MHz, DMSO-d₆) δ 8.74 (dd, J=8.3, 1.6 Hz, 1H), 8.70 (dd, J=4.5, 1.5 Hz, 1H), 8.59 (dd, J=8.1, 1.8 Hz, 1H), 8.52 (d, J=1.6 Hz, 1H), 8.33 (d, J=8.1 Hz, 1H), 7.47 (ddd, J=8.5, 4.6, 1.4 Hz, 1H), 6.04 (tt, J=8.4, 5.5 Hz, 1H), 5.57 (dd, J=48.4, 3.6 Hz, 1H), 5.37 (dd, J=16.4, 3.6 Hz, 1H), 5.08-4.80 (m, 2H), 4.69-4.48 (m, 2H). (Compound 177)

¹H NMR (500 MHz, DMSO-d₆) δ 8.85-8.74 (m, 2H), 8.69 (dd, J=4.5, 1.6 Hz, 1H), 8.59 (d, J=8.5 Hz, 1H), 8.13 (d, J=8.2 Hz, 1H), 7.45 (m 1.5 Hz, 1H), 6.04 (tt, J=8.5, 5.4 Hz, 1H), 5.58 (m, 1.4 Hz, 1H), 5.38 (m, 1.4 Hz, 1H), 5.08-4.77 (m, 2H), 4.77-4.44 (m, 2H). (Compound 178)

¹H NMR (500 MHz, DMSO-d₆) δ 9.21 (d, J=2.0 Hz, 1H), 8.79 (dd, J=8.2, 1.5 Hz, 1H), 8.68 (dd, J=4.4, 1.5 Hz, 1H), 8.54 (d, J=2.0 Hz, 1H), 7.43 (dd, J=8.2, 4.5 Hz, 1H), 5.97 (tt, J=8.2, 5.6 Hz, 1H), 4.75-4.33 (m, 4H), 4.10 (q, J=7.0 Hz, 2H), 1.22 (t, J=7.0 Hz, 3H). (Compound 181)

¹H NMR (500 MHz, DMSO) δ 8.69 (d, J=3.9 Hz, 1H), 8.35 (dd, J=24.0, 8.0 Hz, 2H), 7.90 (d, J=7.9 Hz, 1H), 7.40 (dd, J=7.8, 4.5 Hz, 1H), 6.04 (s, 1H), 5.55 (d, J=48.3 Hz, 1H), 5.36 (dd, J=16.5, 2.9 Hz, 1H), 5.00 (s, 1H), 4.84 (s, 1H), 4.64 (t, J=9.4 Hz, 1H), 4.51 (s, 1H), 2.73 (s, 3H). (Compound 182)

¹H NMR (500 MHz, CDCl₃-d₃) δ 9.13 (s, 1H), 8.65-8.64 (m, 1H), 8.36-8.35 (m, 1H), 8.27-8.25 (m, 1H), 7.36-7.34 (m, 1H), 6.07-6.01 (m, 1H), 5.77-5.67 (m, 1H), 5.19-5.15 (m, 1H), 5.06-5.50 (m, 2H), 4.78-4.68 (m, 2H). (Compound 184)

¹H NMR (500 MHz, CDCl₃-d₃) δ 8.22-8.21 (m, 1H), 8.11-8.10 (m, 2H), 7.77-7.76 (m, 2H), 7.14-7.13 (m, 1H), 6.04-5.98 (m, 1H), 5.76-5.66 (m, 1H), 5.17-5.13 (m, 1H), 5.06-5.02 (m, 1H), 4.96-4.92 (m, 1H), 4.79-4.76 (m, 1H), 4.70-4.66 (m, 1H), 2.70 (s, 3H). (Compound 186)

¹H NMR (500 MHz, DMSO) δ 9.49 (d, J=2.2 Hz, 1H), 8.75 (ddd, J=14.9, 9.3, 2.0 Hz, 3H), 8.35 (d, J=8.2 Hz, 2H), 8.16 (dd, J=8.9, 2.7 Hz, 1H), 7.95 (t, J=14.2 Hz, 2H), 7.55-7.46 (m, 1H), 7.34 (d, J=9.0 Hz, 1H), 5.89-5.66 (m, 1H), 5.49 (dd, J=15.8, 3.8 Hz, 1H), 3.94 (s, 3H). (Compound 188)

¹H NMR (500 MHz, DMSO-d₆) δ 9.96 (s, 1H), 8.88-8.87 (m, 1H), 8.79-8.78 (m, 2H), 8.37-8.35 (m, 2H), 8.32-8.30 (m, 1H), 7.95-7.94 (m, 2H), 7.77-7.75 (m, 1H), 7.55-7.52 (m, 1H), 6.72-6.66 (m, 1H), 6.35-6.32 (m, 1H), 5.86-5.84 (m, 1H). (Compound 189)

¹H NMR (500 MHz, DMSO) δ 9.75 (s, 1H), 9.02 (d, J=8.3 Hz, 1H), 8.89 (d, J=2.2 Hz, 1H), 8.80 (d, J=2.2 Hz, 1H), 8.13 (d, J=8.2 Hz, 1H), 6.00 (dq, J=8.2, 5.5 Hz, 1H), 5.58 (dd, J=48.4, 3.5 Hz, 1H), 5.38 (dd, J=16.6, 3.5 Hz, 1H), 5.08-4.87 (m, 2H), 4.70-4.54 (m, 2H). (Compound 298)

¹H NMR (500 MHz, DMSO) (δ 9.44 (s, 1H), 8.71 (d, J=8.2 Hz, 1H), 8.62 (d, J=8.3 Hz, 1H), 8.04 (d, J=8.2 Hz, 1H), 7.33 (d, J=8.3 Hz, 1H), 6.12-5.92 (m, 1H), 5.57 (dd, J=48.4, 3.4 Hz, 1H), 5.37 (dd, J=16.5, 3.4 Hz, 1H), 4.98 (s, 1H), 4.86 (s, 1H), 4.62 (t, J=9.5 Hz, 1H), 4.51 (dd, J=10.5, 5.2 Hz, 1H), 2.66 (s, 3H). (Compound 199)

¹H NMR (500 MHz, DMSO-d₆) (δ 8.75 (s, 1H), 8.66-8.64 (m, 1H), 8.31-8.30 (m, 2H), 7.90-7.88 (m, 2H), 6.02-6.00 (m, 1H), 5.62-5.51 (m, 1H), 5.39-5.35 (m, 1H), 5.00-4.86 (m, 2H), 4.64-4.51 (m, 2H). (Compound 202)

¹H NMR (500 MHz, DMSO-d₆) δ 7.79 (q, J=8.4 Hz, 4H), 5.55 (d, J=45 Hz, 1H), 5.37 (d, J=15 Hz, 1H), 5.32-5.25 (m, 1H), 4.86-4.79 (m, 3H), 4.71 (q, J=6.1, 5.4 Hz, 1H), 4.48 (t, J=9.4 Hz, 1H), 4.37 (dd, J=10.7, 5.3 Hz, 1H), 3.88 (t, J=5.5 Hz, 2H), 2.78 (dt, J=7.0, 3.3 Hz, 2H). (Compound 208)

¹H NMR (500 MHz, DMSO-d₆) δ 9.09 (d, J=2.0 Hz, 1H), 9.05 (d, J=2.0 Hz, 1H), 8.37 (d, J=8.1 Hz, 2H), 7.92 (d, J=8.2 Hz, 2H), 6.06 (tt, J=8.3, 5.4 Hz, 1H), 5.58 (d, J=45 Hz, 1H), 5.38 (dd, J=16.6, 3.5 Hz, 1H), 5.00 (td, J=9.5, 9.0, 4.2 Hz, 1H), 4.90 (q, J=8.1, 6.0 Hz, 1H), 4.64 (t, J=9.6 Hz, 1H), 4.56 (dd, J=10.9, 5.4 Hz, 1H). (Compound 215)

¹H NMR (500 MHz, DMSO) δ 8.54 (m, 2H), 8.42 (s, 1H), 7.89 (d, J=8.2 Hz, 2H), 6.11 (m, 1H), 5.55 (dd, J=48.4, 3.5 Hz, 1H), 5.36 (dd, J=16.6, 3.5 Hz, 1H), 4.88 (m, 2H), 4.52 (m, 2H), 4.08 (q, J=7.1 Hz, 2H), 1.31 (t, J=7.1 Hz, 3H). (Compound 218)

¹H NMR (500 MHz, DMSO-d₆) δ 8.57 (d, J=8.2 Hz, 2H), 8.42 (s, 1H), 7.90 (d, J=8.2 Hz, 2H), 5.77 (ddd, J=8.1, 5.3, 2.9 Hz, 1H), 5.57 (dd, J=48.4, 3.6 Hz, 1H), 5.38 (dd, J=16.6, 3.5 Hz, 1H), 4.93 (ddt, J=40.9, 9.7, 5.3 Hz, 2H), 4.67-4.48 (m, 2H), 4.03 (s, 3H). (Compound 223)

¹H NMR (500 MHz, DMSO-d₆) (58.53 (d, J=8.1 Hz, 2H), 7.88 (d, J=8.2 Hz, 2H), 6.07 (ddd, J=8.1, 5.3, 2.8 Hz, 1H), 5.55 (dd, J=48.4, 3.5 Hz, 1H), 5.35 (dd, J=16.6, 3.6 Hz, 1H), 4.99-4.72 (m, 2H), 4.64-4.40 (m, 2H), 3.56 (s, 3H), 2.64 (s, 3H). (Compound 225)

¹H NMR (400 MHz, CDCl₃) δ 7.83-7.81 (m, 2H), 7.70-7.68 (m, 2H), 7.32 (d, J=6 MHz, 1H), 7.23-7.20 (m, 1H), 7.17-7.16 (m, 2H), 6.47 (dd, J=1.4 MHz, J=13.5 MHz, 1H), 6.33-6.27 (m, 1H), 5.78 (dd, J=1.4 MHz, J=8.3 MHz, 1H), 5.54-5.48 (m, 1H), 4.87-4.83 (m, 1H), 4.71-4.68 (m, 2H), 4.62-4.59 (m, 1H). (Compound 236)

1H NMR (500 MHz, CDCl₃) δ 8.11-8.09 (dd, J=1.0 MHz, J=4.2 MHz, 1H), 7.83-7.81 (m, 2H), 7.71-7.69 (m, 2H), 7.37 (dd, J=1.0 MHz, J=6.3 MHz, 1H), 7.07-7.04 (m, 1H), 5.69-5.60 (m, 1H), 5.27-5.25 (m, 1H), 5.13 (dd, J=4.0 MHz, J=12.0 MHz, 1H), 4.98-4.96 (m, 1H), 4.66-4.63 (m, 1H), 4.41-4.38 (m, 1H), 1.86 (s, 3H). (Compound 238)

¹H NMR (500 MHz, DMSO) δ 8.16-8.11 (m, 1H), 8.05 (d, J=3.2 Hz, 1H), 7.99 (q, J=8.7 Hz, 4H), 5.54 (dd, J=48.5, 3.5 Hz, 1H), 5.45 (dq, J=8.7, 5.8 Hz, 1H), 5.35 (dd, J=16.6, 3.5 Hz, 1H), 5.05-4.97 (m, 1H), 4.79 (td, J=9.3, 4.2 Hz, 1H), 4.67 (dd, J=10.6, 5.8 Hz, 1H), 4.43 (t, J=9.7 Hz, 1H). (Compound 240)

¹H NMR (500 MHz, DMSO) δ 9.20 (d, J=2.2 Hz, 1H), 8.50 (dd, J=8.5, 2.3 Hz, 1H), 8.12-8.15 (m, 2H), 8.09 (d, J=3.3 Hz, 1H), 5.55 (dd, J=48.5, 3.5 Hz, 1H), 5.48-5.42 (m, 1H), 5.36 (dd, J=16.6, 3.5 Hz, 1H), 5.01 (d, J=3.6 Hz, 1H), 4.81 (td, J=9.2, 4.2 Hz, 1H), 4.67 (dd, J=10.7, 5.8 Hz, 1H), 4.45 (t, J=9.7 Hz, 1H). (Compound 241)

¹H NMR (500 MHz, DMSO) δ 8.01-7.93 (m, 3H), 7.85 (d, J=8.4 Hz, 2H), 7.45 (d, J=1.7 Hz, 1H), 5.53 (dd, J=48.5, 3.5 Hz, 1H), 5.43 (ddd, J=14.7, 7.3, 4.4 Hz, 1H), 5.34 (dd, J=16.6, 3.5 Hz, 1H), 5.08-4.99 (m, 1H), 4.76 (td, J=9.3, 4.2 Hz, 1H), 4.68 (dd, J=10.6, 6.0 Hz, 1H), 4.43-4.36 (m, 1H), 2.31 (s, 3H). (Compound 244)

¹H NMR (500 MHz, DMSO-d₆) δ 8.55 (dd, J=1.9, 1.0 Hz, 1H), 7.99 (d, J=8.4 Hz, 2H), 7.86 (d, J=8.3 Hz, 2H), 7.82 (d, J=1.9 Hz, 1H), 5.59-5.48 (m, 2H), 5.35 (dd, J=16.5, 3.5 Hz, 1H), 5.05 (td, J=10.6, 9.2, 6.0 Hz, 1H), 4.79 (td, J=9.4, 4.2 Hz, 1H), 4.70 (dd, J=10.7, 5.9 Hz, 1H), 4.47-4.40 (m, 1H). (Compound 245)

¹H NMR (500 MHz, DMSO-d₆) δ 7.97 (d, J=8.7 Hz, 2H), 7.84 (d, J=8.3 Hz, 2H), 7.62 (d, J=8.1 Hz, 1H), 7.22 (dd, J=8.1, 1.3 Hz, 1H), 5.53 (d, J=75 Hz, 1H), 5.41 (tt, J=8.8, 5.7 Hz, 1H), 5.34 (d, J=20 Hz, 1H), 5.00 (dt, J=9.6, 4.2 Hz, 1H), 4.77 (td, J=9.3, 3.9 Hz, 1H), 4.65 (dd, J=10.8, 5.8 Hz, 1H), 4.45-4.37 (m, 1H). (Compound 252)

¹H NMR (500 MHz, DMSO-d₆) δ 8.32-8.11 (m, 1H), 8.11-7.95 (m, 4H), 7.87-7.67 (m, 2H), 7.57-7.41 (m, 1H), 6.40 (dt, J=16.9, 10.9 Hz, 1H), 6.15 (ddd, J=16.8, 13.9, 2.2 Hz, 1H), 5.71 (ddd, J=12.8, 10.3, 2.3 Hz, 1H), 4.80-4.53 (m, 2H), 4.50-4.26 (m, 3H). (Compound 294-310)

¹H NMR (500 MHz, CDCl₃) δ 8.56 (dd, J=4.5, 1.4 Hz, 1H), 8.12 (dd, J=8.0, 1.4 Hz, 1H), 7.24 (dd, J=8.1, 4.5 Hz, 1H), 6.39 (dd, J=17.0, 1.8 Hz, 1H), 6.25 (dd, J=17.0, 10.3 Hz, 1H), 5.98-5.89 (m, 1H), 5.72 (dd, J=10.3, 1.7 Hz, 1H), 4.90-4.84 (m, 1H), 4.75-4.68 (m, 1H), 4.66-4.59 (m, 2H), 3.29-3.19 (m, 1H), 3.09-2.97 (m, 2H), 2.95-2.80 (m, 2H). (Compound 309)

¹H NMR (500 MHz, CDCl₃) δ 8.52 (dd, J=4.5, 1.5 Hz, 1H), 8.11 (dd, J=8.0, 1.5 Hz, 1H), 7.20 (dd, J=8.0, 4.5 Hz, 1H), 6.38 (dd, J=17.0, 1.8 Hz, 1H), 6.24 (dd, J=17.0, 10.3 Hz, 1H), 5.96-5.87 (m, 1H), 5.71 (dd, J=10.3, 1.8 Hz, 1H), 4.90-4.82 (m, 1H), 4.73-4.66 (m, 1H), 4.66-4.55 (m, 2H), 1.60-1.52 (m, 1H), 0.99-0.90 (m, 4H). (Compound 315)

¹H NMR (500 MHz, CDCl₃) δ 8.59 (dd, J=4.5, 1.5 Hz, 1H), 8.18 (dd, J=8.0, 1.5 Hz, 1H), 7.61 (s, 1H), 7.53 (s, 1H), 7.28 (dd, J=8.1, 4.5 Hz, 1H), 6.41 (dd, J=17.0, 1.8 Hz, 1H), 6.27 (dd, J=17.0, 10.3 Hz, 1H), 6.03-5.94 (m, 1H), 5.74 (dd, J=10.3, 1.8 Hz, 1H), 4.94-4.84 (m, 1H), 4.79-4.72 (m, 1H), 4.71-4.61 (m, 2H), 3.83 (s, 3H). (Compound 318)

¹H NMR (500 MHz, CDCl₃) δ 8.58 (dd, J=4.5, 1.4 Hz, 1H), 8.22 (dd, J=8.0, 1.4 Hz, 1H), 7.69-7.60 (m, 2H), 7.45-7.35 (m, 3H), 7.27 (dd, J=8.0, 4.5 Hz, 1H), 6.40 (dd, J=17.0, 1.7 Hz, 1H), 6.27 (dd, J=17.0, 10.3 Hz, 1H), 6.03-5.92 (m, 1H), 5.73 (dd, J=10.3, 1.7 Hz, 1H), 4.96-4.87 (m, 1H), 4.79-4.72 (m, 1H), 4.71-4.59 (m, 2H). (Compound 319)

¹H NMR (500 MHz, CDCl₃) δ 8.53 (dd, J=4.5, 1.5 Hz, 1H), 8.13 (dd, J=8.0, 1.5 Hz, 1H), 7.21 (dd, J=8.0, 4.5 Hz, 1H), 5.98-5.91 (m, 1H), 5.67 (dd, J=46.6, 3.1 Hz, 1H), 5.12 (dd, J=15.6, 3.1 Hz, 1H), 5.06-4.99 (m, 1H), 4.93-4.82 (m, 1H), 4.71-4.57 (m, 2H), 3.41 3.30 (m, 1H), 2.46-2.28 (m, 4H), 2.08-1.92 (m, 2H). (Compound 321)

¹H NMR (500 MHz, CDCl₃) δ 8.52 (dd, J=4.5, 1.4 Hz, 1H), 8.11 (dd, J=8.0, 1.3 Hz, 1H), 7.20 (dd, J=8.0, 4.5 Hz, 1H), 5.98-5.88 (m, 1H), 5.66 (dd, J=46.6, 3.0 Hz, 1H), 5.12 (dd, J=15.6, 3.0 Hz, 1H), 5.06-4.96 (m, 1H), 4.93-4.83 (m, 1H), 4.70-4.56 (m, 2H), 1.61 1.52 (m, 1H), 1.00-0.91 (m, 4H). (Compound 322)

¹H NMR (500 MHz, CDCl₃) δ 8.59 (dd, J=4.5, 1.5 Hz, 1H), 8.17 (dd, J=8.0, 1.5 Hz, 1H), 7.65 (s, 1H), 7.53 (s, 1H), 7.28 (dd, J=8.1, 4.5 Hz, 1H), 6.03-5.95 (m, 1H), 5.69 (dd, J=46.7, 3.1 Hz, 1H), 5.14 (dd, J=15.6, 3.1 Hz, 1H), 5.08-5.00 (m, 1H), 4.98-4.88 (m, 1H), 4.75-4.60 (m, 2H), 3.83 (s, 3H). (Compound 323)

¹H NMR (500 MHz, CDCl₃) δ 8.53 (dd, J=4.5, 1.5 Hz, 1H), 8.11 (dd, J=8.0, 1.5 Hz, 1H), 7.21 (dd, J=8.0, 4.5 Hz, 1H), 5.98-5.90 (m, 1H), 5.67 (dd, J=46.6, 3.1 Hz, 1H), 5.12 (dd, J=15.6, 3.1 Hz, 1H), 5.07-4.99 (m, 1H), 4.92-4.84 (m, 1H), 4.72-4.65 (m, 1H), 4.65 4.58 (m, 1H), 2.76-2.67 (m, 1H), 2.01-1.91 (m, 2H), 1.86-1.74 (m, 2H), 1.68-1.53 (m, 4H), 1.45-1.34 (m, 2H). (Compound 325)

¹H NMR (500 MHz, CDCl₃) δ 8.56 (dd, J=4.5, 1.5 Hz, 1H), 8.12 (dd, J=8.0, 1.5 Hz, 1H), 7.24 (dd, J=8.0, 4.5 Hz, 1H), 6.00-5.92 (m, 1H), 5.68 (dd, J=46.7, 3.1 Hz, 1H), 5.13 (dd, J=15.6, 3.1 Hz, 1H), 5.06-4.98 (m, 1H), 4.94-4.85 (m, 1H), 4.70-4.60 (m, 2H), 3.30 3.19 (m, 1H), 3.10-2.98 (m, 2H), 2.96-2.81 (m, 2H). (Compound 326)

¹H NMR (500 MHz, CDCl₃) δ 8.55 (dd, J=4.5, 1.3 Hz, 1H), 8.37 (dd, J=8.0, 1.3 Hz, 1H), 7.60 (d, J=7.4 Hz, 2H), 7.50 (d, J=16.7 Hz, 1H), 7.44-7.35 (m, 3H), 7.32 (t, J=7.3 Hz, 1H), 7.24 (dd, J=8.0, 4.5 Hz, 1H), 6.42 (dd, J=17.0, 1.8 Hz, 1H), 6.30 (dd, J=17.0, 10.3 Hz, 1H), 6.00-5.90 (m, 1H), 5.74 (dd, J=10.3, 1.8 Hz, 1H), 4.93-4.84 (m, 1H), 4.78-4.68 (m, 2H), 4.67-4.60 (m, 1H). (Compound 327)

¹H NMR (500 MHz, CDCl₃) δ 8.55 (dd, J=4.5, 1.5 Hz, 1H), 8.10 (dd, J=8.0, 1.5 Hz, 1H), 7.23 (dd, J=8.0, 4.5 Hz, 1H), 6.39 (dd, J=17.0, 1.8 Hz, 1H), 6.25 (dd, J=17.0, 10.3 Hz, 1H), 5.98-5.89 (m, 1H), 5.72 (dd, J=10.3, 1.8 Hz, 1H), 4.92-4.83 (m, 1H), 4.76-4.68 (m, 1H), 4.67-4.56 (m, 2H), 3.30-3.20 (m, 1H), 2.64-2.52 (m, 1H), 2.43-2.23 (m, 3H), 2.23-2.04 (m, 2H). (Compound 328)

¹H NMR (500 MHz, CDCl₃) δ 8.55 (dd, J=4.5, 1.5 Hz, 1H), 8.10 (dd, J=8.0, 1.5 Hz, 1H), 7.23 (dd, J=8.0, 4.5 Hz, 1H), 5.99-5.91 (m, 1H), 5.68 (dd, J=46.7, 3.1 Hz, 1H), 5.13 (dd, J=15.6, 3.1 Hz, 1H), 5.05-4.98 (m, 1H), 4.93-4.85 (m, 1H), 4.70-4.59 (m, 2H), 3.30 3.21 (m, 1H), 2.64-2.52 (m, 1H), 2.42-2.25 (m, 3H), 2.22-2.03 (m, 2H). (Compound 329)

¹H NMR (500 MHz, CDCl₃) δ 8.50 (dd, J=4.5, 1.4 Hz, 1H), 8.23 (dd, J=8.1, 1.4 Hz, 1H), 7.16 (dd, J=8.0, 4.5 Hz, 1H), 6.69-6.62 (m, 1H), 6.58 (dd, J=16.4, 6.4 Hz, 1H), 6.40 (dd, J=17.0, 1.9 Hz, 1H), 6.27 (dd, J=17.0, 10.3 Hz, 1H), 5.93-5.85 (m, 1H), 5.72 (dd, J=10.3, 1.9 Hz, 1H), 4.87-4.79 (m, 1H), 4.74-4.55 (m, 3H), 2.28-2.18 (m, 1H), 1.92-1.85 (m, 2H), 1.84-1.76 (m, 2H), 1.75-1.67 (m, 1H), 1.43-1.30 (m, 2H), 1.29-1.19 (m, 3H). (Compound 330)

¹H NMR (500 MHz, CDCl₃) δ 8.49 (dd, J=4.5, 1.4 Hz, 1H), 8.22 (dd, J=8.0, 1.4 Hz, 1H), 7.16 (dd, J=8.0, 4.5 Hz, 1H), 6.66 (dd, J=16.5, 0.7 Hz, 1H), 6.58 (dd, J=16.4, 6.4 Hz, 1H), 5.96-5.87 (m, 1H), 5.68 (dd, J=46.6, 3.0 Hz, 1H), 5.12 (dd, J=15.6, 3.0 Hz, 1H), 5.04-4.95 (m, 1H), 4.93-4.79 (m, 1H), 4.74-4.66 (m, 1H), 4.65-4.55 (m, 1H), 2.29-2.18 (m, 1H), 1.94-1.85 (m, 2H), 1.84-1.76 (m, 2H), 1.75-1.67 (m, 1H), 1.42-1.31 (m, 2H), 1.30-1.18 (m, 3H). (Compound 331)

¹H NMR (500 MHz, CDCl₃) δ 8.49 (dd, J=4.5, 1.3 Hz, 1H), 8.15 (dd, J=8.0, 1.3 Hz, 1H), 7.14 (dd, J=8.0, 4.5 Hz, 1H), 6.75 (d, J=16.1 Hz, 1H), 6.40 (dd, J=17.0, 1.8 Hz, 1H), 6.27 (dd, J=17.0, 10.3 Hz, 1H), 6.17 (dd, J=16.1, 9.0 Hz, 1H), 5.92-5.84 (m, 1H), 5.72 (dd, J=10.3, 1.8 Hz, 1H), 4.86-4.80 (m, 1H), 4.73-4.63 (m, 2H), 4.63-4.56 (m, 1H), 1.71-1.62 (m, 1H), 0.95-0.88 (m, 2H), 0.66-0.59 (m, 2H). (Compound 332)

¹H NMR (500 MHz, CDCl₃) δ 8.48 (dd, J=4.5, 1.5 Hz, 1H), 8.15 (dd, J=8.1, 1.5 Hz, 1H), 7.14 (dd, J=8.0, 4.5 Hz, 1H), 6.76 (d, J=16.1 Hz, 1H), 6.18 (dd, J=16.1, 9.1 Hz, 1H), 5.94-5.86 (m, 1H), 5.68 (dd, J=46.6, 3.0 Hz, 1H), 5.12 (dd, J=15.6, 3.0 Hz, 1H), 5.02-4.94 (m, 1H), 4.92-4.82 (m, 1H), 4.73-4.65 (m, 1H), 4.65-4.57 (m, 1H), 1.74-1.59 (m, 1H), 0.95-0.88 (m, 2H), 0.65-0.59 (m, 2H). (Compound 333)

¹H NMR (500 MHz, CDCl₃) δ 8.58 (dd, J=4.5, 1.4 Hz, 1H), 8.21 (dd, J=8.0, 1.3 Hz, 1H), 7.66-7.60 (m, 2H), 7.29-7.24 (m, 1H), 7.13-7.06 (m, 2H), 6.04-5.95 (m, 1H), 5.69 (dd, J=46.7, 3.1 Hz, 1H), 5.13 (dd, J=15.6, 3.1 Hz, 1H), 5.10-5.02 (m, 1H), 4.97-4.88 (m, 1H), 4.76-4.62 (m, 2H). (Compound 334)

¹H NMR (500 MHz, CDCl₃) δ 8.59 (dd, J=4.5, 1.5 Hz, 1H), 8.21 (dd, J=8.0, 1.5 Hz, 1H), 7.46-7.41 (m, 1H), 7.40-7.32 (m, 2H), 7.28 (dd, J=8.0, 4.5 Hz, 1H), 7.15-7.09 (m, 1H), 6.04-5.96 (m, 1H), 5.69 (dd, J=46.7, 3.1 Hz, 1H), 5.14 (dd, J=15.6, 3.1 Hz, 1H), 5.10-5.03 (m, 1H), 4.97-4.89 (m, 1H), 4.75-4.63 (m, 2H). (Compound 335)

¹H NMR (500 MHz, CDCl₃) δ 8.59 (dd, J=4.5, 1.4 Hz, 1H), 8.21 (dd, J=8.0, 1.4 Hz, 1H), 7.64 (d, J=1.6 Hz, 1H), 7.53 (d, J=7.5 Hz, 1H), 7.39 (d, J=8.2 Hz, 1H), 7.33 (t, J=7.8 Hz, 1H), 7.28 (dd, J=8.0, 4.5 Hz, 1H), 6.05-5.95 (m, 1H), 5.69 (dd, J=46.7, 3.1 Hz, 1H), 5.14 (dd, J=15.6, 3.0 Hz, 1H), 5.10-5.02 (m, 1H), 4.97-4.89 (m, 1H), 4.75-4.62 (m, 2H). (Compound 337)

¹H NMR (500 MHz, CDCl₃) δ 8.59 (dd, J=4.5, 1.5 Hz, 1H), 8.23 (dd, J=8.0, 1.5 Hz, 1H), 7.91 (s, 1H), 7.82 (d, J=7.7 Hz, 1H), 7.65 (d, J=7.9 Hz, 1H), 7.54 (t, J=7.8 Hz, 1H), 7.29 (dd, J=8.0, 4.5 Hz, 1H), 6.00 (dq, J=8.2, 5.8 Hz, 1H), 5.69 (dd, J=46.7, 3.1 Hz, 1H), 5.14 (dd, J=15.6, 3.1 Hz, 1H), 5.10-5.02 (m, 1H), 4.98-4.88 (m, 1H), 4.76-4.62 (m, 2H). (Compound 338)

¹H NMR (500 MHz, CDCl₃) δ 8.55 (dd, J=4.5, 1.4 Hz, 1H), 8.34 (dd, J=8.1, 1.3 Hz, 1H), 7.57 (dd, J=8.6, 5.4 Hz, 2H), 7.46 (d, J=16.6 Hz, 1H), 7.31 (d, J=16.6 Hz, 1H), 7.23 (dd, J=8.0, 4.5 Hz, 1H), 7.10 (t, J=8.6 Hz, 2H), 5.96 (dq, J=8.3, 5.8 Hz, 1H), 5.70 (dd, J=46.6, 3.0 Hz, 1H), 5.15 (dd, J=15.6, 3.0 Hz, 1H), 5.06-4.98 (d, J=4.5 Hz, 1H), 4.96-4.88 (m, 1H), 4.77-4.70 (m, 1H), 4.69-4.62 (m, 1H). (Compound 339)

¹H NMR (500 MHz, CDCl₃) δ 8.55 (dd, J=4.5, 1.5 Hz, 1H), 8.34 (dd, J=8.1, 1.5 Hz, 1H), 7.50-7.43 (m, 1H), 7.42-7.32 (m, 3H), 7.31-7.27 (m, 1H), 7.24 (dd, J=8.1, 4.5 Hz, 1H), 7.04-6.97 (m, 1H), 5.96 (tt, J=8.3, 5.8 Hz, 1H), 5.70 (dd, J=46.6, 3.0 Hz, 1H), 5.14 (dd, J=15.6, 3.1 Hz, 1H), 5.07-4.99 (m, 1H), 4.96-4.87 (m, 1H), 4.78-4.70 (m, 1H), 4.69-4.61 (m, 1H). (Compound 340)

¹H NMR (500 MHz, CDCl₃) δ 8.54 (dd, J=4.5, 1.4 Hz, 1H), 8.37 (dd, J=8.1, 1.4 Hz, 1H), 7.68 (td, J=7.7, 1.4 Hz, 1H), 7.64 (d, J=16.9 Hz, 1H), 7.47 (d, J=16.8 Hz, 1H), 7.31-7.26 (m, 1H), 7.24 (dd, J=8.1, 4.5 Hz, 1H), 7.18 (t, J=7.2 Hz, 1H), 7.14-7.08 (m, 1H), 5.96 (tt, J=8.3, 5.8 Hz, 1H), 5.69 (dd, J=46.6, 3.0 Hz, 1H), 5.14 (dd, J=15.6, 3.0 Hz, 1H), 5.06-4.98 (m, 1H), 4.96-4.86 (m, 1H), 4.78-4.70 (m, 1H), 4.69-4.63 (m, 1H). (Compound 341)

1H NMR (500 MHz, DMSO-d₆) δ 8.82 (dd, J=8.1, 1.6 Hz, 1H), 8.64 (dd, J=4.5, 1.5 Hz, 1H), 8.04-7.92 (m, 2H), 7.90-7.75 (m, 3H), 7.71 (d, J=16.8 Hz, 1H), 7.41 (dd, J=8.0, 4.5 Hz, 1H), 5.98 (tt, J=8.5, 5.3 Hz, 1H), 5.57 (dd, J=48.5, 3.5 Hz, 1H), 5.46-5.23 (m, 1H), 5.07-4.72 (m, 2H), 4.70-4.34 (m, 2H). (Compound 344)

¹H NMR (500 MHz, DMSO-d₆) δ 8.85 (dd, J=8.1, 1.7 Hz, 1H), 8.64 (dd, J=4.3, 1.6 Hz, 1H), 8.24-8.11 (m, 2H), 7.86-7.71 (m, 2H), 7.65 (d, J=6.3 Hz, 2H), 7.40 (dd, J=8.1, 4.5 Hz, 1H), 5.98 (tt, J=8.3, 5.3 Hz, 1H), 5.65-5.49 (m, 1H), 5.38 (dd, J=16.6, 3.5 Hz, 1H), 5.01-4.73 (m, 2H), 4.66-4.41 (m, 2H). (Compound 345)

¹H NMR (500 MHz, DMSO-d₆) δ8.70 (dd, J=4.5, 1.5 Hz, 1H), 8.38 (dd, J=8.1, 1.5 Hz, 1H), 7.81 (td, J=7.5, 1.8 Hz, 1H), 7.64-7.50 (m, 1H), 7.50-7.28 (m, 3H), 6.00 (tt, J=8.2, 5.2 Hz, 1H), 5.56 (dd, J=48.4, 3.6 Hz, 1H), 5.37 (dd, J=16.5, 3.6 Hz, 1H), 5.02-4.69 (m, 2H), 4.69-4.28 (m, 2H). (Compound 347)

¹H NMR (500 MHz, DMSO-d₆) δ 8.70 (dd, J=4.5, 1.5 Hz, 1H), 8.38 (dd, J=8.1, 1.4 Hz, 1H), 7.85 (dd, J=7.6, 1.8 Hz, 1H), 7.67 (dd, J=7.9, 1.3 Hz, 1H), 7.60-7.29 (m, 3H), 6.01 (tt, J=8.3, 5.3 Hz, 1H), 5.57 (dd, J=48.4, 3.6 Hz, 1H), 5.37 (dd, J=16.5, 3.5 Hz, 1H), 5.06-4.66 (m, 2H), 4.53 (ddd, J=74.3, 10.9, 7.4 Hz, 2H). (Compound 348)

¹H NMR (500 MHz, DMSO-d₆) δ 8.71 (dt, J=4.6, 1.5 Hz, 1H), 8.28 (dd, J=8.0, 1.5 Hz, 1H), 8.00 (d, J=7.7 Hz, 1H), 7.90 (d, J=7.9 Hz, 1H), 7.80 (t, J=7.6 Hz, 1H), 7.75-7.61 (m, 1H), 7.48 (ddd, J=8.1, 4.6, 1.2 Hz, 1H), 6.01 (tt, J=8.3, 5.3 Hz, 1H), 5.64-5.45 (m, 1H), 5.44-5.29 (m, 1H), 5.04-4.74 (m, 2H), 4.53 (ddd, J=70.5, 10.8, 7.5 Hz, 2H). (Compound 349)

### Examples for Comparison

Prepare the following comparison example (as shown in Table 5) in a similar manner to Examples 1-349 via different reaction starting materials and suitable reagents.

TABLE 5

| Com. EX. | | | Physical Data |
|---|---|---|---|
| No. | Structure | Chemical Name | (LCMS) (M + H)⁺ |
| 1 | 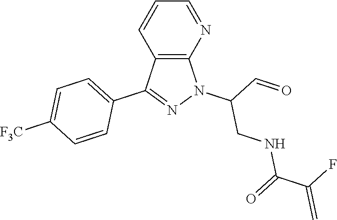 [SMILES: C=C(F)C(=O)NCC(C=O)n1nc(-c2ccc(C(F)(F)F)cc2)c2cccnc21] | 2-fluoro-N-(3-oxo-2-(3-(4- (trifluoromethyl)phenyl)- 1H-pyrazolo[3,4- b]pyridin-1- yl)propyl)acrylamide | 407.1 |
| 2 | 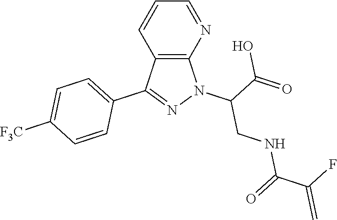 [SMILES: C=C(F)C(=O)NCC(C(=O)O)n1nc(-c2ccc(C(F)(F)F)cc2)c2cccnc21] | 3-(2-fluoroacrylamido)-2- (3-(4- (trifluoromethyl)phenyl)- 1H-pyrazolo[3,4- b]pyridin-1-yl)propanoic acid | 423.1 |
| 3 | 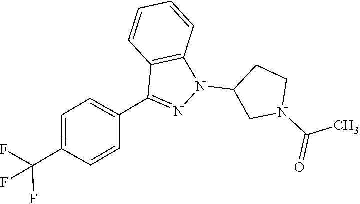 [SMILES: CC(=O)N1CCC(n2nc(-c3ccc(C(F)(F)F)cc3)c3ccccc32)C1] | 1-(3-(3-(4- (trifluoromethyl)phenyl)- 1H-indazol-1- yl)pyrrolidin-1-yl)ethan-1- one | 374.1 |
| 4 | 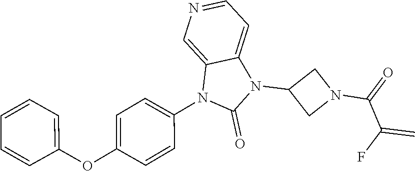 [SMILES: C=C(F)C(=O)N1CC(n2c(=O)n(-c3ccc(Oc4ccccc4)cc3)c3cnccc32)C1] | 1-(1-(2- fluoroacryloyl)azetidin-3- yl)-3-(4-phenoxyphenyl)- 1,3-dihydro-2H- imidazo[4,5-c]pyridin-2- one | 431.1 |
| 5 | 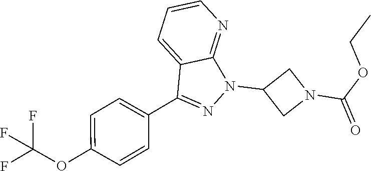 [SMILES: CCOC(=O)N1CC(n2nc(-c3ccc(OC(F)(F)F)cc3)c3cccnc32)C1] | ethyl 3-(3-(4- (trifluoromethoxy)phenyl)- 1H-pyrazolo[3,4- b]pyridin-1-yl)azetidine-1- carboxylate | 407.1 |

### Example a CTGF ELISA Assay

The detection of CTGF expression level can assessed the binding activity of TEAD-YAP/TAZ transcription factor, and the CTGF expression level was quantified by SimpleStep ELISA® kit (Abcam, ab261851).

NCI-H2052 cells (Purchased from ATCC) were cultured in RPMI 1640 medium, adding 10% FBS and 1% Penicillin-Streptomycin Solution and 1 mM Sodium pyruvate. The day before compounds treatment, the cultured cells were washed with PBS and digested with trypsin, then centrifuged and collected. Discarded the supernatant, the cells were resuspended in fresh complete medium. After cells counted, cells were seeded at 6500 cells/well in 96-well plates. And then cells were cultured overnight in incubator (37° C., 5% CO₂).

After the cells were cultured overnight, discarded the culture supernatant, then washed with PBS solution, cells were incubated with 200 μl medium containing compounds in each well. The starting concentration was 10 μM and serial dilution in DMSO and medium were performed to achieve a final DMSO concentration of 0.5% (Final compounds concentration were 10000, 2500, 625, 156, 39.1, 9.77, 2.44, 0.61 nM, 0 nM (0.5% DMSO), The cells were then cultured in incubator (37° C., 5% CO₂). Cultured for 24 hours, the cells were centrifuged at 1500 RPM at 4° C. for 5 minutes, and then 50 μl of culture supernatant were tested for CTGF ELISA assay.

The Human CTGF SimpleStep ELISA® Kit (Abcam, ab261851) employs an affinity tag labeled capture antibody and a reporter conjugated detector antibody which immunocapture the sample analyte in solution. This entire complex (capture antibody/analyte/detector antibody) is in turn immobilized via immunoaffinity of an anti-tag antibody coating the well. To perform the assay, samples or standards are added to the wells, followed by the antibody mix (capture antibody/detector antibody). After incubation, the wells are washed to remove unbound material. TMB Development Solution is added and during incubation is catalyzed by HRP, generating blue coloration. This reaction is then stopped by addition of Stop Solution completing any color change from blue to yellow. Signal is generated proportionally to the amount of bound analyte and the intensity is measured at 450 nm. Firstly, prepare all reagents, samples, and standards as instructed. Add 50 μl standard or sample to appropriate wells. Then add 50 μl of the Antibody Cocktail to each well. Seal the plate and incubate for 1 hour at room temperature on a plate shaker. Wash each well with Wash Buffer three times. Add 100 μl of TMB Development Solution to each well and incubate for 10 minutes in the dark on a plate shaker. Add 100 μl of Stop Solution to each well. Shake plate on a plate shaker for 1 minute to mix. Record the OD at 450 nm. Determine the concentration of the target protein in the sample by the standard curve.

EC₅₀ value was determined by fitting the concentration response curves using GraphPad Prism software. The compounds of the present invention were tested for their capacity to inhibit TEAD-YAP/TAZ interaction according to the fitted curves of CTGF concentration response to compounds.

Data obtained for the Example compounds using the CTGF ELISA assay described above are provided in Table 6.

TABLE 6

| EX No. | EC₅₀(nM) | EX No. | EC₅₀(nM) |
|---|---|---|---|
| 2 | 25 | 186 | 2 |
| 3 | 30.4 | 187 | 2 |
| 4 | 15.5 | 188 | 0.06 |
| 5 | 3 | 189 | 24 |
| 6 | 2 | 196 | 9 |
| 7 | 5 | 197 | 12.6 |
| 9 | 1.4 | 198 | 0.9 |
| 11 | 0.7 | 199 | 20 |
| 13 | 20 | 202 | 8.1 |
| 15 | 35 | 208 | 4.1 |
| 18 | 10 | 209 | 25 |
| 21 | 11 | 217 | 5.7 |
| 22 | 20 | 218 | 0.7 |
| 23 | 16 | 221 | 0.9 |
| 26 | 3.6 | 222 | 0.9 |
| 29 | 12 | 223 | 0.6 |
| 30 | 12 | 225 | 3.6 |
| 33 | 32 | 229 | 20 |
| 34 | 137 | 230 | 6.4 |
| 37 | 42 | 231 | 360 |
| 39 | 14 | 236 | 3 |
| 42 | 14 | 239 | 15 |
| 43 | 11 | 240 | 1.3 |
| 44 | 20 | 242 | 10 |
| 45 | 15 | 243 | 28 |
| 46 | 12 | 244 | 11 |
| 47 | 15 | 245 | 32 |
| 48 | 15 | 252 | 9.8 |
| 50 | 23 | 255 | 44 |
| 51 | 44 | 256 | 0.7 |
| 54 | 32 | 257 | 30 |
| 55 | 16 | 259 | 78 |
| 56 | 70 | 268 | 13 |
| 57 | 82 | 269 | 17 |
| 58 | 17 | 270 | 43 |
| 70 | 34 | 279 | 43 |
| 71 | 12 | 280 | 50 |
| 73 | 7 | 281 | 3 |
| 77 | 6 | 282 | 19 |
| 78 | 21 | 283 | 19 |
| 79 | 7.2 | 284 | 51 |
| 80 | 2 | 294 | 4 |
| 82 | 5 | 306 | 4.8 |
| 87 | 7 | 307 | 3 |
| 89 | 12 | 308 | 5 |
| 94 | 12 | 309 | 53 |
| 95 | 30 | 310 | 15 |
| 96 | 4 | 311 | 20 |
| 98 | 84 | 312 | 6 |
| 100 | 50 | 315 | 16 |
| 101 | 97 | 316 | 4 |
| 102 | 183 | 317 | 8 |
| 103 | 158 | 319 | 3 |
| 104 | 14 | 320 | 39 |
| 108 | 12 | 321 | 11 |
| 111 | 84 | 322 | 10 |
| 112 | 86 | 324 | 20 |
| 120 | 36 | 326 | 19 |
| 121 | 3 | 327 | 2 |
| 122 | 24 | 328 | 18 |
| 123 | 7 | 329 | 12 |
| 124 | 14 | 330 | 3 |
| 125 | 75 | 331 | 6 |
| 127 | 8 | 332 | 37 |
| 128 | 9 | 333 | 24 |
| 129 | 8 | 334 | 2 |
| 130 | 4 | 335 | 3 |
| 131 | 18 | 337 | 4 |
| 132 | 6 | 338 | 8 |
| 160 | 9 | 339 | 8 |
| 161 | 3 | 340 | 2 |
| 165 | 40 | 343 | 4 |
| 171 | 148 | 344 | 1.4 |
| 172 | 0.4 | 345 | 7 |
| 173 | 48 | 346 | 4 |
| 178 | 34 | 347 | 7 |
| 182 | 0.3935 | 348 | 8 |
| 184 | 7.686 | 349 | 19 |
| Com. EX.1 | >10000 | Com. EX.2 | >10000 |
| Com. EX.3 | >10000 | Com. EX.4 | >10000 |
| Com. EX.5 | >10000 | | |

### Example B BRDU assay

PerkinElmer's DELFIA® Cell proliferation kit (PerkinElmer, Cat: AD0200) was used to detect the inhibition on the proliferation of NCI-H226 cells (ATCC, CRL-5826).
a) Seeded 1500/well for NCI-H226 cell into a 96-well plate.
b) After 24 hours, compound was added to wells with 1% FBS medium conditions. The final testing concentrations of test compounds were 20000, 6666.667, 2222.222, 740.741, 246.914, 82.305, 27.435, 9.145, 3.048, 0.102 nM.
c) NCI-H226 cells were incubated for 72 hours in incubator.
d) Diluted BrdU Labeling Reagent 100-fold with medium and added 24 per well. The NCI-H226 cells were incubated 24 h in 5% CO₂ at 37° C. incubator.
e) Detect cell proliferation according BrdU kit, and the luminescence signal value of each well on the multifunctional microplate reader was readed.

Data analysis:
Use GraphPad Prism 6 software to calculate IC₅₀ and plot effect-dose curve of compounds.

Y=Bottom+(Top−Bottom)/(1+10\hat{ }((LogIC₅₀−X)×HillSlope)).
Y was the inhibition %,
X was the log concentration of compounds.

Inhibition %=(signal_{HC}−signal_{comp})/(signal_{HC}−signal_{LC});
HC (high control) was DMSO group,
LC (low control) was 10 uM staurosporine group,
Comp was administration group.

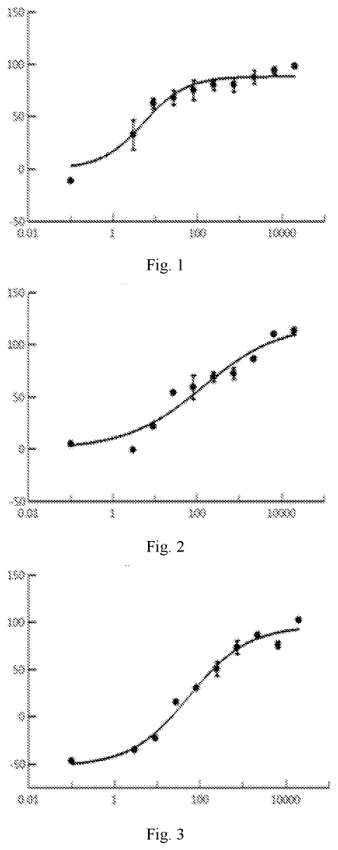

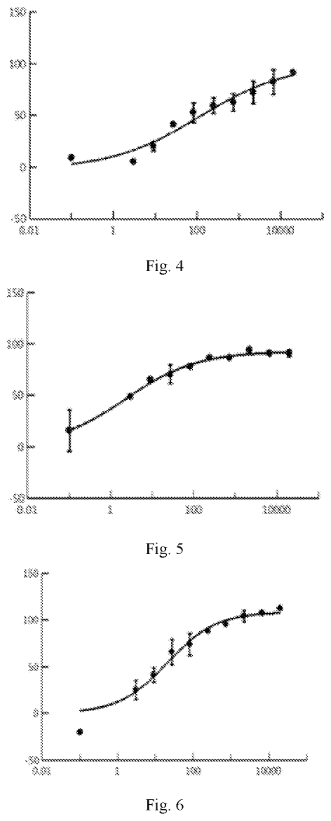

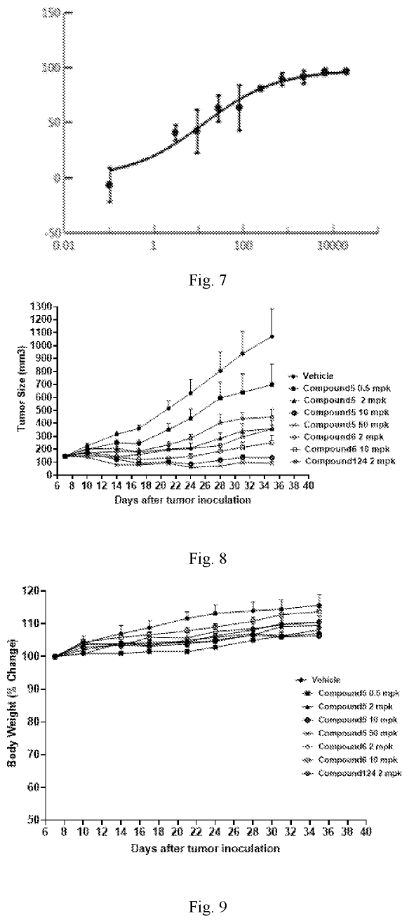

IC₅₀ data obtained for the Example compounds are provided in Table 7, and the inhibition curve in NCI-H226 cell line are as shown in FIG. 1-7, wherein X axis is compound concentration (nm), Y axis is the inhibition %.

TABLE 7
The proliferation inhibition potency (IC₅₀)
of compounds in NCI-H226 cells

| EX No. | IC₅₀(nM) |
|---|---|
| 3 | 19.48 |
| 5 | 5.33 |
| 6 | 140.11 |
| 30 | 55.69 |
| 73 | 114.5 |
| 80 | 2.32 |
| 124 | 20.73 |
| 132 | 12.65 |

### Example C Pharmacokinetics of Compounds in Plasma Following PO Cassette Dosing in Mice

Adult Balb/C female mice (6-7 weeks, Vitalriver) were received to cassette dosing of the test compounds with 10-20% DMSO, 10% Solutol (Kolliphor HS 15, Beijing Fengli Jingqiu Pharmaceutical Co., Ltd.) and 80-70% H₂O as excipients. The mice (n=3) were giving oral administration (intragastric administration) at a dose of 5 mg/kg. Time of blood collection: 30 min, 2 h, 4 h. About 0.1 mL of whole blood was collected from retro-orbital venous plexus, and placed into tubes that contained EDTA as an anticoagulant. The samples were centrifuged at 4° C. and 4000 rpm for 10 min. The plasma was transferred into centrifuge tubes, and stored at −20° C. before being analyzed. Concentrations of test compounds in the plasma samples were analyzed with liquid chromatography-tandem mass spectrometry (LC-MS/MS). Plasma concentration-time data of individual animals was analyzed using Microsoft Excel 2010. Non-compartmental model was introduced in concentration analysis. The pharmacokinetic parameters of the test compounds were calculated using WinNonlin (version 4.1; Pharsight) software. As shown in table 8, the tested compounds showed great pharmacokinetic properties.

TABLE 8

| EX No. | Cmax(ng/mL) | AUC(h*ng/ml) |
|---|---|---|
| 21 | 1062 | 3150 |
| 26 | 1423 | 4641 |
| 47 | 721 | 2507 |
| 58 | 1583 | 4474 |
| 73 | 1187 | 4068 |
| 89 | 1974 | 6480 |
| 104 | 1520 | 5057 |
| 108 | 1197 | 3505 |
| 124 | 7193 | 24308 |
| 128 | 5273 | 19278 |
| 130 | 1960 | 7110 |
| 172 | 1650 | 1650 |
| 173 | 1670 | 5963 |
| 182 | 2163 | 7409 |
| 183 | 3680 | 12750 |
| 218 | 1192 | 3783 |
| 229 | 1042 | 3835 |
| 240 | 1602 | 5877 |
| 241 | 2527 | 8258 |
| 242 | 1767 | 5407 |
| 243 | 1213 | 4010 |
| 268 | 1693 | 6096 |
| 269 | 1250 | 4373 |
| 281 | 1193 | 4204 |
| 307 | 2707 | 8482 |
| 308 | 2613 | 9110 |
| 312 | 2080 | 5794 |
| 331 | 1759 | 5728 |

### Example D In Vivo Pharmacodynamic and Efficacy Study

In vivo pharmacodynamic and efficacy studies of compound 5, compound 6, and compound 124 in the subcutaneous NCI-H226 human lung squamous cell carcinoma xenograft model on BALB/c nude mice.

Method:

Each mouse (D000521 BALB/c-Nu, GemPharmatech Co., Ltd.) was inoculated subcutaneously at the right flank with NCI-H226 tumor cells (ATCC, CRL-5826) (1×107) in 0.2 mL of PBS with Matrigel (Corning, 356234) (1:1) for tumor development. Treatments were started when the average tumor size reached approximately 100-150 mm3. The test article was administered to the mice orally once a day from the day of grouping, total 28 days (QD×28 Days). Body weight change of animals was monitored regularly as an indicator of drug safety. Tumor volumes were measured twice weekly in two dimensions using a caliper, and the volume was expressed in mm3 using the formula “V=(LλW\hat{ }2)/2”, where V is the tumor volume, L is the tumor length (the longest tumor dimension) and W is the tumor width (the longest tumor dimension perpendicular to L). Therapeutic efficacy was evaluated by tumor growth inhibition TGI (%)·TGI (%)=[1−(Ti−T0)/(Vi−V0)]×100; Ti is the average tumor volume of a treatment group on a given day, TO is the average tumor volume of the treatment group on day 0, Vi is the average tumor volume of the vehicle control group on the same day with Ti, and V0 is the average tumor volume of the vehicle group on day 0. The results are shown in table 9, FIG. 8 and FIG. 9.

TABLE 9
Tumor growth inhibition calculation in the NCI-H226
xenograft model based on tumor volume at day 28

| | Dose | | |
|---|---|---|---|
| Group | (mg/kg) | TGI (%) | p value |
| Vehicle | — | — | — |
| Compound 5 | 0.5 mg/kg, QD | 40.3 | 0.2297 |
| Compound 5 | 2 mg/kg, QD | 76.9 | 0.0111 |
| Compound 5 | 10 mg/kg, QD | 101.3 | 0.0011 |
| Compound 5 | 50 mg/kg, QD | 105.9 | 0.0007 |
| Compound 6 | 2 mg/kg, QD | 77.1 | 0.007 |
| Compound 6 | 10 mg/kg, QD | 88.6 | 0.0026 |
| Compound 124 | 2 mg/kg, QD | 67.3 | 0.0284 |

Results

On day 28 post treatment, compound 5 group (2 mg/kg), compound 5 group (10 mg/kg), compound 5 group (50 mg/kg), compound 6 group (2 mg/kg), compound 6 group (10 mg/kg), compound 124 group (2 mg/kg) produced significant anti-tumor activities compared with the vehicle group in tumor volume. The p values were 0.0111, 0.0011, 0.0007, 0.007, 0.0026, and 0.0284, respectively. TGI (%) values were 76.9%, 101.3%, 105.9%, 77.1%, 88.6%, and 67.3%, respectively.

In this model, as shown in FIG. 9, no significant body weight loss was observed during the treatment with daily dosing of compound 5, compound 6, and compound 124.

1. A compound of Formula (I), or a stereoisomer, tautomer, pharmaceutically acceptable salt, prodrug, chelate, non-covalent complex, or solvate thereof, 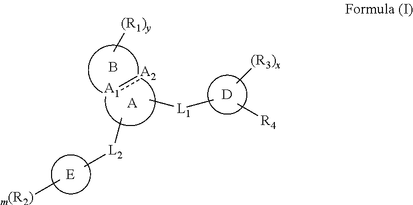
wherein, [symbol]
is a single bond or a double bond;
A₁ and A₂ are independently C or N;
ring B is selected from the group consisting of C₅₋₆ aryl, C₅₋₆ cycloalkyl, 5 to 6-membered heterocyclyl and 5 to 6-membered heteroaryl, wherein the 5 to 6-membered heterocyclyl and 5 to 6-membered heteroaryl comprising 1, 2, 3 or 4 heteroatoms independently selected from N, S and O;
ring A is selected from the group consisting of C₅₋₆ aryl, 5 to 6-membered heteroaryl, 5 to 6-membered heterocyclyl, the 5 to 6-membered heteroaryl and 5 to 6-membered heterocyclyl comprising 1-4 hetero atoms independently selected from N, S and O; wherein the C₅₋₆ aryl, 5 to 6-membered heteroaryl and 5 to 6-membered heterocyclyl are each optionally substituted with 0 to 3 substituents independently selected from the group consisting of oxo, ═NH, hydroxyl, halogen, CN, —NH(C₁₋₆ alkyl), —N—(C₁₋₆ alkyl)₂, C₁₋₆ alkyl, C₂₋₆ alkenyl, C₂₋₆ alkynyl, C₁₋₆ haloalkyl, C₁₋₆ alkoxyl, C₁₋₆ haloalkoxyl, —OC(═O)Rₐ, —C(═O)NRₐR_{b},—C(═O)ORₐ, —C(═O)R_{c}, —S(═O)R_{b}, —S(═O)₂R_{b}, —S(═O)NRₐR_{b}, —S(═O)₂NRₐR_{b};
L₁ is bond, —O—, —S—, —(CH₂)ₜ—, —(CH₂)ₜ—NRₐ—,—NRₐ—(CH₂)ₜ—, —(CH₂)ₜ—O—, —O—(CH₂)ₜ—, —C(═O)—, —C(═O)NRₐ— or —NRₐ—C(═O)—;
ring E is C₅₋₆ aryl, 5 to 10-membered heteroaryl, C₃₋₈ cycloalkyl or 4 to 8-membered heterocyclyl, wherein the 5 to 10-membered heteroaryl and 4 to 8-membered heterocyclyl comprising 1-4 hetero atoms independently selected from N, S and O;
L₂ is bond, —O—, —S—, —NH—, —(CH₂)ₜ—O—, —O—(CH₂)ₜ—, —C(═O)—, —C₁₋₄ alkylene, —C₂₋₄ alkenylene, or —C₂₋₄ alkynylene;
ring D is C₅₋₁₀ aryl, 5 to 10-membered heteroaryl, C₃₋₁₀ cycloalkyl or 4 to 10-membered heterocyclyl, wherein the 5 to 10-membered heteroaryl or 4 to 10-membered heterocyclyl comprising 1, 2, 3 or 4 heteroatoms independently selected from N, S and O;
R₁ is H, oxo, hydroxyl, halogen, CN, —NO₂, —NR_{d}Rₑ, C₁₋₆ alkyl, C₂₋₆ alkenyl, C₂₋₆ alkynyl, C₁₋₆ haloalkyl, C₁₋₆ alkoxyl, —C(═O)NRₐR_{b},—C(═O)ORₐ, —C(═O)R_{c}, —S(═O)R_{b}, —S(═O)₂R_{b}, —S(═O)NRₐR_{b}, —S(═O)₂NRₐR_{b}, —O—(C═O)—Rₐ, —O—(C═O)—NRₐR_{b}, C₁₋₆ haloalkoxyl, C₃₋₆ cycloalkyl, 3 to 6-membered heterocyclyl, C₅₋₆ aryl or 5 to 6-membered heteroaryl, wherein C₁₋₆ alkyl, C₂₋₆ alkenyl, C₂₋₆ alkynyl, C₁₋₆ haloalkyl, C₁₋₆ alkoxyl, C₁₋₆ haloalkoxyl, C₃₋₆ cycloalkyl, 3 to 6-membered heterocyclyl, C₅₋₆ aryl and 5 to 6-membered heteroaryl are each optionally substituted with 0 to 3 substituents independently selected from the group consisting of OH, CN, halogen, C₁₋₆ alkyl, C₂₋₄ alkenyl, C₂₋₄ alkynyl, C₁₋₄ haloalkyl, C₁₋₄ alkoxyl, —NRₐR_{b}, —C(═O)NRₐR_{b}, —OC(═O)Rₐ, —C(═O)ORₐ, —C(═O)Rₐ, —S(═O)R_{b}, —S(═O)₂R_{b}, —S(═O)NRₐR_{b}, —S(═O)₂NRₐR_{b}, —NRₐC(═O)R_{b}, C₁-4 haloalkoxyl, C₃₋₆ cycloalkyl, 3 to 6-membered heterocycloalkyl, C₅₋₆ aryl and 5 to 6-membered heteroaryl; wherein the 5 to 6-membered heteroaryl and 3 to 6-membered heterocyclyl optionally comprising 1, 2 or 3 hetero atoms independently selected from N, S and O;
R₂ is H, hydroxyl, halogen, CN, —NO₂, —NRₐR_{b}, oxo, —C(═O)NRₐR_{b}, —C(═O)ORₐ, —C(═O)Rₐ, —S(═O)R_{b}, —S(═O)₂R_{b}, —S(═O)NRₐR_{b}, —S(═O)₂NRₐR_{b}, —NRₐC(═O)R_{b}, SF₅, C₁₋₆ alkyl, C₂₋₆ alkenyl, C₂₋₆ alkynyl, C₁₋₆ alkoxyl, C₁₋₆ haloalkyl, C₃₋₆ cycloalkyl or 3 to 6-membered heterocyclyl comprising 1, 2 or 3 hetero atoms independently selected from N, S and O; wherein the C₁₋₆ alkyl, C₂₋₆ alkenyl, C₂₋₆ alkynyl, C₁₋₆ alkoxyl, C₃₋₆ cycloalkyl and 3 to 6-membered heterocyclyl are each optionally substituted with 0 to 3 substitutents independently selected from the group consisting of —ORₐ, halogen, CN, C₁₋₄ alkyl, C₁₋₆ haloalkyl, —NRₐR_{b}, oxo, z OC(═O)Rₐ, —C(═O)NRₐR_{b}, —C(═O)ORₐ, —C(═O)Rₐ, —S(═O)R_{b}, —S(═O)₂R_{b}, —S(═O)NRₐR_{b}, —S(═O)₂NRₐR_{b} and —NRₐC(═O)R_{b};
R₃ is H, oxo, halogen, —ORₐ, CN, —NO₂, —NRₐR_{b}, —NRₐC(═O)R_{b}, —C₁₋₄ alkylene-NRₐR_{b}, —C₁₋₄ alkylene-NRₐC(═O)R_{b}, —C(═O)R_{b}, —OC(═O)R 4, —C(═O)ORₐ, —C(═O)NRₐR_{b}, —S(═O)R_{b}, —S(═O)₂R_{b}, —S(═O)NRₐR_{b}, —S(═O)₂NRₐR_{b}, —C₁₋₄ alkylene-C(═O)NRₐR_{b}, C₁₋₆ alkyl, C₂₋₆ alkenyl, C₂₋₆ alkynyl, C₁₋₆ alkoxyl, C₁₋₆ haloalkyl, C₁₋₆ haloalkoxyl, 3 to 6-membered heterocyclyl, C₃₋₆ cycloalkyl, C₅₋₆ aryl or 5 to 6-membered heteroaryl, wherein the 5 to 6-membered heteroaryl and 3 to 6-membered heterocyclyl optionally comprising 1, 2 or 3 hetero atoms independently selected from N, S and O; which C₁₋₆ alkyl, C₂₋₆ alkenyl, C₂₋₆ alkynyl, C₁₋₆ haloalkyl, C₁₋₆ alkoxyl, C₁₋₆ haloalkoxyl, C₃₋₆ cycloalkyl, 3 to 6-membered heterocyclyl, C₅₋₆ aryl and 5 to 6-membered heteroaryl are each optionally substituted with 0 to 3 substitutents independently selected from the group consisting of oxo, hydroxyl, halogen, CN, —NO₂, C₁₋₆ alkyl, —C₁₋₄ alkylene-OH, C₁₋₆ haloalkyl, C₁₋₆ alkoxyl, z S(═O)R_{b}, —S(═O)₂R_{b}, —NRₐR_{b}, —C(═O)R_{b}, —OC(═O)Rₐ, —C(═O)ORₐ, —NRₐC(═O)R_{b}, —C(═O)NRₐR_{b}, —NRₐC(═O)R_{b}, —C₁₋₄ alkylene-NRₐR_{b}, —C₁₋₄ alkylene-NRₐC(═O)R_{b}, C₁₋₄ alkylene-C(═O)NRₐR_{b}, —C₁₋₄ alkylene-OH, C₃₋₆ cycloalkyl, 3 to 6-membered heterocycloalkyl, C₅₋₆ aryl and 5 to 6-membered heteroaryl;
R₄ is 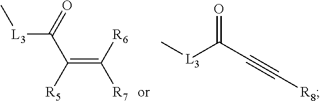
L₃ is bond, —(CH₂)ₜ—NRₐ—, —C₄₋₆ heterocyclyl or —C₄₋₆cycloalkyl-NRₐ—;
R₅, R₆, R₇ and R₅ are independently selected from the group consisting of H, halogen, —ORₐ, CN, —NRₐR_{b}, —C₁₋₆ alkylene-NRₐR_{b}, —C₁₋₆ alkylene-R_{c}, C₁₋₆ alkyl, C₂₋₆ alkenyl, C₂₋₆ alkynyl, C₁₋₆ alkoxyl, C₃₋₆ cycloalkyl, 3 to 6-membered heterocyclyl, C₅₋₆ aryl and 5 to 6-membered heteroaryl, wherein C₁₋₆ alkyl, C₂₋₆ alkenyl, C₂₋₆ alkynyl, C₁₋₆ alkoxyl, C₃₋₆ cycloalkyl, 3 to 6-membered heterocyclyl, C₅₋₆ aryl and 5 to 6-membered heteroaryl are each optionally substituted with 0 to 4 substitutents independently selected from the group consisting of OH, CN, halogen, C₁₋₆ alkyl, C₂₋₄ alkenyl, C₂₋₄ alkynyl, C₁₋₄ haloalkyl, C₁₋₄ alkoxyl, —NRₐR_{b}, C₃₋₆ cycloalkyl, 3 to 6-membered heterocyclyl, C₅₋₆ aryl and 5 to 6-membered heteroaryl, wherein the 5 to 6-membered heteroaryl and 3 to 6-membered heterocyclyl optionally comprising 1, 2 or 3 hetero atoms independently selected from N, S and O;
Rₐ and R_{b} are independently selected from the group consisting of H, CN, hydroxyl, halogen, C₁₋₆ alkyl, C₁₋₆ haloalkyl, C₁₋₄ alkoxyl, C₃₋₆ cycloalkyl, 3 to 6-membered heterocycloalkyl, C₅₋₆ aryl and 5 to 6-membered heteroaryl, wherein the C₁₋₆ alkyl, C₁₋₄ alkoxyl, C₃₋₆ cycloalkyl, 3 to 6-membered heterocycloalkyl, C₅₋₆ aryl and 5 to 6-membered heteroaryl are each optionally substituted with 0 to 4 substitutents independently selected from the group consisting of halogen, CN, —OH, oxo, C₁₋₆ alkyl, C₂₋₆ alkenyl, C₂₋₆ alkynyl, C₁₋₆ haloalkyl, C₁₋₃ alkoxyl, C₁₋₃ haloalkoxyl and C₅₋₆ aryl; wherein the 5 to 6-membered heteroaryl and 3 to 6-membered heterocycloalkyl optionally comprising 1, 2 or 3 hetero atoms independently selected from N, S and O;
R_{c} is 3 to 6-membered heterocyclyl optionally substituted with 0 to 4 substitutents independently selected from the group consisting of halogen, CN, —OH, oxo, C₁₋₆ alkyl, C₂₋₆ alkenyl, C₂₋₆ alkynyl, C₁₋₆ haloalkyl, C₁₋₃ alkoxyl and C₁₋₃ haloalkoxyl;
R_{d} and Rₑ are independently selected from the group consisting of C₁₋₆ alkyl, C₂₋₆ alkenyl, C₂₋₆ alkynyl, C₁₋₆ haloalkyl, C₁₋₆ alkoxyl, —C(═O)NR_{f}R_{f},—C(═O)OR_{f}, —O—C(═O)R_{f}, —C(═O)R_{f}, —S(═O)R_{f}, —S(═O)₂R_{f}, —S(═O)NR_{f}R_{f}, —S(═O)₂NR_{f}R_{f}, —C₁₋₄ alkylene-NR_{f}R_{f}, —C₁₋₄ alkylene-NR_{f}C(═O)R_{f}, —C₁₋₄ alkylene-C(═O)NR_{f}R_{f};
R_{f} is H, C₁₋₆ alkyl, C₂₋₆ alkynyl, C₁₋₆ haloalkyl, C₁₋₆ haloalkoxyl or C₁₋₆ alkoxyl;
t is 1, 2, 3 or 4;
x, y and m are independently 0, 1, 2, 3, 4 or 5.

2. The compound, or the stereoisomer, tautomer, pharmaceutically acceptable salt, prodrug, chelate, non-covalent complex, or solvate of claim 1, wherein L₁ is bond, —O—, —S—, —NRₐ—, —(CH₂)ₜ—, —(CH₂)ₜ—O—, —O—(CH₂)ₜ—, —C(═O)—, —C(═O)NRₐ— or —NRₐ—C(═O)—.

3. The compound or the stereoisomer, tautomer, pharmaceutically acceptable salt, prodrug, chelate, non-covalent complex, or solvate of claim 1, wherein L₂ is bond, —O—, —S—, —NH—, —C(═O)—, —C₂₋₄ alkenylene, or —C₂₋₄ alkynylene.

4. (canceled)

5. (canceled)

6. The compound or the stereoisomer, tautomer, pharmaceutically acceptable salt, prodrug, chelate, non-covalent complex, or solvate of claim 1, wherein ring A is selected from the group consisting of C₅₋₆ aryl, 5 to 6-membered heteroaryl, 5 to 6-membered heterocyclyl, the 5 to 6-membered heteroaryl and 5 to 6-membered heterocyclyl comprising 1-4 hetero atoms independently selected from N, S and O, wherein the 5 to 6-membered heterocyclyl and the 5 to 6-membered heteroaryl are optionally and independently substituted with one or more

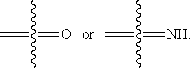

7. (canceled)

8. The compound or the stereoisomer, tautomer, pharmaceutically acceptable salt, prodrug, chelate, non-covalent complex, or solvate of claim 1, wherein ring B is selected from the group consisting of C₅₋₆ aryl, C₅₋₆ cycloalkyl, 5 to 6-membered heteroaryl comprising 1, 2, 3 or 4 N heteroatoms, and 5 to 6-membered heterocyclyl comprising 1, 2, 3 or 4 heteroatoms independently selected from N, S, O.

9. (canceled)

10. The compound or the stereoisomer, tautomer, pharmaceutically acceptable salt, prodrug, chelate, non-covalent complex, or solvate of claim 1, wherein the compound is of Formula (II-1) or Formula (II-2), 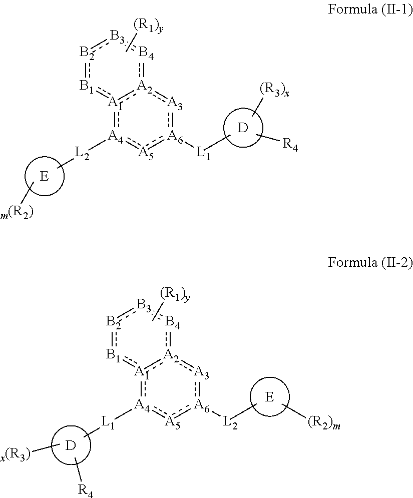
or a stereoisomer, tautomer, pharmaceutically acceptable salt, prodrug, chelate, non-covalent complex, or solvate thereof, wherein,
A₁, A₂, A₄ and A₆ are independently C or N;
A₃ is absent, CH₂, CH, C═O or N;
A₅ is C, CH, C═O, C═NH or N;
B₁, B₂, B₃ and B₄ are independently selected from the group consisting of C, CH, CH₂, C═O, NH or N.

11. The compound of claim 1, or a stereoisomer, tautomer, pharmaceutically acceptable salt, prodrug, chelate, non-covalent complex, or solvate thereof, wherein R₁ is H, oxo, hydroxyl, halogen, CN,—NO₂, —NR_{d}Rₑ, C₁₋₆ alkyl, C₂₋₆ alkenyl, C₂₋₆ alkynyl, C₁₋₆ haloalkyl, C₁₋₆ alkoxyl, —C(═O)NRₐR_{b},—C(═O)ORₐ, —C(═O)R_{c}, —S(═O)R_{b}, —S(═O)₂R_{b}, —S(═O)NRₐR_{b}, —S(═O)₂NRₐR_{b}, —O—(C═O)—Rₐ, —O—(C═O)—NRₐR_{b}, C₁₋₆ haloalkoxyl, C₃₋₅ cycloalkyl, 3 to 5-membered heterocyclyl, wherein C₁₋₆ alkyl, C₂₋₆ alkenyl, C₂₋₆ alkynyl, C₁₋₆ haloalkyl, C₁₋₆ alkoxyl, C₁₋₆ haloalkoxyl, C₃₋₅ cycloalkyl, 3 to 5-membered heterocyclyl are each optionally substituted with 0 to 3 substituents independently selected from the group consisting of OH, CN, halogen, C₁₋₆ alkyl, C₂₋₄ alkenyl, C₂₋₄ alkynyl, C₁₋₄ haloalkyl, C₁₋₄ alkoxyl, —NRₐR_{b}, —C(═O)NRₐR_{b}, —C(═O)ORₐ, —C(═O)Rₐ, —S(═O)R_{b}, —S(═O)₂R_{b}, —S(═O)NRₐR_{b}, —S(═O)₂NRₐR_{b}, —NRₐC(═O)R_{b}, C₁₋₄ haloalkoxyl.

12. The compound of claim 1, or a stereoisomer, tautomer, pharmaceutically acceptable salt, prodrug, chelate, non-covalent complex, or solvate thereof, wherein R₂ is H hydroxyl, halogen, CN, —NO₂, —NRₐR_{b}, oxo, —C(═O)NRₐR_{b}, —C(═O)ORₐ, —C(═O)Rₐ, —S(═O)R_{b}, —S(═O)₂R_{b}, —S(═O)NRₐR_{b}, —S(═O)₂NRₐR_{b}, —NRₐC(═O)R_{b},—SF₅, C₁₋₆ alkyl, C₂₋₆ alkenyl, C₂₋₆ alkynyl, C₁₋₆ alkoxyl, C₁₋₆ haloalkyl, C₃₋₆ cycloalkyl; wherein the C₁₋₆ alkyl, C₂₋₆ alkenyl, C₂₋₆ alkynyl, C₁₋₆ alkoxyl, C₁₋₆ haloalkyl, C₃₋₆ cycloalkyl are each optionally substituted with 0 to 3 substitutents independently selected from the group consisting of —ORₐ, —NH₂, halogen, CN, C₁₋₄ alkyl, C₁₋₆ haloalkyl, —NRₐR_{b}, oxo, —C(═O)NRₐR_{b}, —OC(═O)R a, —C(═O)ORₐ, —C(═O)Rₐ, —S(═O)R_{b}, —S(═O)₂R_{b}, —S(═O)NRₐR_{b}, —S(═O)₂NRₐR_{b} and —NRₐC(═O)R_{b}.

13. (canceled)

14. (canceled)

15. (canceled)

16. The compound or the stereoisomer, tautomer, pharmaceutically acceptable salt, prodrug, chelate, non-covalent complex, or solvate of claim 1, wherein ring E is C₅₋₆ aryl, 5 to 6-membered heteroaryl, C₃₋₈ cycloalkyl or 4 to 8-membered heterocyclyl, wherein the 5 to 6-membered heteroaryl and 4 to 8-membered heterocyclyl comprising 1, 2 or 3 heteroatoms independently selected from N, S and O.

17. (canceled)

18. The compound or the stereoisomer, tautomer, pharmaceutically acceptable salt, prodrug, chelate, non-covalent complex, or solvate of claim 1, wherein ring D is C₅₋₁₀ aryl, 5 to 10-membered heteroaryl, C₃₋₁₀ cycloalkyl or 4 to 10-membered heterocyclyl, wherein the 5 to 10-membered heteroaryl and 4 to 10-membered heterocyclyl comprising 1, 2 or 3 heteroatoms independently selected from N and 0.

19. (canceled)

20. (canceled)

21. The compound or the stereoisomer, tautomer, pharmaceutically acceptable salt, prodrug, chelate, non-covalent complex, or solvate of claim 1, wherein R₃ is H, halogen, —OR a, CN, —C₁₋₄ alkylene-NRₐR_{b}, —C₁₋₄ alkylene-C(═O)NRₐR_{b}, C₁₋₆ alkyl, C₁₋₆ alkoxyl, C₁₋₆ haloalkyl, C₁₋₆ haloalkoxyl, 3 to 6-membered heterocyclyl, C₃₋₆ cycloalkyl, C₅₋₆ aryl or 5 to 6-membered heteroaryl, wherein the 5 to 6-membered heteroaryl and 3 to 6-membered heterocyclyl optionally comprising 1, 2 or 3 hetero atoms independently selected from N, S and O; wherein C₁₋₆ alkyl, C₃₋₆ cycloalkyl, 3 to 6-membered heterocyclyl, C₅₋₆ aryl and 5 to 6-membered heteroaryl are each optionally substituted with 0 to 3 substitutents independently selected from the group consisting of halogen, CN, C₁₋₆ alkyl, C₁₋₆ haloalkyl, —NRₐR_{b}, —C(═O)ORₐ, —C(═O)NRₐR_{b}.

22. (canceled)

23. The compound or the stereoisomer, tautomer, pharmaceutically acceptable salt, prodrug, chelate, non-covalent complex, or solvate of claim 1, wherein L₃ is bond, —NH—,—N—C₁₋₃ alkyl-, —(CH₂)ₜ—NH—, —C₄₋₆ heterocyclyl.

24. (canceled)

25. The compound or the stereoisomer, tautomer, pharmaceutically acceptable salt, prodrug, chelate, non-covalent complex, or solvate of claim 1, wherein R₅, R₆ and R₇ are independently selected from the group consisting of H, halogen, CN, C₁₋₆ alkyl, —C₁₋₆ alkylene-NRₐR_{b}, and —C₁₋₆ alkylene-R_{c}.

26. The compound or the stereoisomer, tautomer, pharmaceutically acceptable salt, prodrug, chelate, non-covalent complex, or solvate of claim 1, wherein R₈ is H, halogen, CN, C₁₋₄ alkyl or —C₁₋₄ alkylene-NRₐR_{b}.

27. The compound or the stereoisomer, tautomer, pharmaceutically acceptable salt, prodrug, chelate, non-covalent complex, or solvate of claim 1, wherein Rₐ and R_{b} are independently selected from the group consisting of H, C₁₋₆ alkyl, C₃₋₆ cycloalkyl, 3 to 6-membered heterocycloalkyl, C₅₋₆ aryl and 5 to 6-membered heteroaryl, wherein the C₁₋₆ alkyl, C₃₋₆ cycloalkyl, 3 to 6-membered heterocycloalkyl, C₅₋₆ aryl and 5 to 6-membered heteroaryl may optionally be substituted with halogen, C₁₋₆ haloalkyl or C₅₋₆ aryl, wherein the 5 to 6-membered heteroaryl and 3 to 6-membered heterocycloalkyl optionally comprising 1, 2 or 3 hetero atoms independently selected from N, S and O.

28. (canceled)

29. (canceled)

30. The compound or the stereoisomer, tautomer, pharmaceutically acceptable salt, prodrug, chelate, non-covalent complex, or solvate of claim 1, wherein R_{c} is 3 to 6-membered heterocycloalkyl which may be substituted with halogen or C₁₋₆ haloalkyl.

31. The compound or the stereoisomer, tautomer, pharmaceutically acceptable salt, prodrug, chelate, non-covalent complex, or solvate of claim 1, wherein R_{d} is H, C₁₋₆ alkyl, and Rₑ is C₁₋₆ alkyl, —C(═O)Rₑ, S(═O)₂R_{b}.

32. (canceled)

33. (canceled)

34. The compound or the stereoisomer, tautomer, pharmaceutically acceptable salt, prodrug, chelate, non-covalent complex, or solvate of claim 1, wherein ring 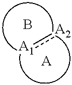
is selected from the group consisting 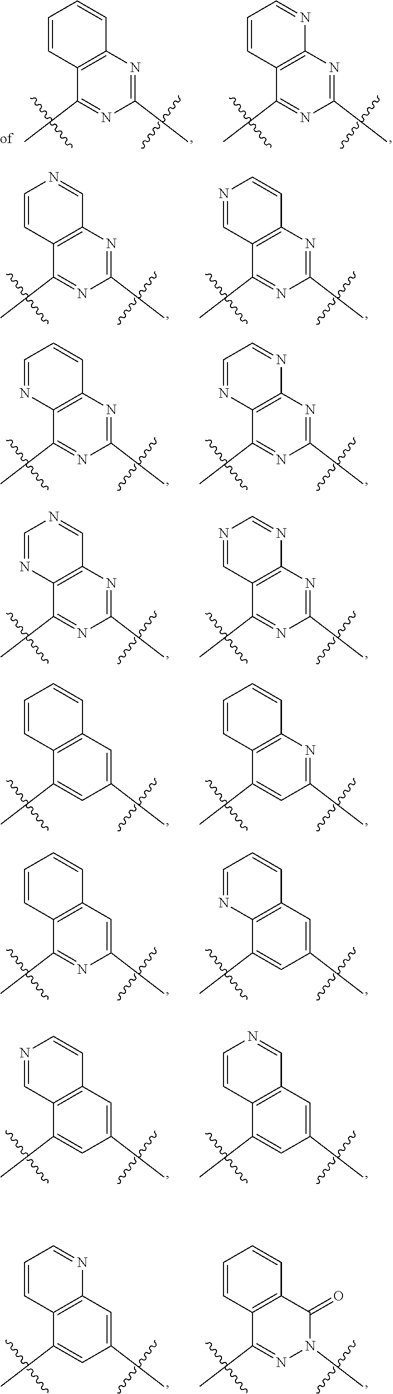 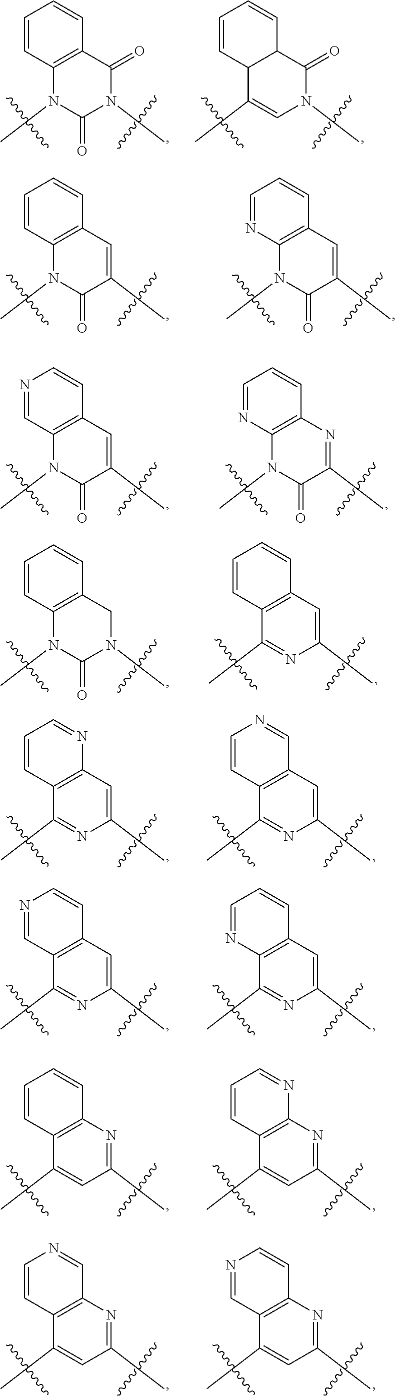 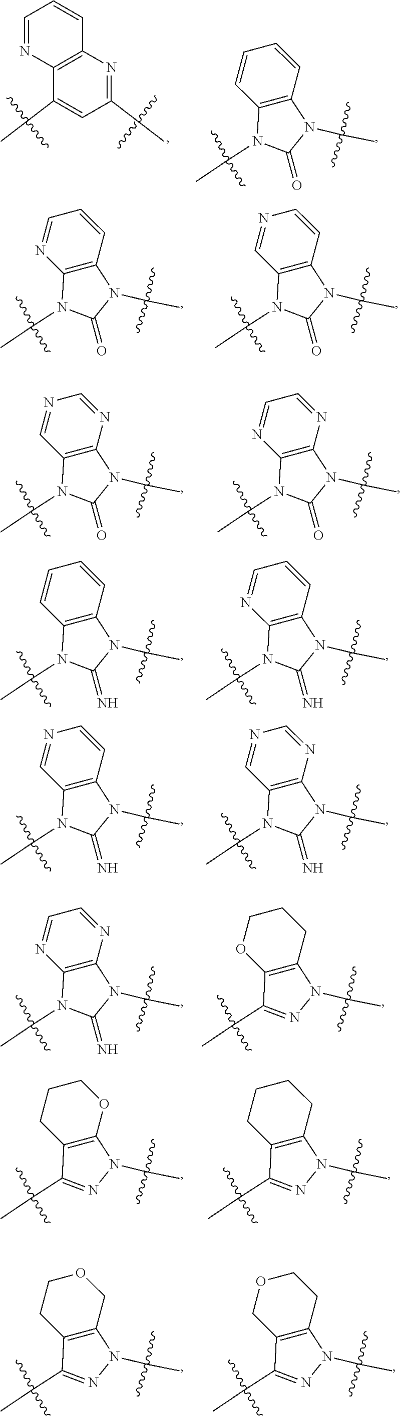 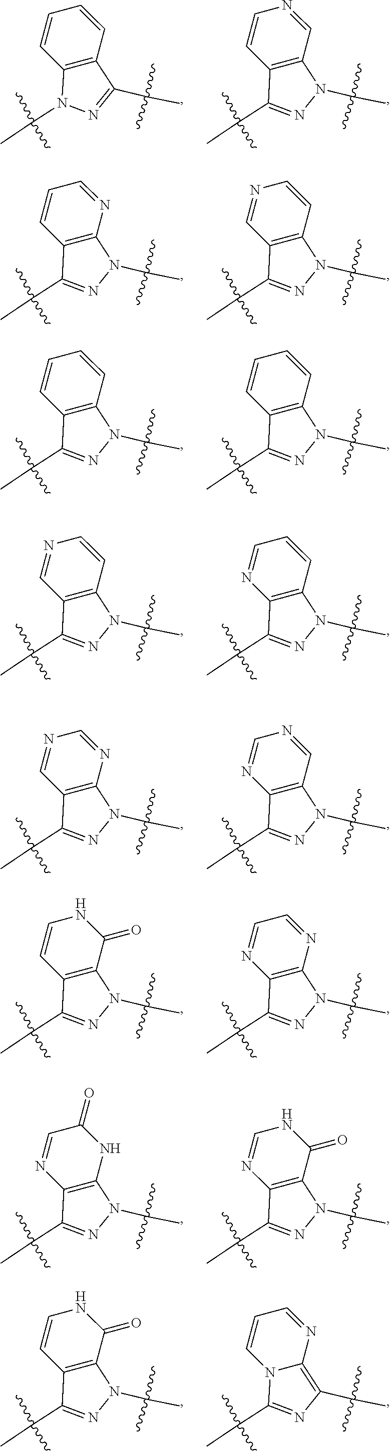 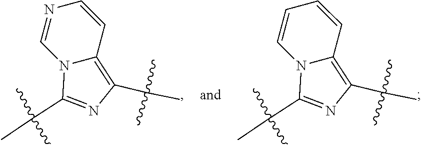 [SMILES: *c1nc(*)n2cnccc12 ; *c1nc(*)n2ccccc12]
wherein the 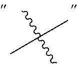
represents a site which is attached to L₁ or L₂.

35. The compound or the stereoisomer, tautomer, pharmaceutically acceptable salt, prodrug, chelate, non-covalent complex, or solvate of claim 1, wherein ring D is selected from the group consisting of

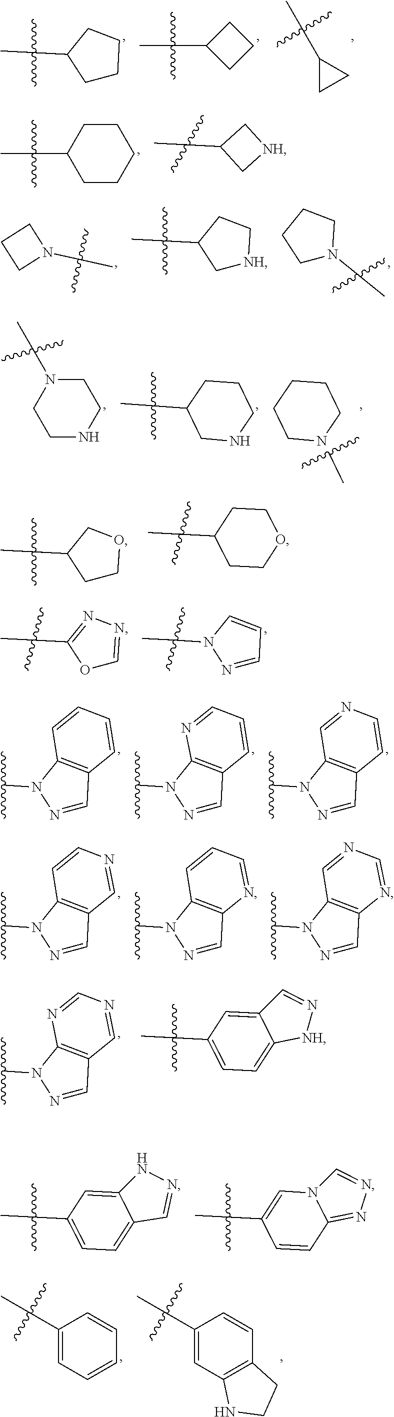

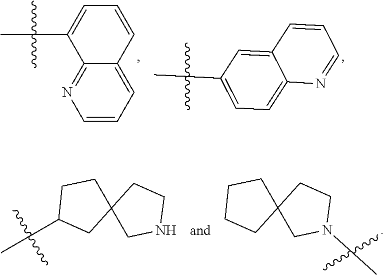 [SMILES: *c1cccc2cccnc12 ; *c1ccc2ncccc2c1 ; *C1CCC2(CCNC2)C1 ; "and" ; *N1CCC2(CCCC2)C1]

36. The compound or the stereoisomer, tautomer, pharmaceutically acceptable salt, prodrug, chelate, non-covalent complex, or solvate of claim 1, wherein ring E is selected from the group consisting of

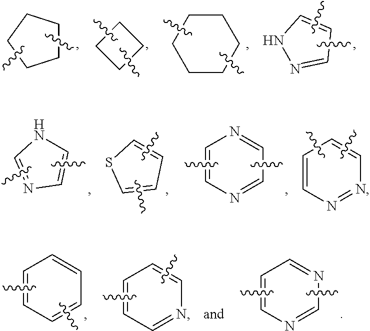

37. The compound of claim 1, or a stereoisomer, tautomer, pharmaceutically acceptable salt, prodrug, chelate, non-covalent complex, or solvate thereof, wherein, the compound is:
1) 1-(1-acryloylpyrrolidin-3-yl)-3-(4-cyclohexylphenyl)-1,6-dihydro-7H-pyrazolo[4,3-d]pyrimidin-7-one;
2) 1-(1-acryloylpyrrolidin-3-yl)-3-(4-(trifluoromethyl)phenyl)-1,6-dihydro-7H-pyrazolo[4,3-d]pyrimidin-7-one;
3) 1-(3-(3-(4-(trifluoromethyl)phenyl)-1H-indazol-1-yl)pyrrolidin-1-yl)prop-2-en-1-one;
4) 1-(3-(1-(4-(trifluoromethyl)phenyl)-1H-indazol-3-yl)pyrrolidin-1-yl)prop-2-en-1-one;
5) 2-fluoro-1-(3-(3-(4-(trifluoromethyl)phenyl)-1H-pyrazolo[3,4-b]pyridin-1-yl)azetidin-1-yl)prop-2-en-1-one;
6) 2-fluoro-1-(3-(3-(4-(trifluoromethyl)phenyl)-1H-pyrazolo[4,3-b]pyridin-1-yl)azetidin-1-yl)prop-2-en-1-one;
7) 1-(1-acryloylpyrrolidin-3-yl)-6-methyl-3-(4-(trifluoromethyl)phenyl)-1,6-dihydro-7H-pyrazolo[4,3-d]pyrimidin-7-one;
8) 2-fluoro-1-(3-methyl-3-(3-(4-(trifluoromethyl)phenyl)-1H-pyrazolo[3,4-b]pyridin-1-yl)azetidin-1-yl)prop-2-en-1-one;
9) 2-fluoro-1-(2-methyl-3-(3-(4-(trifluoromethyl)phenyl)-1H-pyrazolo[3,4-b]pyridin-1-yl)azetidin-1-yl)prop-2-en-1-one;
10) 2-fluoro-N-(2-methyl-5-(3-(4-(trifluoromethyl)phenyl)-1H-pyrazolo[3,4-b]pyridin-1-yl)phenyl)acrylamide;
11) 2-fluoro-1-(3-(6-methyl-3-((4-(trifluoromethyl)phenyl)amino)-1H-pyrazolo[3,4-b]pyridin-1-yl)azetidin-1-yl)prop-2-en-1-one;
12) 2-fluoro-1-(2-hydroxy-3-(3-(4-(trifluoromethyl)phenyl)-1H-pyrazolo[3,4-b]pyridin-1-yl)azetidin-1-yl)prop-2-en-1-one;
13) N-(1-(3-(4-(trifluoromethyl)phenyl)imidazo[1,5-a]pyridin-1-yl)azetidin-3-yl)acrylamide;
14) N-(1-(1-acryloylazetidin-3-yl)-3-(4-(trifluoromethyl) phenyl)-1H-indazol-7-yl) methanesulfonamide;
15) N-(1-(1-acryloylazetidin-3-yl)-3-(4-(trifluoromethyl)phenyl)-1H-indazol-7-yl)acetamide;
16) 1-(3-(4-amino-3-(4-cyclohexylphenyl)-1H-pyrazolo[3,4-d]pyrimidin-1-yl)pyrrolidin-1-yl)prop-2-en-1-one;
17) 1-(3-(3-(4-cyclohexylphenyl)-4-hydroxy-1H-pyrazolo[3,4-d]pyrimidin-1-yl)pyrrolidin-1-yl)prop-2-en-1-one;
18) 1-(3-(3-(4-(trifluoromethyl)phenyl)-1H-pyrazolo[3,4-b]pyrazin-1-yl)pyrrolidin-1-yl)prop-2-en-1-one;
19) 1-(3-(3-(4-(trifluoromethyl)phenyl)-1H-pyrazolo[3,4-d]pyrimidin-1-yl)pyrrolidin-1-yl)prop-2-en-1-one;
20) 1-(3-(6-chloro-3-(4-(trifluoromethyl)phenyl)-1H-pyrazolo[3,4-b]pyrazin-1-yl)pyrrolidin-1-yl)prop-2-en-1-one;
21) 1-(3-(3-(4-(trifluoromethyl)phenyl)-1H-pyrazolo[3,4-b]pyridin-1-yl)pyrrolidin-1-yl)prop-2-en-1-one;
22) 1-(3-(3-(4-(trifluoromethyl)phenyl)-1H-pyrazolo[3,4-c]pyridin-1-yl)pyrrolidin-1-yl)prop-2-en-1-one;
23) 1-(3-(3-(4-(trifluoromethyl)phenyl)-1H-pyrazolo[4,3-c]pyridin-1-yl)pyrrolidin-1-yl)prop-2-en-1-one;
24) 1-(3,3-difluoro-4-(3-(4-(trifluoromethyl)phenyl)-1H-pyrazolo[4,3-b]pyridin-1-yl)pyrrolidin-1-yl)prop-2-en-1-one;
25) 1-((3R,4S)-3-fluoro-4-(3-(4-(trifluoromethyl)phenyl)-1H-pyrazolo[4,3-b]pyridin-1-yl)pyrrolidin-1-yl)prop-2-en-1-one;
26) 1-(3-(3-(4-(trifluoromethyl)phenyl)-1H-pyrazolo[4,3-b]pyridin-1-yl)pyrrolidin-1-yl)prop-2-en-1-one;
27) 1-(3-(3-(5-(trifluoromethyl)pyridin-2-yl)-1H-pyrazolo[3,4-b]pyridin-1-yl)pyrrolidin-1-yl)prop-2-en-1-one;
28) N-(1-(1-(4-(trifluoromethyl)phenyl)-1H-pyrazolo[4,3-b]pyridin-3-yl)pyrrolidin-3-yl)but-2-ynamide;
29) 1-(3-(3-(2-fluoro-4-(trifluoromethyl)phenyl)-1H-pyrazolo[3,4-b]pyridin-1-yl)pyrrolidin-1-yl)prop-2-en-1-one;
30) 1-(3-(3-(4-(trifluoromethyl)phenyl)-1H-pyrazolo[3,4-b]pyridin-1-yl)azetidin-1-yl)prop-2-en-1-one;
31) 1-(3-(3-(4-(trifluoromethyl)phenoxy)-1H-pyrazolo[4,3-b]pyridin-1-yl)azetidin-1-yl)prop-2-en-1-one;
32) 1-(3-(3-(4-(trifluoromethyl)phenoxy)-1H-pyrazolo[3,4-b]pyridin-1-yl)azetidin-1-yl)prop-2-en-1-one;
33) 1-(3-(3-(4-(trifluoromethyl)phenyl)-1H-pyrazolo[3,4-b]pyridin-1-yl)piperidin-1-yl)prop-2-en-1-one;
34) 1-(3-((3-(4-(trifluoromethyl)phenyl)-1H-indazol-1-yl)methyl)pyrrolidin-1-yl)prop-2-en-1-one;
35) N-(1-(1-(4-(trifluoromethyl)phenyl)-1H-pyrazolo[4,3-b]pyridin-3-yl)pyrrolidin-3-yl)acrylamide;
36) (E)-N-(1-(1-(4-(trifluoromethyl)phenyl)-1H-pyrazolo[4,3-b]pyridin-3-yl)pyrrolidin-3-yl)but-2-enamide;
37) N-(3-(3-(4-(trifluoromethyl)phenyl)-1H-indazol-1-yl)cyclopentyl)acrylamide;
38) 1-(3-((3-(4-cyclohexylphenyl)-1H-indazol-1-yl)methyl)pyrrolidin-1-yl)prop-2-en-1-one;
39) 1-(3-(7-methyl-3-(4-(trifluoromethyl)phenyl)-1H-indazol-1-yl)pyrrolidin-1-yl)prop-2-en-1-one;
40) (E)-4-(dimethylamino)-1-(3-(3-(4-(trifluoromethyl)phenyl)-1H-indazol-1-yl)pyrrolidin-1-yl)but-2-en-1-one;
41) (E)-4-(dimethylamino)-N-(1-(1-(4-(trifluoromethyl)phenyl)-1H-pyrazolo[4,3-b]pyridin-3-yl)pyrrolidin-3-yl)but-2-enamide;
42) 1-(1-acryloylpyrrolidin-3-yl)-3-(4-(trifluoromethyl)phenyl)-1H-indazole-7-carboxamide;
43) 1-(4-(1-(4-(trifluoromethyl)phenyl)-1H-indazole-3-carbonyl)piperazin-1-yl)prop-2-en-1-one;
44) 1-(3-(7-methoxy-3-(4-(trifluoromethyl)phenyl)-1H-indazol-1-yl)pyrrolidin-1-yl)prop-2-en-1-one;
45) 1-(3-(7-chloro-3-(4-(trifluoromethyl)phenyl)-1H-indazol-1-yl)pyrrolidin-1-yl)prop-2-en-1-one;
46) 1-(3-(7-(trifluoromethyl)-3-(4-(trifluoromethyl)phenyl)-1H-indazol-1-yl)pyrrolidin-1-yl)prop-2-en-1-one;
47) 1-(3-(6-methyl-3-(4-(trifluoromethyl)phenyl)-1H-indazol-1-yl)pyrrolidin-1-yl)prop-2-en-1-one;
48) 1-(1-acryloylpyrrolidin-3-yl)-3-(4-(trifluoromethyl)phenyl)-1H-indazole-7-carbonitrile;
49) 1-(7-(3-(4-(trifluoromethyl)phenyl)-1H-pyrazolo[3,4-b]pyridin-1-yl)-2-azaspiro[4.4]nonan-2-yl)prop-2-en-1-one;
50) 1-(3-(6-fluoro-3-(4-(trifluoromethyl)phenyl)-1H-indazol-1-yl)pyrrolidin-1-yl)prop-2-en-1-one;
51) 1-(3-(5,6-difluoro-3-(4-(trifluoromethyl)phenyl)-1H-indazol-1-yl)pyrrolidin-1-yl)prop-2-en-1-one;
52) 1-(3-(1-(4-(trifluoromethyl)phenyl)-1H-pyrazolo[3,4-b]pyridin-3-yl)pyrrolidin-1-yl)prop-2-en-1-one;
53) N-(1-(1-(4-(trifluoromethyl)phenyl)-1H-pyrazolo[3,4-b]pyridin-3-yl)pyrrolidin-3-yl)acrylamide;
54) 1-(3-(6-methoxy-3-(4-(trifluoromethyl)phenyl)-1H-indazol-1-yl)pyrrolidin-1-yl)prop-2-en-1-one;
55) 1-(3-(3-(4-(trifluoromethyl)phenyl)-1H-indazol-1-yl)pyrrolidin-1-yl)but-2-yn-1-one;
56) (E)-1-(3-(3-(4-(trifluoromethyl)phenyl)-1H-indazol-1-yl)pyrrolidin-1-yl)but-2-en-1-one;
57) 1-(3-(3-(4-(trifluoromethyl)phenyl)-1H-indazol-1-yl)pyrrolidin-1-yl)prop-2-yn-1-one;
58) 1-(3-(3-(5-(trifluoromethyl)pyridin-2-yl)-1H-indazol-1-yl)pyrrolidin-1-yl)prop-2-en-1-one;
59) 1-(3-(3-(6-(trifluoromethyl)pyridin-3-yl)-1H-indazol-1-yl)pyrrolidin-1-yl)prop-2-en-1-one;
60) 1-(3-(3-(4-(trifluoromethyl)phenyl)-1H-indazol-1-yl)piperidin-1-yl)prop-2-en-1-one;
61) 1-(3-(3-(4-(trifluoromethyl)phenyl)-1H-indazol-1-yl)azetidin-1-yl)prop-2-en-1-one;
62) N-(4-(3-(4-(trifluoromethyl)phenyl)-1H-indazol-1-yl)tetrahydrofuran-3-yl)acrylamide;
63) N-((5-(3-(4-(trifluoromethyl)phenyl)-1H-indazol-1-yl)-1,3,4-oxadiazol-2-yl)methyl)acrylamide;
64) N-(1-(1-(4-(trifluoromethyl)phenyl)-1H-indazole-3-carbonyl)pyrrolidin-3-yl)acrylamide;
65) 1-(3-(1-(4-(trifluoromethyl)phenyl)-1H-pyrazolo[4,3-b]pyridin-3-yl)pyrrolidin-1-yl)prop-2-en-1-one;
66) N-(1-(5-methoxy-1-(4-(trifluoromethyl)phenyl)-1H-pyrazolo[4,3-b]pyridin-3-yl)pyrrolidin-3-yl)acrylamide;
67) 1-(3-(5-(3-(4-(trifluoromethyl)phenyl)-1H-indazol-1-yl)-1,3,4-oxadiazol-2-yl)pyrrolidin-1-yl)prop-2-en-1-one;
68) N-(4-(3-(4-(trifluoromethyl)phenyl)-1H-indazol-1-yl)tetrahydro-2H-pyran-3-yl)acrylamide;
69) N-(1-(5-cyano-1-(4-(trifluoromethyl)phenyl)-1H-pyrazolo[4,3-b]pyridin-3-yl)pyrrolidin-3-yl)acrylamide;
70) 1-(3-(7-fluoro-3-(4-(trifluoromethyl)phenyl)-1H-indazol-1-yl)pyrrolidin-1-yl)prop-2-en-1-one;
71) 2-fluoro-1-(3-(3-(4-(trifluoromethyl)phenyl)-1H-pyrazolo[4,3-b]pyridin-1-yl)pyrrolidin-1-yl)prop-2-en-1-one;
72) N-(1-(5-cyano-1-(4-(trifluoromethyl)phenyl)-1H-pyrazolo[3,4-b]pyridin-3-yl)pyrrolidin-3-yl)acrylamide;
73) 2-methyl-1-(3-(3-(4-(trifluoromethyl)phenyl)-1H-pyrazolo[3,4-b]pyridin-1-yl)azetidin-1-yl)prop-2-en-1-one;
74) N-(1-(5-methoxy-1-(4-(trifluoromethyl)phenyl)-1H-pyrazolo[3,4-b]pyridin-3-yl)pyrrolidin-3-yl)acrylamide;
75) N-(1-(5-methyl-1-(4-(trifluoromethyl)phenyl)-1H-pyrazolo[4,3-b]pyridin-3-yl)pyrrolidin-3-yl)acrylamide;
76) 5-methyl-2-(3-(3-(4-(trifluoromethyl)phenyl)-1H-pyrazolo[3,4-b]pyridin-1-yl)azetidine-1-carbonyl)hex-2-enenitrile;
77) 1-(1-(2-fluoroacryloyl)pyrrolidin-3-yl)-6-methyl-3-(4-(trifluoromethyl)phenyl)-1,6-dihydro-7H-pyrazolo[4,3-d]pyrimidin-7-one;
78) methyl 1-(1-acryloylpyrrolidin-3-yl)-3-(4-(trifluoromethyl)phenyl)-1H-indazole-7-carboxylate;
79) 1-(1-acryloylazetidin-3-yl)-6-methyl-3-(4-(trifluoromethyl)phenyl)-1,6-dihydro-7H-pyrazolo[4,3-d]pyrimidin-7-one;
80) 1-(1-(2-fluoroacryloyl)azetidin-3-yl)-6-methyl-3-(4-(trifluoromethyl)phenyl)-1,6-dihydro-7H-pyrazolo[4,3-d]pyrimidin-7-one;
81) N-(1-(6-methyl-1-(4-(trifluoromethyl)phenyl)-1H-pyrazolo[4,3-b]pyridin-3-yl)pyrrolidin-3-yl)acrylamide;
82) 1-(3-(3-(4-(trifluoromethyl)phenyl)-1H-pyrazolo[4,3-b]pyridin-1-yl)azetidin-1-yl)prop-2-en-1-one;
83) N-(1-(5-methyl-1-(4-(trifluoromethyl)phenyl)-1H-pyrazolo[3,4-b]pyridin-3-yl)pyrrolidin-3-yl)acrylamide;
84) N-(1-(5-chloro-1-(4-(trifluoromethyl)phenyl)-1H-pyrazolo[4,3-b]pyridin-3-yl)pyrrolidin-3-yl)acrylamide;
85) N-(1-(5-chloro-1-(4-(trifluoromethyl)phenyl)-1H-pyrazolo[3,4-b]pyridin-3-yl)pyrrolidin-3-yl)acrylamide;
86) N-(1-(6-chloro-1-(4-(trifluoromethyl)phenyl)-1H-pyrazolo[3,4-b]pyridin-3-yl)pyrrolidin-3-yl)acrylamide;
87) 2-methyl-1-(3-(3-(4-(trifluoromethyl)phenyl)-1H-pyrazolo[4,3-b]pyridin-1-yl)azetidin-1-yl)prop-2-en-1-one;
88) 4-methyl-4-morpholino-2-(3-(3-(4-(trifluoromethyl)phenyl)-1H-pyrazolo[4,3-b]pyridin-1-yl)azetidine-1-carbonyl)pent-2-enenitrile;
89) 1-(3-(5-methoxy-3-(4-(trifluoromethyl)phenyl)-1H-pyrazolo[4,3-b]pyridin-1-yl)pyrrolidin-1-yl)prop-2-en-1-one;
90) N-(1-(1-(4-(trifluoromethyl)phenyl)-1H-pyrazolo[3,4-b]pyridin-3-yl)pyrrolidin-3-yl)acrylamide;
91) N-(1-(1-(4-(trifluoromethyl)phenyl)-1H-pyrazolo[4,3-b]pyridin-3-yl)pyrrolidin-3-yl)propiolamide;
92) N-(1-(1-(4-(trifluoromethyl)phenyl)-1H-pyrazolo[3,4-b]pyridin-3-yl)pyrrolidin-3-yl)propiolamide;
93) 1-(1-acryloylpyrrolidin-3-yl)-3-(4-(trifluoromethyl)phenyl)-1H-pyrazolo[4,3-b]pyridine-5-carbonitrile;
94) 1-(3-(5-methoxy-3-(4-(trifluoromethyl)phenyl)-1H-pyrazolo[3,4-b]pyridin-1-yl)pyrrolidin-1-yl)prop-2-en-1-one;
95) 2-fluoro-1-(3-(5-methoxy-3-(4-(trifluoromethyl)phenyl)-1H-pyrazolo[3,4-b]pyridin-1-yl)pyrrolidin-1-yl)prop-2-en-1-one;
96) 1-(3-(5-methyl-3-(4-(trifluoromethyl)phenyl)-1H-pyrazolo[3,4-b]pyridin-1-yl)pyrrolidin-1-yl)prop-2-en-1-one;
97) N-(3-(1-(4-(trifluoromethyl)phenyl)-1H-pyrazolo[3,4-b]pyridin-3-yl)phenyl)acrylamide;
98) 1-(1-acryloylpyrrolidin-3-yl)-N-isopropyl-3-(6-(trifluoromethyl)pyridin-3-yl)-1H-indazole-7-carboxamide;
99) N-(1-(3-(4-(trifluoromethyl)phenyl)imidazo[1,5-a]pyridin-1-yl)pyrrolidin-3-yl)acrylamide;
100) 1-(1-acryloylpyrrolidin-3-yl)-N-cyclopropyl-3-(6-(trifluoromethyl)pyridin-3-yl)-1H-indazole-7-carboxamide;
101) 1-(1-acryloylpyrrolidin-3-yl)-N-(oxetan-3-yl)-3-(6-(trifluoromethyl)pyridin-3-yl)-1H-indazole-7-carboxamide;
102) 1-(1-acryloylpyrrolidin-3-yl)-N-methyl-3-(6-(trifluoromethyl)pyridin-3-yl)-1H-indazole-7-carboxamide;
103) 1-(1-acryloylpyrrolidin-3-yl)-N,N-dimethyl-3-(4-(trifluoromethyl)phenyl)-1H-indazole-7-carboxamide;
104) 1-(1-acryloylpyrrolidin-3-yl)-N-(3,3-difluorocyclobutyl)-3-(6-(trifluoromethyl)pyridin-3-yl)-1H-indazole-7-carboxamide;
105) 1-(3-(1-(4-(trifluoromethyl)phenyl)imidazo[1,5-a]pyridin-3-yl)pyrrolidin-1-yl)prop-2-en-1-one;
106) N-(1-(6-(4-(trifluoromethyl)phenyl)imidazo[1,5-a]pyrimidin-8-yl)pyrrolidin-3-yl)acrylamide;
107) 1-(1-acryloylpyrrolidin-3-yl)-N-phenyl-3-(4-(trifluoromethyl)phenyl)-1H-indazole-7-carboxamide;
108) 1-(3-(5-chloro-3-(4-(trifluoromethyl)phenyl)-1H-pyrazolo[3,4-b]pyridin-1-yl)pyrrolidin-1-yl)prop-2-en-1-one;
109) 1-(3-(8-(4-(trifluoromethyl)phenyl)imidazo[1,5-a]pyrimidin-6-yl)pyrrolidin-1-yl)prop-2-en-1-one;
110) 1-(3-(3-(4-(trifluoromethyl)phenyl)-7-(4-(trifluoromethyl)piperidine-1-carbonyl)-1H-indazol-1-yl)pyrrolidin-1-yl)prop-2-en-1-one;
111) 1-(1-acryloylpyrrolidin-3-yl)-N-(4,4-difluorocyclohexyl)-3-(4-(trifluoromethyl)phenyl)-1H-indazole-7-carboxamide;
112) 1-(3-(7-(3,3-difluoropyrrolidine-1-carbonyl)-3-(4-(trifluoromethyl)phenyl)-1H-indazol-1-yl)pyrrolidin-1-yl)prop-2-en-1-one;
113) 1-(1-acryloylpyrrolidin-3-yl)-N-(3,3-difluorocyclopentyl)-3-(4-(trifluoromethyl)phenyl)-1H-indazole-7-carboxamide;
114) 1-(1-acryloylpyrrolidin-3-yl)-N-(4-(trifluoromethyl)cyclohexyl)-3-(4-(trifluoromethyl)phenyl)-1H-indazole-7-carboxamide;
115) 1-(1-acryloylpyrrolidin-3-yl)-N-benzyl-3-(4-(trifluoromethyl)phenyl)-1H-indazole-7-carboxamide;
116) 1-(1-acryloylpyrrolidin-3-yl)-N-(tert-butyl)-3-(4-(trifluoromethyl)phenyl)-1H-indazole-7-carboxamide;
117) 1-(3-methyl-4-(3-(4-(trifluoromethyl)phenyl)-1H-pyrazolo[4,3-b]pyridin-1-yl)pyrrolidin-1-yl)prop-2-en-1-one;
118) 1-(7-(3-(4-(trifluoromethyl)phenyl)-1H-pyrazolo[4,3-b]pyridin-1-yl)-5-azaspiro[2.4]heptan-5-yl)prop-2-en-1-one;
119) N-(2-(3-(4-(trifluoromethyl)phenyl)-1H-pyrazolo[4,3-b]pyridin-1-yl)cyclopentyl)acrylamide;
120) 1-(3-(3-(4-cyclopropylphenyl)-1H-pyrazolo[4,3-b]pyridin-1-yl)azetidin-1-yl)prop-2-en-1-one;
121) 1-(3-(6-(dimethylamino)-3-(4-(trifluoromethyl)phenyl)-1H-pyrazolo[3,4-b]pyridin-1-yl)azetidin-1-yl)prop-2-en-1-one;
122) 1-(3-(6-(dimethylamino)-3-(4-(trifluoromethyl)phenyl)-1H-pyrazolo[3,4-b]pyridin-1-yl)azetidin-1-yl)-2-fluoroprop-2-en-1-one;
123) 1-(3-(3-(6-(trifluoromethyl)pyridin-3-yl)-1H-pyrazolo[3,4-b]pyridin-1-yl)azetidin-1-yl)prop-2-en-1-one;
124) 2-fluoro-1-(3-(3-(6-(trifluoromethyl)pyridin-3-yl)-1H-pyrazolo[3,4-b]pyridin-1-yl)azetidin-1-yl)prop-2-en-1-one;
125) 1-(7-(3-(4-(trifluoromethyl)phenyl)-1H-pyrazolo[4,3-b]pyridin-1-yl)-2-azaspiro[4.4]nonan-2-yl)prop-2-en-1-one;
126) 2-fluoro-1-(7-(3-(4-(trifluoromethyl)phenyl)-1H-pyrazolo[4,3-b]pyridin-1-yl)-2-azaspiro[4.4]nonan-2-yl)prop-2-en-1-one;
127) 1-(3-(3-(2-fluoro-4-(trifluoromethyl)phenyl)-1H-pyrazolo[3,4-b]pyridin-1-yl)azetidin-1-yl)prop-2-en-1-one;
128) 2-fluoro-1-(3-(3-(2-fluoro-4-(trifluoromethyl)phenyl)-1H-pyrazolo[3,4-b]pyridin-1-yl)azetidin-1-yl)prop-2-en-1-one;
129) 1-(3-(3-(2-fluoro-4-(trifluoromethyl)phenyl)-1H-pyrazolo[4,3-b]pyridin-1-yl)azetidin-1-yl)prop-2-en-1-one;
130) 2-fluoro-1-(3-(3-(2-fluoro-4-(trifluoromethyl)phenyl)-1H-pyrazolo[4,3-b]pyridin-1-yl)azetidin-1-yl)prop-2-en-1-one;
131) 1-(3-(3-(6-(trifluoromethyl)pyridin-3-yl)-1H-pyrazolo[4,3-b]pyridin-1-yl)azetidin-1-yl)prop-2-en-1-one;
132) 2-fluoro-1-(3-(3-(6-(trifluoromethyl)pyridin-3-yl)-1H-pyrazolo[4,3-b]pyridin-1-yl)azetidin-1-yl)prop-2-en-1-one;
133) N-(3-(4-(trifluoromethyl)phenyl)-1′H-[1,6′-biindazol]-4′-yl)acrylamide;
134) N-(6-(3-(4-(trifluoromethyl)phenyl)-1H-indazol-1-yl)-[1,2,4]triazolo[4,3-a]pyridin-8-yl)acrylamide;
135) N-(3-(3-(4-(trifluoromethyl)phenyl)-1H-indazol-1-yl)phenyl)acrylamide;
136) N-(3-methyl-5-(3-(4-(trifluoromethyl)phenyl)-1H-indazol-1-yl)phenyl)acrylamide;
137) N-(3-methoxy-5-(3-(4-(trifluoromethyl)phenyl)-1H-indazol-1-yl)phenyl)acrylamide;
138) N-(3-chloro-5-(3-(4-(trifluoromethyl)phenyl)-1H-indazol-1-yl)phenyl)acrylamide;
139) 1-(3-(3-(4-(trifluoromethyl)phenyl)-1H-pyrazolo[4,3-b]pyridin-1-yl)azetidin-1-yl)prop-2-yn-1-one;
140) 1-(3-(3-(4-(trifluoromethyl)phenyl)-1H-pyrazolo[3,4-b]pyridin-1-yl)azetidin-1-yl)prop-2-yn-1-one;
141) (E)-2-(3-(3-(4-(trifluoromethyl)phenyl)-1H-pyrazolo[4,3-b]pyridin-1-yl)azetidine-1-carbonyl)but-2-enenitrile;
142) 1-(1-acryloylpyrrolidin-3-yl)-3-(4-(trifluoromethyl)phenyl)-1H-pyrazolo[3,4-b]pyridine-5-carbonitrile;
143) 1-(3-(5-methyl-3-(4-(trifluoromethyl)phenyl)-1H-pyrazolo[4,3-b]pyridin-1-yl)pyrrolidin-1-yl)prop-2-en-1-one;
144) 1-(3-(6-methyl-3-(4-(trifluoromethyl)phenyl)-1H-pyrazolo[4,3-b]pyridin-1-yl)pyrrolidin-1-yl)prop-2-en-1-one;
145) 1-(1-acryloylpyrrolidin-3-yl)-N-(pyridin-2-yl)-3-(4-(trifluoromethyl)phenyl)-1H-indazole-7-carboxamide;
146) 1-(3-(5-chloro-3-(4-(trifluoromethyl)phenyl)-1H-pyrazolo[4,3-b]pyridin-1-yl)pyrrolidin-1-yl)prop-2-en-1-one;
147) 1-(3-(6-chloro-3-(4-(trifluoromethyl)phenyl)-1H-pyrazolo[3,4-b]pyridin-1-yl)pyrrolidin-1-yl)prop-2-en-1-one;
148) 1-(1-acryloylpyrrolidin-3-yl)-3-(4-(trifluoromethyl)phenyl)-N-(5-(trifluoromethyl)pyridin-2-yl)-1H-indazole-7-carboxamide;
149) 1-acryloyl-4-(3-(4-(trifluoromethyl)phenyl)-1H-pyrazolo[4,3-b]pyridin-1-yl)pyrrolidine-3-carbonitrile;
150) 1-(3-(5-methyl-1-(4-(trifluoromethyl)phenyl)-1H-pyrazolo[3,4-b]pyridin-3-yl)pyrrolidin-1-yl)prop-2-en-1-one;
151) N-(3-cyano-5-(3-(4-(trifluoromethyl)phenyl)-1H-indazol-1-yl)phenyl)acrylamide;
152) N-(3-cyano-5-(3-(4-(trifluoromethyl)phenyl)-1H-indazol-1-yl)phenyl)acrylamide;
153) N-(3-cyclopropyl-5-(3-(4-(trifluoromethyl)phenyl)-1H-indazol-1-yl)phenyl)acrylamide;
154) N-(3-(3,3-difluoroazetidin-1-yl)-5-(3-(4-(trifluoromethyl)phenyl)-1H-indazol-1-yl)phenyl)acrylamide;
155) N-(3-(3-methylpyridin-2-yl)-5-(3-(4-(trifluoromethyl)phenyl)-1H-indazol-1-yl)phenyl)acrylamide;
156) N-(3-(3-chloropyridin-2-yl)-5-(3-(4-(trifluoromethyl)phenyl)-1H-indazol-1-yl)phenyl)acrylamide;
157) N-(3-(1H-pyrazol-1-yl)-5-(3-(4-(trifluoromethyl)phenyl)-1H-indazol-1-yl)phenyl)acrylamide;
158) N-(3-morpholino-5-(3-(4-(trifluoromethyl)phenyl)-1H-indazol-1-yl)phenyl)acrylamide;
159) N-(3-(3-(4-(trifluoromethyl)phenyl)-1H-pyrazolo[3,4-b]pyridin-1-yl)phenyl)acrylamide;
160) 2-fluoro-1-(3-(3-(4-(trifluoromethyl)phenyl)-1H-pyrazolo[3,4-b]pyrazin-1-yl)azetidin-1-yl)prop-2-en-1-one;
161) 1-(3-(3-(4-(trifluoromethyl)phenyl)-1H-pyrazolo[3,4-b]pyrazin-1-yl)azetidin-1-yl)prop-2-en-1-one;
162) 2-fluoro-1-(3-fluoro-3-(3-(4-(trifluoromethyl)phenyl)-1H-pyrazolo[3,4-b]pyridin-1-yl)azetidin-1-yl)prop-2-en-1-one;
163) 2-fluoro-1-(2-fluoro-3-(3-(4-(trifluoromethyl)phenyl)-1H-pyrazolo[3,4-b]pyridin-1-yl)azetidin-1-yl)prop-2-en-1-one;
164) 2-fluoro-1-(3-hydroxy-3-(3-(4-(trifluoromethyl)phenyl)-1H-pyrazolo[3,4-b]pyridin-1-yl)azetidin-1-yl)prop-2-en-1-one;
165) 1-(1-(2-fluoroacryloyl)azetidin-3-yl)-3-(4-(trifluoromethyl)phenyl)-1H-pyrazolo[3,4-b]pyridine 7-oxide;
166) ethyl 2-(1-(2-fluoroacryloyl)-3-(3-(4-(trifluoromethyl)phenyl)-1H-pyrazolo[3,4-b]pyridin-1-yl)azetidin-3-yl)acetate;
167) 2-(1-(2-fluoroacryloyl)-3-(3-(4-(trifluoromethyl)phenyl)-1H-pyrazolo[3,4-b]pyridin-1-yl)azetidin-3-yl)acetonitrile;
168) 2-fluoro-1-(3-(fluoromethyl)-3-(3-(4-(trifluoromethyl)phenyl)-1H-pyrazolo[3,4-b]pyridin-1-yl)azetidin-1-yl)prop-2-en-1-one;
169) 2-(1-(2-fluoroacryloyl)-3-(3-(4-(trifluoromethyl)phenyl)-1H-pyrazolo[3,4-b]pyridin-1-yl)azetidin-3-yl)acetamide;
170) 1-(2-fluoroacryloyl)-3-(3-(4-(trifluoromethyl)phenyl)-1H-pyrazolo[3,4-b]pyridin-1-yl)azetidine-3-carbonitrile;
171) 2-fluoro-1-(3-(3-(4-isopropylphenyl)-1H-pyrazolo[3,4-b]pyridin-1-yl)azetidin-1-yl)prop-2-en-1-one;
172) 2-fluoro-1-(3-(3-(4-(trifluoromethoxy)phenyl)-1H-pyrazolo[3,4-b]pyridin-1-yl)azetidin-1-yl)prop-2-en-1-one;
173) 2-fluoro-1-(3-(3-(4-(pentafluoro-16-sulfanyl)phenyl)-1H-pyrazolo[3,4-b]pyridin-1-yl)azetidin-1-yl)prop-2-en-1-one;
174) 2-methyl-1-(3-(3-(4-(trifluoromethyl)phenyl)-1H-pyrazolo[3,4-b]pyridin-1-yl)azetidin-1-yl)prop-2-en-1-one;
175) (E)-1-(3-(3-(4-(trifluoromethyl)phenyl)-1H-pyrazolo[3,4-b]pyridin-1-yl)azetidin-1-yl)but-2-en-1-one;
176) 2-fluoro-1-(3-(3-(3-(trifluoromethyl)phenyl)-1H-pyrazolo[3,4-b]pyridin-1-yl)azetidin-1-yl)prop-2-en-1-one;
177) 5-(1-(1-(2-fluoroacryloyl)azetidin-3-yl)-1H-pyrazolo[3,4-b]pyridin-3-yl)-2-(trifluoromethyl)benzonitrile;
178) 4-(1-(1-(2-fluoroacryloyl)azetidin-3-yl)-1H-pyrazolo[3,4-b]pyridin-3-yl)-2-(trifluoromethyl)benzonitrile;
179) 2-fluoro-1-(3-(3-(6-(trifluoromethyl)pyridin-2-yl)-1H-pyrazolo[3,4-b]pyridin-1-yl)azetidin-1-yl)prop-2-en-1-one;
180) 2-fluoro-1-(3-(3-(2-(trifluoromethyl)pyridin-4-yl)-1H-pyrazolo[3,4-b]pyridin-1-yl)azetidin-1-yl)prop-2-en-1-one;
181) 2-fluoro-1-(3-(3-(5-methyl-6-(trifluoromethyl)pyridin-3-yl)-1H-pyrazolo[3,4-b]pyridin-1-yl)azetidin-1-yl)prop-2-en-1-one;
182) 2-fluoro-1-(3-(3-(2-methyl-6-(trifluoromethyl)pyridin-3-yl)-1H-pyrazolo[3,4-b]pyridin-1-yl)azetidin-1-yl)prop-2-en-1-one;
183) 2-fluoro-1-(3-(3-(2-(trifluoromethyl)pyrimidin-5-yl)-1H-pyrazolo[3,4-b]pyridin-1-yl)azetidin-1-yl)prop-2-en-1-one;
184) 2-fluoro-1-(3-(3-(5-fluoro-6-(trifluoromethyl)pyridin-3-yl)-1H-pyrazolo[3,4-b]pyridin-1-yl)azetidin-1-yl)prop-2-en-1-one;
185) 2-fluoro-1-(3-(3-(2-fluoro-6-(trifluoromethyl)pyridin-3-yl)-1H-pyrazolo[3,4-b]pyridin-1-yl)azetidin-1-yl)prop-2-en-1-one;
186) 2-fluoro-1-(3-(6-methyl-3-(4-(trifluoromethyl)phenyl)-1H-pyrazolo[3,4-b]pyridin-1-yl)azetidin-1-yl)prop-2-en-1-one;
187) 1-(3-(6-chloro-3-(4-(trifluoromethyl)phenyl)-1H-pyrazolo[3,4-b]pyridin-1-yl)azetidin-1-yl)-2-fluoroprop-2-en-1-one;
188) 2-fluoro-N-(2-methoxy-5-(3-(4-(trifluoromethyl)phenyl)-1H-pyrazolo[3,4-b]pyridin-1-yl)phenyl)acrylamide;
189) N-(2-chloro-5-(3-(4-(trifluoromethyl)phenyl)-1H-pyrazolo[3,4-b]pyridin-1-yl)phenyl)acrylamide;
190) N-(2,4-difluoro-5-(3-(4-(trifluoromethyl)phenyl)-1H-pyrazolo[3,4-b]pyridin-1-yl)phenyl)-2-fluoroacrylamide;
191) 2-fluoro-N-(4-fluoro-3-(3-(4-(trifluoromethyl)phenyl)-1H-pyrazolo[3,4-b]pyridin-1-yl)phenyl)acrylamide;
192) N-(2,4-dichloro-5-(3-(4-(trifluoromethyl)phenyl)-1H-pyrazolo[3,4-b]pyridin-1-yl)phenyl)-2-fluoroacrylamide;
193) 2-fluoro-1-(6-(3-(4-(trifluoromethyl)phenyl)-1H-pyrazolo[3,4-b]pyridin-1-yl)indolin-1-yl)prop-2-en-1-one;
194) N-(4-((dimethylamino)methyl)-3-(3-(4-(trifluoromethyl)phenyl)-1H-pyrazolo[3,4-b]pyridin-1-yl)phenyl)-2-fluoroacrylamide;
195) N-(2,4-difluoro-3-(3-(4-(trifluoromethyl)phenyl)-1H-pyrazolo[3,4-b]pyridin-1-yl)phenyl)-2-fluoroacrylamide;
196) 2-fluoro-1-(3-(3-(4-(trifluoromethyl)phenyl)-1H-indazol-1-yl)azetidin-1-yl)prop-2-en-1-one;
197) 2-fluoro-1-(3-(3-(6-(trifluoromethyl)pyridin-3-yl)-1H-indazol-1-yl)azetidin-1-yl)prop-2-en-1-one;
198) 2-fluoro-1-(3-(3-(6-(trifluoromethyl)pyridin-3-yl)-1H-pyrazolo[3,4-b]pyrazin-1-yl)azetidin-1-yl)prop-2-en-1-one;
199) 2-fluoro-1-(3-(6-methyl-3-(6-(trifluoromethyl)pyridin-3-yl)-1H-pyrazolo[3,4-b]pyridin-1-yl)azetidin-1-yl)prop-2-en-1-one;
200) 1-(1-(2-fluoroacryloyl)azetidin-3-yl)-3-(4-(trifluoromethyl)phenyl)-1H-pyrazolo[3,4-b]pyridine-6-carbonitrile;
201) 1-(1-(2-fluoroacryloyl)azetidin-3-yl)-N-methyl-3-(4-(trifluoromethyl)phenyl)-1H-indazole-7-sulfonamide;
202) 2-fluoro-1-(3-(5-fluoro-3-(4-(trifluoromethyl)phenyl)-1H-pyrazolo[3,4-b]pyridin-1-yl)azetidin-1-yl)prop-2-en-1-one;
203) 2-fluoro-1-(3-(6-fluoro-3-(4-(trifluoromethyl)phenyl)-1H-pyrazolo[3,4-b]pyridin-1-yl)azetidin-1-yl)prop-2-en-1-one;
204) 2-fluoro-1-(3-(6-fluoro-3-(4-(trifluoromethyl)phenyl)-1H-pyrazolo[4,3-b]pyridin-1-yl)azetidin-1-yl)prop-2-en-1-one;
205) 1-(3-(6-(difluoromethyl)-3-(4-(trifluoromethyl)phenyl)-1H-pyrazolo[3,4-b]pyridin-1-yl)azetidin-1-yl)-2-fluoroprop-2-en-1-one;
206) 1-(3-(6-chloro-3-(4-(trifluoromethyl)phenyl)-1H-pyrazolo[3,4-b]pyrazin-1-yl)azetidin-1-yl)-2-fluoroprop-2-en-1-one;
207) 2-fluoro-1-(3-(3-(4-(trifluoromethyl)phenyl)-4,5,6,7-tetrahydro-1H-indazol-1-yl)azetidin-1-yl)prop-2-en-1-one;
208) 2-fluoro-1-(3-(3-(4-(trifluoromethyl)phenyl)-6,7-dihydropyrano[4,3-c]pyrazol-1(4H)-yl)azetidin-1-yl)prop-2-en-1-one;
209) 2-fluoro-1-(3-(3-(4-(trifluoromethyl)phenyl)-6,7-dihydropyrano[4,3-c]pyrazol-2(4H)-yl)azetidin-1-yl)prop-2-en-1-one;
210) 1-(3-(4,4-difluoro-3-(4-(trifluoromethyl)phenyl)-4,5,6,7-tetrahydro-1H-indazol-1-yl)azetidin-1-yl)-2-fluoroprop-2-en-1-one;
211) 1-(3-(5-(difluoromethyl)-3-(4-(trifluoromethyl)phenyl)-1H-pyrazolo[3,4-b]pyridin-1-yl)azetidin-1-yl)-2-fluoroprop-2-en-1-one;
212) 1-(3-(5-(difluoromethyl)-3-(4-(trifluoromethyl)phenyl)-1H-pyrazolo[3,4-b]pyridin-1-yl)azetidin-1-yl)-2-fluoroprop-2-en-1-one;
213) 2-fluoro-1-(3-(5-methyl-3-(4-(trifluoromethyl)phenyl)-1H-pyrazolo[3,4-b]pyridin-1-yl)azetidin-1-yl)prop-2-en-1-one;
214) 2-fluoro-1-(3-(5-methoxy-3-(4-(trifluoromethyl)phenyl)-1H-pyrazolo[3,4-b]pyridin-1-yl)azetidin-1-yl)prop-2-en-1-one;
215) 2-fluoro-1-(3-(5-(trifluoromethyl)-3-(4-(trifluoromethyl)phenyl)-1H-pyrazolo[3,4-b]pyridin-1-yl)azetidin-1-yl)prop-2-en-1-one;
216) 2-fluoro-1-(3-(3-(4-(trifluoromethyl)phenyl)-1H-pyrazolo[4,3-d]pyrimidin-1-yl)azetidin-1-yl)prop-2-en-1-one;
217) 2-fluoro-1-(3-(3-(4-(trifluoromethyl)phenyl)-4,5,6,7-tetrahydro-1H-indazol-1-yl)azetidin-1-yl)prop-2-en-1-one;
218) 6-ethyl-1-(1-(2-fluoroacryloyl)azetidin-3-yl)-3-(4-(trifluoromethyl)phenyl)-1,6-dihydro-7H-pyrazolo[4,3-d]pyrimidin-7-one;
219) 1-(1-(2-fluoroacryloyl)azetidin-3-yl)-7-methyl-3-(4-(trifluoromethyl)phenyl)-1,7-dihydro-6H-pyrazolo[3,4-b]pyridin-6-one;
220) 1-(1-(2-fluoroacryloyl)azetidin-3-yl)-6-methyl-3-(4-(trifluoromethyl)phenyl)-1,6-dihydro-7H-pyrazolo[3,4-c]pyridin-7-one;
221) 2-fluoro-1-(3-(6-methoxy-3-(4-(trifluoromethyl)phenyl)-1H-pyrazolo[3,4-b]pyridin-1-yl)azetidin-1-yl)prop-2-en-1-one;
222) 2-fluoro-1-(3-(7-methoxy-3-(4-(trifluoromethyl)phenyl)-1H-pyrazolo[3,4-c]pyridin-1-yl)azetidin-1-yl)prop-2-en-1-one;
223) 2-fluoro-1-(3-(6-methoxy-3-(4-(trifluoromethyl)phenyl)-1H-pyrazolo[3,4-b]pyrazin-1-yl)azetidin-1-yl)prop-2-en-1-one;
224) 1-(1-(2-fluoroacryloyl)azetidin-3-yl)-7-methyl-3-(4-(trifluoromethyl)phenyl)-1,7-dihydro-6H-pyrazolo[3,4-b]pyrazin-6-one;
225) 1-(1-(2-fluoroacryloyl)azetidin-3-yl)-5,6-dimethyl-3-(4-(trifluoromethyl)phenyl)-1,6-dihydro-7H-pyrazolo[4,3-d]pyrimidin-7-one;
226) 2-fluoro-1-(3-(7-methoxy-5-methyl-3-(4-(trifluoromethyl)phenyl)-1H-pyrazolo[4,3-d]pyrimidin-1-yl)azetidin-1-yl)prop-2-en-1-one;
227) 2-fluoro-1-(3-(7-methoxy-3-(4-(trifluoromethyl)phenyl)-1H-pyrazolo[4,3-d]pyrimidin-1-yl)azetidin-1-yl)prop-2-en-1-one;
228) 1-(3-(5-bromo-3-(4-(trifluoromethyl)phenyl)-1H-pyrazolo[3,4-b]pyridin-1-yl)azetidin-1-yl)-2-fluoroprop-2-en-1-one;
229) 1-(1-(2-fluoroacryloyl)azetidin-3-yl)-3-(4-(trifluoromethyl)phenyl)-1,3-dihydro-2H-benzo[d]imidazol-2-one;
230) 2-fluoro-1-(3-(4-(4-(trifluoromethyl)phenyl)quinazolin-2-yl)azetidin-1-yl)prop-2-en-1-one;
231) 1-(3-(2-imino-3-(4-(trifluoromethyl)phenyl)-2,3-dihydro-1H-benzo[d]imidazol-1-yl)azetidin-1-yl)prop-2-en-1-one;
232) N-((5-(2-oxo-3-(4-(trifluoromethyl)phenyl)-2,3-dihydro-1H-benzo[d]imidazol-1-yl)-1,3,4-oxadiazol-2-yl)methyl)acrylamide;
233) 1-(1-acryloylpyrrolidin-3-yl)-3-(4-cyclohexylphenyl)-1,3-dihydro-2H-benzo[d]imidazol-2-one;
234) 1-(1-acryloylpyrrolidin-3-yl)-3-(4-(trifluoromethyl)phenyl)-1,3-dihydro-2H-benzo[d]imidazol-2-one;
235) 1-(3-(2-imino-3-(4-(trifluoromethyl)phenyl)-2,3-dihydro-1H-benzo[d]imidazol-1-yl)pyrrolidin-1-yl)prop-2-en-1-one;
236) 1-(1-acryloylazetidin-3-yl)-3-(4-(trifluoromethyl)phenyl)-1,3-dihydro-2H-benzo[d]imidazol-2-one;
237) 1-(1-(2-fluoroacryloyl)-3-methylazetidin-3-yl)-3-(4-(trifluoromethyl)phenyl)-1,3-dihydro-2H-benzo[d]imidazol-2-one;
238) 3-(1-(2-fluoroacryloyl)-3-methylazetidin-3-yl)-1-(4-(trifluoromethyl)phenyl)-1,3-dihydro-2H-imidazo[4,5-b]pyridin-2-one;
239) 1-(1-(2-fluoroacryloyl)azetidin-3-yl)-3-(4-(trifluoromethyl)phenyl)-1,3-dihydro-2H-imidazo[4,5-b]pyridin-2-one;
240) 1-(1-(2-fluoroacryloyl)azetidin-3-yl)-3-(4-(trifluoromethyl)phenyl)-1,3-dihydro-2H-imidazo[4,5-b]pyrazin-2-one;
241) 1-(1-(2-fluoroacryloyl)azetidin-3-yl)-3-(6-(trifluoromethyl)pyridin-3-yl)-1,3-dihydro-2H-imidazo[4,5-b]pyrazin-2-one;
242) 3-(1-(2-fluoroacryloyl)azetidin-3-yl)-5-methyl-1-(4-(trifluoromethyl)phenyl)-1,3-dihydro-2H-imidazo[4,5-b]pyridin-2-one;
243) 3-(1-(2-fluoroacryloyl)azetidin-3-yl)-5-methyl-1-(6-(trifluoromethyl)pyridin-3-yl)-1,3-dihydro-2H-imidazo[4,5-b]pyridin-2-one;
244) 3-(1-(2-fluoroacryloyl)azetidin-3-yl)-6-methyl-1-(4-(trifluoromethyl)phenyl)-1,3-dihydro-2H-imidazo[4,5-b]pyridin-2-one;
245) 3-(1-(2-fluoroacryloyl)azetidin-3-yl)-6-(trifluoromethyl)-1-(4-(trifluoromethyl)phenyl)-1,3-dihydro-2H-imidazo[4,5-b]pyridin-2-one;
246) 3-(1-(2-fluoroacryloyl)azetidin-3-yl)-6-methoxy-1-(4-(trifluoromethyl)phenyl)-1,3-dihydro-2H-imidazo[4,5-b]pyridin-2-one;
247) 1-(1-(2-fluoroacryloyl)azetidin-3-yl)-3-(4-(trifluoromethyl)phenyl)-1,3-dihydro-2H-imidazo[4,5-c]pyridin-2-one;
248) 3-(1-(2-fluoroacryloyl)azetidin-3-yl)-1-(4-(trifluoromethyl)phenyl)-1,3-dihydro-2H-imidazo[4,5-c]pyridin-2-one;
249) 6-chloro-3-(1-(2-fluoroacryloyl)azetidin-3-yl)-1-(4-(trifluoromethyl)phenyl)-1,3-dihydro-2H-imidazo[4,5-b]pyridin-2-one;
250) 9-(1-(2-fluoroacryloyl)azetidin-3-yl)-7-(4-(trifluoromethyl)phenyl)-7,9-dihydro-8H-purin-8-one;
251) 3-(1-(2-fluoroacryloyl)azetidin-3-yl)-5-methoxy-1-(4-(trifluoromethyl)phenyl)-1,3-dihydro-2H-imidazo[4,5-b]pyridin-2-one;
252) 5-chloro-3-(1-(2-fluoroacryloyl)azetidin-3-yl)-1-(4-(trifluoromethyl)phenyl)-1,3-dihydro-2H-imidazo[4,5-b]pyridin-2-one;
253) 6-fluoro-3-(1-(2-fluoroacryloyl)azetidin-3-yl)-1-(4-(trifluoromethyl)phenyl)-1,3-dihydro-2H-imidazo[4,5-b]pyridin-2-one;
254) N-(1-(2-(4-(trifluoromethyl)phenyl)quinazolin-4-yl)pyrrolidin-3-yl)acrylamide;
255) 2-fluoro-1-(3-((2-(4-(trifluoromethyl)phenyl)quinazolin-4-yl)amino)azetidin-1-yl)prop-2-en-1-one;
256) 2-fluoro-1-(3-(2-(4-(trifluoromethyl)phenyl)quinazolin-4-yl)azetidin-1-yl)prop-2-en-1-one;
257) 2-fluoro-1-(3-(4-(4-(trifluoromethyl)phenyl)quinazolin-2-yl)azetidin-1-yl)prop-2-en-1-one;
258) N-(1-(1-(4-(trifluoromethyl)phenyl)isoquinolin-3-yl)pyrrolidin-3-yl)acrylamide;
259) N-(3-(1-(4-(trifluoromethyl)phenyl)isoquinolin-3-yl)phenyl)acrylamide;
260) 1-(3-(4-(4-(trifluoromethyl)phenyl)quinolin-2-yl)pyrrolidin-1-yl)prop-2-en-1-one;
261) 1-(3-(4-(4-(trifluoromethyl)phenyl)quinazolin-2-yl)pyrrolidin-1-yl)prop-2-en-1-one;
262) 3-(1-acryloylpyrrolidin-3-yl)-1-(4-(trifluoromethyl)phenyl)quinazoline-2,4(1H,3H)-dione;
263) 2-(1-acryloylpyrrolidin-3-yl)-4-(4-(trifluoromethyl)phenyl)phthalazin-1(2H)-one;
264) 2-(1-acryloylpiperidin-3-yl)-4-(4-(trifluoromethyl)phenyl)phthalazin-1(2H)-one;
265) 3-(1-acryloylpyrrolidin-3-yl)-1-(4-(trifluoromethyl)phenyl)-3,4-dihydroquinazolin-2(1H)-one;
266) 3-(5-(1-acryloylpyrrolidin-3-yl)-1,3,4-oxadiazol-2-yl)-1-(4-(trifluoromethyl)phenyl)quinolin-2(1H)-one;
267) 1-(3-(5-(4-(4-(trifluoromethyl)phenyl)quinazolin-2-yl)-1,3,4-oxadiazol-2-yl)pyrrolidin-1-yl)prop-2-en-1-one;
268) N-(1-(2-(4-(trifluoromethyl)phenyl)quinazolin-4-yl)azetidin-3-yl)acrylamide;
269) 2-fluoro-N-(1-(2-(4-(trifluoromethyl)phenyl)quinazolin-4-yl)azetidin-3-yl)acrylamide;
270) 2-fluoro-N-(1-(2-(4-(trifluoromethyl)phenyl)quinazolin-4-yl)pyrrolidin-3-yl)acrylamide;
271) N-(1-(2-(4-(trifluoromethyl)phenyl)pyrido[2,3-d]pyrimidin-4-yl)pyrrolidin-3-yl)acrylamide;
272) N-(1-(2-(4-(trifluoromethyl)phenyl)pyrido[3,4-d]pyrimidin-4-yl)pyrrolidin-3-yl)acrylamide;
273) 1-(4-(2-(4-(trifluoromethyl)phenyl)pyrido[3,4-d]pyrimidin-4-yl)piperazin-1-yl)prop-2-en-1-one;
274) N-(5-methyl-1-(2-(4-(trifluoromethyl)phenyl)pyrido[3,4-d]pyrimidin-4-yl)pyrrolidin-3-yl)acrylamide;
275) N-(1-(2-(4-(trifluoromethyl)phenyl)pyrido[2,3-d]pyrimidin-4-yl)azetidin-3-yl)acrylamide;
276) 2-fluoro-N-(1-(2-(4-(trifluoromethyl)phenyl)pyrido[2,3-d]pyrimidin-4-yl)azetidin-3-yl)acrylamide;
277) 2-fluoro-N-(1-(2-(4-(trifluoromethyl)phenyl)pyrido[2,3-d]pyrimidin-4-yl)pyrrolidin-3-yl)acrylamide;
278) 2-fluoro-N-(1-(2-(4-(trifluoromethyl)phenyl)pyrido[3,4-d]pyrimidin-4-yl)pyrrolidin-3-yl)acrylamide;
279) N-(1-(2-(4-(trifluoromethyl)phenyl)pyrido[3,4-d]pyrimidin-4-yl)azetidin-3-yl)acrylamide;
280) 2-fluoro-N-(1-(2-(4-(trifluoromethyl)phenyl)pyrido[3,4-d]pyrimidin-4-yl)azetidin-3-yl)acrylamide;
281) N-(1-(2-(4-(trifluoromethyl)phenyl)pyrido[3,2-d]pyrimidin-4-yl)azetidin-3-yl)acrylamide;
282) 2-fluoro-N-(1-(2-(4-(trifluoromethyl)phenyl)pyrido[3,2-d]pyrimidin-4-yl)azetidin-3-yl)acrylamide;
283) 1-(3-((2-(4-(trifluoromethyl)phenyl)quinazolin-4-yl)amino)azetidin-1-yl)prop-2-en-1-one;
284) N-(3-(4-(4-(trifluoromethyl)phenoxy)naphthalen-2-yl)phenyl)acrylamide;
285) 1-(3-(2-(4-(trifluoromethyl)phenyl)quinolin-4-yl)pyrrolidin-1-yl)prop-2-en-1-one;
286) 1-(3-(6-(4-(trifluoromethyl)phenyl)quinolin-8-yl)pyrrolidin-1-yl)prop-2-en-1-one;
287) N-(1-(2-(4-(trifluoromethyl)phenyl)pyrido[3,2-d]pyrimidin-4-yl)pyrrolidin-3-yl)acrylamide;
288) 2-fluoro-N-(1-(2-(4-(trifluoromethyl)phenyl)pyrido[3,2-d]pyrimidin-4-yl)pyrrolidin-3-yl)acrylamide;
289) 3-(1-acryloylpyrrolidin-3-yl)-1-(4-(trifluoromethyl)phenyl)-4a,8a-dihydroquinolin-2(1H)-one;
290) 2-(1-acryloylpyrrolidin-3-yl)-4-(4-(trifluoromethyl)phenyl)-4a,8a-dihydroisoquinolin-1(2H)-one;
291) 2-(1-acryloylazetidin-3-yl)-4-(4-(trifluoromethyl)phenyl)phthalazin-1(2H)-one;
292) 3-(1-acryloylazetidin-3-yl)-1-(4-(trifluoromethyl)phenyl)quinazoline-2,4(1H,3H)-dione;
293) 2-(1-acryloylazetidin-3-yl)-4-(4-(trifluoromethyl)phenyl)isoquinolin-1(2H)-one;
294) 1-(3-(4-(4-(trifluoromethyl)phenyl)quinazolin-2-yl)azetidin-1-yl)prop-2-en-1-one;
295) 2-fluoro-N-(1-(3-(4-(trifluoromethyl)phenyl)naphthalen-1-yl)azetidin-3-yl)acrylamide;
296) 2-fluoro-N-(1-(6-(4-(trifluoromethyl)phenyl)quinolin-8-yl)azetidin-3-yl)acrylamide;
297) 1-(1-(2-fluoroacryloyl)azetidin-3-yl)-3-(4-(trifluoromethyl)phenyl)quinolin-2(1H)-one;
298) 1-(1-(2-fluoroacryloyl)azetidin-3-yl)-3-(4-(trifluoromethyl)phenyl)-1,8-naphthyridin-2(1H)-one;
299) 1-(1-(2-fluoroacryloyl)azetidin-3-yl)-3-(4-(trifluoromethyl)phenyl)quinoxalin-2(1H)-one;
300) 4-(1-(2-fluoroacryloyl)azetidin-3-yl)-2-(4-(trifluoromethyl)phenyl)pyrido[2,3-b]pyrazin-3 (4H)-one;
301) 2-fluoro-N-(1-(2-(4-(trifluoromethyl)phenyl)pteridin-4-yl)azetidin-3-yl)acrylamide;
302) 2-fluoro-N-methyl-N-(1-(2-(4-(trifluoromethyl)phenyl)quinazolin-4-yl)azetidin-3-yl)acrylamide;
303) 2-fluoro-N-(1-(7-methoxy-2-(4-(trifluoromethyl)phenyl)quinazolin-4-yl)azetidin-3-yl)acrylamide;
304) 2-fluoro-N-(1-(5-(trifluoromethyl)-2-(4-(trifluoromethyl)phenyl)quinazolin-4-yl)azetidin-3-yl)acrylamide;
305) 2-fluoro-N-(1-(2-(4-(trifluoromethyl)phenyl)pyrido[4,3-d]pyrimidin-4-yl)azetidin-3-yl)acrylamide;
306) 2-fluoro-N-(1-(2-(6-(trifluoromethyl)pyridin-3-yl)pyrido[3,2-d]pyrimidin-4-yl)azetidin-3-yl)acrylamide;
307) 2-fluoro-1-(3-(3-(phenylethynyl)-1H-pyrazolo[3,4-b]pyridin-1-yl)azetidin-1-yl)prop-2-en-1-one;
308) (E)-2-fluoro-1-(3-(3-styryl-1H-pyrazolo[3,4-b]pyridin-1-yl)azetidin-1-yl)prop-2-en-1-one;
309) 1-(3-(3-((3,3-difluorocyclobutyl)ethynyl)-1H-pyrazolo[3,4-b]pyridin-1-yl)azetidin-1-yl)prop-2-en-1-one;
310) N-(3-(3-(4-(trifluoromethyl)phenyl)-1H-pyrazolo[3,4-b]pyridin-1-yl)phenyl)acrylamide;
311) 1-(3-(3-(cyclopentylethynyl)-1H-pyrazolo[3,4-b]pyridin-1-yl)azetidin-1-yl)prop-2-en-1-one;
312) 1-(3-(3-(cyclopentylethynyl)-1H-pyrazolo[3,4-b]pyridin-1-yl)azetidin-1-yl)-2-fluoroprop-2-en-1-one;
313) 1-(3-(3-(pyrimidin-2-ylethynyl)-1H-pyrazolo[3,4-b]pyridin-1-yl)azetidin-1-yl)prop-2-en-1-one;
314) 2-fluoro-1-(3-(3-(pyrimidin-2-ylethynyl)-1H-pyrazolo[3,4-b]pyridin-1-yl)azetidin-1-yl)prop-2-en-1-one;
315) 1-(3-(3-(cyclopropylethynyl)-1H-pyrazolo[3,4-b]pyridin-1-yl)azetidin-1-yl)prop-2-en-1-one;
316) 1-(3-(3-(thiophen-3-ylethynyl)-1H-pyrazolo[3,4-b]pyridin-1-yl)azetidin-1-yl)prop-2-en-1-one;
317) 2-fluoro-1-(3-(3-(thiophen-3-ylethynyl)-1H-pyrazolo[3,4-b]pyridin-1-yl)azetidin-1-yl)prop-2-en-1-one;
318) 1-(3-(3-((1-methyl-1H-imidazol-5-yl)ethynyl)-1H-pyrazolo[3,4-b]pyridin-1-yl)azetidin-1-yl)prop-2-en-1-one;
319) 1-(3-(3-(phenylethynyl)-1H-pyrazolo[3,4-b]pyridin-1-yl)azetidin-1-yl)prop-2-en-1-one;
320) 1-(3-(3-(cyclobutylethynyl)-1H-pyrazolo[3,4-b]pyridin-1-yl)azetidin-1-yl)prop-2-en-1-one;
321) 1-(3-(3-(cyclobutylethynyl)-1H-pyrazolo[3,4-b]pyridin-1-yl)azetidin-1-yl)-2-fluoroprop-2-en-1-one;
322) 1-(3-(3-(cyclopropylethynyl)-1H-pyrazolo[3,4-b]pyridin-1-yl)azetidin-1-yl)-2-fluoroprop-2-en-1-one;
323) 2-fluoro-1-(3-(3-((1-methyl-1H-imidazol-5-yl)ethynyl)-1H-pyrazolo[3,4-b]pyridin-1-yl)azetidin-1-yl)prop-2-en-1-one;
324) 1-(3-(3-(cyclohexylethynyl)-1H-pyrazolo[3,4-b]pyridin-1-yl)azetidin-1-yl)prop-2-en-1-one;
325) 1-(3-(3-(cyclohexylethynyl)-1H-pyrazolo[3,4-b]pyridin-1-yl)azetidin-1-yl)-2-fluoroprop-2-en-1-one;
326) 1-(3-(3-((3,3-difluorocyclobutyl)ethynyl)-1H-pyrazolo[3,4-b]pyridin-1-yl)azetidin-l-yl)-2-fluoroprop-2-en-1-one;
327) (E)-1-(3-(3-styryl-1H-pyrazolo[3,4-b]pyridin-1-yl)azetidin-1-yl)prop-2-en-1-one;
328) 1-(3-(3-((3,3-difluorocyclopentyl)ethynyl)-1H-pyrazolo[3,4-b]pyridin-1-yl)azetidin-1-yl)prop-2-en-1-one;
329) 1-(3-(3-((3,3-difluorocyclopentyl)ethynyl)-1H-pyrazolo[3,4-b]pyridin-1-yl)azetidin-l-yl)-2-fluoroprop-2-en-1-one;
330) (E)-1-(3-(3-(2-cyclohexylvinyl)-1H-pyrazolo[3,4-b]pyridin-1-yl)azetidin-1-yl)prop-2-en-1-one;
331) (E)-1-(3-(3-(2-cyclohexylvinyl)-1H-pyrazolo[3,4-b]pyridin-1-yl)azetidin-1-yl)-2-fluoroprop-2-en-1-one;
332) (E)-1-(3-(3-(2-cyclopropylvinyl)-1H-pyrazolo[3,4-b]pyridin-1-yl)azetidin-1-yl)prop-2-en-1-one;
333) (E)-1-(3-(3-(2-cyclopropylvinyl)-1H-pyrazolo[3,4-b]pyridin-1-yl)azetidin-1-yl)-2-fluoroprop-2-en-1-one;
334) 2-fluoro-1-(3-(3-((4-fluorophenyl)ethynyl)-1H-pyrazolo[3,4-b]pyridin-1-yl)azetidin-1-yl)prop-2-en-1-one;
335) 2-fluoro-1-(3-(3-((3-fluorophenyl)ethynyl)-1H-pyrazolo[3,4-b]pyridin-1-yl)azetidin-1-yl)prop-2-en-1-one;
336) 2-fluoro-1-(3-(3-((2-fluorophenyl)ethynyl)-1H-pyrazolo[3,4-b]pyridin-1-yl)azetidin-1-yl)prop-2-en-1-one;
337) 1-(3-(3-((3-chlorophenyl)ethynyl)-1H-pyrazolo[3,4-b]pyridin-1-yl)azetidin-1-yl)-2-fluoroprop-2-en-1-one;
338) 1-(3-(3-((3-((difluoro-13-methyl)-12-fluoranyl)phenyl)ethynyl)-1H-pyrazolo[3,4-b]pyridin-1-yl)azetidin-1-yl)-2-fluoroprop-2-en-1-one;
339) (E)-2-fluoro-1-(3-(3-(4-fluorostyryl)-1H-pyrazolo[3,4-b]pyridin-1-yl)azetidin-1-yl)prop-2-en-1-one;
340) (E)-2-fluoro-1-(3-(3-(3-fluorostyryl)-1H-pyrazolo[3,4-b]pyridin-1-yl)azetidin-1-yl)prop-2-en-1-one;
341) (E)-2-fluoro-1-(3-(3-(2-fluorostyryl)-1H-pyrazolo[3,4-b]pyridin-1-yl)azetidin-1-yl)prop-2-en-1-one;
342) (E)-1-(3-(3-(4-chlorostyryl)-1H-pyrazolo[3,4-b]pyridin-1-yl)azetidin-1-yl)-2-fluoroprop-2-en-1-one;
343) (E)-2-fluoro-1-(3-(3-(4-(trifluoromethyl)styryl)-1H-pyrazolo[3,4-b]pyridin-1-yl)azetidin-1-yl)prop-2-en-1-one;
344) (E)-4-(2-(1-(1-(2-fluoroacryloyl)azetidin-3-yl)-1H-pyrazolo[3,4-b]pyridin-3-yl)vinyl)benzonitrile;
345) (E)-2-fluoro-1-(3-(3-(3-(trifluoromethyl)styryl)-1H-pyrazolo[3,4-b]pyridin-1-yl)azetidin-1-yl)prop-2-en-1-one;
346) 1-(3-(3-((2,3-difluorophenyl)ethynyl)-1H-pyrazolo[3,4-b]pyridin-1-yl)azetidin-1-yl)-2-fluoroprop-2-en-1-one;
347) 2-fluoro-1-(3-(3-((2-fluorophenyl)ethynyl)-1H-pyrazolo[3,4-b]pyridin-1-yl)azetidin-1-yl)prop-2-en-1-one;
348) 1-(3-(3-((2-chlorophenyl)ethynyl)-1H-pyrazolo[3,4-b]pyridin-1-yl)azetidin-1-yl)-2-fluoroprop-2-en-1-one; or
349) 2-fluoro-1-(3-(3-((2-(trifluoromethyl)phenyl)ethynyl)-1H-pyrazolo[3,4-b]pyridin-1-yl)azetidin-1-yl)prop-2-en-1-one.

38. A pharmaceutical composition comprising a compound of claim 1, or a pharmaceutically acceptable salt or a stereoisomer thereof, and at least one pharmaceutically acceptable carrier or excipient.

39. (canceled)

40. (canceled)

41. (canceled)

42. (canceled)

43. A method for the therapeutic treatment of disease in a subject, which method comprising administering to said subject in need thereof a therapeutically effective amount of a compound of claim 1, or a pharmaceutically acceptable salt or a stereoisomer thereof.

44. The method of claim 43, wherein the disease is colon cancer, gastric cancer, thyroid cancer, lung cancer, leukemia, pancreatic cancer, melanoma, multiple melanoma, brain cancer, renal cancer, liver cancer, squamous cancer, gastrointestinal cancer, mesothelioma, prostate cancer, ovarian cancer or breast cancer.

45. The compound or the stereoisomer, tautomer, pharmaceutically acceptable salt, prodrug, chelate, non-covalent complex, or solvate of claim 1, wherein,
L₁ is bond, —NH— or —C(═O)—;
L₂ is bond, —O—, C₂₋₄ alkenylene, or C₂₋₄ alkynylene;
L₃ is bond or —NH—;
R₁ is H, oxo, hydroxyl, halogen, CN, —NR_{d}Rₑ, C₁₋₆ alkyl, C₁₋₆ haloalkyl, C₁₋₆ alkoxyl, —C(═O)NRₐR_{b},—C(═O)ORₐ, —C(═O)R_{c}, —S(═O)₂NRₐR_{b};
R₂ is H, hydroxyl, halogen, CN, C₁₋₆ alkyl, C₂₋₆ alkenyl, C₂₋₆ alkynyl, C₁₋₆ alkoxyl, C₁₋₆ haloalkyl; wherein the C₁₋₆ alkyl, C₂₋₆ alkenyl, C₂₋₆ alkynyl, C₁₋₆ alkoxyl are each optionally substituted with 0 to 3 substitutents independently selected from the group consisting of hydroxyl, halogen, CN and C₁₋₄ alkyl;
R₃ is H, halogen, CN, C₁₋₃ alkyl, —ORₐ;
R₈ is H, C₁₋₄ alkyl or halogen;
one of R₆ and R₇ is H, and the other is H, C₁₋₄ alkyl or halogen;
R₈ is H, halogen or C₁₋₄ alkyl;
Rₐ and R_{b} are independently selected from the group consisting of H and C₁₋₆ alkyl.
